SEQUENCE LISTING (841 sequences)

SEQ ID NO: 1 - protein, 4 aa, Homo sapiens
ISAA

SEQ ID NO: 2 - protein, 5 aa, Homo sapiens
DISAA

SEQ ID NO: 3 - protein, 6 aa, Homo sapiens
DDISAA

SEQ ID NO: 4 - protein, 7 aa, Homo sapiens
YDDISAA

SEQ ID NO: 5 - protein, 8 aa, Homo sapiens
DYDDISAA

SEQ ID NO: 6 - protein, 4 aa, Homo sapiens
QSLV

SEQ ID NO: 7 - protein, 5 aa, Homo sapiens
IQSLV

SEQ ID NO: 8 - protein, 6 aa, Homo sapiens
EIQSLV

SEQ ID NO: 9 - protein, 7 aa, Homo sapiens
NEIQSLV

SEQ ID NO: 10 - protein, 8 aa, Homo sapiens
RNEIQSLV

SEQ ID NO: 11 - protein, 4 aa, Homo sapiens
STTL

SEQ ID NO: 12 - protein, 5 aa, Homo sapiens
TSTTL

SEQ ID NO: 13 - protein, 6 aa, Homo sapiens
HTSTTL

SEQ ID NO: 14 - protein, 7 aa, Homo sapiens
GHTSTTL

SEQ ID NO: 15 - protein, 8 aa, Homo sapiens
SGHTSTTL

SEQ ID NO: 16 - protein, 4 aa, Homo sapiens
KTRV

SEQ ID NO: 17 - protein, 5 aa, Homo sapiens
PKTRV

SEQ ID NO: 18 - protein, 6 aa, Homo sapiens
RPKTRV

SEQ ID NO: 19 - protein, 7 aa, Homo sapiens
RRPKTRV

SEQ ID NO: 20 - protein, 8 aa, Homo sapiens
SRRPKTRV

SEQ ID NO: 21 - protein, 4 aa, Homo sapiens
LYKL

SEQ ID NO: 22 - protein, 5 aa, Homo sapiens
GLYKL

SEQ ID NO: 23 - protein, 6 aa, Homo sapiens
FGLYKL

SEQ ID NO: 24 - protein, 7 aa, Homo sapiens
FFGLYKL

SEQ ID NO: 25 - protein, 8 aa, Homo sapiens
TFFGLYKL

SEQ ID NO: 26 - protein, 4 aa, Homo sapiens
VTAL

SEQ ID NO: 27 - protein, 5 aa, Homo sapiens
HVTAL

SEQ ID NO: 28 - protein, 6 aa, Homo sapiens
LHVTAL

SEQ ID NO: 29 - protein, 7 aa, Homo sapiens
GLHVTAL

SEQ ID NO: 30 - protein, 8 aa, Homo sapiens
QGLHVTAL

SEQ ID NO: 31 - protein, 4 aa, Homo sapiens
SAQV

SEQ ID NO: 32 - protein, 5 aa, Homo sapiens
SSAQV

SEQ ID NO: 33 - protein, 6 aa, Homo sapiens
SSSAQV

SEQ ID NO: 34 - protein, 7 aa, Homo sapiens
ISSSAQV

SEQ ID NO: 35 - protein, 8 aa, Homo sapiens
SISSSAQV

SEQ ID NO: 36 - protein, 4 aa, Homo sapiens
SSVV

SEQ ID NO: 37 - protein, 5 aa, Homo sapiens
SSSVV

SEQ ID NO: 38 - protein, 6 aa, Homo sapiens
SSSSVV

SEQ ID NO: 39 - protein, 7 aa, Homo sapiens
TSSSSVV

SEQ ID NO: 40 - protein, 8 aa, Homo sapiens
MTSSSSVV

SEQ ID NO: 41 - protein, 4 aa, Homo sapiens
SQGS

SEQ ID NO: 42 - protein, 5 aa, Homo sapiens
LSQGS

SEQ ID NO: 43 - protein, 6 aa, Homo sapiens
QLSQGS

SEQ ID NO: 44 - protein, 7 aa, Homo sapiens
TQLSQGS

SEQ ID NO: 45 - protein, 8 aa, Homo sapiens
ETQLSQGS

SEQ ID NO: 46 - protein, 4 aa, Homo sapiens
LTTF

SEQ ID NO: 47 - protein, 5 aa, Homo sapiens
SLTTF

SEQ ID NO: 48 - protein, 6 aa, Homo sapiens
TSLTTF

SEQ ID NO: 49 - protein, 7 aa, Homo sapiens
ATSLTTF

SEQ ID NO: 50 - protein, 8 aa, Homo sapiens
NATSLTTF

SEQ ID NO: 51 - protein, 4 aa, Homo sapiens
STDL

SEQ ID NO: 52 - protein, 5 aa, Homo sapiens
LSTDL

SEQ ID NO: 53 - protein, 6 aa, Homo sapiens
SLSTDL

SEQ ID NO: 54 - protein, 7 aa, Homo sapiens
DSLSTDL

SEQ ID NO: 55 - protein, 8 aa, Homo sapiens
PDSLSTDL

SEQ ID NO: 56 - protein, 4 aa, Homo sapiens
ITRL

SEQ ID NO: 57 - protein, 5 aa, Homo sapiens
DITRL

SEQ ID NO: 58 - protein, 6 aa, Homo sapiens
RDITRL

SEQ ID NO: 59 - protein, 7 aa, Homo sapiens
GRDITRL

SEQ ID NO: 60 - protein, 8 aa, Homo sapiens
QGRDITRL

SEQ ID NO: 61 - protein, 4 aa, Homo sapiens
HSVI

SEQ ID NO: 62 - protein, 5 aa, Homo sapiens
EHSVI

SEQ ID NO: 63 - protein, 6 aa, Homo sapiens
NEHSVI

SEQ ID NO: 64 - protein, 7 aa, Homo sapiens
QNEHSVI

SEQ ID NO: 65 - protein, 8 aa, Homo sapiens
EQNEHSVI

SEQ ID NO: 66 - protein, 4 aa, Homo sapiens
DSVF

SEQ ID NO: 67 - protein, 5 aa, Homo sapiens
EDSVF

SEQ ID NO: 68 - protein, 6 aa, Homo sapiens
LEDSVF

SEQ ID NO: 69 - protein, 7 aa, Homo sapiens
TLEDSVF

SEQ ID NO: 70 - protein, 8 aa, Homo sapiens
ETLEDSVF

SEQ ID NO: 71 - protein, 4 aa, Homo sapiens
FTSL

SEQ ID NO: 72 - protein, 5 aa, Homo sapiens
KFTSL

SEQ ID NO: 73 - protein, 6 aa, Homo sapiens
VKFTSL

SEQ ID NO: 74 - protein, 7 aa, Homo sapiens
EVKFTSL

SEQ ID NO: 75 - protein, 8 aa, Homo sapiens
REVKFTSL

SEQ ID NO: 76 - protein, 4 aa, Homo sapiens
PIDL

SEQ ID NO: 77 - protein, 5 aa, Homo sapiens
PPIDL

SEQ ID NO: 78 - protein, 6 aa, Homo sapiens
SPPIDL

SEQ ID NO: 79 - protein, 7 aa, Homo sapiens
MSPPIDL

SEQ ID NO: 80 - protein, 8 aa, Homo sapiens
EMSPPIDL

SEQ ID NO: 81 - protein, 4 aa, Homo sapiens
DTEL

SEQ ID NO: 82 - protein, 5 aa, Homo sapiens
ADTEL

SEQ ID NO: 83 - protein, 6 aa, Homo sapiens
VADTEL

SEQ ID NO: 84 - protein, 7 aa, Homo sapiens
VVADTEL

SEQ ID NO: 85 - protein, 8 aa, Homo sapiens
HVVADTEL

SEQ ID NO: 86 - protein, 4 aa, Homo sapiens
KRIV

SEQ ID NO: 87 - protein, 5 aa, Homo sapiens
RKRIV

SEQ ID NO: 88 - protein, 6 aa, Homo sapiens
DRKRIV

SEQ ID NO: 89 - protein, 7 aa, Homo sapiens
GDRKRIV

SEQ ID NO: 90 - protein, 8 aa, Homo sapiens
RGDRKRIV

SEQ ID NO: 91 - protein, 4 aa, Homo sapiens
QTAW

SEQ ID NO: 92 - protein, 5 aa, Homo sapiens
TQTAW

SEQ ID NO: 93 - protein, 6 aa, Homo sapiens
WTQTAW

SEQ ID NO: 94 - protein, 7 aa, Homo sapiens
PWTQTAW

SEQ ID NO: 95 - protein, 8 aa, Homo sapiens
RPWTQTAW

SEQ ID NO: 96 - protein, 4 aa, Homo sapiens
GFRQ

SEQ ID NO: 97 - protein, 5 aa, Homo sapiens
RGFRQ

SEQ ID NO: 98 - protein, 6 aa, Homo sapiens
RRGFRQ

SEQ ID NO: 99 - protein, 7 aa, Homo sapiens
ARRGFRQ

SEQ ID NO: 100 - protein, 8 aa, Homo sapiens
RARRGFRQ

SEQ ID NO: 101 - protein, 4 aa, Homo sapiens
SVKI

SEQ ID NO: 102 - protein, 5 aa, Homo sapiens
ESVKI

SEQ ID NO: 103 - protein, 6 aa, Homo sapiens
IESVKI

SEQ ID NO: 104 - protein, 7 aa, Homo sapiens
GIESVKI

SEQ ID NO: 105 - protein, 8 aa, Homo sapiens
SGIESVKI

SEQ ID NO: 106 - protein, 4 aa, Rattus norvegicus
ETVA

SEQ ID NO: 107 - protein, 5 aa, Rattus norvegicus
KETVA

SEQ ID NO: 108 - protein, 6 aa, Rattus norvegicus
RKETVA

SEQ ID NO: 109 - protein, 7 aa, Rattus norvegicus
QRKETVA

SEQ ID NO: 110 - protein, 8 aa, Rattus norvegicus
TQRKETVA

SEQ ID NO: 111 - protein, 4 aa, Homo sapiens
NLVI

SEQ ID NO: 112 - protein, 5 aa, Homo sapiens
NNLVI

SEQ ID NO: 113 - protein, 6 aa, Homo sapiens
YNNLVI

SEQ ID NO: 114 - protein, 7 aa, Homo sapiens
SYNNLVI

SEQ ID NO: 115 - protein, 8 aa, Homo sapiens
VSYNNLVI

SEQ ID NO: 116 - protein, 4 aa, Homo sapiens
GTSI

SEQ ID NO: 117 - protein, 5 aa, Homo sapiens
RGTSI

SEQ ID NO: 118 - protein, 6 aa, Homo sapiens
DRGTSI

SEQ ID NO: 119 - protein, 7 aa, Homo sapiens
PDRGTSI

SEQ ID NO: 120 - protein, 8 aa, Homo sapiens
DPDRGTSI

SEQ ID NO: 121 - protein, 4 aa, Homo sapiens
EQAL

SEQ ID NO: 122 - protein, 5 aa, Homo sapiens
PEQAL

SEQ ID NO: 123 - protein, 6 aa, Homo sapiens
APEQAL

SEQ ID NO: 124 - protein, 7 aa, Homo sapiens
RAPEQAL

SEQ ID NO: 125 - protein, 8 aa, Homo sapiens
KRAPEQAL

SEQ ID NO: 126 - protein, 4 aa, Homo sapiens
ESEV

SEQ ID NO: 127 - protein, 5 aa, Homo sapiens
LESEV

SEQ ID NO: 128 - protein, 6 aa, Homo sapiens
SLESEV

SEQ ID NO: 129 - protein, 7 aa, Homo sapiens
SSLESEV

SEQ ID NO: 130 - protein, 8 aa, Homo sapiens
ISSLESEV

SEQ ID NO: 131 - protein, 4 aa, Homo sapiens
STVV

SEQ ID NO: 132 - protein, 5 aa, Homo sapiens
VSTVV

SEQ ID NO: 133 - protein, 6 aa, Homo sapiens
SVSTVV

SEQ ID NO: 134 - protein, 7 aa, Homo sapiens
PSVSTVV

SEQ ID NO: 135 - protein, 8 aa, Homo sapiens
DPSVSTVV

SEQ ID NO: 136 - protein, 4 aa, Homo sapiens
WQYA

SEQ ID NO: 137 - protein, 5 aa, Homo sapiens
IWQYA

SEQ ID NO: 138 - protein, 6 aa, Homo sapiens
SIWQYA

SEQ ID NO: 139 - protein, 7 aa, Homo sapiens
WSIWQYA

SEQ ID NO: 140 - protein, 8 aa, Homo sapiens
LWSIWQYA

SEQ ID NO: 141 - protein, 4 aa, Homo sapiens
VSYV

SEQ ID NO: 142 - protein, 5 aa, Homo sapiens
QVSYV

SEQ ID NO: 143 - protein, 6 aa, Homo sapiens
EQVSYV

SEQ ID NO: 144 - protein, 7 aa, Homo sapiens
EEQVSYV

SEQ ID NO: 145 - protein, 8 aa, Homo sapiens
TEEQVSYV

SEQ ID NO: 146 - protein, 4 aa, Homo sapiens
ISSV

SEQ ID NO: 147 - protein, 5 aa, Homo sapiens
RISSV

SEQ ID NO: 148 - protein, 6 aa, Homo sapiens
ERISSV

SEQ ID NO: 149 - protein, 7 aa, Homo sapiens
SERISSV

SEQ ID NO: 150 - protein, 8 aa, Homo sapiens
VSERISSV

SEQ ID NO: 151 - protein, 4 aa, Homo sapiens
QTSI

SEQ ID NO: 152 - protein, 5 aa, Homo sapiens
LQTSI

SEQ ID NO: 153 - protein, 6 aa, Homo sapiens
DLQTSI

SEQ ID NO: 154 - protein, 7 aa, Homo sapiens
GDLQTSI

SEQ ID NO: 155 - protein, 8 aa, Homo sapiens
NGDLQTSI

SEQ ID NO: 156 - protein, 4 aa, Homo sapiens
TTTF

SEQ ID NO: 157 - protein, 5 aa, Homo sapiens
RTTTF

SEQ ID NO: 158 - protein, 6 aa, Homo sapiens
TRTTTF

SEQ ID NO: 159 - protein, 7 aa, Homo sapiens
LTRTTTF

SEQ ID NO: 160 - protein, 8 aa, Homo sapiens
PLTRTTTF

SEQ ID NO: 161 - protein, 4 aa, Homo sapiens
SSNL

SEQ ID NO: 162 - protein, 5 aa, Homo sapiens
VSSNL

SEQ ID NO: 163 - protein, 6 aa, Homo sapiens
NVSSNL

SEQ ID NO: 164 - protein, 7 aa, Homo sapiens
VNVSSNL

SEQ ID NO: 165 - protein, 8 aa, Homo sapiens
SVNVSSNL

SEQ ID NO: 166 - protein, 4 aa, Homo sapiens
EDVL

SEQ ID NO: 167 - protein, 5 aa, Homo sapiens
TEDVL

SEQ ID NO: 168 - protein, 6 aa, Homo sapiens
ETEDVL

SEQ ID NO: 169 - protein, 7 aa, Homo sapiens
GETEDVL

SEQ ID NO: 170 - protein, 8 aa, Homo sapiens
QGETEDVL

SEQ ID NO: 171 - protein, 4 aa, Homo sapiens
TQAV

SEQ ID NO: 172 - protein, 5 aa, Homo sapiens
KTQAV

SEQ ID NO: 173 - protein, 6 aa, Homo sapiens
QKTQAV

SEQ ID NO: 174 - protein, 7 aa, Homo sapiens
AQKTQAV

SEQ ID NO: 175 - protein, 8 aa, Homo sapiens
MAQKTQAV

SEQ ID NO: 176 - protein, 4 aa, Human adenovirus AdenoE4 typ9
ATLV

SEQ ID NO: 177 - protein, 5 aa, Human adenovirus AdenoE4 typ9
IATLV

SEQ ID NO: 178 - protein, 6 aa, Human adenovirus AdenoE4 typ9
KIATLV

SEQ ID NO: 179 - protein, 7 aa, Human adenovirus AdenoE4 typ9
VKIATLV

SEQ ID NO: 180 - protein, 8 aa, Human adenovirus AdenoE4 typ9
SVKIATLV

SEQ ID NO: 181 - protein, 4 aa, Human adenovirus
SSTA

SEQ ID NO: 182 - protein, 5 aa, Human adenovirus
ASSTA

SEQ ID NO: 183 - protein, 6 aa, Human adenovirus
SASSTA

SEQ ID NO: 184 - protein, 7 aa, Human adenovirus
YSASSTA

SEQ ID NO: 185 - protein, 8 aa, Human adenovirus
SYSASSTA

SEQ ID NO: 186 - protein, 4 aa, Human papillomavirus strain #16 (modified)
ETQL

SEQ ID NO: 187 - protein, 5 aa, Human papillomavirus strain #16 (modified)
RETQL

SEQ ID NO: 188 - protein, 6 aa, Human papillomavirus strain #16 (modified)
RRETQL

SEQ ID NO: 189 - protein, 7 aa, Human papillomavirus strain #16 (modified)
TRRETQL

SEQ ID NO: 190 - protein, 8 aa, Human papillomavirus strain #16 (modified)
RTRRETQL

SEQ ID NO: 191 - protein, 4 aa, Human papillomavirus strain #18
ETQV

SEQ ID NO: 192 - protein, 5 aa, Human papillomavirus strain #18
RETQV

SEQ ID NO: 193 - protein, 6 aa, Human papillomavirus strain #18
RRETQV

SEQ ID NO: 194 - protein, 7 aa, Human papillomavirus strain #18
RRRETQV

SEQ ID NO: 195 - protein, 8 aa, Human papillomavirus strain #18
QRRRETQV

SEQ ID NO: 196 - protein, 4 aa, Human papillomavirus strain 33 (modified)
ETAL

SEQ ID NO: 197 - protein, 5 aa, Human papillomavirus strain 33 (modified)
RETAL

SEQ ID NO: 198 - protein, 6 aa, Human papillomavirus strain 33 (modified)
RRETAL

SEQ ID NO: 199 - protein, 7 aa, Human papillomavirus strain 33 (modified)
GRRETAL

SEQ ID NO: 200 - protein, 8 aa, Human papillomavirus strain 33 (modified)
QGRRETAL

SEQ ID NO: 201 - protein, 4 aa, Human papillomavirus strain #35 (cysteine-free) and Human T-lymphotropic virus 1
ETEV

SEQ ID NO: 202 - protein, 5 aa, Human papillomavirus strain #35 (cysteine-free) and Human T-lymphotropic virus 1
RETEV

SEQ ID NO: 203 - protein, 6 aa, Human papillomavirus strain #35 (cysteine-free)
RRETEV

SEQ ID NO: 204 - protein, 7 aa, Human papillomavirus strain #35 (cysteine-free)
TRRETEV

SEQ ID NO: 205 - protein, 8 aa, Human papillomavirus strain #35 (cysteine-free)
PTRRETEV

SEQ ID NO: 206 - protein, 4 aa, Human papillomavirus strain 52 (modified)
VTQV

SEQ ID NO: 207 - protein, 5 aa, Human papillomavirus strain 52 (modified)
RVTQV

SEQ ID NO: 208 - protein, 6 aa, Human papillomavirus strain 52 (modified)
RRVTQV

SEQ ID NO: 209 - protein, 7 aa, Human papillomavirus strain 52 (modified)
GRRVTQV

SEQ ID NO: 210 - protein, 8 aa, Human papillomavirus strain 52 (modified)
QGRRVTQV

SEQ ID NO: 211 - protein, 4 aa, Human papillomavirus strain #57 (modified)
RTSH

SEQ ID NO: 212 - protein, 5 aa, Human papillomavirus strain #57 (modified)
LRTSH

SEQ ID NO: 213 - protein, 6 aa, Human papillomavirus strain #57 (modified)
ALRTSH

SEQ ID NO: 214 - protein, 7 aa, Human papillomavirus strain #57 (modified)
PALRTSH

SEQ ID NO: 215 - protein, 8 aa, Human papillomavirus strain #57 (modified)
APALRTSH

SEQ ID NO: 216 - protein, 4 aa, Human papillomavirus strain 58 (modified)
QTQV

SEQ ID NO: 217 - protein, 5 aa, Human papillomavirus strain 58 (modified)
RQTQV

SEQ ID NO: 218 - protein, 6 aa, Human papillomavirus strain 58 (modified)
RRQTQV

SEQ ID NO: 219 - protein, 7 aa, Human papillomavirus strain 58 (modified)
GRRQTQV

SEQ ID NO: 220 - protein, 8 aa, Human papillomavirus strain 58 (modified)
QGRRQTQV

SEQ ID NO: 221 - protein, 4 aa, Human papillomavirus strain #66 (cysteine-free)
ESTV

SEQ ID NO: 222 - protein, 5 aa, Human papillomavirus strain #66 (cysteine-free)
TESTV

SEQ ID NO: 223 - protein, 6 aa, Human papillomavirus strain #66 (cysteine-free)
ATESTV

SEQ ID NO: 224 - protein, 7 aa, Human papillomavirus strain #66 (cysteine-free)
QATESTV

SEQ ID NO: 225 - protein, 8 aa, Human papillomavirus strain #66 (cysteine-free)
RQATESTV

SEQ ID NO: 226 - protein, 4 aa, Human papillomavirus strain 77 (modified)
QSRQ

SEQ ID NO: 227 - protein, 5 aa, Human papillomavirus strain 77 (modified)
GQSRQ

SEQ ID NO: 228 - protein, 6 aa, Human papillomavirus strain 77 (modified)
GGQSRQ

SEQ ID NO: 229 - protein, 7 aa, Human papillomavirus strain 77 (modified)
GGGQSRQ

SEQ ID NO: 230 - protein, 8 aa, Human papillomavirus strain 77 (modified)
RGGGQSRQ

SEQ ID NO: 231 - protein, 4 aa, Artificial
  PDZ domain signature sequence
GLGF

SEQ ID NO: 232 - protein, 5 aa, Artificial
  linker region between glutathione-S transferase (GST) and PDZ domain in GST-PDZ fusion protein
GIPGN

SEQ ID NO: 233 - protein, 6 aa, Human T-lymphotropic virus 1
FRETEV

SEQ ID NO: 234 - protein, 7 aa, Human T-lymphotropic virus 1
HFRETEV

SEQ ID NO: 235 - protein, 8 aa, Human T-lymphotropic virus 1
KHFRETEV

SEQ ID NO: 236 - protein, 4 aa, Homo sapiens
KDYV

SEQ ID NO: 237 - protein, 5 aa, Homo sapiens
GKDYV

SEQ ID NO: 238 - protein, 6 aa, Homo sapiens
SGKDYV

SEQ ID NO: 239 - protein, 7 aa, Homo sapiens
SSGKDYV

SEQ ID NO: 240 - protein, 8 aa, Homo sapiens
PSSGKDYV

SEQ ID NO: 241 - protein, 4 aa, Homo sapiens
TGYV

SEQ ID NO: 242 - protein, 5 aa, Homo sapiens
LTGYV

SEQ ID NO: 243 - protein, 6 aa, Homo sapiens
SLTGYV

SEQ ID NO: 244 - protein, 7 aa, Homo sapiens
YSLTGYV

SEQ ID NO: 245 - protein, 8 aa, Homo sapiens
SYSLTGYV

SEQ ID NO: 246 - protein, 4 aa, Homo sapiens
KEYV

SEQ ID NO: 247 - protein, 5 aa, Homo sapiens
SKEYV

SEQ ID NO: 248 - protein, 6 aa, Homo sapiens
SSKEYV

SEQ ID NO: 249 - protein, 7 aa, Homo sapiens
NSSKEYV

SEQ ID NO: 250 - protein, 8 aa, Homo sapiens
SNSSKEYV

SEQ ID NO: 251 - protein, 4 aa, Homo sapiens
RDYV

SEQ ID NO: 252 - protein, 5 aa, Homo sapiens
KRDYV

SEQ ID NO: 253 - protein, 6 aa, Homo sapiens
DKRDYV

SEQ ID NO: 254 - protein, 7 aa, Homo sapiens
LDKRDYV

SEQ ID NO: 255 - protein, 8 aa, Homo sapiens
GLDKRDYV

SEQ ID NO: 256 - protein, 4 aa, Homo sapiens
NAYV

SEQ ID NO: 257 - protein, 5 aa, Homo sapiens
KNAYV

SEQ ID NO: 258 - protein, 6 aa, Homo sapiens
DKNAYV

SEQ ID NO: 259 - protein, 7 aa, Homo sapiens
FDKNAYV

SEQ ID NO: 260 - protein, 8 aa, Homo sapiens
QFDKNAYV

SEQ ID NO: 261 - protein, 4 aa, Homo sapiens
HDYV

SEQ ID NO: 262 - protein, 5 aa, Homo sapiens
KHDYV

SEQ ID NO: 263 - protein, 6 aa, Homo sapiens
SKHDYV

SEQ ID NO: 264 - protein, 7 aa, Homo sapiens
PSKHDYV

SEQ ID NO: 265 - protein, 8 aa, Homo sapiens
YPSKHDYV

SEQ ID NO: 266 - protein, 4 aa, Homo sapiens
TTRV

SEQ ID NO: 267 - protein, 5 aa, Homo sapiens
STTRV

SEQ ID NO: 268 - protein, 6 aa, Homo sapiens
HSTTRV

SEQ ID NO: 269 - protein, 7 aa, Homo sapiens
PHSTTRV

SEQ ID NO: 270 - protein, 8 aa, Homo sapiens
LPHSTTRV

SEQ ID NO: 271 - protein, 4 aa, Homo sapiens
AMYV

SEQ ID NO: 272 - protein, 5 aa, Homo sapiens
RAMYV

SEQ ID NO: 273 - protein, 6 aa, Homo sapiens
SRAMYV

SEQ ID NO: 274 - protein, 7 aa, Homo sapiens
MSRAMYV

SEQ ID NO: 275 - protein, 8 aa, Homo sapiens
VMSRAMYV

SEQ ID NO: 276 - protein, 4 aa, Homo sapiens
SLFV

SEQ ID NO: 277 - protein, 5 aa, Homo sapiens
SSLFV

SEQ ID NO: 278 - protein, 6 aa, Homo sapiens
TSSLFV

SEQ ID NO: 279 - protein, 7 aa, Homo sapiens
QTSSLFV

SEQ ID NO: 280 - protein, 8 aa, Homo sapiens
KQTSSLFV

SEQ ID NO: 281 - protein, 4 aa, Gallus gallus
YSLA

SEQ ID NO: 282 - protein, 5 aa, Gallus gallus
IYSLA

SEQ ID NO: 283 - protein, 6 aa, Gallus gallus
AIYSLA

SEQ ID NO: 284 - protein, 7 aa, Gallus gallus
NAIYSLA

SEQ ID NO: 285 - protein, 8 aa, Gallus gallus
VNAIYSLA

SEQ ID NO: 286 - protein, 4 aa, Homo sapiens
QYWV

SEQ ID NO: 287 - protein, 5 aa, Homo sapiens
GQYWV

SEQ ID NO: 288 - protein, 6 aa, Homo sapiens
NGQYWV

SEQ ID NO: 289 - protein, 7 aa, Homo sapiens
KNGQYWV

SEQ ID NO: 290 - protein, 8 aa, Homo sapiens
LKNGQYWV

SEQ ID NO: 291 - protein, 4 aa, Homo sapiens
WLKV

SEQ ID NO: 292 - protein, 5 aa, Homo sapiens
HWLKV

SEQ ID NO: 293 - protein, 6 aa, Homo sapiens
RHWLKV

SEQ ID NO: 294 - protein, 7 aa, Homo sapiens
LRHWLKV

SEQ ID NO: 295 - protein, 8 aa, Homo sapiens
TLRHWLKV

SEQ ID NO: 296 - protein, 4 aa, Homo sapiens
WLAI

SEQ ID NO: 297 - protein, 5 aa, Homo sapiens
HWLAI

SEQ ID NO: 298 - protein, 6 aa, Homo sapiens
QHWLAI

SEQ ID NO: 299 - protein, 7 aa, Homo sapiens
LQHQLAI

SEQ ID NO: 300 - protein, 8 aa, Homo sapiens
QLQHWLAI

SEQ ID NO: 301 - protein, 4 aa, Homo sapiens
TSQF

SEQ ID NO: 302 - protein, 5 aa, Homo sapiens
QTSQF

SEQ ID NO: 303 - protein, 6 aa, Homo sapiens
TQTSQF

SEQ ID NO: 304 - protein, 7 aa, Homo sapiens
FTQTSQF

SEQ ID NO: 305 - protein, 8 aa, Homo sapiens
SFTQTSQF

SEQ ID NO: 306 - protein, 4 aa, Homo sapiens
ETHF

SEQ ID NO: 307 - protein, 5 aa, Homo sapiens
KETHF

SEQ ID NO: 308 - protein, 6 aa, Homo sapiens
EKETHF

SEQ ID NO: 309 - protein, 7 aa, Homo sapiens
TEKETHF

SEQ ID NO: 310 - protein, 8 aa, Homo sapiens
ITEKETHF

SEQ ID NO: 311 - protein, 4 aa, Homo sapiens
PVYI

SEQ ID NO: 312 - protein, 5 aa, Homo sapiens
QPVYI

SEQ ID NO: 313 - protein, 6 aa, Homo sapiens
EQPVYI

SEQ ID NO: 314 - protein, 7 aa, Homo sapiens
QEQPVYI

SEQ ID NO: 315 - protein, 8 aa, Homo sapiens
AQEQPVYI

SEQ ID NO: 316 - protein, 4 aa, Homo sapiens
PLYI

SEQ ID NO: 317 - protein, 5 aa, Homo sapiens
TPLYI

SEQ ID NO: 318 - protein, 6 aa, Homo sapiens
ETPLYI

SEQ ID NO: 319 - protein, 7 aa, Homo sapiens
QETPLYI

SEQ ID NO: 320 - protein, 8 aa, Homo sapiens
GQETPLYI

SEQ ID NO: 321 - protein, 4 aa, Homo sapiens
PVYL

SEQ ID NO: 322 - protein, 5 aa, Homo sapiens
QPVYL

SEQ ID NO: 323 - protein, 6 aa, Homo sapiens
EQPVYL

SEQ ID NO: 324 - protein, 7 aa, Homo sapiens
QEQPVYL

SEQ ID NO: 325 - protein, 8 aa, Homo sapiens
GQEQPVYL

SEQ ID NO: 326 - protein, 4 aa, Homo sapiens
QFYI

SEQ ID NO: 327 - protein, 5 aa, Homo sapiens
HQFYI

SEQ ID NO: 328 - protein, 6 aa, Homo sapiens
FHQFYI

SEQ ID NO: 329 - protein, 7 aa, Homo sapiens
AFHQFYI

SEQ ID NO: 330 - protein, 8 aa, Homo sapiens
LAFHQFYI

SEQ ID NO: 331 - protein, 4 aa, Homo sapiens
QLYI

SEQ ID NO: 332 - protein, 5 aa, Homo sapiens
HQLYI

SEQ ID NO: 333 - protein, 6 aa, Homo sapiens
SHQLYI

SEQ ID NO: 334 - protein, 7 aa, Homo sapiens
ASHQLYI

SEQ ID NO: 335 - protein, 8 aa, Homo sapiens
LASHQLYI

SEQ ID NO: 336 - protein, 4 aa, Homo sapiens
GIPI

SEQ ID NO: 337 - protein, 5 aa, Homo sapiens
VGIPI

SEQ ID NO: 338 - protein, 6 aa, Homo sapiens
TVGIPI

SEQ ID NO: 339 - protein, 7 aa, Homo sapiens
NTVGIPI

SEQ ID NO: 340 - protein, 8 aa, Homo sapiens
VNTVGIPI

SEQ ID NO: 341 - protein, 4 aa, Homo sapiens
YYKV

SEQ ID NO: 342 - protein, 5 aa, Homo sapiens
IYYKV

SEQ ID NO: 343 - protein, 6 aa, Homo sapiens
NIYYKV

SEQ ID NO: 344 - protein, 7 aa, Homo sapiens
ANIYYKV

SEQ ID NO: 345 - protein, 8 aa, Homo sapiens
PANITTKV

SEQ ID NO: 346 - protein, 4 aa, Homo sapiens
SVEV

SEQ ID NO: 347 - protein, 5 aa, Homo sapiens
QSVEV

SEQ ID NO: 348 - protein, 6 aa, Homo sapiens
IQSVEV

SEQ ID NO: 349 - protein, 7 aa, Homo sapiens
QIQSVEV

SEQ ID NO: 350 - protein, 8 aa, Homo sapiens
NQIQSVEV

SEQ ID NO: 351 - protein, 4 aa, Homo sapiens
HDDV

SEQ ID NO: 352 - protein, 5 aa, Homo sapiens
VHDDV

SEQ ID NO: 353 - protein, 6 aa, Homo sapiens
LVHDDV

SEQ ID NO: 354 - protein, 7 aa, Homo sapiens
LLVHDDV

SEQ ID NO: 355 - protein, 8 aa, Homo sapiens
PLLVHDDV

SEQ ID NO: 356 - protein, 4 aa, Homo sapiens
NTVV

SEQ ID NO: 357 - protein, 5 aa, Homo sapiens
RNTVV

SEQ ID NO: 358 - protein, 6 aa, Homo sapiens
HRNTVV

SEQ ID NO: 359 - protein, 7 aa, Homo sapiens
RHRNTVV

SEQ ID NO: 360 - protein, 8 aa, Homo sapiens
YRHRNTVV

SEQ ID NO: 361 - protein, 4 aa, Homo sapiens
ATFV

SEQ ID NO: 362 - protein, 5 aa, Homo sapiens
HATFV

SEQ ID NO: 363 - protein, 6 aa, Homo sapiens
HHATFV

SEQ ID NO: 364 - protein, 7 aa, Homo sapiens
AHHATFV

SEQ ID NO: 365 - protein, 8 aa, Homo sapiens
SAHHATFV

SEQ ID NO: 366 - protein, 4 aa, Homo sapiens
SSEA

SEQ ID NO: 367 - protein, 5 aa, Homo sapiens
QSSEA

SEQ ID NO: 368 - protein, 6 aa, Homo sapiens
SQSSEA

SEQ ID NO: 369 - protein, 7 aa, Homo sapiens
PSQSSEA

SEQ ID NO: 370 - protein, 8 aa, Homo sapiens
GPQQSSEA

SEQ ID NO: 371 - protein, 4 aa, Homo sapiens
ESEI

SEQ ID NO: 372 - protein, 5 aa, Homo sapiens
RESEI

SEQ ID NO: 373 - protein, 6 aa, Homo sapiens
RRESEI

SEQ ID NO: 374 - protein, 7 aa, Homo sapiens
LRRESEI

SEQ ID NO: 375 - protein, 8 aa, Homo sapiens
PLRRESEI

SEQ ID NO: 376 - protein, 4 aa, Homo sapiens
ATRL

SEQ ID NO: 377 - protein, 5 aa, Homo sapiens
NATRL

SEQ ID NO: 378 - protein, 6 aa, Homo sapiens
HNATRL

SEQ ID NO: 379 - protein, 7 aa, Homo sapiens
HHNATRL

SEQ ID NO: 380 - protein, 8 aa, Homo sapiens
AHHNATRL

SEQ ID NO: 381 - protein, 4 aa, Homo sapiens
GLLA

SEQ ID NO: 382 - protein, 5 aa, Homo sapiens
LGLLA

SEQ ID NO: 383 - protein, 6 aa, Homo sapiens
RLGLLA

SEQ ID NO: 384 - protein, 7 aa, Homo sapiens
TRLGLLA

SEQ ID NO: 385 - protein, 8 aa, Homo sapiens
VTRLGLLA

SEQ ID NO: 386 - protein, 4 aa, Homo sapiens
VQLE

SEQ ID NO: 387 - protein, 5 aa, Homo sapiens
RVQLE

SEQ ID NO: 388 - protein, 6 aa, Homo sapiens
PRVQLE

SEQ ID NO: 389 - protein, 7 aa, Homo sapiens
NPRVQLE

SEQ ID NO: 390 - protein, 8 aa, Homo sapiens
QNPRVQLE

SEQ ID NO: 391 - protein, 4 aa, Homo sapiens
DLEI

SEQ ID NO: 392 - protein, 5 aa, Homo sapiens
DDLEI

SEQ ID NO: 393 - protein, 6 aa, Homo sapiens
PDDLEI

SEQ ID NO: 394 - protein, 7 aa, Homo sapiens
RPDDLEI

SEQ ID NO: 395 - protein, 8 aa, Homo sapiens
PRPDDLEI

SEQ ID NO: 396 - protein, 4 aa, Homo sapiens
YQAL

SEQ ID NO: 397 - protein, 6 aa, Homo sapiens
AYAQAL

SEQ ID NO: 398 - protein, 6 aa, Homo sapiens
SAYQAL

SEQ ID NO: 399 - protein, 7 aa, Homo sapiens
PSAYQAL

SEQ ID NO: 400 - protein, 8 aa, Homo sapiens
RPSAYQAL

SEQ ID NO: 401 - protein, 4 aa, Homo sapiens
ESDL

SEQ ID NO: 402 - protein, 5 aa, Homo sapiens
GESDL

SEQ ID NO: 403 - protein, 6 aa, Homo sapiens
YGESDL

SEQ ID NO: 404 - protein, 7 aa, Homo sapiens
LYGESDL

SEQ ID NO: 405 - protein, 8 aa, Homo sapiens
ALYGESDL

SEQ ID NO: 406 - protein, 4 aa, Homo sapiens
ATDL

SEQ ID NO: 407 - protein, 5 aa, Homo sapiens
PATDL

SEQ ID NO: 408 - protein, 6 aa, Homo sapiens
GPATDL

SEQ ID NO: 409 - protein, 7 aa, Homo sapiens
WGPATDL

SEQ ID NO: 410 - protein, 8 aa, Homo sapiens
DWGPATDL

SEQ ID NO: 411 - protein, 4 aa, Homo sapiens
TSPL

SEQ ID NO: 412 - protein, 5 aa, Homo sapiens
PTSPL

SEQ ID NO: 413 - protein, 6 aa, Homo sapiens
GPTSPL

SEQ ID NO: 414 - protein, 7 aa, Homo sapiens
WGPTSPL

SEQ ID NO: 415 - protein, 8 aa, Homo sapiens
DWGPTSPL

SEQ ID NO: 416 - protein, 4 aa, Homo sapiens
DTRL

SEQ ID NO: 417 - protein, 5 aa, Homo sapiens
QDTRL

SEQ ID NO: 418 - protein, 6 aa, Homo sapiens
VQDTRL

SEQ ID NO: 419 - protein, 7 aa, Homo sapiens
EVQDTRL

SEQ ID NO: 420 - protein, 8 aa, Homo sapiens
EEVQDTRL

SEQ ID NO: 421 - protein, 4 aa, Homo sapiens
ESSL

SEQ ID NO: 422 - protein, 5 aa, Homo sapiens
KESSL

SEQ ID NO: 423 - protein, 6 aa, Homo sapiens
PKESSL

SEQ ID NO: 424 - protein, 7 aa, Homo sapiens
PPKESSL

SEQ ID NO: 425 - protein, 8 aa, Homo sapiens
FPPKESSL

SEQ ID NO: 426 - protein, 4 aa, Homo sapiens
EIAV

SEQ ID NO: 427 - protein, 5 aa, Homo sapiens
DEIAV

SEQ ID NO: 428 - protein, 6 aa, Homo sapiens
DDEIAV

SEQ ID NO: 429 - protein, 7 aa, Homo sapiens
LDDEIAV

SEQ ID NO: 430 - protein, 8 aa, Homo sapiens
GLDDEIAV

SEQ ID NO: 431 - protein, 4 aa, Homo sapiens
VSAV

SEQ ID NO: 432 - protein, 5 aa, Homo sapiens
QVSAV

SEQ ID NO: 433 - protein, 6 aa, Homo sapiens
EQVSAV

SEQ ID NO: 434 - protein, 7 aa, Homo sapiens
DEQVSAV

SEQ ID NO: 435 - protein, 8 aa, Homo sapiens
EDEQVSAV

SEQ ID NO: 436 - protein, 4 aa, Homo sapiens
ETDV

SEQ ID NO: 437 - protein, 5 aa, Homo sapiens
LETDV

SEQ ID NO: 438 - protein, 6 aa, Homo sapiens
ALETDV

SEQ ID NO: 439 - protein, 7 aa, Homo sapiens
EALETDV

SEQ ID NO: 440 - protein, 8 aa, Homo sapiens
VEALETDV

SEQ ID NO: 441 - protein, 4 aa, Homo sapiens
PQFV

SEQ ID NO: 442 - protein, 5 aa, Homo sapiens
IPQFV

SEQ ID NO: 443 - protein, 6 aa, Homo sapiens
EIPQFV

SEQ ID NO: 444 - protein, 7 aa, Homo sapiens
DEIPQFV

SEQ ID NO: 445 - protein, 8 aa, Homo sapiens
EDEIPQFV

SEQ ID NO: 446 - protein, 4 aa, Homo sapiens
QSSV

SEQ ID NO: 447 - protein, 5 aa, Homo sapiens
DQSSV

SEQ ID NO: 448 - protein, 6 aa, Homo sapiens
WDQSSV

SEQ ID NO: 449 - protein, 7 aa, Homo sapiens
VWDQSSV

SEQ ID NO: 450 - protein, 8 aa, Homo sapiens
KVWDQSSV

SEQ ID NO: 451 - protein, 4 aa, Homo sapiens
SSSV

SEQ ID NO: 452 - protein, 5 aa, Homo sapiens
FSSSV

SEQ ID NO: 453 - protein, 6 aa, Homo sapiens
PFSSSV

SEQ ID NO: 454 - protein, 7 aa, Homo sapiens
VPFSSSV

SEQ ID NO: 455 - protein, 8 aa, Homo sapiens
VVPFSSSV

SEQ ID NO: 456 - protein, 4 aa, Homo sapiens
VTSV

SEQ ID NO: 457 - protein, 5 aa, Homo sapiens
LVTSV

SEQ ID NO: 458 - protein, 6 aa, Homo sapiens
YLVTSV

SEQ ID NO: 459 - protein, 7 aa, Homo sapiens
SYLVTSV

SEQ ID NO: 460 - protein, 8 aa, Homo sapiens
GSYLVTSV

SEQ ID NO: 461 - protein, 4 aa, Homo sapiens
SVGL

SEQ ID NO: 462 - protein, 5 aa, Homo sapiens
ISVGL

SEQ ID NO: 463 - protein, 6 aa, Homo sapiens
EISVGL

SEQ ID NO: 464 - protein, 7 aa, Homo sapiens
QEISVGL

SEQ ID NO: 465 - protein, 8 aa, Homo sapiens
EQEISVGL

SEQ ID NO: 466 - protein, 4 aa, Homo sapiens
DSWV

SEQ ID NO: 467 - protein, 5 aa, Homo sapiens
PDSWV

SEQ ID NO: 468 - protein, 6 aa, Homo sapiens
SPDSWV

SEQ ID NO: 469 - protein, 7 aa, Homo sapiens
ASPDSWV

SEQ ID NO: 470 - protein, 8 aa, Homo sapiens
PASPDSWV

SEQ ID NO: 471 - protein, 4 aa, Homo sapiens
DVKI

SEQ ID NO: 472 - protein, 5 aa, Homo sapiens
QDVKI

SEQ ID NO: 473 - protein, 6 aa, Homo sapiens
SQDVKI

SEQ ID NO: 474 - protein, 7 aa, Homo sapiens
ESQDVKI

SEQ ID NO: 475 - protein, 8 aa, Homo sapiens
VESQDVKI

SEQ ID NO: 476 - protein, 4 aa, Homo sapiens
LTGL

SEQ ID NO: 477 - protein, 5 aa, Homo sapiens
DLTGL

SEQ ID NO: 478 - protein, 6 aa, Homo sapiens
VDLTGL

SEQ ID NO: 479 - protein, 7 aa, Homo sapiens
IVDLTGL

SEQ ID NO: 480 - protein, 8 aa, Homo sapiens
AIVDLTGL

SEQ ID NO: 481 - protein, 4 aa, Homo sapiens
EYFI

SEQ ID NO: 482 - protein, 5 aa, Homo sapiens
KEYFI

SEQ ID NO: 483 - protein, 6 aa, Homo sapiens
RKEYFI

SEQ ID NO: 484 - protein, 7 aa, Homo sapiens
SRKEYFI

SEQ ID NO: 485 - protein, 8 aa, Homo sapiens
SSRKEYFI

SEQ ID NO: 486 - protein, 4 aa, Homo sapiens
ITKV

SEQ ID NO: 487 - protein, 5 aa, Homo sapiens
QITKV

SEQ ID NO: 488 - protein, 6 aa, Homo sapiens
TQITKV

SEQ ID NO: 489 - protein, 7 aa, Homo sapiens
HTQITKV

SEQ ID NO: 490 - protein, 8 aa, Homo sapiens
QHTQITKV

SEQ ID NO: 491 - protein, 21 aa, Artificial
  HIV Tat-CD3 carboxyl terminus fusion peptide
GYGRKKRRQRRRGPPSSSSGL

SEQ ID NO: 492 - protein, 21 aa, Artificial
  HIV Tat-CLASP1 carboxyl terminus fusion peptide
GYGRKKRRQRRRGSISSSAEV

SEQ ID NO: 493 - protein, 21 aa, Artificial
  HIV Tat-CLASP2 carboxyl terminus fusion peptide
GYGRKKRRQRRRGMTSSSSVV

SEQ ID NO: 494 - protein, 13 aa, Artificial
  truncated HIV Tat peptide
GYGRKKRRQRRRG

SEQ ID NO: 495 - protein, 16 aa, Artificial
  MBPAc1-16 peptide
  MOD_RES (1)..(1): Xaa = acetylated Ala
XSQKRPSQRHGSKYLA

SEQ ID NO: 496 - protein, 341 aa, Artificial
  Dishevelled 1 (DVL1) Construct (N-P) aa 1 - aa 341
MAETKIIYHMDEEETPYLVKLPVAPERVTLADFKNVLSNRPVHAYKFFKSMDQDFGVVKEEIFDDNAKLPCFNGRVVSWLVLVEGAHSDAGSQGTDSHTDLPPPLERTGGIGDSRSPSFQPDVASSRDGMDNETGTESMVSHRRDRARRRNREEAARTNGHPRGDRRRDVGLPPDSASTALSSELESSSFVDSDEDDSTSRLSSSTEQSTSSRLIRKHKRRRRKQRLRQADRASSFSSMTDSTMSLNIITVTLNMERHHFLGICIVGQSNDRGDGGIYIGSIMKGGAVAADGRIEPGDMLLQVNDVNFENMSNDDAVRVLREIVSQTGPISLTVAKCWDPT

SEQ ID NO: 497 - protein, 198 aa, Artificial
  Dishevelled 1 (DVL1) Construct (N) aa 1 - aa 197
MAETKIIYHMDEEETPYLVKLPVAPERVTLADFKNVLSNRPVHAYKFFFKSMDQDFGVVKEEIFDDNAKLPCFNGRVVSWLVLVEGAHSDAGSQGTDSHTDLPPPLERTGGIGDSRSPSFQPDVASSRDGMDNETGTESMVSHRRDRARRRNREEAARTNGHPRGDRRRDVGLPPDSASTALSSELESSSFVDSDEDG

SEQ ID NO: 498 - protein, 97 aa, Artificial
  Dishevelled 1 (DVL1) Construct (P) aa 246 - aa 341
SLNIITVTLNMERHHFLGICIVGQSNDRGDGGIYIGSIMKGGAVAADGRIEPGDMLLQVNDVNFENMSNDDAVRVLREIVSQTGPISLTVAKCWDPT

SEQ ID NO: 499 - DNA, 28 bp, Artificial
  primer 308 DVF (N 128 - N 155)
tcggaattcgtcgcgccatggcggagac

SEQ ID NO: 500 - DNA, 29 bp, Artificial
  primer 311 DVR (N 1004 - N 1032)
gggaattcggtcccagcacttggccacag

SEQ ID NO: 501 - DNA, 28 bp, Artificial
  primer 344 DVF (N 873 - N 900)
ccagaattctcaacatcgtcactgtcac

SEQ ID NO: 502 - DNA, 32 bp, Artificial
  primer 345 DVR (N713 - N744)
tcggaattccatcctcgtccgagtccacaaag

SEQ ID NO: 503 - protein, 427 aa, Artificial
  KIAA 0751 / 41.8 KD Construct (N-J) aa 389 - aa 803
MMYFGGHSLEEDLEWSEPQIKDSGVDTCSSTTLNEEHSHSDKHPVTWQPSKDGDRLIGRILLNKRLKDGSVPRDSGAMLGLKVVGGKMTESGRLCAFITKVKKGSLADTVGHLRPGDEVLEWNGRLLQGATFEEVYNIILESKPEPQVELVVSRPIGDIPRIPDSTHAQLESSSSSFESQKMDRPSISVTSPMSPGMLRDVPQFLSGQLSIKLWFDKVGHQLIVTILGAKDLPSREDGRPRNPYVKIYFLPDRSDKNKRRTKTVKKTLEPKWNQTFIYSPVHRREFRERMLEITLWDQARVREEESEFLGEILIELETALLDDEPHWYKLQTHDVSSLPLPHPSPYMPRRQLHGESPTRRLQRSKRISDSEVSDYDCDDGIGVVSDYRHDGRDLQSSTLSVPEQVMSSNHCSPSGSPHRVDVIGRTT

SEQ ID NO: 504 - protein, 92 aa, Artificial
  KIAA 0751 / 41.8 KD Construct (P) aa 443 - aa 534
LKDGSVPRDSGAMLGLKVVGGKMTESGRLCAFITKVKKGSLADTVGHLRPGDEVLEWNGRLLQGATFEEVYNIILESKPEPQVELVVSRPIA

SEQ ID NO: 505 - DNA, 28 bp, Artificial
  primer 318 KIF (N 1366 - N 1393)
agacaattgaggaaatgatgtactttgg

SEQ ID NO: 506 - DNA, 28 bp, Artificial
  primer 319 KIR (N 1830 - N 1857)
gaacaattgcaataggccttgaaactac

SEQ ID NO: 507 - DNA, 28 bp, Artificial
  primer 320 KIR (N 2640 -N 2667)
acccaattgtagtccttcctataacatc

SEQ ID NO: 508 - DNA, 27 bp, Artificial
  primer 341 KIF (N 1567 - N 1593)
atagaattctaaaagatggaagtgtac

SEQ ID NO: 509 - protein, 251 aa, Artificial
  PAR6 Construct (N-P) aa 1 - aa 251
MARPQRTPARSPDSIVEVKSKFDAEFRRFALPRASVSGFQEFSRLLRAVHQIPGLDVLLGYTDAHGDLLPLTNDDSLHRALASGPPPLRLLVQKREADSSGLAFASNSLQRRKKGLLLRPVAPLRTRPPLLISLPQDFRQVSSVIDVDLLPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 510 - protein, 146 aa, Artificial
  PAR6 Construct (N) aa 1 - aa 147
MARPQRTPARSPDSIVEVKSKFDAEFRRFALPRASVSGFQEFSRLLRAVHQIPGLDVLLGYTDAHGDLLPLTNDDSLHRALASGPPPLRLLVQKREADSSGLAFASNSLQRRKKGLLLRPVAPLRTRPPLLISLPQDRQVSSVIDV

SEQ ID NO: 511 - protein, 97 aa, Artificial
  PAR6 Construct (P) aa 155 - aa 251
RRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 512 - DNA, 29 bp, Artificial
  primer 322 PAF (N 55 - N 82)
cccgaattcgccatggcccggccgcagag

SEQ ID NO: 513 - DNA, 28 bp, Artificial
  primer 324 PAR (N 798 - N 825)
cgtgaattcgctggttggcgggcttgac

SEQ ID NO: 514 - DNA, 30 bp, Artificial
  primer 342 PAF (N 519 - N 548)
gaggaattccgacgggtgcggctgcacaag

SEQ ID NO: 515 - DNA, 31 bp, Artificial
  primer 343 PAR (N 485 - N 516)
gcagaattcccacgtctatgactgaggaaac

SEQ ID NO: 516 - protein, 442 aa, Artificial
  post-synaptic desnsity protein 95 (PSD95) Construct (N-P3) aa 1 - aa 442
MSQRPRAPRSALWLLAPPLLRWAPPLLTVLHSDLFQALLDILDYYEASLSESQKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSR

SEQ ID NO: 517 - DNA, 32 bp, Artificial
  primer 315 PSF (N847 - N876)
agagaattcagagatatgtcccagagaccaag

SEQ ID NO: 518 - DNA, 29 bp, Artificial
  primer 304 PSR (N 2161 - N 2189)
cgagaattctgtactcttctggtttatac

SEQ ID NO: 519 - protein, 84 aa, Artificial
  hCASK (CASK) Construct (P) aa 399 - aa 572
RLVQFQKNTDEPMGITLKMNELNHCIVARIMHGGMIHRQGTLHVGDEIREINGISVANQTVEQLQKMLREMRGSITFKIVPSYR

SEQ ID NO: 520 - DNA, 29 bp, Artificial
  primer 336 CAF (N 1484 - N 1512)
ccagaattcggctggtacagtttcaaaag

SEQ ID NO: 521 - DNA, 29 bp, Artificial
  primer 325 CAR (N 1722 - N 1750)
actgaattcggtaacttggcacaatcttg

SEQ ID NO: 522 - protein, 294 aa, Artificial
  membrane protein, palmitolated 2 (MPP2 / DLG2) Construct (N-SH3) aa 1 - aa 317
MPVAATNSETAMQQVLDNLGSLPSATGAAELDLIFLRGIMESPIVRSLAKAHERLEETKLEAVRDNNLELVQEILRDLAQLAEQSSTAAELAHILQEPHFQSLLETHDSVASKTYETPPPSPGLDPTFSNQPVPPDAVRMVGIRKTAGEHLGVTFRVEGGELVIARILHGGMVAQQGLLHVGDIIKEVNGQPVGSDPRALQELLRNASGSVILKILPSYQEPHLPRQVFVKCHFDYDPARDSLIPCKEAGLRFNAGDLLQIVNQDDANWWQACHVEGGSAGLIPSQLLEEKRKG

SEQ ID NO: 523 - DNA, 27 bp, Artificial
  primer 305 MF (N 58 - N 84)
agagaattcagagcccttgcctccttc

SEQ ID NO: 524 - DNA, 28 bp, Artificial
  primer 306 MR (N 798 - N 825)
tgagaattcctttccgcttctcctccag

SEQ ID NO: 525 - protein, 121 aa, Artificial
  Tax interaction protein 1 (TIP-1) Construct (N-C) aa 3 - aa 125
YIPGQPVTAVVQRVEIHKLRQGENLILGFSIGGGIDQDPSQNPFSEDKTDKGIYVTRVSEGGPAEIAGLQSGDKIMQVNGWDMTMVTHDQARKRLTKRSEEVVRLLVTRQSLQKAVQQSML

SEQ ID NO: 526 - DNA, 21 bp, Artificial
  primer 1318 TIP R3-1 (N 336 - N 356)
cagtccatgctgtcggatccg

SEQ ID NO: 527 - DNA, 21 bp, Artificial
  primer 1317 TIP R5-1
gtcggaattccctacatcccg

SEQ ID NO: 528 - protein, 4 aa, Human papillomavirus strain 63
LYII

SEQ ID NO: 529 - protein, 4 aa, Human papillomavirus strain 80
GSIE

SEQ ID NO: 530 - DNA, 24 bp, Artificial
  primer 8PSF1 (N1150 - N1173)
tcggatccttgagggggagatgga

SEQ ID NO: 531 - DNA, 24 bp, Artificial
  primer 11PSR2 (N2191 - N2168)
tcggaattcgctatactcttctgg

SEQ ID NO: 532 - DNA, 23 bp, Artificial
  primer 1DF1 (N815 - N837)
tcggatccaggttaatggctcag

SEQ ID NO: 533 - DNA, 24 bp, Artificial
  primer 3DR2 (N1850 - N1827)
tcggaattcgacgtgactcttcgg

SEQ ID NO: 534 - protein, 20 aa, Homo sapiens
VISKATPALPTVSISSSAEV

SEQ ID NO: 535 - protein, 20 aa, Homo sapiens
ISGTPTSTMVHGMTSSSSVV

SEQ ID NO: 536 - protein, 18 aa, Homo sapiens
SPQPDSTDNDDYDDISAA

SEQ ID NO: 537 - protein, 20 aa, Homo sapiens
QATSRNGHSARQHVVADTEL

SEQ ID NO: 538 - protein, 20 aa, Homo sapiens
SSPTYRALLLQGPSQSSSEA

SEQ ID NO: 539 - protein, 20 aa, Homo sapiens
IVFIIHSFLSAKVTRLGLLA

SEQ ID NO: 540 - protein, 20 aa, Gallus gallus
TEGNESSEATSPVNAIYSLA

SEQ ID NO: 541 - protein, 20 aa, Rattus norvegicus
SFTSILTCHQRRTQRKETVA

SEQ ID NO: 542 - protein, 20 aa, Homo sapiens
GPDGIITVNDFTQNPRVQLE

SEQ ID NO: 543 - protein, 19 aa, Homo sapiens
KKGTYLTDETHREVKFTSL

SEQ ID NO: 544 - protein, 20 aa, Homo sapiens
PSSRASSRASSRPRPDDLEI

SEQ ID NO: 545 - protein, 20 aa, Homo sapiens
LHNQASVPLEPRPLRRESEI

SEQ ID NO: 546 - protein, 19 aa, Homo sapiens
GGGGGSGGGGGSGIESVKI

SEQ ID NO: 547 - protein, 20 aa, Homo sapiens
RIAYSLLGLKDQVNTVGIPI

SEQ ID NO: 548 - protein, 20 aa, Homo sapiens
QPTPTLGLNLGNDPDRGTSI

SEQ ID NO: 549 - protein, 20 aa, Homo sapiens
LNETTETQRTLLNGDLQTSI

SEQ ID NO: 550 - protein, 20 aa, Homo sapiens
VDPNSPAAKKKYVSYNNLVI

SEQ ID NO: 551 - protein, 20 aa, Homo sapiens
ATDYLVQPFMDQLAFHQFYI

SEQ ID NO: 552 - protein, 20 aa, Homo sapiens
KTMPAAMFRLLTGQETPLYI

SEQ ID NO: 553 - protein, 20 aa, Homo sapiens
STDNLVRPFMDTLASHQLYI

SEQ ID NO: 554 - protein, 20 aa, Homo sapiens
KTMPAAMYRLLTAQEQPVYI

SEQ ID NO: 555 - protein, 20 aa, Homo sapiens
ALVLIAFCIIRRRPSAYQAL

SEQ ID NO: 556 - protein, 20 aa, Homo sapiens
VPGALDYAAFSSALYGESDL

SEQ ID NO: 557 - protein, 20 aa, Homo sapiens
VHDAESSDEDGYDWGPATDL

SEQ ID NO: 558 - protein, 20 aa, Homo sapiens
KDITSDSENSNFRNEIQSLV

SEQ ID NO: 559 - protein, 20 aa, Homo sapiens
PIPAGGCTFSGIFPTLTSPL

SEQ ID NO: 560 - protein, 20 aa, Homo sapiens
PPATPSPRLALPAHHNATRL

SEQ ID NO: 561 - protein, 20 aa, Homo sapiens
KPQIAALKEETEEEVQDTRL

SEQ ID NO: 562 - protein, 20 aa, Homo sapiens
IVTVVTMVTNVDFPPKESSL

SEQ ID NO: 563 - protein, 20 aa, Homo sapiens
KTMPAATYRLLTGQEQPVYL

SEQ ID NO: 564 - protein, 20 aa, Homo sapiens
LLNVLQRQELGDGLDDEIAV

SEQ ID NO: 565 - protein, 20 aa, Homo sapiens
PYQSQGFSTEEDEDEQVSAV

SEQ ID NO: 566 - protein, 20 aa, Homo sapiens
INSVGSTASSSQPLLVHDDV

SEQ ID NO: 567 - protein, 20 aa, Homo sapiens
EDPKDRPSAAHIVEALETDV

SEQ ID NO: 568 - protein, 20 aa, Homo sapiens
TQGFPGPATWRRISSLESEV

SEQ ID NO: 569 - protein, 20 aa, Homo sapiens
ILNSIQVMRAQMNQIQSVEV

SEQ ID NO: 570 - protein, 20 aa, Homo sapiens
PRLKRMQQFEDLEDEIPQFV

SEQ ID NO: 571 - protein, 20 aa, Homo sapiens
GPVPGEPAKPKTSAHHATFV

SEQ ID NO: 572 - protein, 20 aa, Homo sapiens
PVYIVQEMPPQSPANIYYKV

SEQ ID NO: 573 - protein, 20 aa, Homo sapiens
YSQPSARSEGEFKQTSSFLV

SEQ ID NO: 574 - protein, 20 aa, Homo sapiens
ENLELPVNPSSVVSERISSV

SEQ ID NO: 575 - protein, 20 aa, Homo sapiens
AGAVRTPLSQVNKVWDQSSV

SEQ ID NO: 576 - protein, 20 aa, Homo sapiens
RNIEEVYVGGKQVVPFSSSV

SEQ ID NO: 577 - protein, 20 aa, Homo sapiens
ESSGTQSPKRHSGSYLVTSV

SEQ ID NO: 578 - protein, 20 aa, Homo sapiens
SLKPGTVLPPPPYRHRNTVV

SEQ ID NO: 579 - protein, 20 aa, Homo sapiens
ERASSVYTRSTGEQEISVGL

SEQ ID NO: 580 - protein, 20 aa, Homo sapiens
HPTDITGLPNLSDPSVSTVV

SEQ ID NO: 581 - protein, 20 aa, Homo sapiens
PYSELNYETSHYPASPDSWV

SEQ ID NO: 582 - protein, 20 aa, Homo sapiens
ELLQFCRTPNPALKNGQYWV

SEQ ID NO: 583 - protein, 20 aa, Homo sapiens
TREDIYVNYPTFSRRPKTRV

SEQ ID NO: 584 - protein, 20 aa, Homo sapiens
GGEDFKTTNPSKQFDKNAYV

SEQ ID NO: 585 - protein, 20 aa, Homo sapiens
DGGARTEDEVQSYPSKHDYV

SEQ ID NO: 586 - protein, 20 aa, Homo sapiens
SYPTPRPYPKPAPSSGKDYV

SEQ ID NO: 587 - protein, 20 aa, Homo sapiens
LGYSIPSRSGASGLDKRDYV

SEQ ID NO: 588 - protein, 20 aa, Homo sapiens
KAGYRAPRSYPKSNSSKEYV

SEQ ID NO: 589 - protein, 20 aa, Homo sapiens
PGQPPKVKSEFNSYSLTGYV

SEQ ID NO: 590 - protein, 20 aa, Homo sapiens
SSPDSSYQGKGFVMSRAMYV

SEQ ID NO: 591 - protein, 19 aa, Homo sapiens
SSKSKSSEESQTFFGLYKL

SEQ ID NO: 592 - protein, 20 aa, Homo sapiens
DTLLLTENEGDKTEEQVSYV

SEQ ID NO: 593 - protein, 20 aa, Homo sapiens
RELVDRGEVRQFTLRHWLKV

SEQ ID NO: 594 - protein, 20 aa, Homo sapiens
HDFRRAFKKILARGDRKRIV

SEQ ID NO: 595 - protein, 20 aa, Homo sapiens [sequence omitted]

SEQ ID NO: 596 - protein, 20 aa, Homo sapiens
DFRPSFKHILFRRARRGFRQ

SEQ ID NO: 597 - protein, 20 aa, Homo sapiens
EALQPEPGRKRIPLTRTTTF

SEQ ID NO: 598 - protein, 20 aa, Homo sapiens
YSATYSELEDPGEMSPPIDL

SEQ ID NO: 599 - protein, 20 aa, Homo sapiens
LAVLAYSITLVMLWSIWQYA

SEQ ID NO: 600 - protein, 20 aa, Homo sapiens
QQYQQRQSVIFHKRAPEQAL

SEQ ID NO: 601 - protein, 20 aa, Homo sapiens
RDSFHRSSFRKAETQLSQGS

SEQ ID NO: 602 - protein, 20 aa, Homo sapiens
HHLVAQRDIRQFQLQHWLAI

SEQ ID NO: 603 - protein, 20 aa, Homo sapiens
DAKLKSDGTIAAITEKETHF

SEQ ID NO: 604 - protein, 20 aa, Homo sapiens
NGGFAVDKSDTISFTQTSQF

SEQ ID NO: 605 - protein, 20 aa, Homo sapiens
TALRIKKTLAKMVESQDVKI

SEQ ID NO: 606 - protein, 20 aa, Homo sapiens
RPMESNPDTEGAQGETEDVL

SEQ ID NO: 607 - protein, 20 aa, Homo sapiens
ESKSFTRSTVDTMAQKTQAV

SEQ ID NO: 608 - protein, 19 aa, Homo sapiens [sequence omitted]

SEQ ID NO: 609 - protein, 19 aa, Homo sapiens
KDSRPSFVGSSSGHTSTTL

SEQ ID NO: 610 - protein, 19 aa, Homo sapiens
ARHRVTSYTSSSVNVSSNL

SEQ ID NO: 611 - protein, 19 aa, Homo sapiens
AWDDSARAAGGQGLHVTAL

SEQ ID NO: 612 - protein, 20 aa, Homo sapiens
AAWDDSARAAGGQGLHVTAL

SEQ ID NO: 613 - protein, 20 aa, Homo sapiens
NSYVRDDAIFIKAIVDLTGL

SEQ ID NO: 614 - protein, 20 aa, Homo sapiens
GTSDMKDLVGNIEQNEHSVI

SEQ ID NO: 615 - protein, 18 aa, Homo sapiens
TFAAGFNSTGLPHSTTRV

SEQ ID NO: 616 - protein, 19 aa, Homo sapiens
QGDPALQDAGDSSRKEYFI

SEQ ID NO: 617 - protein, 20 aa, Homo sapiens
LASKSAEEGKQIPDSLSTDL

SEQ ID NO: 618 - protein, 20 aa, Homo sapiens
PSWRRSSLSESENATSLTTF

SEQ ID NO: 619 - protein, 20 aa, Human T-lymphotropic virus 1
QISPGGLEPPSEKHFRETEV

SEQ ID NO: 620 - protein, 20 aa, Homo sapiens
KENDYESISDLQQGRDITRL

SEQ ID NO: 621 - protein, 20 aa, Homo sapiens
DSDPENEPFDEDQHTQITKV

SEQ ID NO: 622 - protein, 20 aa, Human adenovirus
VDSERRPHFPQFSYSASSTA

SEQ ID NO: 623 - protein, 20 aa, Human papillomavirus strain #66 (cysteine-free)
TGSALQAWRHTSRQATESTV

SEQ ID NO: 624 - protein, 20 aa, Human papillomavirus strain #57 (cysteine-free)
HAMNAAPRAMENAPALRTSH

SEQ ID NO: 625 - protein, 19 aa, Human papillomavirus strain #16 (modified)
TGRGMSGGRSSRTRRETQL

SEQ ID NO: 626 - protein, 19 aa, Human papillomavirus strain #18
SGGNRARQERLQRRRETQV

SEQ ID NO: 627 - protein, 20 aa, Human papillomavirus strain 33 (modified)
AAGGRSARGGRLQGRRETAL

SEQ ID NO: 628 - protein, 20 aa, Human papillomavirus strain 52 (modified)
SEGGRPTRGPRLQGRRVTQV

SEQ ID NO: 629 - protein, 20 aa, Human papillomavirus strain 58 (modified)
AVGGRPARGGRLQGRRQTQV

SEQ ID NO: 630 - protein, 20 aa, Human papillomavirus strain 77 (modified)
GGGRGSGLAGGSRGGGQSRQ

SEQ ID NO: 631 - protein, 20 aa, Human papillomavirus strain #35 (cysteine-free)
GRWTGRAMSAWKPTRRETEV

SEQ ID NO: 632 - protein, 20 aa, Human adenovirus AdenoE4 typ9
VGTLLLERVIFPSVKIATLV

SEQ ID NO: 633 - protein, 87 aa, Homo sapiens
RDMAEAHKEAMSRKLGQSESQGPPRAFAKVNSISPGSPSIAGLQVDDEIVEFGSVNTQNFQSLHNIGSVVQHSEGALAPTILLSVSM

SEQ ID NO: 634 - protein, 93 aa, Homo sapiens
LRKEPEIITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 635 - protein, 105 aa, Homo sapiens
LIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTNSS

SEQ ID NO: 636 - protein, 132 aa, Homo sapiens
GISSLGRKTPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESKEANSS

SEQ ID NO: 637 - protein, 105 aa, Homo sapiens
QSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSNSS

SEQ ID NO: 638 - protein, 101 aa, Homo sapiens
LGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 639 - protein, 74 aa, Homo sapiens
QTVILPGPAAWGFRLSGGIDFNQPLVITRITPGSKAAAANLCPGDVILAIDGFGTESMTHADGQDRIKAAEFIV

SEQ ID NO: 640 - protein, 85 aa, Homo sapiens
ILVEVQLSGGAPWGFTLKGGREHGEPLVITKIEEGSKAAAVDKLLAGDEIVGINDIGLSGFRQEAICLVKGSHKTLKLVVKRNSS

SEQ ID NO: 641 - protein, 104 aa, Homo sapiens
REKPLFTRDASQLKGTFLSTTLKKSNMGFGFTIIGGDEPDEFLQVKSVIPDGPAAQDGKMETGDVIVYINEVCVLGHTHADVVKLFQSVPIGQSVNLVLCRGYP

SEQ ID NO: 642 - protein, 91 aa, Homo sapiens
LSGATQAELMTLTIVKGAQGFGFTIADSPTGQRVKQILDIQGCPGLCEGDLIVEINQQNVQNLSHTEVVDILKDCPIGSETSLIIHRGGFF

SEQ ID NO: 643 - protein, 93 aa, Homo sapiens
HYKELDVHLRRMESGFGFRILGGDEPGQPILIGAVIAMGSADRDGRLHPGDELVYVDGIPVAGKTHRYVIDLMHHAARNGQVNLTVRRKVLCG

SEQ ID NO: 644 - protein, 106 aa, Homo sapiens
EGRGISSHSLQTSDAVIHRKENEGFGFVIISSLNRPESGSTITVPHKIGRIIDGSPADRCAKLKVGDRILAVNGQSIINMPHADIVKLIKDAGLSVTLRIIPQEEL

SEQ ID NO: 645 - protein, 98 aa, Homo sapiens
LSDYRQPQDFDYFTVDMEKGAKGFGFSIRGGREYKMDLYVLRLAEDGPAIRNGRMRVGDQIIEINGESTRDMTHARAIELIKSGGRRVRLLLKRGTGQ

SEQ ID NO: 646 - protein, 90 aa, Homo sapiens
HESVIGRNPEGQLGFELKGGAENGQFPYLGEVKPGKVAYESGSKLVSEELLLEVNETPVAGLTIRDVLAVIKHCKDPLRLKCVKQGGIHR

SEQ ID NO: 647 - protein, 103 aa, Homo sapiens
IQKKNHWTSRVHECTVKRGPQGELGVTVLGGAEHGEFPYVGAVAAVEAAGLPGGGEGPRLGEGELLLEVQGVRVSGLPRYDVLGVIDSCKEAVTFKAVRQGGR

SEQ ID NO: 648 - protein, 104 aa, Homo sapiens
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDDPN

SEQ ID NO: 649 - protein, 92 aa, Homo sapiens
PATQPELITVHIVKGPMGFGFTIADSPGGGGQRVKQIVDSPRCRGLKEGDLIVEVNKKNVQALTHNQVVDMLVECPKGSEVTLLVQRGGNLS

SEQ ID NO: 650 - protein, 102 aa, Homo sapiens
PDYQEQDIFLWRKETGFGFRILGGNEPGEPIYIGHIVPLGAADTDGRLRSGDELICVDGTPVIGKSHQLVVQLMQQAAKQGHVNLTVRRKVVFAVPKTENSS

SEQ ID NO: 651 - protein, 112 aa, Homo sapiens
GVVSTVVQPYDVEIRRGENEGFGFVIVSSVSRPEAGTTFAGNACVAMPHKIGRIIEGSPADRCGKLKVGDRILAVNGCSITNKSHSDIVNLIKEAGNTVTLRIIPGDESSNA

SEQ ID NO: 652 - protein, 91 aa, Homo sapiens
QATQEQDFYTVELERGAKGFGFSLRGGREYNMDLYVLRLAEDGPAERCGKMRIGDEILEINGETTKNMKHSRAIELIKNGGRRVRLFLKRG

SEQ ID NO: 653 - protein, 126 aa, Homo sapiens
NLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLV

SEQ ID NO: 654 - protein, 100 aa, Homo sapiens
ILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRL

SEQ ID NO: 655 - protein, 90 aa, Homo sapiens
TRVRLVQFQKNTDEPMGITLKMNELNHCIVARIMHGGMIHRQGTLHVGDEIREINGISVANQTVEQLQKMLREMRGSITFKIVPSYRTQS

SEQ ID NO: 656 - protein, 88 aa, Homo sapiens
LEQKAVLEQVQLDSPLGLEIHTTSNCQHFVSQVDTQVPTDSRLQIQPGDEVVQINEQVVVGWPRKNMVRELLREPAGLSLVLKKIPIP

SEQ ID NO: 657 - protein, 92 aa, Homo sapiens
QRKLVTVEKQDNETFGFEIQSYRPQNQNACSSEMFTLICKIQEDSPAHCAGLQAGDVLANINGVSTEGFTYKQVVDLIRSSGNLLTIETLNG

SEQ ID NO: 658 - protein, 101 aa, Homo sapiens
IQVNGTDADYEYEEITLERGNSGLGFSIAGGTDNPHIGDDSSIFITKIITGGAAAQDGRLRVNDCILQVNEVDVRDVTHSKAVEALKEAGSIVRLYVKRRN

SEQ ID NO: 659 - protein, 95 aa, Homo sapiens
IQLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGN

SEQ ID NO: 660 - protein, 85 aa, Homo sapiens
ILHRGSTGLGFNIVGGEDGEGIFISFILAGGPADLSGELRKGDRIISVNSVDLRAASHEQAAAALKNAGQAVTIVAQYRPEEYSR

SEQ ID NO: 661 - protein, 101 aa, Homo sapiens
ISYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRR

SEQ ID NO: 662 - protein, 94 aa, Homo sapiens
ISVVEIKLFKGPKGLGFSIAGGVGNQHIPGDNSIYVTKIIDGGAAQKDGRLQVGDRLLMVNNYSLEEVTHEEAVAILKNTSEVVYLKVGNPTTI

SEQ ID NO: 663 - protein, 95 aa, Homo sapiens
IWAVSLEGEPRKVVLHKGSTGLGFNIVGGEDGEGIFVSFILAGGPADLSGELQRGDQILSVNGIDLRGASHEQAAAALKGAGQTVTIIAQYQPED

SEQ ID NO: 664 - protein, 102 aa, Homo sapiens
GIPYVEEPRHVKVQKGSEPLGISIVSGEKGGIYVSKVTVGSIAHQAGLEYGDQLLEFNGINLRSATEQQARLIIGQQCDTITILAQYNPHVHQLRNSSZLTD

SEQ ID NO: 665 - protein, 103 aa, Homo sapiens
GILAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFIVTD

SEQ ID NO: 666 - protein, 93 aa, Homo sapiens
LNIVTVTLNMERHHFLGISIVGQSNDRGDGGIYIGSIMKGGAVAADGRIEPGDMLLQVNDVNFENMSNDDAVRVLREIVSQTGPISLTVAKCW

SEQ ID NO: 667 - protein, 100 aa, Homo sapiens
LNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQNS

SEQ ID NO: 668 - protein, 95 aa, Homo sapiens
IITVTLNMEKYNFLGISIVGQSNERGDGGIYIGSIMKGGAVAADGRIEPGDMLLQVNEINFENMSNDDAVRVLREIVHKPGPITLTVAKCWDPSP

SEQ ID NO: 669 - protein, 92 aa, Homo sapiens
TTQQIDLQGPGPWGFRLVGRKDFEQPLAISRVTPGSKAALANLCIGDVITAIDGENTSNMTHLEAQNRIKGCTDNLTLTVARSEHKVWSPLV

SEQ ID NO: 670 - protein, 89 aa, Homo sapiens
IFMDSFKVVLEGPAPWGFRLQGGKDFNVPLSISRLTPGGKAAQAGVAVGDWVLSIDGENAGSLTHIEAQNKIRACGERLSLGLSRAQPV

SEQ ID NO: 671 - protein, 100 aa, Homo sapiens
QGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 672 - protein, 87 aa, Homo sapiens
ILCCLEKGPNGYGFHLHGEKGKLGQYIRLVEPGSPAEKAGLLAGDRLVEVNGENVEKETHQQVVSRIRAALNAVRLLVVDPEFIVTD

SEQ ID NO: 673 - protein, 92 aa, Homo sapiens
IRLCTMKKGPSGYGFNLHSDKSKPGQFIRSVDPDSPAEASGLRAQDRIVEVNGVCMEGKQHGDVVSAIRAGGDETKLLVVDRETDEFFMNSS

SEQ ID NO: 674 - protein, 107 aa, Homo sapiens
KNPSGELKTVTLSKMKQSLGISISGGIESKVQPMVKIEKIFPGGAAFLSGALQAGFELVAVDGENLEQVTHQRAVDTIRRAYRNKAREPMELVVRVPGPSPRPSPSD

SEQ ID NO: 675 - protein, 97 aa, Homo sapiens
EGHSHPRVVELPKTEEGLGFNIMGGKEQNSPIYISRIIPGGIADRHGGLKRGDQLLSVNGVSVEGEHHEKAVELLKAAQGKVKLVVRYTPKVLEEME

SEQ ID NO: 676 - protein, 97 aa, Homo sapiens
GQYGGETVKIVRIEKARDIPLGATVRNEMDSVIISRIVKGGAAEKSGLLHEGDEVLEINGIEIRGKDVNEVFDLLSDMHGTLTFVLIPSQQIKPPPA

SEQ ID NO: 677 - protein, 98 aa, Homo sapiens
ILAHVKGIEKEVNVYKSEDSLGLTITDNGVGYAFIKRIKDGGVIDSVKTICVGDHIESINGENIVGWRHYDVAKKLKELKKEELFTMKLIEPKKAFEI

SEQ ID NO: 678 - protein, 104 aa, Homo sapiens
KPSQASGHFSVELVRGYAGFGLTLGGGRDVAGDTPLAVRGLLKDGPAQRCGRLEVGDLVLHINGESTQGLTHAQAVERIRAGGPQLHLVIRRPLETHPGKPRGV

SEQ ID NO: 679 - protein, 83 aa, Homo sapiens
VVELMKKEGTTLGLTVSGGIDKDGKPRVSNLRQGGIAARSDQLDVGDYIKAVNGINLAKFRHDEIISLLKNVGERVVLEVEYE

SEQ ID NO: 680 - protein, 110 aa, Homo sapiens
RSSVIFRTVEVTLHKEGNTFGFVIRGGAHDDRNKSRPVVITCVRPGGPADREGTIKPGDRLLSVDGIRLLGTTHAEAMSILKQCGQEAALLIEYDVSVMDSVATASGNSS

SEQ ID NO: 681 - protein, 106 aa, Homo sapiens
HVATASGPLLVEVAKTPGASLGVALTTSMCCNKQVIVIDKIKSASIADRCGALHVGDHILSIDGTSMEYCTLAEATQFLANTTDQVKLEILPHHQTRLALKGPNSS

SEQ ID NO: 682 - protein, 97 aa, Homo sapiens
TETTEVVLTADPVTGFGIQLQGSVFATETLSSPPLISYIEADSPAERCGVLQIGDRVMAINGIPTEDSTFEEASQLLRDSSITSKVTLEIEFDVAES

SEQ ID NO: 683 - protein, 101 aa, Homo sapiens
AESVIPSSGTFHVKLPKKHNVELGITISSPSSRKPGDPLVISDIKKGSVAHRTGTLELGDKLLAIDNIRLDNCSMEDAVQILQQCEDLVKLKIRKDEDNSD

SEQ ID NO: 684 - protein, 90 aa, Homo sapiens
IYTVELKRYGGPLGITISGTEEPFDPIIISSLTKGGLAERTGAIHIGDRILAINSSSLKGKPLSEAIHLLQMAGETVTLKIKKQTDAQSA

SEQ ID NO: 685 - protein, 95 aa, Homo sapiens
IMSPTPVELHKVTLYKDSDMEDFGFSVADGLLEKGVYVKNIRPAGPGDLGGLKPYDRLLQVNHVRTRDFDCCLVVPLIAESGNKLDLVISRNPLA

SEQ ID NO: 686 - protein, 71 aa, Homo sapiens
LALPRDGQGRLGFEVDAEGFVTHVERFTFAETAGLRPGARLLRVCGQTLPSLRPEAAAQLLRSAPKVCVTV

SEQ ID NO: 687 - protein, 95 aa, Homo sapiens
AKAKWRQVVLQKASRESPLQFSLNGGSEKGFGIFVEGVEPGSKAADSGLKRGDQIMEVNGQNFENITFMKAVEILRNNTHLALTVKTNIFVFKEL

SEQ ID NO: 688 - protein, 89 aa, Homo sapiens
LENVIAKSLLIKSNEGSYGFGLEDKNKVPIIKLVEKGSNAEMAGMEVGKKIFAINGDLVFMRPFNEVDCFLKSCLNSRKPLRVLVSTKP

SEQ ID NO: 689 - protein, 82 aa, Homo sapiens
PRETVKIPDSADGLGFQIRGFGPSVVHAVGRGTVAAAAGLHPGQCIIKVNGINVSKETHASVIAHVTACRKYRRPTKQDSIQ

SEQ ID NO: 690 - protein, 100 aa, Homo sapiens
EDFCYVFTVELERGPSGLGMGLIDGMHTHLGAPGLYIQTLLPGSPAAADGRLSLGDRILEVNGSSLLGLGYLRAVDLIRHGGKKMRFLVAKSDVETAKKI

SEQ ID NO: 691 - protein, 104 aa, Homo sapiens
IWQIEYIDIERPSTGGLGFSVVALRSQNLGKVDIFVKDVQPGSVADRDQRLKENDQILAINHTPLDQNISHQQAIALLQQTTGSLRLIVAREPVHTKSSTSSSE

SEQ ID NO: 692 - protein, 78 aa, Homo sapiens
PGHVEEVELINDGSGLGFGIVGGKTSGVVVRTIVPGGLADRDGRLQTGDHILKIGGTNVQGMTSEQVAQVLRNCGNSS

SEQ ID NO: 693 - protein, 111 aa, Homo sapiens
PGSDSSLFETYNVELVRKDGQSLGIRIVGYVGTSHTGEASGIYVKSIIPGSAAYHNGHIQVNDKIVAVDGVNIQGFANHDVVEVLRNAGQVVHLTLVRRKTSSSTSRIHRD

SEQ ID NO: 694 - protein, 96 aa, Homo sapiens
NSDDAELQKYSKLLPIHTLRLGVEVDSFDGHHYISSIVSGGPVDTLGLLQPEDELLEVNGMQLYGKSRREAVSFLKEVPPPFTLVCCRRLFDDEAS

SEQ ID NO: 695 - protein, 102 aa, Homo sapiens
LSSPEVKIVELVKDCKGLGFSILDYQDPLDPTRSVIVIRSLVADGVAERSGGLLPGDRLVSVNEYCLDNTSLAEAVEILKAVPPGLVHLGICKPLVEFIVTD

SEQ ID NO: 696 - protein, 119 aa, Homo sapiens
PNFSHWGPPRIVEIFREPNVSLGISIVVGQTVIKRLKNGEELKGIFIKQVLEDSPAGKTNALKTGDKILEVSGVDLQNASHSEAVEAIKNAGNPVVFIVQSLSSTPRVIPNVHNKANSS

SEQ ID NO: 697 - protein, 99 aa, Homo sapiens
PGELHIIELEKDKNGLGLSLAGNKDRSRMSIFVVGINPEGPAAADGRMRIGDELLEINNQILYGRSHQNASAIIKTAPSKVKLVFIRNEDAVNQMANSS

SEQ ID NO: 698 - protein, 93 aa, Homo sapiens
PATCPIVPGQEMIIEISKGRSGLGLSIVGGKDTPLNAIVIHEVYEEGAAARDGRLWAGDQILEVNGVDLRNSSHEEAITALRQTPQKVRLVVY

SEQ ID NO: 699 - protein, 103 aa, Homo sapiens
ILTLTILRQTGGLGISIAGGKGSTPYKGDDEGIFISRVSEEGPAARAGVRVGDKLLEVNGVALQGAEHHEAVEALRGAGTAVQMRVWRERMVEPENAEFIVTD

SEQ ID NO: 700 - protein, 97 aa, Homo sapiens
PLRQRHVACLARSERGLGFSIAGGKGSTPYRAGDAGIFVSRIAEGGAAHRAGTLQVGDRVLSINGVDVTEARHDHAVSLLTAASPTIALLLEREAGG

SEQ ID NO: 701 - protein, 106 aa, Homo sapiens
ILEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAEFIVTD

SEQ ID NO: 702 - protein, 105 aa, Homo sapiens
RELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVS

SEQ ID NO: 703 - protein, 91 aa, Homo sapiens
PHQPIVIHSSGKNYGFTIRAIRVYVGDSDIYTVHHIVWNVEEGSPACQAGLKAGDLITHINGEPVHGLVHTEVIELLLKSGNKVSITTTPF

SEQ ID NO: 704 - protein, 105 aa, Homo sapiens
ILACAAKAKRRLMTLTKPSREAPLPFILLGGSEKGFGIFVDSVDSGSKATEAGLKRGDQILEVNGQNFENIQLSKAMEILRNNTHLSITVKTNLFVFKELLTNSS

SEQ ID NO: 705 - protein, 88 aa, Homo sapiens
IPPAPRKVEMRRDPVLGFGFVAGSEKPVVVRSVTPGGPSEGKLIPGDQIVMINDEPVSAAPRERVIDLVRSCKESILLTVIQPYPSPK

SEQ ID NO: 706 - protein, 101 aa, Homo sapiens
LNKRTTMPKDSGALLGLKVVGGKMTDLGRLGAFITKVKKGSLADVVGHLRAGDEVLEWNGKPLPGATNEEVYNIILESKSEPQVEIIVSRPIGDIPRIHRD

SEQ ID NO: 707 - protein, 79 aa, Homo sapiens
QRCVIIQKDQHGFGFTVSGDRIVLVQSVRPGGAAMKAGVKEGDRIIKVNGTMVTNSSHLEVVKLIKSGAYVALTLLGSS

SEQ ID NO: 708 - protein, 87 aa, Homo sapiens
ILVQRCVIIQKDDNGFGLTVSGDNPVFVQSVKEDGAAMRAGVQTGDRIIKVNGTLVTHSNHLEVVKLIKSGSYVALTVQGRPPGNSS

SEQ ID NO: 709 - protein, 79 aa, Homo sapiens
SVEMTLRRNGLGQLGFHVNYEGIVADVEPYGYAWQAGLRQGSRLVEICKVAVATLSHEQMIDLLRTSVTVKVVIIPPHD

SEQ ID NO: 710 - protein, 96 aa, Homo sapiens
LKVMTSGWETVDMTLRRNGLGQLGFHVKYDGTVAEVEDYGFAWQAGLRQGSRLVEICKVAVVTLTHDQMIDLLRTSVTVKVVIIPPFEDGTPRRGW

SEQ ID NO: 711 - protein, 105 aa, Homo sapiens
HYIFPHARIKITRDSKDHTVSGNGLGIRIVGGKEIPGHSGEIGAYIAKILPGGSAEQTGKLMEGMQVLEWNGIPLTSKTYEEVQSIISQQSGEAEICVRLDLNML

SEQ ID NO: 712 - protein, 103 aa, Homo sapiens
LCGSLRPPIVIHSSGKKYGFSLRAIRVYMGDSDVYTVHHVVWSVEDGSPAQEAGLRAGDLITHINGESVLGLVHMDVVELLLKSGNKISLRTTALENTSIKVG

SEQ ID NO: 713 - protein, 86 aa, Homo sapiens
SYSVTLTGPGPWGFRLQGGKDFNMPLTISRITPGSKAAQSQLSQGDLVVAIDGVNTDTMTHLEAQNKIKSASYNLSLTLQKSKNSS

SEQ ID NO: 714 - protein, 91 aa, Homo sapiens
ISRDSGAMLGLKVVGGKMTESGRLCAFITKVKKGSLADTVGHLRPGDEVLEWNGRLLQGATFEEVYNIILESKPEPQVELVVSRPIAIHRD

SEQ ID NO: 715 - protein, 101 aa, Homo sapiens
ISALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENSS

SEQ ID NO: 716 - protein, 94 aa, Homo sapiens
FSDMRISINQTPGKSLDFGFTIKWDIPGIFVASVEAGSPAEFSQLQVDDEIIAINNTKFSYNDSKEWEEAMAKAQETGHLVMDVRRYGKAGSPE

SEQ ID NO: 717 - protein, 98 aa, Homo sapiens
QSAHLEVIQLANIKPSEGLGMYIKSTYDGLHVITGTTENSPADRCKKIHAGDEVIQVNHQTVVGWQLKNLVNALREDPSGVILTLKKRPQSMLTSAPA

SEQ ID NO: 718 - protein, 100 aa, Homo sapiens
ILTQTLIPVRHTVKIDKDTLLQDYGFHISESLPLTVVAVTAGGSAHGKLFPGDQILQMNNEPAEDLSWERAVDILREAEDSLSITVVRCTSGVPKSSNSS

SEQ ID NO: 719 - protein, 93 aa, Homo sapiens
GLRSPITIQRSGKKYGFTLRAIRVYMGDTDVYSVHHIVWHVEEGGPAQEAGLCAGDLITHVNGEPVHGMVHPEVVELILKSGNKVAVTTTPFE

SEQ ID NO: 720 - protein, 107 aa, Homo sapiens
QGEETKSLTLVLHRDSGSLGFNIIGGRPSVDNHDGSSSEGIFVSKIVDSGPAAKEGGLQIHDRIIEVNGRDLSRATHDQAVEAFKTAKEPIVVQVLRRTPRTKMFTP

SEQ ID NO: 721 - protein, 101 aa, Homo sapiens
QEMDREELELEEVDLYRMNSQDKLGLTVCYRTDDEDDIGIYISEIDPNSIAAKDGRIREGDRIIQINGIEVQNREEAVALLTSEENKNFSLLIARPELQLD

SEQ ID NO: 722 - protein, 91 aa, Homo sapiens
RSFQYVPVQLQGGAPWGFTLKGGLEHCEPLTVSKIEDGGKAALSQKMRTGDELVNINGTPLYGSRQEALILIKGSFRILKLIVRRRNAPVS

SEQ ID NO: 723 - protein, 102 aa, Homo sapiens
ILEKLELFPVELEKDEDGLGISIIGMGVGADAGLEKLGIFVKTVTEGGAAQRDGRIQVNDQIVEVDGISLVGVTQNFAATVLRNTKGNVRFVIGREKPGQVS

SEQ ID NO: 724 - protein, 113 aa, Homo sapiens
KDVNVYVNPKKLTVIKAKEQLKLLEVLVGIIHQTKWSWRRTGKQGDGERLVVHGLLPGGSAMKSGQVLIGDVLVAVNDVDVTTENIERVLSCIPGPMQVKLTFENAYDVKRET

SEQ ID NO: 725 - protein, 90 aa, Homo sapiens
TRGCETVEMTLRRNGLGQLGFHVNFEGIVADVEPFGFAWKAGLRQGSRLVEICKVAVATLTHEQMIDLLRTSVTVKVVIIQPHDDGSPRR

SEQ ID NO: 726 - protein, 96 aa, Homo sapiens
VENILAKRLLILPQEEDYGFDIEEKNKAVVVKSVQRGSLAEVAGLQVGRKIYSINEDLVFLRPFSEVESILNQSFCSRRPLRLLVATKAKEIIKIP

SEQ ID NO: 727 - protein, 103 aa, Homo sapiens
PDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNH

SEQ ID NO: 728 - protein, 100 aa, Homo sapiens
LQGGDEKKVNLVLGDGRSLGLTIRGGAEYGLGIYITGVDPGSEAEGSGLKVGDQILEVNWRSFLNILHDEAVRLLKSSRHLILTVKDVGRLPHARTTVDE

SEQ ID NO: 729 - protein, 87 aa, Homo sapiens
WTSGAHVHSGPCEEKCGHPGHRQPLPRIVTIQRGGSAHNCGQLKVGHVILEVNGLTLRGKEHREAARIIAEAFKTKDRDYIDFLDSL

SEQ ID NO: 730 - protein, 100 aa, Homo sapiens
ELRRAELVEIIVETEAQTGVSGINVAGGGKEGIFVRELREDSPAARSLSLQEGDQLLSARVFFENFKYEDALRLLQCAEPYKVSFCLKRTVPTGDLALRP

SEQ ID NO: 731 - protein, 102 aa, Homo sapiens
PSQLKGVLVRASLKKSTMGFGFTIIGGDRPDEFLQVKNVLKDGPAAQDGKIAPGDVIVDINGNCVLGHTHADVVQMFQLVPVNQYVNLTLCRGYPLPDDSED

SEQ ID NO: 732 - protein, 100 aa, Homo sapiens
ASSGSSQPELVTIPLIKGPKGFGFAIADSPTGQKVKMILDSQWCQGLQKGDIIKEIYHQNVQNLTHLQVVEVLKQFPVGADVPLLILRGGPPSPTKTAKM

SEQ ID NO: 733 - protein, 143 aa, Homo sapiens
LYEDKPPLTNTFLISNPRTTADPRILYEDKPPNTKDLDVFLRKQESGFGFRVLGGDGPDQSIYIGAIIPLGAAEKDGRLRAADELMCIDGIPVKGKSHKQVLDLMTTAARNGHVLLTVRRKIFYGEKQPEDDSGSPGIHRELT

SEQ ID NO: 734 - protein, 102 aa, Homo sapiens
PAPQEPYDVVLQRKENEGFGFVILTSKNKPPPGVIPHKIGRVIEGSPADRCGKLKVGDHISAVNGQSIVELSHDNIVQLIKDAGVTVTLTVIAEEEHHGPPS

SEQ ID NO: 735 - protein, 98 aa, Homo sapiens
QNLGCYPVELERGPRGFGFSLRGGKEYNMGLFILRLAEDGPAIKDGRIHVGDQIVEINGEPTQGITHTRAIELIQAGGNKVLLLLRPGTGLIPDHGLA

SEQ ID NO: 736 - protein, 84 aa, Homo sapiens
ITVVELIKKEGSTLGLTISGGTDKDGKPRVSNLRPGGLAARSDLLNIGDYIRSVNGIHLTRLRHDEIITLLKNVGERVVLEVEY

SEQ ID NO: 737 - protein, 92 aa, Homo sapiens
ILDVSLYKEGNSFGFVLRGGAHEDGHKSRPLVLTYVRPGGPADREGSLKVGDRLLSVDGIPLHGASHATALATLRQCSHEALFQVEYDVATP

SEQ ID NO: 738 - protein, 102 aa, Homo sapiens
IHTVANASGPLMVEIVKTPGSALGISLTTTSLRNKSVITIDRIKPASVVDRSGALHPGDHILSIDGTSMEHCSLLEATKLLASISEKVRLEILPVPQSQRPL

SEQ ID NO: 739 - protein, 103 aa, Homo sapiens
IQIVHTETTEVVLCGDPLSGFGLQLQGGIFATETLSSPPLVCFIEPDSPAERCGLLQVGDRVLSINGIATEDGTMEEANQLLRDAALAHKVVLEVEFDVAESV

SEQ ID NO: 740 - protein, 103 aa, Homo sapiens
IQFDVAESVIPSSGTFHVKLPKKRSVELGITISSASRKRGEPLIISDIKKGSVAHRTGTLEPGDKLLAIDNIRLDNCPMEDAVQILRQCEDLVKLKIRKDEDN

SEQ ID NO: 741 - protein, 94 aa, Homo sapiens
IQTTGAVSYTVELKRYGGPLGITISGTEEPFDPIVISGLTKRGLAERTGAIHVGDRILAINNVSLKGRPLSEAIHLLQVAGETVTLKIKKQLDR

SEQ ID NO: 742 - protein, 105 aa, Homo sapiens
ILEMEELLLPTPLEMHKVTLHKDPMRHDFGFSVSDGLLEKGVYVHTVRPDGPAHRGGLQPFDRVLQVNHVRTRDFDCCLAVPLLAEAGDVLELIISRKPHTAHSS

SEQ ID NO: 743 - protein, 91 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQT

SEQ ID NO: 744 - protein, 84 aa, Homo sapiens
SNYSVSLVGPAPWGFRLQGGKDFNMPLTISSLKDGGKAAQANVRIGDVVLSIDGINAQGMTHLEAQNKIKGCTGSLNMTLQRAS

SEQ ID NO: 745 - protein, 82 aa, Homo sapiens
IHSVTLRGPSPWGFRLVGRDFSAPLTISRVHAGSKASLAALCPGDLIQAINGESTELMTHLEAQNRIKGCHDHLTLSVSRPE

SEQ ID NO: 746 - protein, 133 aa, Homo sapiens
TLVEHSKLYCGHCYYQTVVTPVIEQILPDSPGSHLPHTVTLVSIPASSHGKRGLSVSIDPPHGPPGCGTEHSHTVRVQGVDPGCMSPDVKNSIHVGDRILEINGTPIRNVPLDEIDLLIQETSRLLQLTLEHD

SEQ ID NO: 747 - protein, 92 aa, Homo sapiens
PYSVTLISMPATTEGRRGFSVSVESACSNYATTVQVKEVNRMHISPNNRNAIHPGDRILEINGTPVRTLRVEEVEDAISQTSQTLQLLIEHD

SEQ ID NO: 748 - protein, 74 aa, Homo sapiens
VCYRTDDEEDLGIYVGEVNPNSIAAKDGRIREGDRIIQINGVDVQNREEAVAILSQEENTNISLLVARPESQLA

SEQ ID NO: 749 - protein, 98 aa, Homo sapiens
SENCKDVFIEKQKGEILGVVIVESGWGSILPTVIIANMMHGGPAEKSGKLNIGDQIMSINGTSLVGLPLSTCQSIIKGLKNQSRVKLNIVRCPPVNSS

SEQ ID NO: 750 - protein, 92 aa, Homo sapiens
LRCPPVTTVLIRRPDLRYQLGFSVQNGIICSLMRGGIAERGGVRVGHRIIEINGQSVVATPHEKIVHILSNAVGEIHMKTMPAAMYRLLNSS

SEQ ID NO: 751 - protein, 103 aa, Homo sapiens
LSNSDNCREVHLEKRRGEGLGVALVESGWGSLLPTAVIANLLHGGPAERSGALSIGDRLTAINGTSLVGLPLAACQAAVRETKSQTSVTLSIVHCPPVTTAIM

SEQ ID NO: 752 - protein, 92 aa, Homo sapiens
LVHCPPVTTAIIHRPHAREQLGFCVEDGIICSLLRGGIAERGGIRVGHRIIEINGQSVVATPHARIIELLTEAYGEVHIKTMPAATYRLLTG

SEQ ID NO: 753 - protein, 86 aa, Homo sapiens
RKVRLIQFEKVTEEPMGITLKLNEKQSCTVARILHGGMIHRQGSLHVGDEILEINGTNVTNHSVDQLQKAMKETKGMISLKVIPNQ

SEQ ID NO: 754 - protein, 89 aa, Homo sapiens
PVPPDAVRMVGIRKTAGEHLGVTFRVEGGELVIARILHGGMVAQQGLLHVGDIIKEVNGQPVGSDPRALQELLRNASGSVILKILPNYQ

SEQ ID NO: 755 - protein, 99 aa, Homo sapiens
QGRHVEVFELLKPPSGGLGFSVVGLRSENRGELGIFVQEIQEGSVAHRDGRLKETDQILAINGQALDQTITHQQAISILQKAKDTVQLVIARGSLPQLV

SEQ ID NO: 756 - protein, 97 aa, Homo sapiens
PVHWQHMETIELVNDGSGLGFGIIGGKATGVIVKTILPGGVADQHGRLCSGDHILKIGDTDLAGMSSEQVAQVLRQCGNRVKLMIARGAIEERTAPT

SEQ ID NO: 757 - protein, 98 aa, Homo sapiens
QESETFDVELTKNVQGLGITIAGYIGDKKLEPSGIFVKSITKSSAVEHDGRIQIGDQIIAVDGTNLQGFTNQQAVEVLRHTGQTVLLTLMRRGMKQEA

SEQ ID NO: 758 - protein, 92 aa, Homo sapiens
LNYEIVVAHVSKFSENSGLGISLEATVGHHFIRSVLPEGPVGHSGKLFSGDELLEVNGITLLGENHQDVVNILKELPIEVTMVCCRRTVPPT

SEQ ID NO: 759 - protein, 100 aa, Homo sapiens
WEAGIQHIELEKGSKGLGFSILDYQDPIDPASTVIIIRSLVPGGIAEKDGRLLPGDRLMFVNDVNLENSSLEEAVEALKGAPSGTVRIGVAKPLPLSPEE

SEQ ID NO: 760 - protein, 99 aa, Homo sapiens
RNVSKESFERTINIAKGNSSLGMTVSANKDGLGMIVRSIIHGGAISRDGRIAIGDCILSINEESTISVTNAQARAMLRRHSLIGPDIKITYVPAEHLEE

SEQ ID NO: 761 - protein, 112 aa, Homo sapiens
LNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRKSPLPSLL

SEQ ID NO: 762 - protein, 95 aa, Homo sapiens
LTGELHMIELEKGHSGLGLSLAGNKDRSRMSVFIVGIDPNGAAGKDGRLQIADELLEINGQILYGRSHQNASSIIKCAPSKVKIIFIRNKDAVNQ

SEQ ID NO: 763 - protein, 94 aa, Homo sapiens
LSSFKNVQHLELPKDQGGLGIAISEEDTLSGVIIKSLTEHGVAATDGRLKVGDQILAVDDEIVVGYPIEKFISLLKTAKMTVKLTIHAENPDSQ

SEQ ID NO: 764 - protein, 95 aa, Homo sapiens
LPGCETTIEISKGRTGLGLSIVGGSDTLLGAIIIHEVYEEGAACKDGRLWAGDQILEVNGIDLRKATHDEAINVLRQTPQRVRLTLYRDEAPYKE

SEQ ID NO: 765 - protein, 98 aa, Homo sapiens
KEEEVCDTLTIELQKKPGKGLGLSIVGKRNDTGVFVSDIVKGGIADADGRLMQGDQILMVNGEDVRNATQEAVAALLKCSLGTVTLEVGRIKAGPFHS

SEQ ID NO: 766 - protein, 96 aa, Homo sapiens
LQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSV

SEQ ID NO: 767 - protein, 91 aa, Homo sapiens
LGPPQCKSITLERGPDGLGFSIVGGYGSPHGDLPIYVKTVFAKGAASEDGRLKRGDQIIAVNGQSLEGVTHEEAVAILKRTKGTVTLMVLS

SEQ ID NO: 768 - protein, 93 aa, Homo sapiens
IQYEEIVLERGNSGLGFSIAGGIDNPHVPDDPGIFITKIIPGGAAAMDGRLGVNDCVLRVNEVEVSEVVHSRAVEALKEAGPVVRLVVRRRQN

SEQ ID NO: 769 - protein, 90 aa, Homo sapiens
ITLLKGPKGLGFSIAGGIGNQHIPGDNSIYITKIIEGGAAQKDGRLQIGDRLLAVNNTNLQDVRHEEAVASLKNTSDMVYLKVAKPGSLE

SEQ ID NO: 770 - protein, 119 aa, Homo sapiens
ILLHKGSTGLGFNIVGGEDGEGIFVSFILAGGPADLSGELRRGDRILSVNGVNLRNATHEQAAAALKRAGQSVTIVAQYRPEEYSRFESKIHDLREQMMNSSMSSGSGSLRTSEKRSLE

SEQ ID NO: 771 - protein, 90 aa, Homo sapiens
IQPNVISVRLFKRKVGGLGFLVKERVSKPPVIISDLIRGGAAEQSGLIQAGDIILAVNGRPLVDLSYDSALEVLRGIASETHVVLILRGP

SEQ ID NO: 772 - protein, 107 aa, Homo sapiens
QANSDESDIIHSVRVEKSPAGRLGFSVRGGSEHGLGIFVSKVEEGSSAERAGLCVGDKITEVNGLSLESTTMGSAVKVLTSSSRLHMMVRRMGRVPGIKFSKEKNSS

SEQ ID NO: 773 - protein, 106 aa, Homo sapiens
PSDTSSEDGVRRIVHLYTTSDDFCLGFNIRGGKEFGLGIYVSKVDHGGLAEENGIKVGDQVLAANGVRFDDISHSQAVEVLKGQTHIMLTIKETGRYPAYKEMNSS

SEQ ID NO: 774 - protein, 115 aa, Homo sapiens
KIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITV

SEQ ID NO: 775 - protein, 101 aa, Homo sapiens
LLTEEEINLTRGPSGLGFNIVGGTDQQYVSNDSGIYVSRIKENGAAALDGRLQEGDKILSVNGQDLKNLLHQDAVDLFRNAGYAVSLRVQHRLQVQNGIHS

SEQ ID NO: 776 - protein, 94 aa, Homo sapiens
PVDAIRILGIHKRAGEPLGVTFRVENNDLVIARILHGGMIDRQGLLHVGDIIKEVNGHEVGNNPKELQELLKNISGSVTLKILPSYRDTITPQQ

SEQ ID NO: 777 - protein, 93 aa, Homo sapiens
DDMVKLVEVPNDGGPLGIHVVPFSARGGRTLGLLVKRLEKGGKAEHENLFRENDCIVRINDGDLRNRRFEQAQHMFRQAMRTPIIWFHVVPAA

SEQ ID NO: 778 - protein, 94 aa, Homo sapiens
GKRLNIQLKKGTEGLGFSITSRDVTIGGSAPIYVKNILPRGAAIQDGRLKAGDRLIEVNGVDLVGKSQEEVVSLLRSTKMEGTVSLLVFRQEDA

SEQ ID NO: 779 - protein, 103 aa, Homo sapiens
TPDGTREFLTFEVPLNDSGSAGLGVSVKGNRSKENHADLGIFVKSIINGGAASKDGRLRVNDQLIAVNGESLLGKTNQDAMETLRRSMSTEGNKRGMIQLIVA

SEQ ID NO: 780 - protein, 102 aa, Homo sapiens
LPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 781 - protein, 111 aa, Homo sapiens
IDVDLVPETHRRVRLHRHGCEKPLGFYIRDGASVRVTPHGLEKVPGIFISRMVPGGLAESTGLLAVNDEVLEVNGIEVAGKTLDQVTDMMIANSHNLIVTVKPANQRNNVV

SEQ ID NO: 782 - protein, 100 aa, Homo sapiens
RSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEFH

SEQ ID NO: 783 - protein, 102 aa, Homo sapiens
IPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDEF

SEQ ID NO: 784 - protein, 103 aa, Homo sapiens
PEQIMGKDVRLLRIKKEGSLDLALEGGVDSPIGKVVVSAVYERGAAERHGGIVKGDEIMAINGKIVTDYTLAEADAALQKAWNQGGDWIDLVVAVCPPKEYDD

SEQ ID NO: 785 - protein, 103 aa, Homo sapiens
LTSTFNPRECKLSKQEGQNYGFFLRIEKDTEGHLVRVVEKCSPAEKAGLQDGDRVLRINGVFVDKEEHMQVVDLVRKSGNSVTLLVLDGDSYEKAGSPGIHRD

SEQ ID NO: 786 - protein, 92 aa, Homo sapiens
RLCYLVKEGGSYGFSLKTVQGKKGVYMTDITPQGVAMRAGVLADDHLIEVNGENVEDASHEEVVEKVKKSGSRVMFLLVDKETDKREFIVTD

SEQ ID NO: 787 - protein, 112 aa, Homo sapiens
QFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAEFIVTD

SEQ ID NO: 788 - protein, 95 aa, Homo sapiens
PDTTEEVDHKPKLCRLAKGENGYGFHLNAIRGLPGSFIKEVQKGGPADLAGLEDEDVIIEVNGVNVLDEPYEKVVDRIQSSGKNVTLLVZGKNSS

SEQ ID NO: 789 - protein, 89 aa, Homo sapiens
PTVPGKVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 790 - protein, 98 aa, Homo sapiens
SQGVGPIRKVLLLKEDHEGLGISITGGKEHGVPILISEIHPGQPADRCGGLHVGDAILAVNGVNLRDTKHKEAVTILSQQRGEIEFEVVYVAPEVDSD

SEQ ID NO: 791 - protein, 97 aa, Homo sapiens
IHVTILHKEEGAGLGFSLAGGADLENKVITVHRVFPNGLASQEGTIQKGNEVLSINGKSLKGTTHHDALAILRQAREPRQAVIVTRKLTPEEFIVTD

SEQ ID NO: 792 - protein, 98 aa, Homo sapiens
TAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSK

SEQ ID NO: 793 - protein, 99 aa, Homo sapiens
LEYEEITLERGNSGLGFSIAGGTDNPHIGDDPSIFITKIIPGGAAAQDGRLRVNDSILFVNEVDVREVTHSAAVEALKEAGSIVRLYVMRRKPPAENSS

SEQ ID NO: 794 - protein, 105 aa, Homo sapiens
HVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYL

SEQ ID NO: 795 - protein, 97 aa, Homo sapiens
REDIPREPRRIVIHRGSTGLGFNIVGGEDGEGIFISFILAGGPADLSGELRKGDQILSVNGVDLRNASHEQAAIALKNAGQTVTIIAQYKPEFIVTD

SEQ ID NO: 796 - protein, 88 aa, Homo sapiens
LIRITPDEDGKFGFNLKGGVDQKMPLVVSRINPESPADTCIPKLNEGDQIVLINGRDISEHTHDQVVMFIKASRESHSRELALVIRRR

SEQ ID NO: 797 - protein, 88 aa, Homo sapiens
IRMKPDENGRFGFNVKGGYDQKMPVIVSRVAPGTPADLCVPRLNEGDQVVLINGRDIAEHTHDQVVLFIKASCERHSGELMLLVRPNA

SEQ ID NO: 798 - protein, 106 aa, Homo sapiens
PEREITLVNLKKDAKYGLGFQIIGGEKMGRLDLGIFISSVAPGGPADFHGCLKPGDRLISVNSVSLEGVSHHAAIEILQNAPEDVTLVISQPKEKISKVPSTPVHL

SEQ ID NO: 799 - protein, 95 aa, Homo sapiens
GDIFEVELAKNDNSLGISVTGGVNTSVRHGGIYVKAVIPQGAAESDGRIHKGDRVLAVNGVSLEGATHKQAVETLRNTGQVVHLLLEKGQSPTSK

SEQ ID NO: 800 - protein, 104 aa, Homo sapiens
TEENTFEVKLFKNSSGLGFSFSREDNLIPEQINASIVRVKKLFAGQPAAESGKIDVGDVILKVNGASLKGLSQQEVISALRGTAPEVFLLLCRPPPGVLPEIDT

SEQ ID NO: 801 - protein, 98 aa, Homo sapiens
ELEVELLITLIKSEKASLGFTVTKGNQRIGCYVHDVIQDPAKSDGRLKPGDRLIKVNDTDVTNMTHTDAVNLLRAASKTVRLVIGRVLELPRIPMLPH

SEQ ID NO: 802 - protein, 94 aa, Homo sapiens
MLPHLLPDITLTCNKEELGFSLCGGHDSLYQVVYISDINPRSVAAIEGNLQLLDVIHYVNGVSTQGMTLEEVNRALDMSLPSLVLKATRNDLPV

SEQ ID NO: 803 - protein, 93 aa, Homo sapiens
RPSPPRVRSVEVARGRAGYGFTLSGQAPCVLSCVMRGSPADFVGLRAGDQILAVNEINVKKASHEDVVKLIGKCSGVLHMVIAEGVGRFESCS

SEQ ID NO: 804 - protein, 104 aa, Homo sapiens
ISFSANKRWTPPRSIRFTAEEGDLGFTLRGNAPVQVHFLDPYCSASVAGAREGDYIVSIQLVDCKWLTLSEVMKLLKSFGEDEIEMKVVSLLDSTSSMHNKSAT

SEQ ID NO: 805 - protein, 109 aa, Homo sapiens
RGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYV

SEQ ID NO: 806 - protein, 111 aa, Homo sapiens
EEKTVVLQKKDNEGFGFVLRGAKADTPIEEFTPTPAFPALQYLESVDEGGVAWQAGLRTGDFLIEVNNENVVKVGHRQVVNMIRQGGNHLVLKVVTVTRNLDPDDTARKKA

SEQ ID NO: 807 - protein, 110 aa, Homo sapiens
SDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDG

SEQ ID NO: 808 - protein, 83 aa, Homo sapiens
IRLCRLVRGEQGYGFHLHGEKGRRGQFIRRVEPGSPAEAAALRAGDRLVEVNGVNVEGETHHQVVQRIKAVEGQTRLLVVDQN

SEQ ID NO: 809 - protein, 84 aa, Homo sapiens
IRHLRKGPQGYGFNLHSDKSRPGQYIRSVDPGSPAARSGLRAQDRLIEVNGQNVEGLRHAEVVASIKAREDEARLLVVDPETDE

SEQ ID NO: 810 - protein, 92 aa, Homo sapiens
PGVREIHLCKDERGKTGLRLRKVDQGLFVQLVQANTPASLVGLRFGDQLLQIDGRDCAGWSSHKAHQVVKKASGDKIVVVVRDRPFQRTVTM

SEQ ID NO: 811 - protein, 90 aa, Homo sapiens
PFQRTVTMHKDSMGHVGFVIKKGKIVSLVKGSSAARNGLLTNHYVCEVDGQNVIGLKDKKIMEILATAGNVVTLTIIPSVIYEHIVEFIV

SEQ ID NO: 812 - protein, 88 aa, Homo sapiens
LEIKQGIREVILCKDQDGKIGLRLKSIDNGIFVQLVQANSPASLVGLRFGDQVLQINGENCAGWSSDKAHKVLKQAFGEKITMRIHRD

SEQ ID NO: 813 - protein, 75 aa, Homo sapiens
RDRPFERTITMHKDSTGHVGFIFKNGKITSIVKDSSAARNGLLTEHNICEINGQNVIGLKDSQIADILSTSGNSS

SEQ ID NO: 814 - protein, 94 aa, Homo sapiens
QRRRVTVRKADAGGLGISIKGGRENKMPILISKIFKGLAADQTEALFVGDAILSVNGEDLSSATHDEAVQVLKKTGKEVVLEVKYMKDVSPYFK

SEQ ID NO: 815 - protein, 89 aa, Homo sapiens
IRVVKQEAGGLGISIKGGRENRMPILISKIFPGLAADQSRALRLGDAILSVNGTDLRQATHDQAVQALKRAGKEVLLEVKFIREFIVTD

SEQ ID NO: 816 - protein, 101 aa, Homo sapiens
EPFYSGERTVTIRRQTVGGFGLSIKGGAEHNIPVVVSKISKEQRAELSGLLFIGDAILQINGINVRKCRHEEVVQVLRNAGEEVTLTVSFLKRAPAFLKLP

SEQ ID NO: 817 - protein, 99 aa, Homo sapiens
SHQGRNRRTVTLRRQPVGGLGLSIKGGSEHNVPVVISKIFEDQAADQTGMLFVGDAVLQVNGIHVENATHEEVVHLLRNAGDEVTITVEYLREAPAFLK

SEQ ID NO: 818 - protein, 91 aa, Homo sapiens
RGETKEVEVTKTEDALGLTITDNGAGYAFIKRIKEGSIINRIEAVCVGDSIEAINDHSIVGCRHYEVAKMLRELPKSQPFTLRLVQPKRAF

SEQ ID NO: 819 - protein, 88 aa, Homo sapiens
HSIHIEKSDTAADTYGFSLSSVEEDGIRRLYVNSVKETGLASKKGLKAGDEILEINNRAADALNSSMLKDFLSQPSLGLLVRTYPELE

SEQ ID NO: 820 - protein, 97 aa, Homo sapiens
PLNVYDVQLTKTGSVCDFGFAVTAQVDERQHLSRIFISDVLPDGLAYGEGLRKGNEIMTLNGEAVSDLDLKQMEALFSEKSVGLTLIARPPDTKATL

SEQ ID NO: 821 - protein, 103 aa, Homo sapiens
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQK

SEQ ID NO: 822 - protein, 86 aa, Homo sapiens
RKEVEVFKSEDALGLTITDNGAGYAFIKRIKEGSVIDHIHLISVGDMIEAINGQSLLGCRHYEVARLLKELPRGRTFTLKLTEPRK

SEQ ID NO: 823 - protein, 91 aa, Homo sapiens
HSHPRVVELPKTDEGLGFNVMGGKEQNSPIYISRIIPGGVAERHGGLKRGDQLLSVNGVSVEGEHHEKAVELLKAAKDSVKLVVRYTPKVL

SEQ ID NO: 824 - protein, 96 aa, Homo sapiens
ISNQKRGVKVLKQELGGLGISIKGGKENKMPILISKIFKGLAADQTQALYVGDAILSVNGADLRDATHDEAVQALKRAGKEVLLEVKYMREATPYV

SEQ ID NO: 825 - protein, 110 aa, Homo sapiens
IHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRNSS

SEQ ID NO: 826 - protein, 94 aa, Homo sapiens
IPPVTTVLIKRPDLKYQLGFSVQNGIICSLMRGGIAERGGVRVGHRIIEINGQSVVATAHEKIVQALSNSVGEIHMKTMPAAMFRLLTGQENSS

SEQ ID NO: 827 - protein, 101 aa, Homo sapiens
IWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPNSS

SEQ ID NO: 828 - protein, 95 aa, Homo sapiens
ISSQPAKPTKVTLVKSRKNEEYGLRLASHIFVKEISQDSLAARDGNIQEGDVVLKINGTVTENMSLTDAKTLIERSKGKLKMVVQRDRATLLNSS

SEQ ID NO: 829 - protein, 90 aa, Homo sapiens
IRMKLVKFRKGDSVGLRLAGGNDVGIFVAGVLEDSPAAKEGLEEGDQILRVNNVDFTNIIREEAVLFLLDLPKGEEVTILAQKKKDVFSN

SEQ ID NO: 830 - protein, 96 aa, Homo sapiens
LIWEQYTVTLQKDSKRGFGIAVSGGRDNPHFENGETSIVISDVLPGGPADGLLQENDRVVMVNGTPMEDVLHSFAVQQLRKSGKVAAIVVKRPRKV

SEQ ID NO: 831 - protein, 79 aa, Homo sapiens
RVLLMKSRANEEYGLRLGSQIFVKEMTRTGLATKDGNLHEGDIILKINGTVTENMSLTDARKLIEKSRGKLQLVVLRDS

SEQ ID NO: 832 - protein, 90 aa, Homo sapiens
HAPNTKMVRFKKGDSVGLRLAGGNDVGIFVAGIQEGTSAEQEGLQEGDQILKVNTQDFRGLVREDAVLYLLEIPKGEMVTILAQSRADVY

SEQ ID NO: 833 - protein, 106 aa, Homo sapiens
IPGNSTIWEQHTATLSKDPRRGFGIAISGGRDRPGGSMVVSDVVPGGPAEGRLQTGDHIVMVNGVSMENATSAFAIQILKTCTKMANITVKRPRRIHLPAEFIVTD

SEQ ID NO: 834 - protein, 98 aa, Homo sapiens
QDVQMKPVKSVLVKRRDSEEFGVKLGSQIFIKHITDSGLAARHRGLQEGDLILQINGVSSQNLSLNDTRRLIEKSEGKLSLLVLRDRGQFLVNIPNSS

SEQ ID NO: 835 - protein, 104 aa, Homo sapiens
RGYSPDTRVVRFLKGKSIGLRLAGGNDVGIFVSGVQAGSPADGQGIQEGDQILQVNDVPFQNLTREEAVQFLLGLPPGEEMELVTQRKQDIFWKMVQSEFIVTD

SEQ ID NO: 836 - protein, 5 aa, Artificial
  flexible polylinker
GGGGS

SEQ ID NO: 837 - protein, 14 aa, Artificial
  linker
EGKSSGSGSESKVD

SEQ ID NO: 838 - protein, 18 aa, Artificial
  linker
KESGSVSSEQLAQFRSLD

SEQ ID NO: 839 - DNA, 33 bp, Artificial
  sequence unique to vector pDsRED1-N1(+ATG)
attgccaccatgggaattctggatccgggagat

SEQ ID NO: 840 - protein, 15 aa, Artificial
  linker between cloned insert and red fluorescent protein (RFP)
LQSTVPRARDPPVAT

SEQ ID NO: 841 - protein, 10 aa, Artificial
  linker between cloned insert and red fluorescent protein (RFP)
LDPGYPPVAT

(19) United States
(12) Patent Application Publication
(10) Pub. No.: US 2005/0282743 A1
(43) Pub. Date: Dec. 22, 2005

(54) Molecular interactions in cells

(75) Inventors: Peter S. Lu, Mountain View, CA (US); Joshua . Rabinowitz, Mountain View, CA (US); Johannes Schweizer, Mountain View, CA (US); Deanna Marie Carrick, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 10/485,788

(22) PCT Filed: Aug. 2, 2002
(86) PCT No.: PCT/US02/24655

§ 371 Date: Aug. 2, 2004

Related U.S. Application Data
(60) Provisional application No. 60/309,841, filed on Aug. 3, 2001.
(60) Provisional application No. 60/360,016, filed on Feb. 28, 2002.

(51) Int. Cl. A61K 38/54

(52) U.S. Cl. 514/12

(57) ABSTRACT

The invention provides reagents and methods for inhibiting or enhancing interactions between proteins in cells, particularly interactions between a PDZ protein and a PL protein. Reagents and methods that are provided are useful for treatment of a variety of diseases and conditions in a variety of cell types.

### CROSS-REFERENCES TO RELATED APPLICATIONS

This application is the U.S. National Phase of PCT/US02/24655, filed Aug. 2, 2002, which claims the benefit of U.S. Provisional Application No. 60/309,841, filed Aug. 3, 2001 and U.S. Provisional Application No. 60/360,061, filed Feb. 25, 2002, each of which is incorporated herein by reference in its entirety for all purposes.

### FIELD OF THE INVENTION

Peptides and peptide analogues, and methods for using such compositions, to regulate various biological functions of cells are provided. For example, certain peptides and peptide analogues which are provided are utilized in methods for modulating a biological function in certain cells by antagonizing or promoting binding between a protein having a PDZ domain and a protein that binds a PDZ domain. Also provided are methods for identifying compounds that modulate the interactions between specific PDZ domains and their ligands.

### BACKGROUND

PDZ domains of proteins are named after three prototypical proteins: post-synaptic density protein 95 (PSD95), Drosophila large disc protein (Dlg1) and Zonula Occludin 1 protein (ZO-1; Gomperts et al., 1996, Cell 84:659-662). PDZ domains contain the signature sequence GLGF (SEQ ID NO:231). The first PDZ proteins were identified as functioning to concentrate receptors at neuronal synapses or tight junctions. In the nervous system, typical PDZ domain-containing proteins contain three PDZ domains, one SH3 domain and one guanylate kinase domain. Examples of intracellular PDZ domain-containing proteins include LIN-2, LIN-7 and LIN-10 at the pre-synapse, and PSD95 at the post-synapse. PDZ domains have been shown to bind the carboxyl termini of transmembrane proteins in neuronal cells. Songyang et al. reported that proteins capable of binding PDZ domains contain a carboxyl terminal motif sequence of E-S/T-X-V/I (Songyang et al., 1997, Science 275:73). X-ray crystallography studies have revealed the contact points between the motif sequence and PDZ domains (Doyle et al., 1996, Cell 88:1067-1076).

The role of PDZ domain:PDZ ligand (PL) interactions in human disease has only recently begun to be studied. Deletions that remove the PL of the human Cystic Fibrosis Transmembrane Conductance regulator (CFTR) have been correlated with an increase in Cystic Fibrosis and underscore the importance of proper PDZ:PL function (Benharouga et al 2001, J. Cell. Biol. 153:957-70). Mouse gene disruptions in the PDZ domain-containing protein Shroom result in neural tube defects, a precursor to such disorders as exencephaly, acrania, facial clefting and spina bifida (Hildebrand and Soriano, 1999, Cell 99:485-497). In a similar manner, knockout mice at the Cypher gene locus (another PDZ domain-containing protein) result in a severe form of congenital myopathy and post-natally (Zhou et al 2001, J. Cell Biol. 155:605-12).

Given the paucity of information regarding the role that PDZ proteins play in biological functions and their role in disease, further information on interactions involving proteins with PDZ domains would be useful in understanding a number of different biological functions in cells and for the treatment of human disorders.

### SUMMARY

Methods and compositions for modulating biological function in a variety of cell types (e.g., hematopoietic, neuronal, brain, stem, epidermal and epithelial) are provided herein. These methods and compositions can be utilized to treat various maladies including, but not limited to, diseases such as immune disorders, nervous system disorders and muscle disorders, for example. More specifically, these methods and compositions are for modulating binding between certain PDZ proteins and PL protein binding pairs as shown in TABLE 7. Other methods and compositions are for modulating binding between PDZ protein and PL protein binding pairs as listed in TABLE 12.

Certain methods involve introducing into the cell an agent that alters binding between a PDZ protein and a PL protein in the cell, whereby the biological function is modulated in the cell, and wherein the PDZ protein and PL protein are a binding pair as specified in TABLE 7 or TABLE 12. In some of these methods, the agent is a polypeptide comprising at least the two, three or four carboxy-terminal residues of the PL protein.

The PDZ proteins and PL proteins that have been identified as interacting can be classified into a number of different groups, and provide an indication of the diverse functions that can be modulated using the methods and compounds that are provided herein. For example, the PDZ proteins can be: 1) an enzyme such as a protein kinase, a guanalyte kinase, a tyrosine phosphatase or a serine phosphatase, 2) a LIM protein, 3) a guanine exchange factor, or 4) a viral oncogene interacting protein. Likewise, PL proteins can be 1) a T-cell surface receptor or a B-cell surface receptor, 2) a natural killer surface receptor, a monocyte cell surface receptor, or a granulocyte cell surface receptor, 3) an endothelial cell surface receptor, 4) a G-protein linked receptor or a regulator of G-protein signaling, 5) an adhesion protein or a tight junction integral membrane protein, 6) a viral oncogene, 7) neuron membrane transport protein, 8) a receptor kinase, 9) an ion channel or transporter protein, or 10) a tumor suppressor protein.

Modulation can be conducted in vitro or in vivo. If done in vitro, the cell into which the agent is introduced can be a cell within a cell culture.

Screening methods to identify compounds that modulate binding between PDZ proteins and PL peptides or proteins are also provided. Some screening methods involve contacting under suitable binding conditions (i) a PDZ-domain polypeptide having a sequence from a PDZ protein, and (ii) a PL peptide, wherein the PL peptide comprises a C-terminal sequence of the PL protein, the PDZ-domain polypeptide and the PL peptide are a binding pair as specified in TABLES 7 or 12; and contacting is performed in the presence of the test compound. Presence or absence of complex is then detected. The presence of the complex at a level that is statistically significantly higher in the presence of the test compound than in the absence of test compound is an indication that the test compound is an agonist, whereas, the presence of the complex at a level that is statistically significantly lower in the presence of the test compound than in the absence of test compound is an indication that the test compound is an antagonist.

Modulators of binding between a PDZ protein and a PL protein are also described herein. In certain instances, the modulator is (a) a peptide comprising at least 3 residues of a C-terminal sequence of a PL protein, and wherein the PDZ protein and the PL protein are a binding pair as specified in TABLES 7 or 12; or (b) a peptide mimetic of the peptide of section (a); or (c) a small molecule having similar functional activity with respect to the PDZ and PL protein binding pair as the peptide of section (a). The modulator can be either an agonist or antagonist. Such modulators can be formulated as a pharmaceutical composition.

Methods of treating a disease correlated with binding between a PDZ protein and a PL protein are also disclosed herein, the method comprising administering a therapeutically effective amount of a modulator as provided herein, wherein the PDZ protein and the PL protein are a binding pair as specified in TABLES 7 or 12. As indicated supra, such methods can be used to treat a variety of diseases including, but not limited to, neurological disease, an immune response disease, a muscular disease, or a cancer. The methods can be used to treat humans and non-human animals, including for example, cattle, swine, sheep, dogs, cats, horses and the like.

### BRIEF DESCRIPTION OF THE DRAWINGS

FIGS. 1A and 1B shows the results of introduction of a Tat-CD3 fusion peptide on T cell activation. Antigen-specific T cell activation was measured by cytokine production. Fusion peptides containing tat and a T cell surface molecule carboxyl terminus inhibited γ-interferon (IFN) production by a T cell line in response to myelin basic protein (MBP) stimulation. The level of inhibition was determined by first subtracting the binding of the labeled peptide to GST alone from the binding to the fusion protein and dividing by the signal in the absence of competitor peptide.

FIGS. 2A, 2B and 2C show binding and competition assays with the PDZ ligands of CD95 (Fas) and Tax for the PDZ domain of TIP-1. FIG. 2A shows a titration of Tax and CD95 PDZ ligands against a constant amount of TIP-1 protein. FIG. 2B shows the ability of an unlabeled 8 amino acid peptide corresponding to the C-terminus of Tax to inhibit the binding of 20 uM CD95 to TIP-1. FIG. 2C shows the ability of an unlabeled 8 amino acid peptide corresponding to the C-terminus of CD95 to inhibit the binding of 1 uM Tax to TIP-1.

### DESCRIPTION

I. Definitions

A “fusion protein” or “fusion polypeptide” as used herein refers to a composite protein, i.e., a single contiguous amino acid sequence, made up of two (or more) distinct, heterologous polypeptides that are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art.

A “fusion protein construct” as used herein is a polynucleotide encoding a fusion protein.

As used herein, the term “PDZ domain” refers to protein sequence (i.e., modular protein domain) of approximately 90 amino acids, characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF (SEQ ID NO:231) repeats. PDZ domains generally appear to maintain a core consensus sequence (Doyle, D. A., 1996, Cell 85: 1067-76).

PDZ domains are found in diverse membrane-associated proteins including members of the MAGUK family of guanylate kinase homologs, several protein phosphatases and kinases, neuronal nitric oxide synthase, and several dystrophin-associated proteins, collectively known as syntrophins.

Exemplary PDZ domain-containing proteins and PDZ domain sequences are shown in TABLE 9. The term “PDZ domain” also encompasses variants (e.g., naturally occurring variants) of the sequences of TABLE 9 (e.g., polymorphic variants, variants with conservative substitutions, and the like). Typically, PDZ domains are substantially identical to those shown in TABLE 9, e.g., at least about 70%, at least about 80%, or at least about 90% amino acid residue identity when compared and aligned for maximum correspondence.

As used herein, the term “PDZ protein” refers to a naturally occurring protein containing a PDZ domain. Exemplary PDZ proteins include CASK, MPP1, DLG1, PSD95, NeDLG, TIP-33, SYN1a, TIP-43, LDP, LIM, LIMK1, LIMK2, MPP2, NOS1, AF6, PTN-4, prIL16, 41.8kD, KIAA0559, RGS12, KIAA0316, DVL1, TIP-40, TIAM1, MINT1, KIAA0303, CBP, MINT3, TIP-2, KIAA0561, and those listed in TABLE 9.

As used herein, the term “PDZ-domain polypeptide” refers to a polypeptide containing a PDZ domain, such as a fusion protein including a PDZ domain sequence, a naturally occurring PDZ protein, or an isolated PDZ domain peptide.

As used herein, the term “PL protein” or “PDZ Ligand protein” refers to a naturally occurring protein that forms a molecular complex with a PDZ-domain, or to a protein whose carboxy-terminus, when expressed separately from the full length protein (e.g., as a peptide fragment of 4-25 residues, e.g., 8, 10, 12, 14 or 16 residues), forms such a molecular complex. The molecular complex can be observed in vitro using the “A assay” or “G assay” described infra, or in vivo. Exemplary PL proteins listed in TABLE 8 are demonstrated to bind specific PDZ proteins. This definition is not intended to include anti-PDZ antibodies and the like.

As used herein, a “PL sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence”).

As used herein, a “PL peptide” is a peptide of having a sequence from, or based on, the sequence of the C-terminus of a PL protein. Exemplary PL peptides (biotinylated) are listed in TABLE 8.

As used herein, a “PL fusion protein” is a fusion protein that has a PL sequence as one domain, typically as the C-terminal domain of the fusion protein. An exemplary PL fusion protein is a tat-PL sequence fusion.

As used herein, the term “PL inhibitor peptide sequence” refers to a PL peptide amino acid sequence that (in the form of a peptide or PL fusion protein) inhibits the interaction between a PDZ domain polypeptide and a PL peptide (e.g., in an A assay or a G assay).

As used herein, a “PDZ-domain encoding sequence” means a segment of a polynucleotide encoding a PDZ domain. In various embodiments, the polynucleotide is DNA, RNA, single stranded or double stranded.

As used herein, the terms “antagonist” and “inhibitor,” when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

As used herein, the terms “agonist” and “enhancer,” when used in the context of modulating a binding interaction (such as the binding of a PDZ domain sequence to a PL sequence), are used interchangeably and refer to an agent that increases the binding of the, e.g., PL sequence (e.g., PL peptide) and the, e.g., PDZ domain sequence (e.g., PDZ protein, PDZ domain peptide).

As used herein, the terms “peptide mimetic,” “peptidomimetic,” and “peptide analog” are used interchangeably and refer to a synthetic chemical compound that has substantially the same structural and/or functional characteristics of a PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a PDZ domain and/or inhibiting a PL-PDZ interaction.

Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (DCC) or N,N=-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R═—N—C—N—R═) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines, to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4,pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypeptide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R- or S-form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids can be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention are composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in TABLE 1, below. It is to be understood that TABLE 1 is for illustrative purposes only and does not purport to be an exhaustive list of amino acid residues which can comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| | Genetically | |
|---|---|---|
| Classification | Encoded | Genetically Non-Encoded |
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | M, G, P | |
| Aliphatic | A, V, L, I | t-BuA, t-BuG, MeIle, Nle, MeVal, Cha, |
| | | bAla, MeGly, Aib |
| Hydrophilic | | |
| Acidic | D, E | |
| Basic | H, K, R | Dpr, Orn, hArg, Phe(p-NH₂), DBU, |
| | | A₂BU |
| Polar | Q, N, S, T, Y | Cit, AcLys, MSO, hSer |
| Cysteine-Like | C | Pen, hCys, p-methyl Cys |

As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical or chemical means. Labels useful in the present invention include biotin for staining with labeled streptavidin conjugate, magnetic beads (e.g., Dynabeads™), fluorescent dyes (e.g., fluorescein, Texas red, rhodamine, green fluorescent protein, enhanced green fluorescent protein, and the like), radiolabels (e.g.,³H, ¹²⁵I, ³⁵S, ¹⁴C, or ³²P), enzymes ( e.g., hydrolases, particularly phosphatases such as alkaline phosphatase, esterases and glycosidases, or oxidoreductases, particularly peroxidases such as horse radish peroxidase, and others commonly used in ELISAs), substrates, cofactors, inhibitors, chemiluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels can be detected using photographic film or scintillation counters, fluorescent markers can be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and colorimetric labels are detected by simply visualizing the colored label. Thus, a label is any composition detectable by spectroscopic, photochemical, biochemical, immunochemical, electrical, optical or chemical means. The label can be coupled directly or indirectly to the desired component of the assay according to methods well known in the art. Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal generating system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody. The molecules can also be conjugated directly to signal generating compounds, e.g., by conjugation with an enzyme or fluorophore. Means of detecting labels are well known to those of skill in the art. Thus, for example, where the label is a radioactive label, means for detection include a scintillation counter, photographic film as in autoradiography, or storage phosphor imaging. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Also, simple colorimetric labels can be detected by observing the color associated with the label. It will be appreciated that when pairs of fluorophores are used in an assay, it is often preferred that they have distinct emission patterns (wavelengths) so that they can be easily distinguished.

As used herein, the term “substantially identical” in the context of comparing amino acid sequences, means that the sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty=−12, gap length penalty=−2; and width=16.

As used herein, “hematopoietic cells” include leukocytes including lymphocytes (T cells, B cells and NK cells), monocytes, and granulocytes (i.e., neutrophils, basophils and eosinophils), macrophages, dendritic cells, megakaryocytes, reticulocytes, erythrocytes, and CD34⁺ stem cells.

As used herein, the terms “test compound” or “test agent” are used interchangeably and refer to a candidate agent that can have enhancer/agonist, or inhibitor/antagonist activity, e.g., inhibiting or enhancing an interaction such as PDZ-PL binding. The candidate agents or test compounds can be any of a large variety of compounds, both naturally occurring and synthetic, organic and inorganic, and including polymers (e.g., oligopeptides, polypeptides, oligonucleotides, and polynucleotides), small molecules, antibodies (as broadly defined herein), sugars, fatty acids, nucleotides and nucleotide analogs, analogs of naturally occurring structures (e.g., peptide mimetics, nucleic acid analogs, and the like), and numerous other compounds. In certain embodiment, test agents are prepared from diversity libraries, such as random or combinatorial peptide or non-peptide libraries. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026. By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

As used herein, a “plurality” of PDZ proteins (or corresponding PDZ domains or PDZ fusion polypeptides) has its usual meaning. In some embodiments, the plurality is at least 5, and often at least 25, at least 40, or at least 60 different PDZ proteins. In some embodiments, the plurality is selected from the list of PDZ polypeptides listed in Table 9. In some embodiments, the plurality of different PDZ proteins are from (i.e., expressed in) a particular specified tissue or a particular class or type of cell. In some embodiments, the plurality of different PDZ proteins represents a substantial fraction (e.g., typically at least 50%, more often at least 80%) of all of the PDZ proteins known to be, or suspected of being, expressed in the tissue or cell(s), e.g., all of the PDZ proteins known to be present in lymphocytes or hematopoetic cells. In some embodiments, the plurality is at least 50%, usually at least 80%, at least 90% or all of the PDZ proteins disclosed herein as being expressed in a particular cell.

When referring to PL peptides (or the corresponding proteins, e.g., corresponding to those listed in TABLE 8, or elsewhere herein) a “plurality” can refer to at least 5, at least 10, and often at least 25 PLs such as those specifcally listed herein, or to the classes and percentages set forth supra for PDZ domains.

II. Overview

The present inventors have identified a number of interactions between PDZ proteins and PL proteins that can play a significant role in the biological function of certain cells and in a variety of physiological systems. As used herein, the term “biological function” in the context of a cell, refers to a detectable biological activity normally carried out by the cell, e.g., a phenotypic change such as proliferation, cell activation (e.g., T cell activation, B cell activation, T-B cell conjugate formation), cytokine release, degranulation, tyrosine phosphorylation, ion (e.g., calcium) flux, metabolic activity, apoptosis, changes in gene expression, maintenance of cell structure, cell migration, adherence to a substrate, signal transduction, cell-cell interactions, and others described herein or known in the art.

Because the interactions involve proteins that are involved in diverse physiological systems, the methods provided herein can be utilized broadly or selectively to modulate a number of different biological functions. Methods are also disclosed herein for determining whether a test compound acts as a modulator of binding between a particular PDZ protein and PL protein binding pair. Both agonists and antagonists of the binding pairs can be identified by such screening methods. Modulators so identified can subsequently be formulated as a pharmaceutical composition and used in the treatment of various diseases that are correlated with binding between a particular PDZ protein and PL protein or set of such proteins.

III. PDZ Protein and PL Protein Interactions

TABLE 7 and TABLE 12 (located at the end of the specification) list PDZ proteins and PL proteins which the current inventors have identified as binding to one another using assay methods described infra. Each page of TABLE 7 and 12 includes seven columns. The columns in each table are numbered from left to right such that the left-most column in each table is column 1 and the right-most column in each table is column 7. Thus, the first column in each table is labeled “AVC ID”; this column simply lists an internal reference number used to refer to the carboxyl-terminal amino acids of the PL proteins listed in the second column. Thus, the second column labeled “PL” lists the various PL peptides that were identified as binding a PDZ protein. All PL peptides were biotinylated at the amino-terminus and the sequences of these PL peptides are presented in TABLE 8 (see end of specification).

The PDZ protein (or proteins) that interact(s) with a PL peptide are listed in the fourth column of each table that is labeled “PDZ”. This column provides the gene name for the PDZ portion of the GST-PDZ fusion that interacts with the PDZ-ligand to the left. For PDZ domain-containing proteins with multiple domains, the domain number is listed to the right of the PDZ (i.e., in column 5 labeled “PDZ Domain”), and indicates the PDZ domain number when numbered from the amino-terminus to the carboxy-terminus.

The third column labeled “Peptide Optimal Concentration” in the tables is a number reflective of the binding interaction between the PL protein and PDZ protein. If a ‘0’ is listed, this is an indication that an interaction was observed using a PL peptide concentration of 10 uM in the assay; any other value listed is indicative of the Kd (dissociation constant) in uM determined by titration of the PL peptide on the concentration of PDZ protein listed in TABLE 7 and 12 (see infra for methods for determining Kd). The column labeled “Protein Optimal Concentration” refers to the protein concentration used to assay PL interaction (in ug/ml); a ‘0’ is indicative of 5 ug/ml protein concentration; any other value represents the concentration (in ug/ml) used to determine the dissociation constant for a given interaction.

Finally, the seventh column labeled “Classification” is another measure of the level of binding. In particular, it provides an absorbance value at 450 nm which indicates the amount of PL peptide bound to the PDZ protein. The following numerical values have the following meanings: ‘1’—A₄₅₀ nm 0-1; ‘2’—A₄₅₀ nm 1-2; ‘3’—A₄₅₀ nm 2-3; ‘4’—A₄₅₀ nm 3-4; ‘5’—A₄₅₀ nm of 4 more than 2× repeated; ‘0’—A₄₅₀ nm 0, i.e., not found to interact. Thus, higher numbers indicate stronger interactions.

Further information regarding these PL proteins and PDZ proteins is provided in TABLES 8 and 9. In particular, TABLE 8 provides a listing of the amino acid sequences of peptides used in the assays. When numbered from left to right, the first column labeled “AVC ID” provides the internal designation number used to refer to a particular PL protein and correlates with the designation used in TABLE 7 or TABLE 12. The column labeled “AVC Name” provides the name of the gene containing a predicted PDZ ligand. The third column labeled “Sequence” is the amino acid sequence of the PL protein used in the assay. The final two columns labeled “Accession No. and GI list the Genbank accession number or GI number corresponding to the sequence and gene name. As indicated supra, all peptides are biotinylated at the amino terminus and the amino acid sequences correspond to the C-terminal sequence of the gene name listed to the left.

TABLE 9 (located at the end of the specification) lists the sequences of the PDZ domains cloned into a vector (PGEX-3X vector) for production of GST-PDZ fusion proteins (Pharmacia) (see section VI (A)) below). More specifically, the first column (left to right) entitled “Gene Name” lists the name of the gene containing the PDZ domain. The second column labeled “GI” is a unique Genbank identifier for the gene used to design primers for PCR amplification of the listed sequence. The next column labeled “Domain Number” indicates the Pfam-predicted PDZ domain number, as numbered from the amino-terminus of the gene to the carboxy-terminus. The last column entitled “Sequence” provides the actual amino acid sequence inserted into the GST-PDZ expression vector as determined by DNA sequencing of the constructs.

As discussed in detail herein, the PDZ proteins listed in TABLE 7 and 12 are naturally occurring proteins containing a PDZ domain. Only significant interactions are presented in TABLE 7 and 12. Thus, the present invention is particularly directed to the detection and modulation of interactions between a PDZ protein and PL protein pair listed in TABLE 7 or in 12. As used herein the phrase “protein pair” or ‘protein binding pair” when used in reference to a PDZ protein and PL protein refers to a PL protein and PDZ protein listed in TABLE 7 or 12 which bind to one another. It should be understood that TABLE 7 and 12 are set up to show that certain PL proteins bind to a plurality of PDZ proteins. For example, in TABLE 7, PL protein CD46 (page 2 of TABLE 2) binds to the PDZ proteins KIAA0973, Mint 1, KIAA807, BAI-1, KIA0807(S), and PL protein CX43 binds to PDZ proteins ZO-2 and ZO-1.

IV. Classification of Interactions

A. General

The interactions summarized in TABLE 7 and 12 can occur in a wide variety of cell types. Examples of such cells include hematopoietic, stem, neuronal, muscle, epidermal, epithelial, endothelial, and cells from essentially any tissue such as liver, lung, placenta, uterus, kidney, ovaries, testes, stomach, colon and intestine. Because the interactions disclosed herein can occur in such a wide variety of cell types, these interactions can also play an important role in a variety of biological functions. Consequently, modulation of the interactions between PDZ proteins and PL proteins that are described herein can be utilized to regulate biological function in a wide range of cells.

In certain methods disclosed herein, the PL protein is expressed or up-regulated upon cell activation (e.g., in activated B lymphocytes, T lymphocytes) or upon entry into mitosis (e.g., up-regulation in rapidly proliferating cell populations).

B. Exemplary PDZ Classification

The PDZ proteins identified herein as interacting with particular PL proteins can be grouped into a number of different categories. Thus, as described in greater detail below, the methods and reagents that are provided herein can be utilized to modulate PDZ interactions, and thus biological functions, that are regulated or otherwise involve the following classes of proteins. It should be recognized, however, that modulation of the interactions that are identified herein can be utilized to affect biological functions involving other protein classes.

1. Protein Kinases

A number of protein kinases contain PDZ domains. Protein kinases are widely involved in cellular metabolism and regulation of protein activity through phosphorylation of amino acids on proteins. An example of this the regulation of signal transduction pathways such as T cell activation throuh the T cell Receptor, where ZAP-70 kinase function is required for transmission of the activation signal to downstream effector molecules. These molecules include, but are not limited to KIAA0303, KIAA0561, KIAA0807, KIAA0973, and CASK.

2. Guanalyte Kinases

A number of guanalyte kinases contain PDZ domains. These molecules include, but are not limited to Atrophin 1, CARD11, CARD14, DLG1, DLG2, DLG5, FLJ12615, MPP1, MPP2, NeDLG, p55T, PSD95, ZO-1, ZO-2, and ZO-3.

3. Guanine Exchange Factors

A number of guanine exchange factors contain PDZ domains. Guanine exchange factors regulate signal transduction pathways and other biological processes through facilitating the exchange of differentially phosphorylated guanine residues. These molecules include, but are not limited to GTPase, Guanine Exchange, KIAA0313, KIAA0380, KIAA0382, KIAA1389, KIAA1415, TIAM1, and TIAM2.

4. LIM PDZ's

A number of LIM PDZ's contain PDZ domains. These molecules include, but are not limited to Alpha Actinin 2, ELFIN1, ENIGMA, HEMBA 1003117, KIAA0613, KIAA0858, KIAA0631, LIM Mystique, LIM protein, LIM-RIL, LIMK1, LIMK2, and LU-1.

5. Proteins Containing Only PDZ Domains

A number of proteins contain PDZ domains without any other predicted functional domains. These include, but are not limited to 26s subunit p27, AIPC, Cytohesin Binding, EZRIN Binding Protein, FLJ00011, FLJ20075, FLJ21687, GRIP1, HEMBA1000505, KIAA0545, KIAA0967, KIAA1202, KIAA1284, KIAA1526, KIAA1620, KIAA1719, MAGGI1, Novel PDZ gene, Outer Membrane, PAR3, PAR6, PAR6 Gamma, PDZ-73, PDZK1, PICK1, PIST, prIL16, Shank 1, SIP1, SITAC-18, SYNTENIN, Syntrophin gamma 2, TIP1, TIP2, and TIP43.

6. Tyrosine Phosphatases

A number of Tyrosine phosphatases contain PDZ domains. Tyrosine phosphatases regulate biological processes such as signal transduction pathways through removal of phosphate groups required for function of the target protein. Examples of such enzymes include, but are not limited to PTN-3, PNT-4, and PTPL1.

7. Serine Proteases

A number of Serine Proteases contain PDZ domains. Proteases affect biological molecules by cleaving them to either activate or repress their functional ability. These enzymes have a variety of functions, including roles in digestion, blood coagulation and lysis of blood clots. These include, but are not limited to Novel Serine Protease, and Serine Protease.

8. Viral Oncogene Interacting Proteins That Contain PDZ Domains

A number of TAX interacting proteins contain PDZ domains. Many of these also bind to multiple viral oncoproteins such as Adenovirus E4, Papillomavirus E6, and HBV protein X. These include, but are not limited to AIPC, Connector Enhancer, DLG1, DLG2, ERBIN, FLJ00011, FLJ11215, HEMBA 1003117, INADL, KIAA0147, KIAA0807, KIAA1526, KIAA1634, LIMK1, LIM Mystique, LIM-RIL, MUPP1, NeDLG, Outer Membrane, PSD95, PTN-4, PTPL1, Syntrophin gamma 1, Syntrophin gamma 2, TAX2-like protein, TIP2, TIP1, TIP33 and TIP43.

9. Proteins Containing RA and/or RHA and/or DIL and/or IGFBP and/or WW and/or L27 and/or SAM and/or PH and/or DIX and/or DIP and/or Dishevelled and/or LRR and/or Hormone 3 and/or C2 and/or RPH3A and/or zf-TRAF and/or zf-C3HC4 and/or PID and/or NO_Synthase and/or Flavodoxin and/or FAD Binding and/or NAD Binding and/or Kazal, and/or Trypsin an/or RBD and/or RGS and/or GoLoco and/or HR1 and/or BR01 That Contain PDZ Domains

A number o proteins containing RA and/or RHA and/or DIL and/or IGFBP and/or WW and/or L27 and/or SAM and/or PH and/or DIX and/or DIP and/or Dishevelled and/or LRR and/or Hormone 3 and/or C2 and/or RPH3A and/or zf-TRAF and/or zf-C3HC4 and/or PID and/or NO_Synthase and/or Flavodoxin and/or FAD binding and/or NAD binding and/or Kazal, and/or Trypsin an/or RBD and/or RGS and/or GoLoco and/or HR1 and/or BR01 contain PDZ domains. These include, but are not limited to AF6, APXL-1, BAI-1 Associated, DVL1, DVL2, DVL3, KIAA0417, KIAA0316, KIAA0340, KIAA0559, KIAA0751, KIAA0902, KIAA1095, KIA1222, KIAA1634, MINT1, NOS1, RGS12, Rhophilin-like, Shank3, Syntrophin 1 alpha, Syntrophin beta 2, and X-11 beta.

C. Exemplary PL Classification

The PL proteins involved in the interactions listed in TABLE 7 and 12 are from a number of different classes. Consequently, the methods and reagents that are disclosed herein can be utilized to to modulate interactions involving the following classes of PL proteins to modulate a bioloigcal function in cells. The following classes, however, should not be considered exhaustive of the the types of classes of proteins whose activity can be modulated using the methods and reagents that are provided herein.

1. PL Sequences of T Cell Surface Receptors

A number of surface receptors expressed by T cells contain a PL motif sequence (PL sequence). These molecules include, but are not limited to, CD6, CD95, CDw128B (IL8 R), DNAM-1, Fas ligand (FasL), LPAP (Barclay et al., 1997, The Leucocyte Antigen Facts Book, second edition, Academic Press), CLASP-1, CLASP-2, CLASP-5, BLR-1 (CXCR5), DOCK2, PAG, and Mannose Receptor.

The C-terminal core sequence of CD6 is ISAA (SEQ ID NO:1). When naturally-occurring residues are added or removed from the core sequence, AA, SAA, DISAA (SEQ ID NO:2), DDISAA (SEQ ID NO:3), YDDISAA (SEQ ID NO:4), and DYDDISAA (SEQ ID NO:5) may also be used to target a PDZ domain-containing protein in T cells.

The C-terminal core sequence of CD95 is QSLV (SEQ ID NO:6). When naturally-occurring residues are added or removed from the core sequence, LV, SLV, IQSLV (SEQ ID NO:7), EIQSLV (SEQ ID NO:8), NEIQSLV (SEQ ID NO:9), and RNEIQSLV (SEQ ID NO:10) may also be used to target a PDZ domain-containing protein in T cells.

The C-terminal core sequence of CDw128B is STTL (SEQ ID NO:11). When naturally-occurring residues are added or removed from the core sequence, TL, TTL, TSTTL (SEQ ID NO:12), HTSTTL (SEQ ID NO:13), GHTSTTL (SEQ ID NO:14), and SGHTSTTL (SEQ ID NO:15) may also be used to target a PDZ domain-containing protein in T cells.

The C-terminal core sequence of DNAM-1 is KTRV (SEQ ID NO:16). When naturally-occurring residues are added or removed from the core sequence, RV, TRV, PKTRV (SEQ ID NO:17), RPKTRV (SEQ ID NO:18), RRPKTRV (SEQ ID NO:19), and SRRPKTRV (SEQ ID NO:20) may also be used to target a PDZ domain-containing protein in T cells.

The C-terminal core sequence of FasL is LYKL (SEQ ID NO:21). When naturally-occurring residues are added or removed from the core sequence, KL, YKL, GLYKL (SEQ ID NO:22), FGLYKL (SEQ ID NO:23), FFGLYKL (SEQ ID NO:24), and TFFGLYKL (SEQ ID NO:25) may also be used to target a PDZ domain-containing protein in T cells.

The C-terminal core sequence of LPAP is VTAL (SEQ ID NO:26). When naturally-occurring residues are added or removed from the core sequence, AL, TAL, HVTAL (SEQ ID NO:27), LHVTAL (SEQ ID NO:28), GLHVTAL (SEQ ID NO:29), and QGLHVTAL (SEQ ID NO:30) may also be used to target a PDZ domain-containing protein in T cells.

The C-terminal core sequence of CLASP-1 is SAQV (SEQ ID NO:31). When naturally-occurring residues are added or removed from the core sequence, QV, AQV, SSAQV (SEQ ID NO:32), SSSAQV (SEQ ID NO:33), ISSSAQV (SEQ ID NO:34), and SISSSAQV (SEQ ID NO:35) may also be used to target a PDZ domain-containing protein in T cells.

The C-terminal core sequence of CLASP-2 is SSVV (SEQ ID NO:36). When naturally-occurring residues are added or removed from the core sequence, VV, SVV, SSSVV (SEQ ID NO:37), SSSSVV (SEQ ID NO:38), TSSSSVV (SEQ ID NO:39), and MTSSSSVV (SEQ ID NO:40) may also be used to target a PDZ domain-containing protein in T cells.

The C-terminal core sequence of CLASP-5 is SQGS (SEQ ID NO:41). When naturally-occurring residues are added or removed from the core sequence, GS, QGS, LSQGS (SEQ ID NO:42), QLSQGS (SEQ ID NO:43), TQLSQGS (SEQ ID NO:44), and ETQLSQGS (SEQ ID NO:45) may also be used to target a PDZ domain-containing protein in T cells.

The C-terminal core sequence of BLR-1 is LTTF (SEQ ID NO:46). When naturally-occurring residues are added or removed from the core sequence, TF, TTF, SLTTF (SEQ ID NO:47), TSLTTF (SEQ ID NO:48), ATSLTTF (SEQ ID NO:49), and NATSLTTF (SEQ ID NO:50) may also be used to target a PDZ domain-containing protein in T cells.

The C-terminal core sequence of DOCK2 is STDL (SEQ ID NO:51). When naturally-occurring residues are added or removed from the core sequence, DL, TDL, LSTDL (SEQ ID NO:52), SLSTDL (SEQ ID NO:53), DSLSTDL (SEQ ID NO:54), and PDSLSTDL (SEQ ID NO:55) may also be used to target a PDZ domain-containing protein in T cells.

The C-terminal core sequence of PAG is ITRL (SEQ ID NO:56). When naturally-occurring residues are added or removed from the core sequence, RL, TRL, DITRL (SEQ ID NO:57), RDITRL (SEQ ID NO:58), GRDITRL (SEQ ID NO:59), and QGRDITRL (SEQ ID NO:60) may also be used to target a PDZ domain-containing protein in T cells.

The C-terminal core sequence of Mannose Receptor is HSVI (SEQ ID NO:61). When naturally-occurring residues are added or removed from the core sequence, VI, SVI, EHSVI (SEQ ID NO:62), NEHSVI (SEQ ID NO:63), QNEHSVI (SEQ ID NO:64), and EQNEHSVI (SEQ ID NO:65) may also be used to target a PDZ domain-containing protein in T cells.

2. PL Sequences of B Cell Surface Receptors

A number of surface receptors expressed by B cells contain a PL motif sequence (PL sequence). These molecules include, but are not limited to, CD95, CDW125 (modified) (IL5R), DNAM-1, LPAP (Barclay et al., 1997, The Leucocyte Antigen Facts Book, second edition, Academic Press), CLASP-1, CLASP-2, CLASP-5, and BLR-1. The specific motif sequences of CD95, DNAM-1, LPAP, CLASP-1, CLASP-2, CLASP-5, and BLR-1 have been described in the preceding paragraphs.

The C-terminal core sequence of CDW125 is DSVF (SEQ ID NO:66). When naturally-occurring residues are added or removed from the core sequence, VF, SVF, EDSVF (SEQ ID NO:67), LEDSVF (SEQ ID NO:68), TLEDSVF (SEQ ID NO:69), and ETLEDSVF (SEQ ID NO:70) may also be used to target a PDZ domain-containing protein in B cells.

3. PL Sequences of Natural Killer Cell Surface Receptors

A number of surface receptors expressed by NK cells contain a PL motif sequence (PL sequence). These molecules include, but are not limited to, DNAM1. The specific motif sequence of DNAM-1 has been described in the preceding paragraphs.

4. PL Sequences of Monocyte Surface Receptors

A number of surface receptors expressed by cells of the monocytic lineage (monocytes and macrophages) contain a PL motif sequence (PL sequence). These molecules include, but are not limited to, CD46, CD95, CDw128, DNAM-1, Mannose receptor, and FcεRIβ. The specific motif sequences of CD95, CDw128B, DNAM-1, and Mannose receptor have been described in the preceding paragraphs.

The C-terminal core sequence of CD46 is FTSL (SEQ ID NO:71). When naturally-occurring residues are added or removed from the core sequence, SL, TSL, KFTSL (SEQ ID NO:72), VKFTSL (SEQ ID NO:73), EVKFTSL (SEQ ID NO:74), and REVKFTSL (SEQ ID NO:75) may also be used to target a PDZ domain-containing protein in monocytes.

The C-terminal core sequence of FcεRIβ is PIDL (SEQ ID NO:76). When naturally-occurring residues are added or removed from the core sequence, DL, IDL, PPIDL (SEQ ID NO:77), SPPIDL (SEQ ID NO:78), MSPPIDL (SEQ ID NO:79), and EMSPPIDL (SEQ ID NO:80) may also be used to target a PDZ domain-containing protein in monocytes.

5. PL Sequences of Granulocyte Surface Receptors

A number of surface receptors expressed by granulocytes contain a PL motif sequence (PL sequence). These molecules include, but are not limited to, CD95, CDW125, and FcεRIβ. The specific motif sequences of CD95, CDW125, and FcεRIβ have been described in the preceding paragraphs.

6. PL Sequences of Endothelial Cell Surface Receptors

A number of surface receptors expressed by endothelial cells contain a PL motif sequence (PL sequence). These molecules include, but are not limited to, CD34, and CD46. The specific motif sequence of CD46 has been described in the preceding paragraphs.

The C-terminal core sequence of CD34 is DTEL (SEQ ID NO:81). When naturally-occurring residues are added or removed from the core sequence, EL, TEL, ADTEL (SEQ ID NO:82), VADTEL (SEQ ID NO:83), VVADTEL (SEQ ID NO:84), and HVVADTEL (SEQ ID NO:85) may also be used to target a PDZ domain-containing protein in endothelial cells.

7. PL Sequences of G-Protein Linked Receptors

A number of G-protein linked receptors contain a PL motif sequence (PL sequence). These molecules include, but are not limited to, alpha-2A Adrenergic receptor, alpha-2B Adrenergic receptor, alpha-2C Adrenergic receptor, GLUR2, GluR5-2 (rat), GLUR7, GluR delta-2, muscarinic Ach receptor M4, NMDA Glutamate Receptor 2C (cysteine-free), NMDA R2C, Serotonin receptor 3a, serotonin receptor 5-HT-2B, serotonin receptor 5-HT-2C, SSTR2 (somatostatin receptor 2), somatostatin receptor 4, IL-8RA, parathyroid hormone receptor 2, and C5 Anaphylatoxin receptor.

The C-terminal core sequence of alpha-2A Adrenergic receptor is KRIV (SEQ ID NO:86). When naturally-occurring residues are added or removed from the core sequence, IV, RIV, RKRIV (SEQ ID NO:87), DRKRIV (SEQ ID NO:85), GDRKRIV (SEQ ID NO:89), and RGDRKRIV (SEQ ID NO:90) may also be used to target a PDZ domain-containing protein in cells.

The C-terminal core sequence of alpha-2B Adrenergic receptor is QTAW (SEQ ID NO:91). When naturally-occurring residues are added or removed from the core sequence, AW, TAW, TQTAW (SEQ ID NO:92), WTQTAW (SEQ ID NO:93), PWTQTAW (SEQ ID NO:94), and RPWTQTAW (SEQ ID NO:95) may also be used to target a PDZ domain-containing protein in cells.

The C-terminal core sequence of alpha-2C Adrenergic receptor is GFRQ (SEQ ID NO:96). When naturally-occurring residues are added or removed from the core sequence, RQ, FRQ, RGFRQ (SEQ ID NO:97), RRGFRQ (SEQ ID NO:98), ARRGFRQ (SEQ ID NO:99), and RARRGFRQ (SEQ ID NO:100) may also be used to target a PDZ domain-containing protein in cells.

The C-terminal core sequence of GLUR2 is SVKI (SEQ ID NO:101). When naturally-occurring residues are added or removed from the core sequence, KI, VKI, ESVKI (SEQ ID NO:102), IESVKI (SEQ ID NO:103), GIESVKI (SEQ ID NO:104), and SGIESVKI (SEQ ID NO:105) may also be used to target a PDZ domain-containing protein in cells.

The C-terminal core sequence of GLUR5-2 is ETVA (SEQ ID NO:106). When naturally-occurring residues are added or removed from the core sequence, VA, TVA, KETVA (SEQ ID NO:107), RKETVA (SEQ ID NO:108), QRKETVA (SEQ ID NO:109), and TQRKETVA (SEQ ID NO:110) may also be used to target a PDZ domain-containing protein in cells.

The C-terminal core sequence of GLUR7 is NLVI (SEQ ID NO:111). When naturally-occurring residues are added or removed from the core sequence, VI, LVI, NNLVI (SEQ ID NO:112), YNNLVI (SEQ ID NO:113), SYNNLVI (SEQ ID NO:114), and VSYNNLVI (SEQ ID NO:115) may also be used to target a PDZ domain-containing protein in cells.

The C-terminal core sequence of GluR delta-2 is GTSI (SEQ ID NO:116). When naturally-occurring residues are added or removed from the core sequence, SI, TSI, RGTSI (SEQ ID NO:117), DRGTSI (SEQ ID NO:118), PDRGTSI (SEQ ID NO:119), and DPDRGTSI (SEQ ID NO:120) may also be used to target a PDZ domain-containing protein in cells.

The C-terminal core sequence of muscarinic Ach receptor M4 is EQAL (SEQ ID NO:121). When naturally-occurring residues are added or removed from the core sequence, AL, QAL, PEQAL (SEQ ID NO:122), APEQAL (SEQ ID NO:123), RAPEQAL (SEQ ID NO:124), and KRAPEQAL (SEQ ID NO:125) may also be used to target a PDZ domain-containing protein in cells.

The C-terminal core sequence of NMDA Glutamate Receptor 2C is ESEV (SEQ ID NO:126). When naturally-occurring residues are added or removed from the core sequence, EV, SEV, LESEV (SEQ ID NO:127), SLESEV (SEQ ID NO:128), SSLESEV (SEQ ID NO:129), and ISSLESEV (SEQ ID NO:130) may also be used to target a PDZ domain-containing protein in cells.

The C-terminal core sequence of NMDA R2C is STVV (SEQ ID NO:131). When naturally-occurring residues are added or removed from the core sequence, VV, TVV, VSTVV (SEQ ID NO:132), SVSTVV (SEQ ID NO:133), PSVSTVV (SEQ ID NO:134), and DPSVSTVV (SEQ ID NO:135) may also be used to target a PDZ domain-containing protein in cells.

The C-terminal core sequence of Serotonin receptor 3a is WQYA (SEQ ID NO:136). When naturally-occurring residues are added or removed from the core sequence, YA, QYA, IWQYA (SEQ ID NO:137), SIWQYA (SEQ ID NO:138), WSIWQYA (SEQ ID NO:139), and LWSIWQYA (SEQ ID NO:140) may also be used to target a PDZ domain-containing protein in cells.

The C-terminal core sequence of serotonin receptor 5-HT-2B is VSYV (SEQ ID NO:141). When naturally-occurring residues are added or removed from the core sequence, YV, SYV, QVSYV (SEQ ID NO:141), EQVSYV (SEQ ID NO:143), EEQVSYV (SEQ ID NO:144), and TEEQVSYV (SEQ ID NO:145) may also be used to target a PDZ domain-containing protein in cells.

The C-terminal core sequence of serotonin receptor 5-HT-2C is ISSV (SEQ ID NO:146). When naturally-occurring residues are added or removed from the core sequence, SV, SSV, RISSV (SEQ ID NO:147), ERISSV (SEQ ID NO:148), SERISSV (SEQ ID NO:149), and VSERISSV (SEQ ID NO:150) may also be used to target a PDZ domain-containing protein in cells.

The C-terminal core sequence of SSTR 2 is QTSI (SEQ ID NO:151). When naturally-occurring residues are added or removed from the core sequence, SI, TSI, LQTSI (SEQ ID NO:152), DLQTSI (SEQ ID NO:153), GDLQTSI (SEQ ID NO:154), and NGDLQTSI (SEQ ID NO:155) may also be used to target a PDZ domain-containing protein in cells.

The C-terminal core sequence of somatostatin receptor 4 is TTTF (SEQ ID NO:156). When naturally-occurring residues are added or removed from the core sequence, TF, TTF, RTTTF (SEQ ID NO:157), TRTTTF (SEQ ID NO:158), LTRTTTF (SEQ ID NO:159), and PLTRTTTF (SEQ ID NO:160) may also be used to target a PDZ domain-containing protein in cells.

The C-terminal core sequence of IL-8RA is SSNL (SEQ ID NO:161). When naturally-occurring residues are added or removed from the core sequence, NL, SNL, VSSNL (SEQ ID NO:162), NVSSNL (SEQ ID NO:163), VNVSSNL (SEQ ID NO:164), and SVNVSSNL (SEQ ID NO:165) may also be used to target a PDZ domain-containing protein in cells.

The C-terminal core sequence of parathyroid hormone receptor 2 is EDVL (SEQ ID NO:166). When naturally-occurring residues are added or removed from the core sequence, VL, DVL, TEDVL (SEQ ID NO:167), ETEDVL (SEQ ID NO:168), GETEDVL (SEQ ID NO:169), and QGETEDVL (SEQ ID NO:170) may also be used to target a PDZ domain-containing protein in cells.

The C-terminal core sequence of C5 Anaphylatoxin receptor is TQAV (SEQ ID NO:171). When naturally-occurring residues are added or removed from the core sequence, AV, QAV, KTQAV (SEQ ID NO:172), QKTQAV (SEQ ID NO:173), AQKTQAV (SEQ ID NO:174), and MAQKTQAV (SEQ ID NO:175) may also be used to target a PDZ domain-containing protein in cells.

8. PL Sequences of Viral Oncogenes

A number of viral oncogenes and viral oncogene homologues contain a PL motif sequence (PL sequence). These molecules include, but are not limited to, AdenoE4 typ9, AKT1, HPV E6 #16 (Modified), HPV E6 #18, HPV E6 33 (modified), HPV E6 #35 (cysteine-free), HPV E6 52 (modified), HPV E6 #57 (cysteine-free), HPV E6 58 (modified), HPV E6 #66 (cysteine-free), HPV E6 77 (modified), and TAX.

The C-terminal core sequence of AdenoE4 typ9 is ATLV (SEQ ID NO:176). When naturally-occurring residues are added or removed from the core sequence, LV, TLV, IATLV (SEQ ID NO:177), KIATLV (SEQ ID NO:178), VKIATLV (SEQ ID NO:179), and SVKIATLV (SEQ ID NO:180) may also be used to target a PDZ domain-containing protein in cells.

The C-terminal core sequence of AKT1 is SSTA (SEQ ID NO:181). When naturally-occurring residues are added or removed from the core sequence, TA, STA, ASSTA (SEQ ID NO:182), SASSTA (SEQ ID NO:183), YSASSTA (SEQ ID NO:184), and SYSASSTA (SEQ ID NO:185) may also be used to target a PDZ domain-containing protein in cells.

The C-terminal core sequence of HPV E6 #16 is ETQL (SEQ ID NO:186). When naturally-occurring residues are added or removed from the core sequence, QL, TQL, RETQL (SEQ ID NO:187), RRETQL (SEQ ID NO:188), TRRETQL (SEQ ID NO:189), and RTRRETQL (SEQ ID NO:190) may also be used to target a PDZ domain-containing protein in cells.

The C-terminal core sequence of HPV E6 #18 is ETQV (SEQ ID NO:191). When naturally-occurring residues are added or removed from the core sequence, QV, TQV, RETQV (SEQ ID NO:192), RRETQV (SEQ ID NO:193), RRRETQV (SEQ ID NO:194), and QRRRETQV (SEQ ID NO:195) may also be used to target a PDZ domain-containing protein in cells.

The C-terminal core sequence of HPV E6 33 is ETAL (SEQ ID NO:196). When naturally-occurring residues are added or removed from the core sequence, AL, TAL, RETAL (SEQ ID NO:197), RRETAL (SEQ ID NO:198), GRRETAL (SEQ ID NO:199), and QGRRETAL (SEQ ID NO:200) may also be used to target a PDZ domain-containing protein in cells.

The C-terminal core sequence of HPVE6 #35 is ETEV (SEQ ID NO:201). When naturally-occurring residues are added or removed from the core sequence, EV, TEV, RETEV (SEQ ID NO:202), RRETEV (SEQ ID NO:203), TRRETEV (SEQ ID NO:204), and PTRRETEV (SEQ ID NO:205) may also be used to target a PDZ domain-containing protein in cells.

The C-terminal core sequence of HPV E6 52 is VTQV (SEQ ID NO:206). When naturally-occurring residues are added or removed from the core sequence, QV, TQV, RVTQV (SEQ ID NO:207), RRVTQV (SEQ ID NO:208), GRRVTQV (SEQ ID NO:209), and QGRRVTQV (SEQ ID NO:210) may also be used to target a PDZ domain-containing protein in cells.

The C-terminal core sequence of HPV E6 #57 is RTSH (SEQ ID NO:211). When naturally-occurring residues are added or removed from the core sequence, SH, TSH, LRTSH (SEQ ID NO:212), ALRTSH (SEQ ID NO:213), PALRTSH (SEQ ID NO:214), and APALRTSH (SEQ ID NO:215) may also be used to target a PDZ domain-containing protein in cells.

The C-terminal core sequence of HPV E6 58 is QTQV (SEQ ID NO:216). When naturally-occurring residues are added or removed from the core sequence, QV, TQV, RQTQV (SEQ ID NO:217), RRQTQV (SEQ ID NO:218), GRRQTQV (SEQ ID NO:219), and QGRRQTQV (SEQ ID NO:220) may also be used to target a PDZ domain-containing protein in cells.

The C-terminal core sequence of HPV E6 #66 is ESTV (SEQ ID NO:221). When naturally-occurring residues are added or removed from the core sequence, TV, STV, TESTV (SEQ ID NO:222), ATESTV (SEQ ID NO:223), QATESTV (SEQ ID NO:224), and RQATESTV (SEQ ID NO:225) may also be used to target a PDZ domain-containing protein in cells.

The C-terminal core sequence of HPV E6 77 is QSRQ (SEQ ID NO:226). When naturally-occurring residues are added or removed from the core sequence, RQ, SRQ, GQSRQ (SEQ ID NO:227), GGQSRQ (SEQ ID NO:228), GGGQSRQ (SEQ ID NO:229), and RGGGQSRQ (SEQ ID NO:230) may also be used to target a PDZ domain-containing protein in cells.

The C-terminal core sequence of TAX is ETEV (SEQ ID NO:201). When naturally-occurring residues are added or removed from the core sequence, EV, TEV, RETEV (SEQ ID NO:202), FRETEV (SEQ ID NO:233), HFRETEV (SEQ ID NO:234), and KHFRETEV (SEQ ID NO:235) may also be used to target a PDZ domain-containing protein in cells.

9. PL Sequences of Tight Junction Integral Membrane Proteins

A number of tight junction integral membrane proteins contain a PL motif sequence (PL sequence). These molecules include, but are not limited to, Claudin 1, Claudin 2, Claudin 7, Claudin 9, Claudin 10, and Claudin 18.

The C-terminal core sequence of Claudin 1 is KDYV (SEQ ID NO:236). When naturally-occurring residues are added or removed from the core sequence, YV, DYV, GKDYV (SEQ ID NO:237), SGKDYV (SEQ ID NO:238), SSGKDYV (SEQ ID NO:239), and PSSGKDYV (SEQ ID NO:240) may also be used to target a PDZ domain-containing protein in cells.

The C-terminal core sequence of Claudin 2 is TGYV (SEQ ID NO:241). When naturally-occurring residues are added or removed from the core sequence, YV, GYV, LTGYV (SEQ ID NO:242), SLTGYV (SEQ ID NO:243), YSLTGYV (SEQ ID NO:244), and SYSLTGYV (SEQ ID NO:245) may also be used to target a PDZ domain-containing protein in cells.

The C-terminal core sequence of Claudin 7 is KEYV (SEQ ID NO:246). When naturally-occurring residues are added or removed from the core sequence, YV, EYV, SKEYV (SEQ ID NO:247), SSKEYV (SEQ ID NO:248), NSSKEYV (SEQ ID NO:245), and SNSSKEYV (SEQ ID NO:250) may also be used to target a PDZ domain-containing protein in cells.

The C-terminal core sequence of Claudin 9 is RDYV (SEQ ID NO:251). When naturally-occurring residues are added or removed from the core sequence, YV, DYV, KRDYV (SEQ ID NO:252), DKRDYV (SEQ ID NO:253), LDKRDYV (SEQ ID NO:254), and GLDKRDYV (SEQ ID NO:255) may also be used to target a PDZ domain-containing protein in cells.

The C-terminal core sequence of Claudin 10 is NAYV (SEQ ID NO:256). When naturally-occurring residues are added or removed from the core sequence, YV, AYV, KNAYV (SEQ ID NO:257), DKNAYV (SEQ ID NO:258), FDKNAYV (SEQ ID NO:259), and QFDKNAYV (SEQ ID NO:260) may also be used to target a PDZ domain-containing protein in cells.

The C-terminal core sequence of Claudin 18 is HDYV (SEQ ID NO:261). When naturally-occurring residues are added or removed from the core sequence, YV, DYV, KHDYV (SEQ ID NO:262), SKHDYV (SEQ ID NO:263), PSKHDYV (SEQ ID NO:264), and YPSKHDYV (SEQ ID NO:265) may also be used to target a PDZ domain-containing protein in cells.

10. PL Sequences of Cell Adhesion Molecules

A number of cell adhesion molecules contain a PL motif sequence (PL sequence). As used herein, an adhesion protein is a cell surface protein involved in cell-cell interaction by direct contact with cell surface molecules (e.g., transmembrane proteins or surface proteins) on a different cell. Thus, when a cell expressing a PL adhesion protein contacts an appropriate other cell, the PL adhesion protein localizes at the interface of the two cells and directly contacts a cell surface molecule on the second cell. A cell-cell interface is a region where the plasma membranes of two different cells are in close (generally <10 nm, often about 1 nm) apposition. Typically, direct molecular contact means interaction of molecules at distances where Van der Walls forces are significant, generally less than about 1 nm. Inhibition or modulation can occur in a variety of cell types including, endothelial cells, epithelial cells, keratinocytes, hepatocytes and cardiac myocytes.

These molecules include, but are not limited to, Neuroligin, Nectin 2, JAM (junctional adhesion molecule), neurofascin (chicken), and CSPG4 (chondroitin sulfate proteoglycan 4, melanoma-associated).

The C-terminal core sequence of Neuroligin is TTRV (SEQ ID NO:266). When naturally-occurring residues are added or removed from the core sequence, RV, TRV, STTRV (SEQ ID NO:267), HSTTRV (SEQ ID NO:268), PHSTTRV (SEQ ID NO:269), and LPHSTTRV (SEQ ID NO:270) may also be used to target a PDZ domain-containing protein in cells.

The C-terminal core sequence of Nectin 2 is AMYV (SEQ ID NO:271). When naturally-occurring residues are added or removed from the core sequence, YV, MYV, RAMYV (SEQ ID NO:272), SRAMYV (SEQ ID NO:273), MSRAMYV (SEQ ID NO:274), and VMSRAMYV (SEQ ID NO:275) may also be used to target a PDZ domain-containing protein in cells.

The C-terminal core sequence of JAM is SLFV (SEQ ID NO:276). When naturally-occurring residues are added or removed from the core sequence, FV, LFV, SSLFV (SEQ ID NO:277), TSSLFV (SEQ ID NO:278), QTSSLFV (SEQ ID NO:279), and KQTSSLFV (SEQ ID NO:280) may also be used to target a PDZ domain-containing protein in cells.

The C-terminal core sequence of neurofascin is YSLA (SEQ ID NO:281). When naturally-occurring residues are added or removed from the core sequence, LA, SLA, IYSLA (SEQ ID NO:282), AIYSLA (SEQ ID NO:283), NAIYSLA (SEQ ID NO:284), and VNAIYSLA (SEQ ID NO:285) may also be used to target a PDZ domain-containing protein in cells.

The C-terminal core sequence of CSPG4 is QYWV (SEQ ID NO:286). When naturally-occurring residues are added or removed from the core sequence, WV, YWV, GQYWV (SEQ ID NO:287), NGQYWV (SEQ ID NO:288), KNGQYWV (SEQ ID NO:289), and LKNGQYWV (SEQ ID NO:290) may also be used to target a PDZ domain-containing protein in cells.

11. PL Sequences of Neuron Membrane Transport and Organization Molecules

A number of neuron membrane transport and organization molecules contain a PL motif sequence (PL sequence). These molecules include, but are not limited to, Dopamine transporter, noradrenaline transporter, glutamate transporter 3, GABA transporter 3, MINT-1, MINT-2, MINT-3, presenilin-1, and presenilin-2.

The C-terminal core sequence of Dopamine transporter is WLKV (SEQ ID NO:291). When naturally-occurring residues are added or removed from the core sequence, KV, LKV, HWLKV (SEQ ID NO:292), RHWLKV (SEQ ID NO:293), LRHWLKV (SEQ ID NO:294), and TLRHWLKV (SEQ ID NO:295) may also be used to target a PDZ domain-containing protein in cells.

The C-terminal core sequence of noradrenaline transporter is WLAI (SEQ ID NO:296). When naturally-occurring residues are added or removed from the core sequence, AI, LAI, HWLAI (SEQ ID NO:297), QHWLAI (SEQ ID NO:298), LQHWLAI (SEQ ID NO:299), and QLQHWLAI (SEQ ID NO:300) may also be used to target a PDZ domain-containing protein in cells.

The C-terminal core sequence of glutamate transporter 3 is TSQF (SEQ ID NO:301). When naturally-occurring residues are added or removed from the core sequence, QF, SQF, QTSQF (SEQ ID NO:302), TQTSQF (SEQ ID NO:303), FTQTSQF (SEQ ID NO:304), and SFTQTSQF (SEQ ID NO:305) may also be used to target a PDZ domain-containing protein in cells.

The C-terminal core sequence of GABA transporter 3 is ETHF (SEQ ID NO:306). When naturally-occurring residues are added or removed from the core sequence, HF, THF, KETHF (SEQ ID NO:307), EKETHF (SEQ ID NO:308), TEKETHF (SEQ ID NO:309), and ITEKETHF (SEQ ID NO:310) may also be used to target a PDZ domain-containing protein in cells.

The C-terminal core sequence of MINT-1 is PVYI (SEQ ID NO:311). When naturally-occurring residues are added or removed from the core sequence, YI, VYI, QPVYI (SEQ ID NO:312), EQPVYI (SEQ ID NO:313), QEQPVYI (SEQ ID NO:314), and AQEQPVYI (SEQ ID NO:315) may also be used to target a PDZ domain-containing protein in cells.

The C-terminal core sequence of MINT-2 is PLYI (SEQ ID NO:316). When naturally-occurring residues are added or removed from the core sequence, YI, LYI, TPLYI (SEQ ID NO:317), ETPLYI (SEQ ID NO:318), QETPLYI (SEQ ID NO:319), and GQETPLYI (SEQ ID NO:320) may also be used to target a PDZ domain-containing protein in cells.

The C-terminal core sequence of MINT-3 is PVYL (SEQ ID NO:321). When naturally-occurring residues are added or removed from the core sequence, YL, VYL, QPVYL (SEQ ID NO:322), EQPVYL (SEQ ID NO:323), QEQPVYL (SEQ ID NO:324), and GQEQPVYL (SEQ ID NO:325) may also be used to target a PDZ domain-containing protein in cells.

The C-terminal core sequence of presenilin-1 is QFYI (SEQ ID NO:326). When naturally-occurring residues are added or removed from the core sequence, YI, FYI, HQFYI (SEQ ID NO:327), FHQFYI (SEQ ID NO:328), AFHQFYI (SEQ ID NO:329), and LAFHQFYI (SEQ ID NO:330) may also be used to target a PDZ domain-containing protein in cells.

The C-terminal core sequence of presenilin-2 is QLYI (SEQ ID NO:331). When naturally-occurring residues are added or removed from the core sequence, YI, LYI, HQLYI (SEQ ID NO:332), SHQLYI (SEQ ID NO:333), ASHQLYI (SEQ ID NO:334), and LASHQLYI (SEQ ID NO:335) may also be used to target a PDZ domain-containing protein in cells.

12. PL Sequences of Receptor Kinases

A number of receptor kinases contain a PL motif sequence (PL sequence). These molecules include, but are not limited to, ephrin A2, ephrin B1, ephrin B2, c-kit receptor, and ErbB-4 receptor.

The C-terminal core sequence of ephrin A2 is GIPI (SEQ ID NO:336). When naturally-occurring residues are added or removed from the core sequence, PI, IPI, VGIPI (SEQ ID NO:337), TVGIPI (SEQ ID NO:338), NTVGIPI (SEQ ID NO:339), and VNTVGIPI (SEQ ID NO:340) may also be used to target a PDZ domain-containing protein in cells.

The C-terminal core sequence of ephrin B1 is YYKV (SEQ ID NO:341). When naturally-occurring residues are added or removed from the core sequence, KV, YKV, IYYKV (SEQ ID NO:342), NIYYKV (SEQ ID NO:343), ANIYYKV (SEQ ID NO:344), and PANIYYKV (SEQ ID NO:345) may also be used to target a PDZ domain-containing protein in cells.

The C-terminal core sequence of ephrin B2 is SVEV (SEQ ID NO:346). When naturally-occurring residues are added or removed from the core sequence, EV, VEV, QSVEV (SEQ ID NO:347), IQSVEV (SEQ ID NO:348), QIQSVEV (SEQ ID NO:349), and NQIQSVEV (SEQ ID NO:350) may also be used to target a PDZ domain-containing protein in cells.

The C-terminal core sequence of c-kit receptor is HDDV (SEQ ID NO:351). When naturally-occurring residues are added or removed from the core sequence, DV, DDV, VHDDV (SEQ ID NO:352), LVHDDV (SEQ ID NO:353), LLVHDDV (SEQ ID NO:354), and PLLVHDDV (SEQ ID NO:355) may also be used to target a PDZ domain-containing protein in cells.

The C-terminal core sequence of ErbB-4 receptor is NTVV (SEQ ID NO:356). When naturally-occurring residues are added or removed from the core sequence, VV, TVV, RNTVV (SEQ ID NO:357), HRNTVV (SEQ ID NO:358), RHRNTVV (SEQ ID NO:359), and YRHRNTVV (SEQ ID NO:360) may also be used to target a PDZ domain-containing protein in cells.

13. PL Sequences of Regulators of G-Protein Signaling

A number of regulators of G-protein signaling contain a PL motif sequence (PL sequence). These molecules include, but are not limited to, RGS12 (regulator of G-protein signaling 12), and GAIP (G-alpha interacting protein) RGS 19.

The C-terminal core sequence of RGS12 is ATFV (SEQ ID NO:361). When naturally-occurring residues are added or removed from the core sequence, FV, TFV, HATFV (SEQ ID NO:362), HHATFV (SEQ ID NO:363), AHHATFV (SEQ ID NO:364), and SAHHATFV (SEQ ID NO:365) may also be used to target a PDZ domain-containing protein in cells.

The C-terminal core sequence of GAIP (G-alpha interacting protein) RGS 19 is SSEA (SEQ ID NO:366). When naturally-occurring residues are added or removed from the core sequence, EA, SEA, QSSEA (SEQ ID NO:367), SQSSEA (SEQ ID NO:368), PSQSSEA (SEQ ID NO:369), and GPSQSSEA (SEQ ID NO:370) may also be used to target a PDZ domain-containing protein in cells.

14. PL Sequences of Ion Channels and Transporters

A number of regulators of ion channels and transporters contain a PL motif sequence (PL sequence). As used herein, an ion channel protein means a transmembrane protein that itself catalyzes the passage of an ion from aqueous solution on one side of a lipid bilayer membrane to aqueous solution on the other side (e.g., by forming a small pore in the membrane). These molecules include, but are not limited to, Kir2.1 (inwardly rect. K+ channel), and Na+/Pi contransporter 2.

The C-terminal core sequence of Kir2.1 is ESEI (SEQ ID NO:371). When naturally-occurring residues are added or removed from the core sequence, EI, SEI, RESEI (SEQ ID NO:372), RRESEI (SEQ ID NO:373), LRRESEI (SEQ ID NO:374), and PLRRESEI (SEQ ID NO:375) may also be used to target a PDZ domain-containing protein in cells.

The C-terminal core sequence of Na+/Pi contransporter 2 is ATRL (SEQ ID NO:376). When naturally-occurring residues are added or removed from the core sequence, RL, TRL, NATRL (SEQ ID NO:377), HNATRL (SEQ ID NO:378), HHNATRL (SEQ ID NO:379), and AHHNATRL (SEQ ID NO:380) may also be used to target a PDZ domain-containing protein in cells.

15. PL Sequences of Tumor Suppressor Proteins, Cell Viability Associated Proteins, Receptors, and Critical Regulators

A number of tumor suppressor proteins, cell viability associated proteins, receptors, and critical regulators contain a PL motif sequence (PL sequence). These molecules include, but are not limited to, alpha-1-syntrophin, ropporin, CX43 (connexin 43), CD68, a-actinin 2, zona occludens 3 (ZO-3), KIA 1481, CFTCR (cystic fibrosis transmembrane conductance regulator), ActRIIA, CAPON (carboxyl-terminal PDZ ligand of neuronal nitric oxide synthase) mRNA, RA-GEF (ras/rap1A-assoc.-GEF), PDZ-binding kinase (PBK), RhoGAP (PTPL1-associated), CITRON protein, Nedasin (s-form), APC-adenomatous polyposis coli protein, CKR5 (HIV Co-receptor), catenin-delta 2, bone morphogenetic protein receptor, TRAF2, Glycophorin C, and PTEN.

The C-terminal core sequence of alpha-1-syntrophin is GLLA (SEQ ID NO:381). When naturally-occurring residues are added or removed from the core sequence, LA, LLA, LGLLA (SEQ ID NO:382), RLGLLA (SEQ ID NO:383), TRLGLLA (SEQ ID NO:384), and VTRLGLLA (SEQ ID NO:385) may also be used to target a PDZ domain-containing protein in cells.

The C-terminal core sequence of ropprin is VQLE (SEQ ID NO:386). When naturally-occurring residues are added or removed from the core sequence, LE, QLE, RVQLE (SEQ ID NO:387), PRVQLE (SEQ ID NO:388), NPRVQLE (SEQ ID NO:389), and QNPRVQLE (SEQ ID NO:390) may also be used to target a PDZ domain-containing protein in cells.

The C-terminal core sequence of CX43 (connexin 43) is DLEI (SEQ ID NO:391). When naturally-occurring residues are added or removed from the core sequence, EI, LEI, DDLEI (SEQ ID NO:392), PDDLEI (SEQ ID NO:393), RPDDLEI (SEQ ID NO:394), and PRPDDLEI (SEQ ID NO:395) may also be used to target a PDZ domain-containing protein in cells.

The C-terminal core sequence of CD68 is YQAL (SEQ ID NO:396). When naturally-occurring residues are added or removed from the core sequence, AL, QAL, AYQAL (SEQ ID NO:397), SAYQAL (SEQ ID NO:398), PSAYQAL (SEQ ID NO:399), and RPSAYQAL (SEQ ID NO:400) may also be used to target a PDZ domain-containing protein in cells.

The C-terminal core sequence of a-actinin 2 is ESDL (SEQ ID NO:401). When naturally-occurring residues are added or removed from the core sequence, DL, SDL, GESDL (SEQ ID NO:402), YGESDL (SEQ ID NO:403), LYGESDL (SEQ ID NO:404), and ALYGESDL (SEQ ID NO:405) may also be used to target a PDZ domain-containing protein in cells.

The C-terminal core sequence of zona occludens 3 (ZO-3) is ATDL (SEQ ID NO:406). When naturally-occurring residues are added or removed from the core sequence, DL, TDL, PATDL (SEQ ID NO:407), GPATDL (SEQ ID NO:408), WGPATDL (SEQ ID NO:409), and DWGPATDL (SEQ ID NO:410) may also be used to target a PDZ domain-containing protein in cells.

The C-terminal core sequence of KIA 1481 is TSPL (SEQ ID NO:411). When naturally-occurring residues are added or removed from the core sequence, PL, SPL, PTSPL (SEQ ID NO:412), GPTSPL (SEQ ID NO:413), WGPTSPL (SEQ ID NO:414), and DWGPTSPL (SEQ ID NO:415) may also be used to target a PDZ domain-containing protein in cells.

The C-terminal core sequence of CFTCR (cystic fibrosis transmembrane conductance regulator) is DTRL (SEQ ID NO:416). When naturally-occurring residues are added or removed from the core sequence, RL, TRL, QDTRL (SEQ ID NO:417), VQDTRL (SEQ ID NO:418), EVQDTRL (SEQ ID NO:419), and EEVQDTRL (SEQ ID NO:420) may also be used to target a PDZ domain-containing protein in cells.

The C-terminal core sequence of ActRIIA is ESSL (SEQ ID NO:421). When naturally-occurring residues are added or removed from the core sequence, SL, SSL, KESSL (SEQ ID NO:422), PKESSL (SEQ ID NO:423), PPKESSL (SEQ ID NO:424), and FPPKESSL (SEQ ID NO:425) may also be used to target a PDZ domain-containing protein in cells.

The C-terminal core sequence of CAPON (carboxy-terminal PDZ ligand of neuronal nitric oxide synthase) mRNA is EIAV (SEQ ID NO:426). When naturally-occurring residues are added or removed from the core sequence, AV, IAV, DEIAV (SEQ ID NO:427), DDEIAV (SEQ ID NO:428), LDDEIAV (SEQ ID NO:429), and GLDDEIAV (SEQ ID NO:430) may also be used to target a PDZ domain-containing protein in cells.

The C-terminal core sequence of RA-GEF (ras/rap1A-assoc.-GEF) is VSAV (SEQ ID NO:431). When naturally-occurring residues are added or removed from the core sequence, AV, SAV, QVSAV (SEQ ID NO:432), EQVSAV (SEQ ID NO:433), DEQVSAV (SEQ ID NO:434), and EDEQVSAV (SEQ ID NO:435) may also be used to target a PDZ domain-containing protein in cells.

The C-terminal core sequence of PDZ-binding kinase (PBK) is ETDV (SEQ ID NO:436). When naturally-occurring residues are added or removed from the core sequence, DV, TDV, LETDV (SEQ ID NO:437), ALETDV (SEQ ID NO:438), EALETDV (SEQ ID NO:439), and VEALETDV (SEQ ID NO:440) may also be used to target a PDZ domain-containing protein in cells.

The C-terminal core sequence of RhoGAP 1 (PTPL1-associated) is PQFV (SEQ ID NO:441). When naturally-occurring residues are added or removed from the core sequence, FV, QFV, IPQFV (SEQ ID NO:442), EIPQFV (SEQ ID NO:443), DEIPQFV (SEQ ID NO:444), and EDEIPQFV (SEQ ID NO:445) may also be used to target a PDZ domain-containing protein in cells.

The C-terminal core sequence of CITRON protein is QSSV (SEQ ID NO:446). When naturally-occurring residues are added or removed from the core sequence, SV, SSV, DQSSV (SEQ ID NO:447), WDQSSV (SEQ ID NO:448), VWDQSSV (SEQ ID NO:449), and KVWDQSSV (SEQ ID NO:450) may also be used to target a PDZ domain-containing protein in cells.

The C-terminal core sequence of Nedasin (s-form) is SSSV (SEQ ID NO:451). When naturally-occurring residues are added or removed from the core sequence, SV, SSV, FSSSV (SEQ ID NO:452), PFSSSV (SEQ ID NO:453), VPFSSSV (SEQ ID NO:454), and VVPFSSSV (SEQ ID NO:455) may also be used to target a PDZ domain-containing protein in cells.

The C-terminal core sequence of APC-adenomatous polyposis coli protein is VTSV (SEQ ID NO:456). When naturally-occurring residues are added or removed from the core sequence, SV, TSV, LVTSV (SEQ ID NO:457), YLVTSV (SEQ ID NO:458), SYLVTSV (SEQ ID NO:459), and GSYLVTSV (SEQ ID NO:460) may also be used to target a PDZ domain-containing protein in cells.

The C-terminal core sequence of CKR5 (HIV Co-receptor) is SVGL (SEQ ID NO:461). When naturally-occurring residues are added or removed from the core sequence, GL, VGL, ISVGL (SEQ ID NO:462), EISVGL (SEQ ID NO:463), QEISVGL (SEQ ID NO:464), and EQEISVGL (SEQ ID NO:465) may also be used to target a PDZ domain-containing protein in cells.

The C-terminal core sequence of cantenin-delta 2 is DSWV (SEQ ID NO:466). When naturally-occurring residues are added or removed from the core sequence, WV, SWV, PDSWV (SEQ ID NO:467), SPDSWV (SEQ ID NO:468), ASPDSWV (SEQ ID NO:469), and PASPDSWV (SEQ ID NO:470) may also be used to target a PDZ domain-containing protein in cells.

The C-terminal core sequence of bone morphogenetic protein receptor is DVKI (SEQ ID NO:471). When naturally-occurring residues are added or removed from the core sequence, KI, VKI, QDVKI (SEQ ID NO:472), SQDVKI (SEQ ID NO:473), ESQDVKI (SEQ ID NO:474), and VESQDVKI (SEQ ID NO:475) may also be used to target a PDZ domain-containing protein in cells.

The C-terminal core sequence of TRAF2 is LTGL (SEQ ID NO:476). When naturally-occurring residues are added or removed from the core sequence, GL, TGL, DLTGL (SEQ ID NO:477), VDLTGL (SEQ ID NO:478), IVDLTGL (SEQ ID NO:479), and AIVDLTGL (SEQ ID NO:480) may also be used to target a PDZ domain-containing protein in cells.

The C-terminal core sequence of Glycophorin C is EYFI (SEQ ID NO:481). When naturally-occurring residues are added or removed from the core sequence, FI, YFI, KEYFI (SEQ ID NO:482), RKEYFI (SEQ ID NO:483), SRKEYFI (SEQ ID NO:484), and SSRKEYFI (SEQ ID NO:485) may also be used to target a PDZ domain-containing protein in cells.

The C-terminal core sequence of PTEN is ITKV (SEQ ID NO-486). When naturally-occurring residues are added or removed from the core sequence, KV, TKV, QITKV (SEQ ID NO:487), TQITKV (SEQ ID NO:488), HTQITKV (SEQ ID NO:489), and QHTQITKV (SEQ ID NO:490) may also be used to target a PDZ domain-containing protein in cells.

16. Others

The PL proteins that have been identified herein as interacting with particular PDZ proteins also include intracellular proteins, and cytokine receptors, and adaptor proteins. As used herein, an intercellular (i.e., cytosolic) protein has the normal meaning in the art and refers to a protein that is not membrane bound, e.g., has no transmembrane domain. The term cytokine receptor as used herein a cytokine receptor has the normal meaning in the art and refers to a membrane protein with an extracellular domain that specifically binds a cytokine. As used herein, an adaptor protein means a molecule (e.g., protein) that contributes to the formation of a multimolecular complex by binding two or more other biomolecules. The binding of the two or more other molecules by the adaptor molecule/protein generally involves direct molecular contact between the adaptor protein and each of the two or more other molecules.

V. Detection of PDZ Domain-Containing Proteins

As noted supra, the present inventors have identified a number of PDZ protein and PL protein interactions that can play a role in modulation of a number of biological functions in a variety of cell types. A comprehensive list of PDZ domain-containing proteins was retrieved from the Sanger Centre database (Pfam) searching for the keyword, “PDZ”. The corresponding cDNA sequences were retrieved from GenBank using the NCBI “entrez” database (hereinafter, “GenBank PDZ protein cDNA sequences”). The DNA portion encoding PDZ domains was identified by alignment of cDNA and protein sequence using CLUSTALW. Based on the DNA/protein alignment information, primers encompassing the PDZ domains were designed. The expression of certain PDZ-containing proteins in cells was detected by polymerase chain reaction (“PCR”) amplification of cDNAs obtained by reverse transcription (“RT”) of cell-derived RNA (i.e., “RT-PCR”). PCR, RT-PCR and other methods for analysis and manipulation of nucleic acids are well known and are described generally in Sambrook et al., (1989) MOLECULAR CLONING: A LABORATORY MANUAL, 2ND ED., VOLS. 1-3, Cold Spring Harbor Laboratory hereinafter, “Sambrook”); and Ausubel et al., CURRENT PROTOCOLS IN MOLECULAR BIOLOGY, Greene Publishing and Wiley-Interscience, New York (1997), as supplemented through January 1999 (hereinafter “Ausubel”).

Samples of cDNA for those sequences identified through the foregoing search were obtained and then amplified. In general, a sample of the cDNA (typically, ⅕ of a 20 μl reaction) was used to conduct PCR. PCR was conducted using primers designed specifically to amplify PDZ domain-containing regions of PDZ proteins of interest. Oligonucleotide primers were designed to amplify one or more PDZ-encoding domains. The DNA sequences encoding the various PDZ domains of interest were identified by inspection (i.e., conceptual translation of the PDZ protein cDNA sequences obtained from GenBank, followed by alignment with the PDZ domain amino acid sequence). TABLE 9 shows the PDZ-encoded domains amplified, and the GenBank accession number of the PDZ-domain containing proteins. To facilitate subsequent cloning of PDZ domains, the PCR primers included endonuclease restriction sequences at their ends to allow ligation with pGEX-3X cloning vector (Pharmacia, GenBank XXI13852 ) in frame with glutathione-S transferase (GST).

VI. Assays for Detection of Interactions Between PDZ-Domain Polypeptides and Candidate PDZ Ligand Proteins (PL Proteins)

Two complementary assays, termed “A′ and “G,”” were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A′” assay is named for the fact that in one embodiment an avidin surface is used to immobilize the peptide). In the “G” assay, the PDZ-domain polypeptide is immobilized and binding of a soluble PL peptide is detected (The “G” assay is named for the fact that in one embodiment a GST-binding surface is used to immobilize the PDZ-domain polypeptide). Preferred embodiments of these assays are described in detail infra. However, it will be appreciated by ordinarily skilled practitioners that these assays can be modified in numerous ways while remaining useful for the purposes of the present invention.

A. Production of Fusion Proteins Containing PDZ-Domains

GST-PDZ domain fusion proteins were prepared for use in the assays of the invention. PCR products containing PDZ encoding domains (as described supra) were subcloned into an expression vector to permit expression of fusion proteins containing a PDZ domain and a heterologous domain (i.e., a glutathione-S transferase sequence, “GST”). PCR products (i.e., DNA fragments) representing PDZ domain encoding DNA was extracted from agarose gels using the “sephaglas” gel extraction system (Pharmacia) according to the manufacturer's recommendations.

As noted supra, PCR primers were designed to include endonuclease restriction sites to facilitate ligation of PCR fragments into a GST gene fusion vector (pGEX-3X; Pharmacia, GenBank accession no. XXU13852) in-frame with the glutathione-S transferase coding sequence. This vector contains an IPTG inducible lacZ promoter. The pGEX-3X vector was linearized using Bam HI and Eco RI or, in some cases, Eco RI or Sma I, and dephosphorylated. For most cloning approaches, double digestion with Bam HI and Eco RI was performed, so that the ends of the PCR fragments to clone were Bam HI and Eco RI. In some cases, restriction endonuclease combinations used were Bgl II and Eco RI, Bam HI and Mfe I, or Eco RI only, Sma I only, or BamHI only. When more than one PDZ domain was cloned, the DNA portion cloned represents the PDZ domains and the cDNA portion located between individual domains. Precise locations of cloned fragments used in the assays are indicated in TABLE 9. DNA linker sequences between the GST portion and the PDZ domain containing DNA portion vary slightly, dependent on which of the above described cloning sites and approaches were used. As a consequence, the amino acid sequence of the GST-PDZ fusion protein varies in the linker region between GST and PDZ domain. Protein linker sequences corresponding to different cloning sites/approaches are shown below. Linker sequences (vector DNA encoded) are bold, PDZ domain containing gene derived sequences are in italics.

| | |
|---|---|
| 1) | GST-BamHI/BamHI-PDZ domain insert |
| | Gly--Ile-PDZ domain insert |
| 2) | GST-BamHI/BglII-PDZ domain insert |
| | Gly-Ile-PDZ domain insert |
| 3) | GST-EcoRI/EcoI-PDZ domain insert |
| | Gly-Ile-Pro-Gly--Asn-PDZ domain insert |
| | (SEQ ID NO: 232) |
| 4) | GST--SmaI/SmaI-PDZ domain insert |
| | Gly-Ile-Pro-PDZ domain insert |

The PDZ-encoding PCR fragment and linearized pGEX-3X vector were ethanol precipitated and resuspended in 10 ul standard ligation buffer. Ligation was performed for 4-10 hours at 7° C. using T4 DNA ligase. It will be understood that some of the resulting constructs include very short linker sequences and that, when multiple PDZ domains were cloned, the constructs included some DNA located between individual PDZ domains.

The ligation products were transformed in DH5α or BL-21 E.coli bacteria strains. Colonies were screened for presence and identity of the cloned PDZ domain containing DNA as well as for correct fusion with the glutathione S-transferase encoding DNA portion by PCR and by sequence analysis. Positive clones were tested in a small-scale assay for expression of the GST/PDZ domain fusion protein and, if expressing, these clones were subsequently grown up for large scale preparations of GST/PDZ fusion protein.

GST-PDZ domain fusion protein was overexpressed following addition of IPTG to the culture medium and purified. Detailed procedure of small scale and large-scale fusion protein expression and purification are described in “GST Gene Fusion System” (second edition, revision 2; published by Pharmacia). In brief, a small culture (50 mls) containing a bacterial strain (DH5α, BL21 or JM109) with the fusion protein construct was grown overnight in 2×YT media at 37° C. with the appropriate antibiotic selection (100 ug/ml ampicillin; a.k.a. 2×YT-amp). The overnight culture was poured into a fresh preparation of 2×YT-amp (typically 1 liter) and grown until the optical density (OD) of the culture was between 0.5 and 0.9 (approximately 2.5 hours). IPTG (isopropyl β-D-thiogalactopyranoside) was added to a final concentration of 1.0 mM to induce production of GST fusion protein, and culture was grown an additional 1 hour. All following steps, including centrifugation, were performed on ice or at 4° C. Bacteria were collected by centrifugation (4500 g) and resuspended in Buffer A− (50 mM Tris, pH 8.0, 50 mM dextrose, 1 mM EDTA, 200 uM phenylmethylsulfonylfluoride). An equal volume of Buffer A+ (Buffer A−, 4 mg/ml lysozyme) was added and incubated on ice for 3 min to lyse bacteria, or until lysis had begun. An equal volume of Buffer B (10 mM Tris, pH 8.0, 50 mM KCl, 1 mM EDTA. 0.5% Tween-20, 0.5% NP40 (a.k.a. IGEPAL CA-630), 200 uM phenylmethylsulfonylfluoride) was added and incubated for an additional 20 min on ice. The bacterial cell lysate was centrifuged (×20,000 g), and supernatant was run over a column containing 20 ml Sepharose CL-4B (Pharmacia) “precolumn beads,” i.e., sepharose beads without conjugated glutathione that had been previously washed with 3 bed volumes PBS. The flow-through was added to glutathione Sepharose 4B (Pharmacia, cat no. 17-0765-01) previously swelled (rehydrated) in 1× phosphate-buffered saline (PBS) and incubated while rotating for 30 min-1 hr. The supernatant-Sepharose slurry was poured into a column and washed with at least 20 bed volumes of 1×PBS. GST fusion protein was eluted off the glutathione sepharose by applying 0.5-1.0 ml aliquots of 5 mM glutathione and collected as separate fractions. Concentrations of fractions were determined by reading absorbance at 280 nm and calculating concentration using the absorbance and extinction coefficient. Those fractions containing the highest concentration of fusion protein were pooled and an equal volume of 70% glycerol was added to a final concentration of 35% glycerol. Fusion proteins were assayed for size and quality by SDS gel electrophoresis (PAGE) as described in “Sambrook.” Fusion protein aliquots were stored at minus 80° C. and at minus 20° C.

B. Identification of Candidate PL Proteins and Synthesis of Peptides

Certain PDZ domains are bound by the C-terminal residues of PDZ-binding proteins. To identify PL proteins the C-terminal residues of sequences were visually inspected for sequences that one might predict would bind to PDZ-domain containing proteins (see, e.g., Doyle et al., 1996, Cell 85, 1067; Songyang et al., 1997, Science 275, 73), including the additional consenses for PLs identified at Arbor Vita Corporation (TABLE 8, and data not shown). TABLE 8 lists some of these proteins, and provides corresponding C-terminal sequences and GenBank accession numbers.

Synthetic peptides of defined sequence (e.g., corresponding to the carboxyl-termini of the indicated proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein were prepared by the FMOC (see, e.g., Guy and Fields, 1997, Meth. Enz. 289:67-83; Wellings and Atherton, 1997, Meth. Enz.289:44-67). In some cases (e.g., for use in the A and G assays of the invention), peptides were labeled with biotin at the amino-terminus by reaction with a four-fold excess of biotin methyl ester in dimethylsulfoxide with a catalytic amount of base. The peptides were cleaved from the resin using a halide containing acid (e.g. trifluoroacetic acid) in the presence of appropriate antioxidants (e.g. ethanedithiol) and excess solvent lyophilized.

Following lyophilization, peptides can be redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroacetic acid in a base solvent of water plus 0.1% trifluoroacetic acid. After HPLC purification, the identities of the peptides are confirmed by MALDI cation-mode mass spectrometry. As noted, exemplary biotinylated peptides are provided in TABLE 8.

C. Detecting PDZ-PL Interactions

The present inventors were able in part to identify the interactions summarized in TABLE 7 and TABLE 12 by developing new high throughput screening assays which are described in greater detail infra. Various other assay formats known in the art can be used to select ligands that are specifically reactive with a particular protein. For example, solid-phase ELISA immunoassays, immunoprecipitation, Biacore, and Western blot assays can be used to identify peptides that specifically bind PDZ-domain polypeptides. As discussed supra, two different, complementary assays were developed to detect PDZ-PL interactions. In each, one binding partner of a PDZ-PL pair is immobilized, and the ability of the second binding partner to bind is determined. These assays, which are described infra, can be readily used to screen for hundreds to thousand of potential PDZ-ligand interactions in a few hours. Thus these assays can be used to identify yet more novel PDZ-PL interactions in hematopoietic cells. In addition, they can be used to identify antagonists of PDZ-PL interactions (see infra).

In some assays, fusion proteins are used in the assays and devices of the invention. Methods for constructing and expressing fusion proteins are well known. Fusion proteins generally are described in Ausubel et al., supra, Kroll et al., 1993, DNA Cell. Biol. 12:441, and Imai et al., 1997, Cell 91:521-30. Usually, the fusion protein includes a domain to facilitate immobilization of the protein to a solid substrate (“an immobilization domain”). Often, the immobilization domain includes an epitope tag (i.e., a sequence recognized by an antibody, typically a monoclonal antibody) such as polyhistidine (Bush et al, 1991, J. Biol Chem 266:13811-14), SEAP (Berger et al, 1988, Gene 66:1-10), or M1 and M2 flag (see, e.g, U.S. Pat. Nos. 5,011,912; 4,851,341; 4,703,004; 4,782,137). In an embodiment, the immobilization domain is a GST coding region. It will be recognized that, in addition to the PDZ-domain and the particular residues bound by an immobilized antibody, protein A, or otherwise contacted with the surface, the protein (e.g., fusion protein), will contain additional residues. In some embodiments these are residues naturally associated with the PDZ-domain (i.e., in a particular PDZ-protein) but they can include residues of synthetic (e.g., poly(alanine)) or heterologous origin (e.g., spacers of, e.g., between 10 and 300 residues).

PDZ domain-containing polypeptide used in these methods are typically made by (1) constructing a vector (e.g., plasmid, phage or phagemid) comprising a polynucleotide sequence encoding the desired polypeptide, (2) introducing the vector into an suitable expression system (e.g., a prokaryotic, insect, mammalian, or cell free expression system), (3) expressing the fusion protein and (4) optionally purifying the fusion protein.

Generally, expression of the protein comprises inserting the coding sequence into an appropriate expression vector (i.e., a vector that contains the necessary elements for the transcription and translation of the inserted coding sequence required for the expression system employed, e.g., control elements including enhancers, promoters, transcription terminators, origins of replication, a suitable initiation codon (e.g., methionine), open reading frame, and translational regulatory signals (e.g., a ribosome binding site, a termination codon and a polyadenylation sequence. Depending on the vector system and host utilized, any number of suitable transcription and translation elements, including constitutive and inducible promoters, can be used.

The coding sequence of the fusion protein includes a PDZ domain and an immobilization domain as described elsewhere herein. Polynucleotides encoding the amino acid sequence for each domain can be obtained in a variety of ways known in the art; typically the polynucleotides are obtained by PCR amplification of cloned plasmids, cDNA libraries, and cDNA generated by reverse transcription of RNA, using primers designed based on sequences determined by the practitioner or, more often, publicly available (e.g., through GenBank). The primers include linker regions (e.g., sequences including restriction sites) to facilitate cloning and manipulation in production of the fusion construct. The polynucleotides corresponding to the PDZ and immobilization regions are joined in-frame to produce the fusion protein-encoding sequence.

The fusion proteins can be expressed as secreted proteins (e.g., by including the signal sequence encoding DNA in the fusion gene; see, e.g., Lui et al, 1993, PNAS USA, 90:8957-61) or as nonsecreted proteins.

In certain assays, the PDZ-containing proteins are immobilized on a solid surface. The substrate to which the polypeptide is bound can have any of a variety of forms, e.g., a microtiter dish, a test tube, a dipstick, a microcentrifuge tube, a bead, a spinnable disk, and the like. Suitable materials include glass, plastic (e.g., polyethylene, PVC, polypropylene, polystyrene, and the like), protein, paper, carbohydrate, lipip monolayer or supported lipid bilayer, and other solid supports. Other materials that can be employed include ceramics, metals, metalloids, semiconductive materials, cements and the like.

In other assays, the fusion proteins are organized as an array. The term “array,” as used herein, refers to an ordered arrangement of immobilized fusion proteins, in which particular different fusion proteins (i.e., having different PDZ domains) are located at different predetermined sites on the substrate. Because the location of particular fusion proteins on the array is known, binding at that location can be correlated with binding to the PDZ domain situated at that location. Immobilization of fusion proteins on beads (individually or in groups) is another particularly useful approach. In some instances, individual fusion proteins are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular PDZ domain can be determined; similarly, the effect of test compounds (i.e., agonists and antagonists of binding) can be determined.

Methods for immobilizing proteins are known, and include covalent and noncovalent methods. One suitable immobilization method is antibody-mediated immobilization. According to this method, an antibody specific for the sequence of an “immobilization domain” of the PDZ-domain containing protein is itself immobilized on the substrate (e.g., by adsorption). One advantage of this approach is that a single antibody can be adhered to the substrate and used for immobilization of a number of polypeptides (sharing the same immobilization domain). For example, an immobilization domain consisting of poly-histidine (Bush et al, 1991, J. Biol Chem 266:13811-14) can be bound by an anti-histidine monoclonal antibody (R&D Systems, Minneapolis, Minn.); an immobilization domain consisting of secreted alkaline phosphatase (“SEAP”) (Berger et al, 1988, Gene 66: 1-10) can be bound by anti-SEAP (Sigma Chemical Company, St. Louis, Mo.); an immobilization domain consisting of a FLAG epitope can be bound by anti-FLAG. Other ligand-antiligand immobilization methods are also suitable (e.g., an immobilization domain consisting of protein A sequences (Harlow and Lane, 1988, Antibodies A Laboratory Manual, Cold Spring Harbor Laboratory; Sigma Chemical Co., St. Louis, Mo.) can be bound by IgG; and an immobilization domain consisting of streptavidin can be bound by biotin (Harlow & Lane, supra; Sigma Chemical Co., St. Louis, Mo.). In a preferred embodiment, the immobilization domain is a GST moiety, as described herein.

When antibody-mediated immobilization methods are used, glass and plastic are especially useful substrates. The substrates can be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities can be used (e.g., more than 10 or 100 polypeptides per cm²). See, e.g., MacBeath et al, 2000, Science 289:1760-63.

Typically, antibodies are bound to substrates (e.g., glass substrates) by adsorption. Suitable adsorption conditions are well known in the art and include incubation of 0.5-50 ug/ml (e.g., 10 ug/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours.

Proteins can be covalently bound or noncovalently attached through nonspecific bonding. If covalent bonding between the fusion protein and the surface is desired, the surface will usually be polyfunctional or be capable of being polyfunctionalized. Functional groups which can be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

“A Assay” Detection of PDZ-Ligand Binding Using Immobilized PL Peptide.

In this particular assay, biotinylated candidate PL peptides are immobilized on an avidin-coated surface. The binding of PDZ-domain fusion protein to this surface is then measured. In certain assays, the PDZ-domain fusion protein is a GST/PDZ fusion protein and the assay is carried out as follows:

(1) Avidin is bound to a surface, e.g. a protein binding surface. In one embodiment, avidin is bound to a polystyrene 96 well plate (e.g., Nunc Polysorb (cat #475094) by addition of 100 uL per well of 20 ug/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 uL per well of PBS containing 2 g per 100 mL protease-free bovine serum albumin (“PBS/BSA”) for 2 hours at 4° C. The plate is then washed 3 times with PBS by repeatedly adding 200 uL per well of PBS to each well of the, plate and then dumping the contents of the plate into a waste container and tapping the plate gently on a dry surface.

(2) Biotinylated PL peptides (or candidate PL peptides, e.g., see TABLE 8) are immobilized on the surface of wells of the plate by addition of 50 uL per well of 0.4 uM peptide in PBS/BSA for 30 minutes at 4° C. Usually, each different peptide is added to at least eight different wells so that multiple measurements (e.g. duplicates and also measurements using different (GST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein (prepared as described supra) is allowed to react with the surface by addition of 50 uL per well of a solution containing 5 ug/mL GST/PDZ-domain fusion protein in PBS/BSA for 2 hours at 4° C. As a negative control, GST alone (i.e. not a fusion protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one assay format, 50 uL per well of an anti-GST antibody in PBS/BSA (e.g. 2.5 ug/mL of polyclonal goat-anti-GST antibody, Pierce) is added to the plate and allowed to react for 20 minutes at 4° C. The plate is washed 3 times with PBS and a second, detectably labeled antibody is added. In another assay, 50 uL per well of 2.5 ug/mL of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by the addition of 100 uL per well of 1M sulfuric acid and the optical density (O.D.) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value<0.05, more typically a p-value<0.01, and most typically a p-value<0.001 or less.

As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in some assays, specific binding of the PDZ protein to the plate-bound PL peptide is determined by comparing the mean signal (“mean S”) and standard error of the signal (“SE”) for a particular PL-PDZ combination with the mean B1 and/or mean B2.

“G Assay”—Detection of PDZ-Ligand Binding Using Immobilized PDZ-Domain Fusion Polypeptide

In other assays, a GST/PDZ fusion protein is immobilized on a surface (“G” assay). The binding of labeled PL peptide (e.g., as listed in TABLE 8) to this surface is then measured. Typically, the assay is carried out as follows:

(1) A PDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods known to one of skill in the art, although some care must be taken that the process of binding the fusion protein to the plate does not alter the ligand-binding properties of the PDZ domain. In some instances, the GST/PDZ fusion protein is bound via an anti-GST antibody that is coated onto the 96-well plate. Adequate binding to the plate can be achieved when:
a. 100 uL per well of 5 ug/mL goat anti-GST polyclonal antibody (Pierce) in PBS is added to a polystyrene 96-well plate (e.g., Nunc Polysorb) at 4° C. for 12 hours.
b. The plate is blocked by addition of 200 uL per well of PBS/BSA for 2 hours at 4° C.
c. The plate is washed 3 times with PBS.
d. 50 uL per well of 5 ug/mL GST/PDZ fusion protein) or, as a negative control, GST polypeptide alone (i.e. not a fusion protein) in PBS/BSA is added to the plate for 2 hours at 4° C.
e. The plate is again washed 3 times with PBS.

(2) Biotinylated PL peptides are allowed to react with the surface by addition of 50 uL per well of 20 uM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors known to one of skill in the art. In some assays, 100 uL per well of 0.5 ug/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by addition of 100 uL per well of 1M sulfuric acid, and the absorbance of each well of the plate is read at 450 nm.

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with-repeated measurements of the background will result in a p-value<0.05, more typically a p-value<0.01, and most typically a p-value<0.001 or less. As noted, in an embodiment of the “G” assay, the signal from binding of a given PL peptide to immobilized (surface bound) GST polypeptide alone is one suitable negative control (sometimes referred to as “B 1”). Because all measurement are done in multiples (i.e. at least duplicate) the arithmetic mean (or, equivalently, average.) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in some instances, specific binding of the PDZ protein to the platebound peptide is determined by comparing the mean signal (“mean S”) and standard error of the signal (“SE”) for a particular PL-PDZ combination with the mean B1.

“G′ Assay” and “G″ Assay”

Two specific modifications of the specific conditions described supra for the “G assay” can be utilized. The modified assays use lesser quantities of labeled PL peptide and have slightly different biochemical requirements for detection of PDZ-ligand binding compared to the specific assay conditions described supra.

For convenience, the assay conditions described in this section are referred to as the “G′ assay” and the “G″ assay,” with the specific conditions described in the preceding section on G assays being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 uM instead of 20 uM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have found to be generally true for PDZ-ligand binding) the reaction entropy is negative (i.e. the entropy of the products is less than the entropy of the reactants). In contrast, the PDZ-PL binding signal can be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT ln(Kd)=delta H−T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “G⁰ assay” generally have a rapid dissociation rate at 25° C. (t½<10 minutes) and a negative reaction entropy, while interactions that are detected similarly strongly in the “G″ assay” generally have a slower dissociation rate at 25° C. (t½>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) can itself dissociate slowly and thus be of high affinity.

In this manner, variation of the temperature and duration of step (2) of the “G assay” can be used to provide insight into the kinetics and thermodynamics of the PDZ-ligand binding reaction and into design of inhibitors of the reaction.

Assay Variations

As discussed supra, it will be appreciated that many of the steps in the above-described assays can be varied, for example, various substrates can be used for binding the PL and PDZ-containing proteins; different types of PDZ containing fusion proteins can be used; different labels for detecting PDZ/PL interactions can be employed; and different ways of detection can be used.

The PDZ-PL detection assays can employ a variety of surfaces to bind the PL and PDZ-containing proteins. For example, a surface can be an “assay plate” which is formed from a material (e.g. polystyrene) which optimizes adherence of either the PL protein or PDZ-containing protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, and the like.

For example, the assay plate can be a “microtiter” plate. The term “microtiter” plate when used herein refers to a multiwell assay plate, e.g., having between about 30 to 200 individual wells, usually 96 wells. Alternatively, high-density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. One of skill is aware of various techniques for indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:

(i) Horseradish peroxidase (HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) β-D-galactosidase (β D-Gal) with a chromogenic substrate (e.g. p-nitrophenyl-β-D-galactosidase) or fluorogenic substrate 4-methylumbelliferyl- β-D-galactosidase.

Numerous other enzyme-substrate combinations are available to those skilled in the art. For a general review of these, see U.S. Pat. Nos. 4,275,149 and 4,318,980, both of which are herein incorporated by reference.

Further, it will be appreciated that, although, for convenience, the present discussion primarily refers antagonists of PDZ-PL interactions, agonists of PDZ-PL interactions can be identified using the methods disclosed herein or readily apparent variations thereof.

VII. Results of PDZ-PL Interaction Assays

TABLE 7 and TABLE 12, supra, shows the results of assays in which specific binding was detected using the “G′” assay described herein.

VIII. Measurement of PDZ-Ligand Binding Affinity

The “A” and “G” assays described supra can be used to determine the “apparent affinity” of binding of a PDZ ligand peptide to a PDZ-domain polypeptide. Apparent affinity is determined based on the concentration of one molecule required to saturate the binding of a second molecule (e.g., the binding of a ligand to a receptor). Two particularly useful approaches for quantitation of apparent affinity of PDZ-ligand binding are provided infra.

(1) A GST/PDZ fusion protein, as well as GST alone as a negative control, are bound to a surface (e.g., a 96-well plate) and the surface blocked and washed as described supra for the “G” assay.

(2) 50 uL per well of a solution of biotinylated PL peptide (e.g. as shown in TABLE 8) is added to the surface in increasing concentrations in PBS/BSA (e.g. at 0.1 uM, 0.33 uM, 1 uM, 3.3 uM, 10 uM, 33 uM, and 100 uM). In some instances, the PL peptide is allowed to react with the bound GST/PDZ fusion protein (as well as the GST alone negative control) for 10 minutes at 4° C. followed by 20 minutes at 25° C. The plate is washed 3 times with ice cold PBS to remove unbound labeled peptide.

(3) The binding of the PL peptide to the immobilized PDZ-domain polypeptide is detected as described supra for the “G” assay.

(4) For each concentration of peptide, the net binding signal is determined by subtracting the binding of the peptide to GST alone from the binding of the peptide to the GST/PDZ fusion protein. The net binding signal is then plotted as a function of ligand concentration and the plot is fit (e.g. by using the Kaleidagraph software package curve fitting algorithm; Synergy Software) to the following equation, where “Signal_{[ligand]}” is the net binding signal at PL peptide concentration “[ligand],” “Kd” is the apparent affinity of the binding event, and “Saturation Binding” is a constant determined by the curve fitting algorithm to optimize the fit to the experimental data:
Signal_{[ligand]}=Saturation Binding×([ligand]/([ligand]+Kd))

For reliable application of the above equation, it is necessary that the highest peptide ligand concentration successfully tested experimentally be greater than, or at least similar to, the calculated Kd (equivalently, the maximum observed binding should be similar to the calculated saturation binding). In cases where satisfying the above criteria proves difficult, an alternative approach (infra) can be used.

Approach 2:

(1) A fixed concentration of a PDZ-domain polypeptide and increasing concentrations of a labeled PL peptide (labeled with, for example, biotin or fluorescein, see TABLE 9 for representative peptide amino acid sequences) are mixed together in solution and allowed to react. In certain assays, peptide concentrations are 0.1 uM, 1 uM, 10 uM, 100 uM, 1 mM. In other assays, appropriate reaction times can range from 10 minutes to 2 days at temperatures ranging from 4° C. to 37° C. In some instances, the identical reaction can also be carried out using a non-PDZ domain-containing protein as a control (e.g., if the PDZ-domain polypeptide is fusion protein, the fusion partner can be used).

(2) PDZ-ligand complexes can be separated from unbound labeled peptide using a variety of methods known in the art. For example, the complexes can be separated using high performance size-exclusion chromatography (HPSEC, gel filtration) (Rabinowitz et al., 1998, Immunity 9:699), affinity chromatography (e.g., using glutathione Sepharose beads), and affinity absorption (e.g., by binding to an anti-GST-coated plate as described supra).

(3) The PDZ-ligand complex is detected based on presence of the label on the peptide ligand using a variety of methods and detectors known to one of skill in the art. For example, if the label is fluorescein and the separation is achieved using HPSEC, an in-line fluorescence detector can be used. The binding can also be detected as described supra for the G assay.

(4) The PDZ-ligand binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:
Signal_{[Ligand]}=Saturation Binding×([ligand]/([ligand+Kd])

Measurement of the affinity of a labeled peptide ligand binding to a PDZ-domain polypeptide is useful because knowledge of the affinity (or apparent affinity) of this interaction allows rational design of inhibitors of the interaction with known potency. The potency of inhibitors in inhibition would be similar to (i.e., within one-order of magnitude of) the apparent affinity of the labeled peptide ligand binding to the PDZ-domain.

Thus, one method of determining the apparent affinity of binding between a PDZ domain and a ligand involves immobilizing a polypeptide comprising the PDZ domain and a non-PDZ domain on a surface, contacting the immobilized polypeptide with a plurality of different concentrations of the ligand, determining the amount of binding of the ligand to the immobilized polypeptide at each of the concentrations of ligand, and calculating the apparent affinity of the binding based on that data. Typically, the polypeptide comprising the PDZ domain and a non-PDZ domain is a fusion protein. In some instances, the e.g., fusion protein is GST-PDZ fusion protein, but other polypeptides can also be used (e.g., a fusion protein including a PDZ domain and any of a variety of epitope tags, biotinylation signals and the like), so long as the polypeptide can be immobilized in an orientation that does not abolish the ligand binding properties of the PDZ domain, e.g., by tethering the polypeptide to the surface via the non-PDZ domain via an anti-domain antibody and leaving the PDZ domain as the free end. It was discovered, for example, reacting a PDZ-GST fusion polypeptide directly to a plastic plate provided suboptimal results. The calculation of binding affinity itself can be determined using any suitable equation (e.g., as shown supra; also see Cantor and Schimmel (1980) BIOPHYSICAL CHEMISTRY WH Freeman & Co., San Francisco) or software.

Thus, in certain methods, the polypeptide is immobilized by binding the polypeptide to an immobilized immunoglobulin that binds the non-PDZ domain (e.g., an anti-GST antibody when a GST-PDZ fusion polypeptide is used). In some instances, the step of contacting the ligand and PDZ-domain polypeptide is carried out under the conditions provided supra in the description of the “G” assay. It will be appreciated that binding assays are conveniently carried out in multiwell plates (e.g., 24-well, 96-well plates, or 384 well plates).

The present method has considerable advantages over other methods for measuring binding affinities PDZ-PL affinities, which typically involve contacting varying concentrations of a GST-PDZ fusion protein to a ligand-coated surface. For example, some previously described methods for determining affinity (e.g., using immobilized ligand and GST-PDZ protein in solution) did not account for oligomerization state of the fusion proteins used, resulting in potential errors of more than an order of magnitude.

Although not sufficient for quantitative measurement of PDZ-PL binding affinity, an estimate of the relative strength of binding of different PDZ-PL pairs can be made based on the absolute magnitude of the signals observed in the “G assay.” This estimate reflects several factors, including biologically relevant aspects of the interaction, including the affinity and the dissociation rate. For comparisons of different ligands binding to a given PDZ domain-containing protein, differences in absolute binding signal likely relate primarily to the affinity and/or dissociation rate of the interactions of interest.

IX. Assays to Identify Novel PDZ Domain Binding Moieties and to Identify Modulator of PDZ Protein-PL Protein Binding

Although described supra primarily in terms of identifying interactions between PDZ-domain polypeptides and PL proteins, the assays described supra and other assays can also be used to identify the binding of other molecules (e.g., peptide mimetics, small molecules, and the like) to PDZ domain sequences. For example, using the assays disclosed herein, combinatorial and other libraries of compounds can be screened, e.g., for molecules that specifically bind to PDZ domains. Screening of libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In certain assays, screening can be carried out by contacting the library members with a PDZ-domain polypeptide immobilized on a solid support (e.g. as described supra in the “G” assay) and harvesting those library members that bind to the protein. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove.

In other assays, the two-hybrid system for selecting interacting proteins in yeast (Fields and Song, 1989, Nature 340:245-246; Chien et al., 1991, Proc. Natl. Acad. Sci. USA 88:9578-9582) is used to identify molecules that specifically bind to a PDZ domain-containing protein. Furthermore, the identified molecules are further tested for their ability to inhibit transmembrane receptor interactions with a PDZ domain.

In one aspect of the invention, antagonists of an interaction between a PDZ protein and a PL protein are identified. In one embodiment, a modification of the “A” assay described supra is used to identify antagonists. In one embodiment, a modification of the “G” assay described supra is used to identify antagonists.

Screening assays such as these can be used to detect molecules that specifically bind to PDZ domains. Such molecules are useful as agonists or antagonists of PDZ-protein-mediated cell function (e.g., cell activation, e.g., T cell activation, vesicle transport, cytokine release, growth factors, transcriptional changes, cytoskeleton rearrangement, cell movement, chemotaxis, and the like). Thus assays to detect molecules that specifically bind to PDZ domain-containing proteins are provided. For example, recombinant cells expressing PDZ domain-encoding nucleic acids can be used to produce PDZ domains in these assays and to screen for molecules that bind to the domains. Molecules are contacted with the PDZ domain (or fragment thereof) under conditions conducive to binding, and then molecules that specifically bind to such domains are identified. Methods that can be used to carry out the foregoing are commonly known in the art.

It will be appreciated by the ordinarily skilled practitioner that, in some assays, antagonists are identified by conducting the A or G assays in the presence and absence of a known or candidate antagonist. When decreased binding is observed in the presence of a compound, that compound is identified as an antagonist. Increased binding in the presence of a compound signifies that the compound is an agonist.

For example, in one assay, a test compound can be identified as an inhibitor (antagonist) of binding between a PDZ protein and a PL protein by contacting a PDZ domain polypeptide and a PL peptide in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding.

In certain assays, the “G” assay is used in the presence or absence of a candidate inhibitor. In one embodiment, the “A” assay is used in the presence or absence of a candidate inhibitor.

In other assays (in which a G assay is used), one or more PDZ domain-containing GST-fusion proteins are bound to the surface of wells of a 96-well plate as described supra (with appropriate controls including nonfusion GST protein). All fusion proteins are bound in multiple wells so that appropriate controls and statistical analysis can be done. A test compound in BSA/PBS (typically at multiple different concentrations) is added to wells. Immediately thereafter, 30 uL of a detectably labeled (e.g., biotinylated) peptide known to bind to the relevant PDZ domain (see, e.g., TABLE 7 and TABLE 12) is added in each of the wells at a final concentration of, e.g., between about 2 uM and about 40 uM, typically 5 uM, 15 uM, or 25 uM. This mixture is then allowed to react with the PDZ fusion protein bound to the surface for 10 minutes at 4° C. followed by 20 minutes at 25° C. The surface is washed free of unbound peptide three times with ice cold PBS and the amount of binding of the peptide in the presence and absence of the test compound is determined. Usually, the level of binding is measured for each set of replica wells (e.g. duplicates) by subtracting the mean GST alone background from the mean of the raw measurement of peptide binding in these wells.

In certain assays, the A assay is carried out in the presence or absence of a test candidate to identify inhibitors of PL-PDZ interactions.

In some approaches, a test compound is determined to be a specific inhibitor of the binding of the PDZ domain (P) and a PL (L) sequence when, at a test compound concentration of less than or equal to 1 mM (e.g., less than or equal to: 500 uM, 100 uM, 10 uM, 1 uM, 100 nM or 1 nM), the binding of P to L in the presence of the test compound is less than about 50% of the binding in the absence of the test compound (in various embodiments, less than about 25%, less than about 10%, or less than about 1%). Preferably, the net signal of binding of P to L in the presence of the test compound plus six (6) times the standard error of the signal in the presence of the test compound is less than the binding signal in the absence of the test compound.

In one approach, assays for an inhibitor are carried out using a single PDZ protein-PL protein pair (e.g., a PDZ domain fusion protein and a PL peptide). In a related approach, the assays are carried out using a plurality of pairs, such as a plurality of different pairs listed in TABLE 7 or TABLE 12.

In some instances, it is desirable to identify compounds that, at a given concentration, inhibit the binding of one PL-PDZ pair, but do not inhibit (or inhibit to a lesser degree) the binding of a specified second PL-PDZ pair. These antagonists can be identified by carrying out a series of assays using a candidate inhibitor and different PL-PDZ pairs (e.g., as shown in the matrix of TABLE 7 or TABLE 12) and comparing the results of the assays. All such pairwise combinations are contemplated (e.g., test compound inhibits binding of PL₁ to PDZ₁ to a greater degree than it inhibits binding of PL₁ to PDZ₂ or PL₂ to PDZ₂). Importantly, it will be appreciated that, based on the data provided in TABLE 7 and TABLE 12 and disclosed elsehwere herein (and additional data that can be generated using the methods described herein) inhibitors with different specificities can readily be designed.

For example, the Ki (“potency”) of an inhibitor of a PDZ-PL interaction can be determined. Ki is a measure of the concentration of an inhibitor required to have a biological effect. For example, administration of an inhibitor of a PDZ-PL interaction in an amount sufficient to result in an intracellular inhibitor concentration of at least between about 1 and about 100 Ki is expected to inhibit the biological response mediated by the target PDZ-PL interaction. The Kd measurement of PDZ-PL binding as determined using the methods supra can be used in determining Ki.

Thus, certain methods of determining the potency (Ki) of an inhibitor or suspected inhibitor of binding between a PDZ domain and a ligand involve immobilizing a polypeptide comprising the PDZ domain and a non-PDZ domain on a surface, contacting the immobilized polypeptide with a plurality of different mixtures of the ligand and inhibitor, wherein the different mixtures comprise a fixed amount of ligand and different concentrations of the inhibitor, determining the amount of ligand bound at the different concentrations of inhibitor, and calculating the Ki of the binding based on the amount of ligand bound in the presence of different concentrations of the inhibitor. In some instances, the polypeptide is immobilized by binding the polypeptide to an immobilized immunoglobulin that binds the non-PDZ domain. This method, which is based on the “G” assay described supra, is particularly suited for high-throughput analysis of the Ki for inhibitors of PDZ-ligand interactions. Further, using this method, the inhibition of the PDZ-ligand interaction itself is measured, without distortion of measurements by avidity effects.

Typically, at least a portion of the ligand is detectably labeled to permit easy quantitation of ligand binding.

It will be appreciated that the concentration of ligand and concentrations of inhibitor are selected to allow meaningful detection of inhibition. Thus, the concentration of the ligand whose binding is to be blocked is close to or less than its binding affinity (e.g., in other instances less than the 5×Kd of the interaction, in other instances less than 2×Kd, and in still other instances less than 1×Kd). Thus, the ligand is typically present at a concentration of less than 2 Kd (e.g., between about 0.01 Kd and about 2 Kd) and the concentrations of the test inhibitor typically range from 1 nM to 100 uM (e.g. a 4-fold dilution series with highest concentration 10 uM or 1 mM). In a preferred embodiment, the Kd is determined using the assay disclosed supra.

The Ki of the binding can be calculated by any of a variety of methods routinely used in the art, based on the amount of ligand bound in the presence of different concentrations of the inhibitor. In an illustrative embodiment, for example, a plot of labeled ligand binding versus inhibitor concentration is fit to the equation:
S_{inhibitor}=S₀*Ki/([I]+Ki)
where S_{inhibitor} is the signal of labeled ligand binding to immobilized PDZ domain in the presence of inhibitor at concentration [I] and S₀ is the signal in the absence of inhibitor (i.e., [I]=0). Typically [I] is expressed as a molar concentration.

In certain methods, an enhancer (sometimes referred to as, augmentor or agonist) of binding between a PDZ domain and a ligand is identified by immobilizing a polypeptide comprising the PDZ domain and a non-PDZ domain on a surface, contacting the immobilized polypeptide with the ligand in the presence of a test agent and determining the amount of ligand bound, and comparing the amount of ligand bound in the presence of the test agent with the amount of ligand bound by the polypeptide in the absence of the test agent. At least two-fold (often at least 5-fold) greater binding in the presence of the test agent compared to the absence of the test agent indicates that the test agent is an agent that enhances the binding of the PDZ domain to the ligand. As noted supra, agents that enhance PDZ-ligand interactions are useful for disruption (dysregulation) of biological events requiring normal PDZ-ligand function (e.g., cancer cell division and metastasis, and activation and migration of immune cells).

The “potency” or “K_{enhancer}” of an enhancer of a PDZ-ligand interaction can also be determined. For example, the K_{enhancer} of an enhancer of a PDZ-PL interaction can be determined, e.g., using the Kd of PDZ-PL binding as determined using the methods described supra. K_{enhancer} is a measure of the concentration of an enhancer expected to have a biological effect. For example, administration of an enhancer of a PDZ-PL interaction in an amount sufficient to result in an intracellular inhibitor concentration of at least between about 0.1 and about 100 K_{enhancer} (e.g., between about 0.5 and about 50 K_{enhancer}) is expected to disrupt the biological response mediated by the target PDZ-PL interaction.

Thus, in one aspect the invention provides a method of determining the potency (K_{enhancer}) of an enhancer or suspected enhancer of binding between a PDZ domain and a ligand by immobilizing a polypeptide comprising the PDZ domain and a non-PDZ domain on a surface, contacting the immobilized polypeptide with a plurality of different mixtures of the ligand and enhancer, wherein the different mixtures comprise a fixed amount of ligand, at least a portion of which is detectably labeled, and different concentrations of the enhancer, determining the amount of ligand bound at the different concentrations of enhancer, and calculating the potency (K_{enhancer}) of the enhancer from the binding based on the amount of ligand bound in the presence of different concentrations of the enhancer. Typically, at least a portion of the ligand is detectably labeled to permit easy quantitation of ligand binding. This method, which is based on the “G” assay described supra, is particularly suited for high-throughput analysis of the K_{enhancer} for enhancers of PDZ-ligand interactions.

It will be appreciated that the concentration of ligand and concentrations of enhancer are selected to allow meaningful detection of enhanced binding. Thus, the ligand is typically present at a concentration of between about 0.01 Kd and about 0.5 Kd and the concentrations of the test agent/enhancer typically range from 1 nM to 1 mM (e.g. a 4-fold dilution series with highest concentration 10 uM or 1 mM). In a preferred embodiment, the Kd is determined using the assay disclosed supra.

The potency of the binding can be determined by a variety of standard methods based on the amount of ligand bound in the presence of different concentrations of the enhancer or augmentor. For example, a plot of labeled ligand binding versus enhancer concentration can be fit to the equation:
S([E])=S(0)+(S(0)*(D_{enhancer}−1)*[E]/([E]+K_{enhancer})
where “K_{enhancer}” is the potency of the augmenting compound, and “D_{enhancer}” is the fold-increase in binding of the labeled ligand obtained with addition of saturating amounts of the enhancing compound, [E] is the concentration of the enhancer. It will be understood that saturating amounts are the amount of enhancer such that further addition does not significantly increase the binding signal. Knowledge of “K_{enhancer}” is useful because it describes a concentration of the augmenting compound in a target cell that will result in a biological effect due to dysregulation of the PDZ-PL interaction. Typical therapeutic concentrations are between about 0.1 and about 100 K_{enhancer}.
X. Identification of Pharmaceutical Compounds that Inhibit PDZ-PL Proteins

For certain of the PDZ proteins and PL proteins shown to bind together and for which Kd values had been obtained, additional testing was conducted to determine whether certain pharmaceutical compounds would act to antagonize or agonize the interactions. Assays were conducted as for the G′ assay described supra both in the presence and absence of test compound, except that 50 ul of a 10 uM solution of the biotinylated PL peptide is allowed to react with the surface bearing the PDZ-domain polypeptide instead of a 20 uM solution as specified in step (2) of the assay.

Results from such studies are shown in TABLES 10A and 10B. In both tables, the first column (left to right) entitled “PDZ domain” lists the gene name of GST-PDZ domain fusion (see TABLE 9). Entries having two numbers separated by a slash indicate which PDZ domain was utilized. For example, in TABLE 10A, the entry for ZO-3 is 1/3. This means that PDZ domain 1 of 3 was used.

The second column labeled “PL” indicates the name of the PDZ ligand (see TABLES 10A and 10B) interacting with the PDZ domain. The third column entitled “Drug” lists the common or trade name of pharmaceutical compound tested and found to modulate the specific PDZ-PL interaction (suppliers and chemical information are listed in TABLE 11). The final column with the heading “Change in OD” indicates the change in absorbance at 450 nm of the assay in the absence (first number) or presence (second number) of chemical compound.

TABLE 11 provides the generic and commercial names for the compounds tested, as well as the Sigma Chemical Company catalog number. The molecular weight is listed in grams/mole. The final column in TABLE 11 lists 200 times the therapeutic dose as listed in the Physicians Desk Reference and is listed in mg/ml. Stock solutions were made fresh at these concentrations and used in the assay at 10 times the therapeutic dose.

XI. Global Analysis of PDZ-PL Interactions

Certain analyses involve determining the affinity for a particular ligand and a plurality of PDZ proteins. Typically the plurality is at least 5, and often at least 25, or at least 40 different PDZ proteins. In certain analyses, the plurality of different PDZ proteins are from a particular tissue (e.g., central nervous system, spleen, cardiac muscle, kidney) or a particular class or type of cell, (e.g., a hematopoietic cell, a lymphocyte, a neuron) and the like. In some instances, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in lymphocytes. For example, in some analyses, the plurality is at least 50%, usually at least 80%, at least 90% or all of the PDZ proteins disclosed herein as being expressed in hematopoietic cells.

The binding of a ligand to the plurality of PDZ proteins is determined in some analyses. Using this method, it is possible to identify a particular PDZ domain bound with particular specificity by the ligand. The binding can be designated as “specific” if the affinity of the ligand to the particular PDZ domain is at least 2-fold that of the binding to other PDZ domains in the plurality (e.g., present in that cell type). The binding is deemed “very specific” if the affinity is at least 10-fold higher than to any other PDZ in the plurality or, alternatively, at least 10-fold higher than to at least 90%, more often 95% of the other PDZs in a defined plurality. Similarly, the binding is deemed “exceedingly specific” if it is at least 100-fold higher. For example, a ligand could bind to 2 different PDZs with an affinity of 1 uM and to no other PDZs out of a set 40 with an affinity of less than 100 uM. This would constitute specific binding to those 2 PDZs. Similar measures of specificity are used to describe binding of a PDZ to a plurality of PLs.

It will be recognized that high specificity PDZ-PL interactions generally represent potentially more valuable targets for achieving a desired biological effect. The ability of an inhibitor or enhancer to act with high specificity is often desirable. In particular, the most specific PDZ-ligand interactions are also the best therapeutic targets, allowing specific inhibition of the interaction.

Identifying a high specificity interaction between a particular PDZ domain and a ligand known or suspected of binding at least one PDZ domain can be achieved with various methods. Certain methods involve providing a plurality of different immobilized polypeptides, each of said polypeptides comprising a PDZ domain and a non-PDZ domain; determining the affinity of the ligand for each of said polypeptides, and comparing the affinity of binding of the ligand to each of said polypeptides, wherein an interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain.

In related methods, the affinity of binding of a specific PDZ domain to a plurality of ligands (or suspected ligands) is determined. For example, in one embodiment, the invention provides a method of identifying a high specificity interaction between a PDZ domain and a particular ligand known or suspected of binding at least one PDZ domain, by providing an immobilized polypeptide comprising the PDZ domain and a non-PDZ domain; determining the affinity of each of a plurality of ligands for the polypeptide, and comparing the affinity of binding of each of the ligands to the polypeptide, wherein an interaction between a particular ligand and the PDZ domain is deemed to have high specificity when the ligand binds an immobilized polypeptide comprising the PDZ domain with at least 2-fold higher affinity than other ligands tested. Thus, the binding may be designated as “specific” if the affinity of the PDZ to the particular PL is at least 2-fold that of the binding to other PLs in the plurality (e.g., present in that cell type). The binding is deemed “very specific” if the affinity is at least 10-fold higher than to any other PL in the plurality or, alternatively, at least 10-fold higher than to at least 90%, more often 95% of the other PLs in a defined plurality. Similarly, the binding is deemed “exceedingly specific” if it is at least 100-fold higher. Typically the plurality is at least 5 different ligands, more often at least 10.

A. Use of Array for Global Predictions

The inventors have found that valuable information can be ascertained by analysis (e.g., simultaneous analysis) of a large number of PDZ-PL interactions. Certain analyses encompass all of the PDZ proteins expressed in a particular tissue (e.g., spleen) or type or class of cell (e.g., hematopoietic cell, neuron, lymphocyte, B cell, T cell and the like). Alternatively, the analysis encompasses at least about 5, or at least about 10, or at least about 12, or at least about 15 and often at least 50 different polypeptides, up to about 60, about 80, about 100, about 150, about 200, or even more different polypeptides; or a substantial fraction (e.g., typically a majority, more often at least 80%) of all of the PDZ proteins known to be, or suspected of being, expressed in the tissue or cell(s), e.g., all of the PDZ proteins known to be present in lymphocytes.

It will be recognized that the arrays and methods described herein are directed to the analysis of PDZ and PL interactions, and involve selection of such proteins for analysis. While the devices and methods disclosed herein can include or involve a small number of control polypeptides, they typically do not include significant numbers of proteins or fusion proteins that do not include either PDZ or PL domains (e.g., typically, at least about 90% of the arrayed or immobilized polypeptides in a method or device of the invention is a PDZ or PL sequence protein, more often at least about 95%, or at least about 99%).

It will be apparent from this disclosure that analysis of the relatively large number of different interactions preferably takes place simultaneously. In this context, “simultaneously” means that the analysis of several different PDZ-PL interactions (or the effect of a test agent on such interactions) is assessed at the same time. Typically the analysis is carried out in a high throughput (e.g., robotic) fashion. One advantage of this method of simultaneous analysis is that it permits rigorous comparison of multiple different PDZ-PL interactions. For example, as explained in detail elsewhere herein, simultaneous analysis (and use of the arrays described infra) facilitates, for example, the direct comparison of the effect of an agent (e.g., an potential interaction inhibitor) on the interactions between a substantial portion of PDZs and/or PLs in a tissue or cell.

Accordingly, an array of immobilized polypeptide comprising the PDZ domain and a non-PDZ domain on a surface can be utilized in binding analyses. Typically, the array comprises at least about 5, or at least about 10, or at least about 12, or at least about 15 and often at least 50 different polypeptides. In one preferred embodiment, the different PDZ proteins are from a particular tissue (e.g., central nervous system, spleen, cardiac muscle, kidney) or a particular class or type of cell, (e.g., a hematopoietic cell, a lymphocyte, a neuron) and the like. In a most preferred embodiment, the plurality of different PDZ proteins represents a substantial fraction (e.g., typically a majority, more often at least 60%, 70% or 80%) of all of the PDZ proteins known to be, or suspected of being, expressed in the tissue or cell(s), e.g., all of the PDZ proteins known to be present in lymphocytes.

Certain arrays include a plurality, usually at least 5, 10, 25, 50 PDZ proteins present in a particular cell of interest. In this context, “array” refers to an ordered series of immobilized polypeptides in which the identity of each polypeptide is associated with its location. In some instances, the plurality of polypeptides are arrayed in a “common” area such that they can be simultaneously exposed to a solution (e.g., containing a ligand or test agent). For example, the plurality of polypeptides can be on a slide, plate or similar surface, which can be plastic, glass, metal, silica, beads or other surface to which proteins can be immobilized. In other instances, the different immobilized polypeptides are situated in separate areas, such as different wells of multi-well plate (e.g., a 24-well plate, a 96-well plate, a 384 well plate, and the like). It will be recognized that a similar advantage can be obtained by using multiple arrays in tandem.

B. Analysis of PDZ-PL Inhibition Profile

Some methods involve determining if a test compound inhibits any PDZ-ligand interaction in large set of PDZ-ligand interaction (e.g., a plurality of the PDZ-ligands interactions described in TABLE 7 or TABLE 12; a majority of the PDZ-ligands identified in a particular cell or tissue as described supra (e.g., lymphocytes) and the like). In one embodiment, the PDZ domains of interest are expressed as GST-PDZ fusion proteins and immobilized as described herein. For each PDZ domain, a labeled ligand that binds to the domain with a known affinity is identified as described herein.

For any known or suspected modulator (e.g., inhibitor) of a PDL-PL interaction(s), it is useful to know which interactions are inhibited (or augmented). For example, an agent that inhibits all PDZ-PL interactions in a cell (e.g., a lymphocyte) will have different uses than an agent that inhibits only one, or a small number, of specific PDZ-PL interactions. The profile of PDZ interactions inhibited by a particular agent is referred to as the “inhibition profile” for the agent, and is described in detail below. The profile of PDZ interactions enhanced by a particular agent is referred to as the “enhancement profile” for the agent. It will be readily apparent to one of skill guided by the description of the inhibition profile how to determine the enhancement profile for an agent. Thus, methods for determining the PDZ interaction (inhibition/enhancement) profile of an agent in a single assay are provided.

Certain methods involve determining the PDZ-PL inhibition profile of a compound by providing (i) a plurality of different immobilized polypeptides, each of said polypeptides comprising a PDZ domain and a non-PDZ domain and (ii) a plurality of corresponding ligands, wherein each ligand binds at least one PDZ domain in (i), then contacting each of said immobilized polypeptides in (i) with a corresponding ligand in (ii) in the presence and absence of a test compound, and determining for each polypeptide-ligand pair whether the test compound inhibits binding between the immobilized polypeptide and the corresponding ligand.

Typically the plurality is at least 5, and often at least 25, or at least 40 different PDZ proteins. In certain analyses, the plurality of different ligands and the plurality of different PDZ proteins are from the same tissue or a particular class or type of cell, e.g., a hematopoietic cell, a lymphocyte, a neuron and the like. In some instances, the plurality of different PDZs represents a substantial fraction (e.g., at least 80%) of all of the PDZs known to be, or suspected of being, expressed in the tissue or cell(s), e.g., all of the PDZs known to be present in lymphocytes (for example, at least 80%, at least 90% or all of the PDZs disclosed herein as being expressed in hematopoietic cells).

In certain instances, the inhibition profile is determined as follows: A plurality (e.g., all known) PDZ domains expressed in a cell (e.g., lymphocytes) are expressed as GST-fusion proteins and immobilized without altering their ligand binding properties as described supra. For each PDZ domain, a labeled ligand that binds to this domain with a known affinity is identified. If the set of PDZ domains expressed in lymphocytes is denoted by {P1 . . . Pn}, any given PDZ domain Pi binds a (labeled) ligand Li with affinity K_{d}i. To determine the inhibition profile for a test agent “compound X” the “G” assay (supra) can be performed as follows in 96-well plates with rows A-H and columns 1-12. Column 1 is coated with P1 and washed. The corresponding ligand L1 is added to each washed coated well of column 1 at a concentration 0.5 K_{d}1 with (rows B, D, F, H) or without (rows A, C, E, F) between about 1 and about 1000 uM) of test compound X. Column 2 is coated with P2, and L2 (at a concentration 0.5 K_{d}2) is added with or without inhibitor X. Additional PDZ domains and ligands are similarly tested.

Compound X is considered to inhibit the binding of Li to Pi if the average signal in the wells of column i containing X is less than half the signal in the equivalent wells of the column lacking X. Thus, in this single assay one determines the full set of lymphocyte PDZs that are inhibited by compound X.

In some embodiments, the test compound X is a mixture of compounds, such as the product of a combinatorial chemistry synthesis as described supra. In some embodiments, the test compound is known to have a desired biological effect, and the assay is used to determine the mechanism of action (i.e., if the biological effect is due to modulating a PDZ-PL interaction).

It will be apparent that an agent that modulates only one, or a few PDZ-PL interactions, in a panel (e.g., a panel of all known PDZs lymphocytes, a panel of at least 10, at least 20 or at least 50 PDZ domains) is a more specific modulator than an agent that modulate many or most interactions. Typically, an agent that modulates less than 20% of PDZ domains in a panel (e.g., TABLE 7 or TABLE 12) is deemed a “specific” inhibitor, less than 6% a “very specific” inhibitor, and a single PDZ domain a “maximally specific” inhibitor.

It will also be appreciated that “compound X” can be a composition containing mixture of compounds (e.g., generated using combinatorial chemistry methods) rather than a single compound.

Several variations of this assay can be utilized:

In some assays, the assay above is performed using varying concentrations of the test compound X, rather than fixed concentration. This allows determination of the Ki of the X for each PDZ as described above.

In other assays, instead of pairing each PDZ Pi with a specific labeled ligand Li, a mixture of different labeled ligands is created that such that for every PDZ at least one of the ligands in the mixture binds to this PDZ sufficiently to detect the binding in the “G” assay. This mixture is then used for every PDZ domain.

In some instances, compound X is known to have a desired biological effect, but the chemical mechanism by which it has that effect is unknown. The assays of the invention can then be used to determine if compound X has its effect by binding to a PDZ domain.

In certain assays, PDZ-domain containing proteins are classified in to groups based on their biological function, e.g. into those that regulate chemotaxis versus those that regulate transcription. An optimal inhibitor of a particular function (e.g., including but not limited to an anti-chemotactic agent, an anti-T cell activation agent, cell-cycle control, vesicle transport, apoptosis, etc.) will inhibit multiple PDZ-ligand interactions involved in the function (e.g., chemotaxis, activation) but few other interactions. Thus, the assay is used in one embodiment in screening and design of a drug that specifically blocks a particular function. For example, an agent designed to block chemotaxis might be identified because, at a given concentration, the agent inhibits 2 or more PDZs involved in chemotaxis but fewer than 3 other PDZs, or that inhibits PDZs involved in chemotaxis with a Ki>10-fold better than for other PDZs. Thus, methods can be designed to identify an agent that inhibits a first selected PDZ-PL interaction or plurality of interactions, while not inhibiting a second selected PDZ-PL interaction or plurality of interactions. The two (or more) sets of interactions can be selected on the basis of the known biological function of the PDZ proteins, the tissue specificity of the PDZ proteins, or any other criteria. Moreover, the assay can be used to determine effective doses (i.e., drug concentrations) that result in desired biological effects while avoiding undesirable effects.

C. Side Effects of PDZ-PL Modulator Interactions

Methods can also be conducted to determine likely side effects of a therapeutic that inhibits PDZ-ligand interactions. Such methods entail identifying those target tissues, organs or cell types that express PDZ proteins and ligands that are disrupted by a specified inhibitor. If, at a therapeutic dosage, a drug intended to have an effect in one organ system (e.g., hematopoietic system) disrupts PDZ-PL interactions in a different system (e.g., CNS) it can be predicted that the drug will have effects (“side effects”) on the second system. It will be apparent that the information obtained from this assay will be useful in the rational design and selection of drugs that do not have the side-effect.

In certain methods, for example, a comprehensive PDZ protein set is obtained. A “perfectly comprehensive” PDZ protein set is defined as the set of all PDZ proteins expressed in the subject animal (e.g., humans). A comprehensive set can be obtained by analysis of, for example, the human genome sequence. However, a “perfectly comprehensive” set is not required and any reasonably large set of PDZ domain proteins (e.g., the set of all known PDZ proteins; or the set listed in TABLE 9) will provide valuable information.

Thus, some methods involve some of all of the following steps:

a) For each PDZ protein, determine the tissues in which it is highly expressed. This can be done experimentally, although the information generally will be available in the scientific literature;

b) For each PDZ protein (or as many as possible), identify the cognate PL(s) bound by the PDZ protein;

c) Determine the Ki at which the test agent inhibits each PDZ-PL interaction, using the methods described supra;

d) From this information it is possible to calculate the pattern of PDZ-PL interactions disrupted at various concentrations of the test agent.

By correlating the set of PDZ-PL interactions disrupted with the expression pattern of the members of that set, it will be possible to identify the tissues likely affected by the agent.

Additional steps can also be carried out, including determining whether a specified tissue or cell type is exposed to an agent following a particular route of administration. This can be determined using basis pharmacokinetic methods and principles.

D. Modulation of Activities

The PDZ binding moieties and PDZ protein-PL protein binding antagonists of the invention are used to modulate biological activities or functions of cells (e.g., hematopoietic cells, such as T cells and B cells and the like), endothelial cells, and other immune system cells, as described herein, and for treatment of diseases and conditions in human and nonhuman animals (e.g., experimental models). Exemplary biological activities are listed supra.

When administered to patients, the compounds identified utilizing the methods described herein (e.g., PL-PDZ interaction inhibitors) are useful for treating (ameliorating symptoms of) a variety of diseases and conditions, including diseases characterized by inflammatory and humoral immune responses, e.g., inflammation, allergy (e.g., systemic anaphylaxis, hypersensitivity responses, drug allergies, insect sting allergies; inflammatory bowel diseases, ulcerative colitis, ileitis and enteritis; psoriasis and inflammatory dermatoses, scleroderma; respiratory allergic diseases such as asthma, allergic rhinitis, hypersensitivity lung diseases, and the like vasculitis, rh incompatibility, transfusion reactions, drug sensitivities, PIH, atopic dermatitis, eczema, rhinnitis; autoimmune diseases, such as arthritis (rheumatoid and psoriatic), multiple sclerosis, systemic lupus erythematosus, insulin-dependent diabetes, glomerulonephritis, scleroderma, MCTD, IDDM, Hashimoto thyroiditis, Goodpasture syndrome, psoriasis and the like, osteoarthritis, polyarthritis, graft rejection (e.g., allograft rejection, e.g., renal allograft rejection, graft-vs-host disease, transplantation rejection (cardiac, kidney, lung, liver, small bowel, cornea, pancreas, cadaver, autologous, bone marrow, xenotransplantation)), atherosclerosis, angiogenesis-dependent disorders, cancers (e.g., melanomas and breast cancer, prostrate cancer, leukemias, lymphomas, metastatic disease), infectious diseases (e.g., viral infection, such as HIV, measles, parainfluenza, virus-mediated cell fusion,), ischemia (e.g., post-myocardial infarction complications, joint injury, kidney, scleroderma).

E. Agonists and Antagonists of PDZ-PL Interactions

As described herein, interactions between PDZ proteins and PL proteins in cells (e.g., hematopoietic cells, e.g., T cells and B cells) can be disrupted or inhibited by the administration of inhibitors or antagonists. Inhibitors can be identified using screening assays described herein. In some instances, the motifs disclosed herein are used to design inhibitors. In other instances, the antagonists of the invention have a structure (e.g., peptide sequence) based on the C-terminal residues of PL-domain proteins listed in TABLE 8. In some embodiments, the antagonists have a structure (e.g., peptide sequence) based on a PL motif disclosed herein.

The PDZ/PL antagonists and antagonists can be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds. Although, for convenience, the present discussion primarily refers antagonists of PDZ-PL interactions, it will be recognized that PDZ-PL interaction agonists can also be use in the methods disclosed herein.

In one aspect, the peptides and peptide mimetics or analogues of the invention contain an amino acid sequence that binds a PDZ domain in a cell of interest. In one embodiment, the antagonists comprise a peptide that has a sequence corresponding to the carboxy-terminal sequence of a PL protein listed in TABLE 8, e.g., a peptide listed TABLE 8. Typically, the peptide comprises at least the C-terminal two (3), three (3) or four (4) residues of the PL protein, and often the inhibitory peptide comprises more than four residues (e.g., at least five, six, seven, eight, nine, ten, twelve or fifteen residues) from the PL protein C-terminus.

In some instances, the inhibitor is a peptide, e.g., having a sequence of a PL C-terminal protein sequence.

In some embodiments, the antagonist is a fusion protein comprising such a sequence. Fusion proteins containing a transmembrane transporter amino acid sequence are particularly useful.

In other instances, the inhibitor is conserved variant of the PL C-terminal protein sequence having inhibitory activity.

In some embodiments, the antagonist is a peptide mimetic of a PL C-terminal sequence.

In some embodiments, the inhibitor is a small molecule (i.e., having a molecular weight less than 1 kD).

F. Peptide Antagonists

Certain antagonists comprise a peptide that has a sequence of a PL protein carboxy-terminus listed in TABLE 8. The peptide comprises at least the C-terminal two (2) residues of the PL protein, and typically, the inhibitory peptide comprises more than two residues (e.g, at least three, four, five, six, seven, eight, nine, ten, twelve or fifteen residues) from the PL protein C-terminus. The peptide can be any of a variety of lengths (e.g., at least 2, at least 3, at least 4, at least 5, at least 6, at least 8, at least 10, or at least 20 residues) and can contain additional residues not from the PL protein. It will be recognized that short PL peptides are sometime used in the rational design of other small molecules with similar properties.

Although most often, the residues shared by the inhibitory peptide with the PL protein are found at the C-terminus of the peptide. However, in some embodiments, the sequence is internal. Similarly, in some cases, the inhibitory peptide comprises residues from a PL sequence that is near, but not at the c-terminus of a PL protein (see, Gee et al., 1998, J Biological Chem. 273:21980-87).

Sometime the PL protein carboxy-terminus sequence is referred to as the “core PDZ motif sequence” referring to the ability of the short sequence to interact with the PDZ domain. For example, in an embodiment, the “core PDZ motif sequence” contains the last four C-terminus amino acids. As described above, the four amino acid core of a PDZ motif sequence can contain additional amino acids at its amino terminus to further increase its binding affinity and/or stability. Thus, in one embodiment, the PDZ motif sequence peptide can be from four amino acids up to 15 amino acids. It is preferred that the length of the sequence to be 6-10 amino acids. More preferably, the PDZ motif sequence contains 8 amino acids. Additional amino acids at the amino terminal end of the core sequence can be derived from the natural sequence in each hematopoietic cell surface receptor or a synthetic linker. The additional amino acids can also be conservatively substituted. When the third residue from the C-terminus is S, T or Y, this residue can be phosphorylated prior to the use of the peptide.

The peptide and nonpeptide inhibitors can be small, e.g., fewer than ten amino acid residues in length if a peptide. Further, it is reported that a limited number of ligand amino acids directly contact the PDZ domain (generally less than eight) (Kozlov et al., 2000, Biochemistry 39, 2572; Doyle et al., 1996, Cell 85, 1067) and that peptides as short as the C-terminal three amino acids often retain similar binding properties to longer (>15) amino acids peptides (Yanagisawa et al., 1997, J. Biol. Chem. 272, 8539).

G. Peptide Variants

Having identified PDZ binding peptides and PDZ-PL interaction inhibitory sequences, variations of these sequences can be made and the resulting peptide variants can be tested for PDZ domain binding or PDZ-PL inhibitory activity. In certain instances, the variants have the same or a different ability to bind a PDZ domain as the parent peptide. Typically, such amino acid substitutions are conservative, i.e., the amino acid residues are replaced with other amino acid residues having physical and/or chemical properties similar to the residues they are replacing. Preferably, conservative amino acid substitutions are those wherein an amino acid is replaced with another amino acid encompassed within the same designated class.

H. Peptide Mimetics

Having identified PDZ binding peptides and PDZ-PL interaction inhibitory sequences, peptide mimetics can be prepared using routine methods, and the inhibitory activity of the mimetics can be confirmed using the assays of the invention. Thus, certain antagonists are a peptide mimetic of a PL C-terminal sequence. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

I. Small Molecules

In some embodiments, the inhibitor is a small molecule (i.e., having a molecular weight less than 1 kD). Methods for screening small molecules are well known in the art and include those described supra.

XII Preparation of Peptides

A. Chemical Synthesis

The peptides or analogues thereof that are described herein, can be prepared using virtually any art-known technique for the preparation of peptides and peptide analogues. For example, the peptides can be prepared in linear form using conventional solution or solid phase peptide syntheses and cleaved from the resin followed by purification procedures (Creighton, 1983, Protein Structures And Molecular Principles, W.H. Freeman and Co., N.Y.). Suitable procedures for synthesizing the peptides described herein are well known in the art. The composition of the synthetic peptides can be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention can be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

B. Recombinant Synthesis

If the peptide is composed entirely of gene-encoded amino acids, or a portion of it is so composed, the peptide or the relevant portion can also be synthesized using conventional recombinant genetic engineering techniques. For recombinant production, a polynucleotide sequence encoding a linear form of the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems can be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used; when cloning in insect cell systems, promoters such as the baculovirus polyhedron promoter can be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) can be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) can be used; when generating cell lines that contain multiple copies of expression product, SV40-, BPV- and EBV-based vectors can be used with an appropriate selectable marker.

In cases where plant expression vectors are used, the expression of sequences encoding the peptides of the invention can be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) can be used; alternatively, plant promoters such as the small subunit of RUBISCO (Coruzzi et al., 1984, EMBO J.3:1671-1680; Broglie et al., 1984, Science 224:838-843) or heat shock promoters, e.g., soybean hsp17.5-E or hsp17.3-B (Gurley et al., 1986, Mol. Cell. Biol. 6:559-565) can be used. These constructs can be introduced into planleukocytes using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that can be used to produce the peptides of the invention, Autographa californica nuclear polyhidrosis virus (AcNPV) is used as a vector to express the foreign genes. The virus grows in Spodoptera frugiperda cells. A coding sequence can be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system can be found in Current Protocols in Molecular Biology, Vol. 2, Ausubel et al., eds., Greene Publish. Assoc. & Wiley Interscience.

In mammalian host cells, a number of viral based expression systems can be utilized. In cases where an adenovirus is used as an expression vector, a coding sequence can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. USA 81:3655-3659). Alternatively, the vaccinia 7.5 K promoter can be used, (see, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear peptides of the invention will be apparent to those having skill in the art.

Purification of the Peptides and Peptide Analogues

The peptides and peptide analogues that are provided can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art. The purified peptides can be identified by assays based on their physical or functional properties, including radioactive labeling followed by gel electrophoresis, radioimmuno-assays, ELISA, bioassays, and the like.

For affinity chromatography purification, any antibody which specifically binds the peptides or peptide analogues can be used. For the production of antibodies, various host animals, including but not limited to rabbits, mice, rats, etc., can be immunized by injection with a peptide. The peptide can be attached to a suitable carrier, such as BSA or KLH, by means of a side chain functional group or linkers attached to a side chain functional group. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide can be prepared using any technique which provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments which contain deletions of specific binding sites can be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries can be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Immunoaffinity Chromatography of Proteins 34:723-731.

XIII. Uses of PDZ Domain Binding and Antagonist Compounds

The PDZ domain-containing proteins dislcosed herein are involved in a number of biological functions, including, but not limited to, vesicular trafficking, tumor suppression, signal transduction, protein sorting, establishment of membrane polarity, apoptosis, regulation of immune response and organization of synapse formation. In general, this family of proteins has a common function of facilitating the assembly of multi-protein complexes, often serving as a bridge between several proteins, or regulating the function of other proteins. Additionally, as also noted supra, these proteins are found in essentially all cell types.

Consequently, modulation of these interactions can be utilized to control a wide variety of biological conditions and physiological conditions. In particular, modulation of interactions such as those disclosed herein can be utilized to control movement of vesicles within a cell, inhibition of tumor formation, as well as in the treatment of immune disorders, neurological disorders, muscular disorders, and intestinal disorders.

Certain compounds which modulate binding of the PDZ proteins and PL proteins can be used to inhibit leukocyte activation, which is manifested in measurable events including but not limited to, cytokine production, cell adhesion, expansion of cell numbers, apoptosis and cytotoxicity. Thus, some compounds of the invention can be used to treat diverse conditions associated with undesirable leukocyte activation, including but not limited to, acute and chronic inflammation, graft-versus-host disease, transplantation rejection, hypersensitivities and autoimmunity such as multiple sclerosis, rheumatoid arthritis, peridontal disease, systemic lupus erythematosis, juvenile diabetes mellitis, non-insulin-dependent diabetes, and allergies, and other conditions listed herein.

More specifically, in view of the various classes the PDZ and PL proteins identified herein fall into (see Section IV), the compounds can be utilized to regulate biological functions involving protein kinases, guanalyte kinases, guanine exchange factors, LIM PDZs, tyrosine phosphatases, serine proteases, viral oncogene interacting proteins, T-cell surface receptors, B-cell surface receptors, natural killer cell receptors, monocyte surface receptors, monocyte surface receptors, granulocyte surface receptors, endothelial cell surface receptors, G-protein linked receptors, tight junction integral membrane proteins, cell adhesion molecules, neuron transport and organization molecules, regulators of G-protein signaling, ion channels and transporters and tumor associated proteins and receptors.

XIV. Formulation and Route of Administration

A. Introduction of Agonists or Antagonists (e.g., Peptides and Fusion Proteins) into Cells

In certain methods, PDZ-PL antagonists are introduced into a cell to modulate (i.e., increase or decrease) a biological function or activity of the cell. Many small organic molecules readily cross the cell membranes (or can be modified by one of skill using routine methods to increase the ability of compounds to enter cells, e.g., by reducing or eliminating charge, increasing lipophilicity, conjugating the molecule to a moiety targeting a cell surface receptor such that after interacting with the receptor). Methods for introducing larger molecules, e.g., peptides and fusion proteins are also well known, including, e.g., injection, liposome-mediated fusion, application of a hydrogel, conjugation to a targeting moiety conjugate endocytozed by the cell, electroporation, and the like).

In some instances, the antagonist or agent is a fusion polypeptide or derivatized polypeptide. A fusion or derivatized protein can include a targeting moiety that increases the ability of the polypeptide to traverse a cell membrane or causes the polypeptide to be delivered to a specified cell type (e.g., liver cells or tumor cells) preferentially or cell compartment (e.g., nuclear compartment) preferentially. Examples of targeting moieties include lipid tails, amino acid sequences such as antennapoedia peptide or a nuclear localization signal (NLS; e.g., Xenopus nucleoplasmin Robbins et al., 1991, Cell 64:615).

In certain approaches, a peptide sequence or peptide analog, determined to inhibit a PDZ domain-PL protein binding by an assay described herein, is introduced into a cell by linking the sequence to an amino acid sequence that facilitates its transport through the plasma membrane (a “transmembrane transporter sequence”). Peptides with a desired activity can be used directly or fused to a transmembrane transporter sequence to facilitate their entry into cells. In the case of such a fusion peptide, each peptide can be fused with a heterologous peptide at its amino terminus directly or by using a flexible polylinker such as the pentamer G-G-G-G-S (SEQ ID NO:836) repeated 1 to 3 times. Such linker has been used in constructing single chain antibodies (scFv) by being inserted between V_{H} and V_{L} (Bird et al., 1988, Science 242:423-426; Huston et al., 1988, Proc. Natl. Acad. Sci. U.S.A. 85:5979-5883). The linker is designed to enable the correct interaction between two beta-sheets forming the variable region of the single chain antibody. Other linkers which can be used include Glu-Gly-Lys-Ser-Ser-Gly-Ser-Gly-Ser-Glu-Ser-Lys-Val-Asp (SEQ ID NO:837) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO:838) (Bird et al., 1988, Science 242:423-426).

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606), 70 KDa heat shock protein (Fujihara, 1999, EMBO J. 18:411-419) and transportan (Pooga et al., 1998, FASEB J. 12:67-77). In a preferred embodiment of the invention, a truncated HIV tat peptide having the sequence of GYGRKKRRQRRRG (SEQ ID NO:494) is used.

It is preferred that a transmembrane transporter sequence is fused to a hematopoietic cell surface receptor carboxyl terminal sequence at its amino-terminus with or without a linker. Generally, the C-terminus of a PDZ motif sequence (PL sequence) must be free in order to interact with a PDZ domain. The transmembrane transporter sequence can be used in whole or in part as long as it is capable of facilitating entry of the peptide into a cell.

In certain methods, a hematopoietic cell surface receptor C-terminal sequence can be used alone when it is delivered in a manner that allows its entry into cells in the absence of a transmembrane transporter sequence. For example, the peptide can be delivered in a liposome formulation or using a gene therapy approach by delivering a coding sequence for the PDZ motif alone or as a fusion molecule into a target cell.

Active compounds can also be administered via liposomes, which serve to target the conjugates to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide or conjugate of the invention can be directed to the site of lymphoid cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the immune cells, a ligand to be incorporated into the liposome can include, e.g., antibodies or fragments thereof specific for cell surface determinants of the desired immune system cells. A liposome suspension containing a peptide or conjugate can be administered intravenously, locally, topically, etc. in a dose which varies according to, inter alia, the manner of administration, the conjugate being delivered, and the stage of the disease being treated.

In order to specifically deliver a PDZ motif sequence (PL sequence) peptide into a specific cell type, the peptide can be linked to a cell-specific targeting moiety, which include but are not limited to, ligands for diverse leukocyte surface molecules such as growth factors, hormones and cytokines, as well as antibodies or antigen-binding fragments thereof. Since a large number of cell surface receptors have been identified in leukocytes, ligands or antibodies specific for these receptors can be used as cell-specific targeting moieties. For example, interleukin-2, B7-1 (CD80), B7-2 (CD86) and CD40 or peptide fragments thereof can be used to specifically target activated T cells (The Leucocyte Antigen Facts Book, 1997, Barclay et al. (eds.), Academic Press). CD28, CTLA-4 and CD40L or peptide fragments thereof can be used to specifically target B cells. Furthermore, Fc domains can be used to target certain Fc receptor-expressing cells such as monocytes.

Antibodies are the most versatile cell-specific targeting moieties because they can be generated against any cell surface antigen. Monoclonal antibodies have been generated against leukocyte lineage-specific markers such as certain CD antigens. Antibody variable region genes can be readily isolated from hybridoma cells by methods well known in the art. However, since antibodies are assembled between two heavy chains and two light chains, it is preferred that a scFv be used as a cell-specific targeting moiety in the present invention. Such scFv are comprised of V_{H} and V_{L} domains linked into a single polypeptide chain by a flexible linker peptide.

The PDZ motif sequence (PL sequence) can be linked to a transmembrane transporter sequence and a cell-specific targeting moiety to produce a tri-fusion molecule. This molecule can bind to a leukocyte surface molecule, passes through the membrane and targets PDZ domains. Alternatively, a PDZ motif sequence (PL sequence) can be linked to a cell-specific targeting moiety that binds to a surface molecule that internalizes the fusion peptide.

In another approach, microspheres of artificial polymers of mixed amino acids (proteinoids) have been used to deliver pharmaceuticals. For example, U.S. Pat. No. 4,925,673 describes drug-containing proteinoid microsphere carriers as well as methods for their preparation and use. These proteinoid microspheres are useful for the delivery of a number of active agents. Also see, U.S. Pat. Nos. 5,907,030 and 6,033,884, which are incorporated herein by reference.

B. Introduction of Polynucleotides into Cells

By introducing gene sequences into cells, gene therapy can be used to treat conditions in which leukocytes are activated to result in deleterious consequences. In one embodiment, a polynucleotide that encodes a PL sequence peptide of the invention is introduced into a cell where it is expressed. The expressed peptide then inhibits the interaction of PDZ proteins and PL proteins in the cell.

Thus, in one embodiment, the polypeptides of the invention are expressed in a cell by introducing a nucleic acid (e.g., a DNA expression vector or mRNA) encoding the desired protein or peptide into the cell. Expression can be either constitutive or inducible depending on the vector and choice of promoter. Methods for introduction and expression of nucleic acids into a cell are well known in the art and described herein.

In a specific embodiment, nucleic acids comprising a sequence encoding a peptide disclosed herein, are administered to a human subject. In this embodiment of the invention, the nucleic acid produces its encoded product that mediates a therapeutic effect. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises a coding sequence that is part of an expression vector. In particular, such a nucleic acid has a promoter operably linked to the coding sequence, said promoter being inducible or constitutive, and, optionally, tissue-specific. In another specific embodiment, a nucleic acid molecule is used in which the coding sequence and any other desired sequences are flanked by regions that promote homologous recombination at a desired site in the genome, thus providing for intrachromosomal expression of the nucleic acid (Koller and Smithies, 1989, Proc. Natl. Acad. Sci. USA 86:8932-8935; Zijlstra et al., 1989, Nature 342:435-438).

Delivery of the nucleic acid into a patient can be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses have the advantage of being capable of infecting non-dividing cells (Kozarsky and Wilson, 1993, Current Opinion in Genetics and Development 3:499-503). Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Furthermore, adenoviral vectors with modified tropism can be used for cell specific targeting (WO98/40508). Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The coding sequence to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and can be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a targeted mRNA, especially its C-terminus are also within the scope of the invention. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a nucleotide sequence, are preferred.

The antisense oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of target RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features such as secondary structure that may render the oligonucleotide sequence unsuitable. The suitability of candidate targets can also be evaluated by testing their accessibility to hybridization with complementary oligonucleotides, using ribonuclease protection assays.

The anti-sense RNA and DNA molecules and ribozymes of the invention can be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules can be generated by in vitro and in vivo transcription of DNA sequences encoding the RNA molecule. Such DNA sequences can be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phospho-diesterase linkages within the oligodeoxyribonucleotide backbone.

C. Other Pharmaceutical Compositions

The compounds of the invention can be administered to a subject per se or in the form of a sterile composition or a pharmaceutical composition. Pharmaceutical compositions comprising the compounds of the invention can be manufactured by means of conventional mixing, dissolving, granulating, dragee-making, levigating, emulsifying, encapsulating, entrapping or lyophilizing processes. Pharmaceutical compositions can be formulated in conventional manner using one or more physiologically acceptable carriers, diluents, excipients or auxiliaries that facilitate processing of the active peptides or peptide analogues into preparations which can be used pharmaceutically. Proper formulation is dependent upon the route of administration chosen.

For topical administration the compounds of the invention can be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents.

Alternatively, the compounds can be in powder form for constitution with a suitable vehicle, e.g., sterile pyrogen-free water, before use.

For transmucosal administration, penetrants appropriate to the barrier to be permeated are used in the formulation. Such penetrants are generally known in the art. This route of administration can be used to deliver the compounds to the nasal cavity.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents can be added, such as the cross-linked polyvinylpyrrolidone, agar, or alginic acid or a salt thereof such as sodium alginate.

If desired, solid dosage forms can be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added.

For buccal administration, the compounds can take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can also be formulated in rectal or vaginal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver peptides and peptide analogues of the invention. Certain organic solvents such as dimethylsulfoxide also can be employed, although usually at the cost of greater toxicity. Additionally, the compounds can be delivered using a sustained-release system, such as semipermeable matrices of solid polymers containing the therapeutic agent. Various of sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

As the compounds of the invention can contain charged side chains or termini, they can be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

D. Effective Dosages

The compounds of the invention will generally be used in an amount effective to achieve the intended purpose. The compounds of the invention or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “inhibitory amount” or “inhibitory concentration” of a PL-PDZ binding inhibitor is an amount that reduces binding by at least about 40%, preferably at least about 50%, often at least about 70%, and even as much as at least about 90%. Binding can be measured in vitro (e.g., in an A assay or G assay) or in situ.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval can be adjusted individually to provide plasma levels of the compounds that are sufficient to maintain therapeutic effect. Usual patient dosages for administration by injection range from about 0.1 to 5 mg/kg/day, preferably from about 0.5 to 1 mg/kg/day. Therapeutically effective serum levels can be achieved by administering multiple doses each day.

In cases of local administration or selective uptake, the effective local concentration of the compounds can not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy can be repeated intermittently while symptoms detectable or even when they are not detectable. The therapy can be provided alone or in combination with other drugs. In the case of conditions associated with leukocyte activation such as transplantation rejection and autoimmunity, the drugs that can be used in combination with the compounds of the invention include, but are not limited to, steroid and non-steroid anti-inflammatory agents.

E. Toxicity

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### EXAMPLE 1

### TAT T-Cell Surface Receptor Carboxyl Terminus Fusion Peptides Inhibit T-Cell Activation

Materials And Methods

Peptide Synthesis

All peptides were chemically synthesized by standard procedures. For example, the Tat-CD3 carboxyl terminus fusion peptide, (GYGRKKRRQRRRGPPSSSSGL, SEQ ID NO:491); Tat-CLASP1 carboxyl terminus fusion peptide, (GYGRKKRRQRRRGSISSSAEV, SEQ ID NO:492); Tat-CLASP2 carboxyl terminus fusion peptide, (GYGRKKRRQRRRGMTSSSSVV, SEQ ID NO:493); and Tat peptide, (GYGRKKRRQRRRG, SEQ ID NO:494); were dissolved at 1 mM in PBS, pH 7, or dH2O. Stock MBPAc1-16 peptide, (AcASQKRPSQRHGSKYLA, SEQ ID NO:495), was dissolved at 5 mM. All peptides were aliquoted and stored at −80° C. until tested.

Cell Cultures Cells were maintained and tested in RPMI 1640 media supplemented with 10% fetal calf serum (HyClone), 2 mM glutamine, 10 mM Hepes, 100 U/ml penicillin, 100 μg/ml streptomycin, 0.1 mM non-essential amino acids, 1 mM sodium pyruvate, and 50 μM beta mercaptoethanol.

T Cell Stimulation Assay

Supernatants were assayed for cytokine production following activation of T cell lines. Mouse T cell lines were stimulated using two different methods, either with antigen and antigen presenting cells or anti-mouse CD3.

Antigen-specific mouse T cells, BR4.2, were activated with the N-terminal 16 amino acid sequences of myelin basic protein (MBPAc1-16) and syngenic mouse splenocytes in 96-well plates. Mitomycin C-treated antigen presenting cells, 2×10⁵ B10.BR, were added to each row of serially diluted MBPAc1-16 ranging from 0 to 200 μM. Next, 10 μM Tat-peptides or media alone was added to each row. Finally, 2×10⁴ MBPAc1-16-specific T cell, pre-loaded with 10 μM Tat-peptides (see above), were added to all wells (Rabinowitz et al., 1997, Proc. Natl. Acad. Sci. U.S.A., 94:8702-8707). Cells were activated during an overnight incubation at 5% CO2, 37° C. Cell supernatant was collected and stored at −80° C. until assayed for cytokine production. The final volume was 200 μl/well.

Antibody against mouse CD3 (Pharmigen #145-2C11) was coated overnight at 4° C. using 96-well flat bottom Elisa plates at a final concentration of 0.5 μg/ml, diluted in PBS. Just prior to use, plates were washed three times with 200 μl/well PBS to remove excess anti-CD3. To ensure that cells were given sufficient time to transduce Tat-peptides before activation, T cells (5×10⁵ cells/ml) were pre-treated with or without 10 μM Tat-peptides for two hours at 5% CO2, 37° C. and then diluted in media with or without 10 μM Tat-peptides to a final concentration of 2×10⁴ cells per well in a final volume of 200 μl. Cells were then treated as described above.

Cytokine ELISA

IFNγ was measured from cell supernatants, described above, at ambient temperature using the Endogen, Inc. ELISA protocol 3. Briefly, 96-well, flat bottom, high binding ELISA plates were preincubated overnight with coating antibody (MM700). Plates were washed with 50 mM TRIS, 0.2% tween-20, pH 8 and they blocked for one hour with PBS plus 2% BSA. Washed plates were then incubated one hour with 25 μl of cell supernatant and 25 μl blocking buffer, or with 50 μl IFNγ standard. The presence of IFNγ was detected with a biotin-labeled anti-mouse IFNγ monoclonal antibody (MM700B, Endogen, Inc.,). Quantitative amounts of detection antibody are revealed with horseradish peroxidase-conjugated streptavidin. The enzymatic, color, substrate for HRP, tetramethylbenzidine (TMB), was developed for up to 30 minutes and stopped with 1.0 M H₂SO₄. The absorbance at 450 nm was measured using a microtiter plate reader (Thermo Max, Molecular Devices) and the concentration of unknown IFNγ from cell supernatants was calculated from a standard curve generated by Softmax Pro software (Molecular Devices).

Results

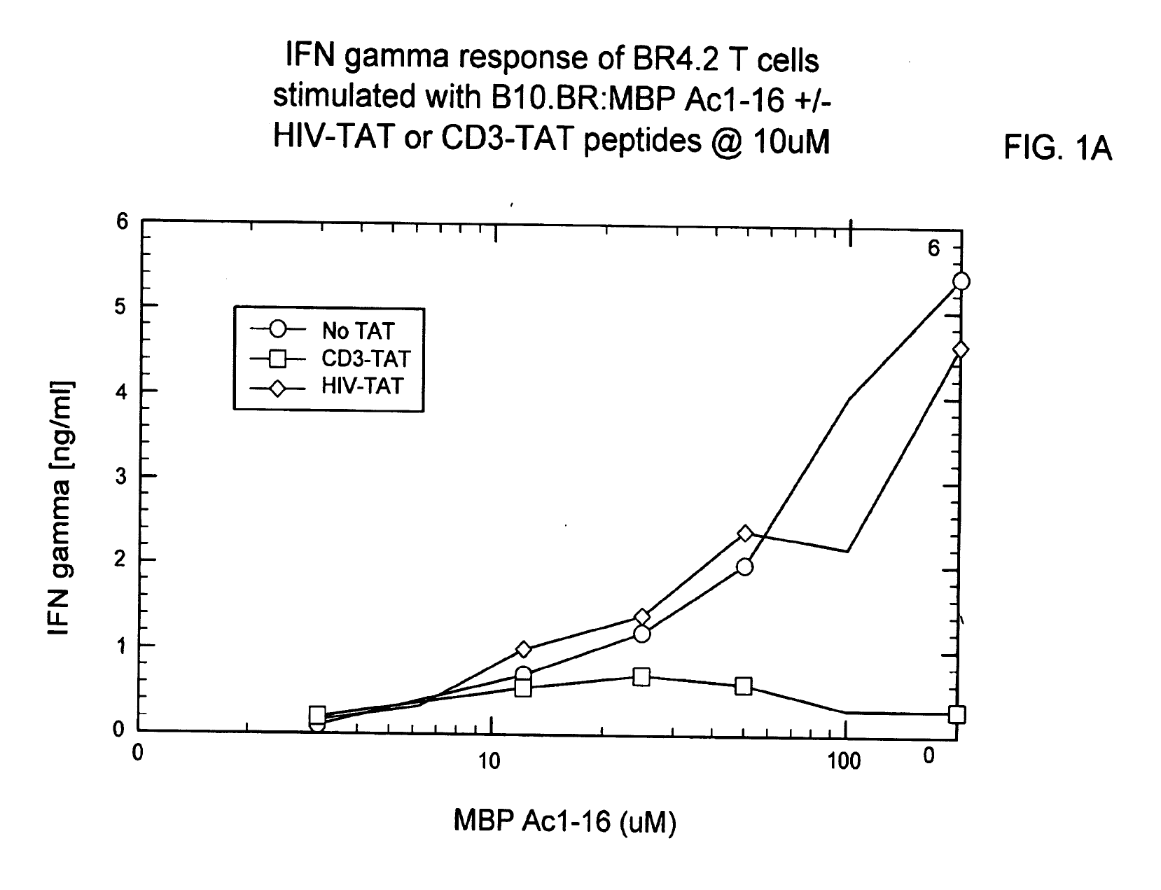

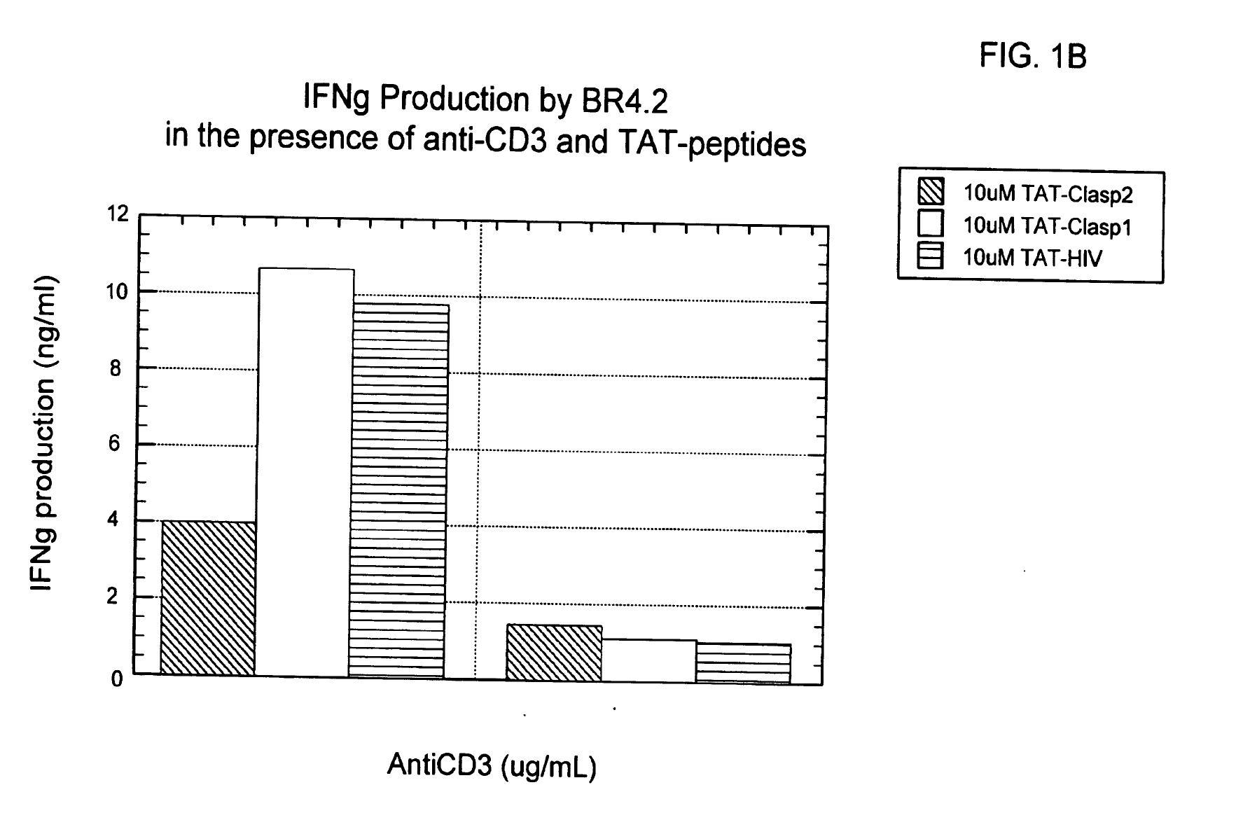

Peptides containing Tat transporter sequences linked to C-terminal sequences of various PLs were testing for their ability to inhibit T cell activation. FIG. 1A shows that the Tat-CD3 fusion peptide inhibits T cell activation mediated by peptide:MHC as compared to controls of Tat-peptide alone or no peptide. FIG. 1B shows that Tat-CLASP2 carboxyl terminus fusion peptide inhibited T cell activation mediated by monoclonal anti-CD3 as compared to Tat-peptide alone. Tat-CLASP1 fusion peptide did not inhibit T cell activation in this experiment. These results indicate that peptides containing potential inhibitory sequences can be transported into T cells through transporter peptide such as Tat to disrupt surface receptor organization mediated by PDZ proteins. Disruption of PDZ-mediated surface receptor organization leads to blockage of T cell activation in response to antigen.

### EXAMPLE 2

### Generation of Eukaryotic Expression Constructs Bearing DNA Fragments that Encode PDZ Domain-Containing Genes or Portions of PDZ Domain Genes

This example describes the cloning of PDZ domain containing genes or portions of PDZ domain containing genes were into eukaryotic expression vectors in fusion with red fluorescent protein (RFP).

A. Strategy

DNA fragments corresponding to PDZ domain containing genes were generated by RT-PCR from jurkat cell line (transformed T-cells) derived RNA. Primers were designed to create restriction nuclease recognition sites at the PCR fragment's ends, to allow cloning of those fragments into the appropriate vectors. Subsequent to RT-PCR, DNA samples were submitted to agarose gel electrophoresis. Bands corresponding to the expected size were excised. DNA was extracted and treated with appropriate restriction endonuclease. DNA samples were purified once more by gel electrophoresis, and gel extracted DNA fragments were coprecipitated and ligated with the appropriate linearized cloning vector. After transformation into E. coli, bacterial colonies were screened by PCR for the presence and correct orientation of insert. Positive clones were picked for large scale DNA preparation and the insert including the flanking vector sites were sequenced to ensure correct sequence of fragments and junctions between the vectors and fusion proteins.

B. Vectors:

Cloning vectors were pDsRED1-N1 (purchased from CLONTECH, # 6921-1) and pDsRED1-N1(+ATG), a derivative of pDsRED1-N1 generated by recombinant DNA technology.

DNA fragments to clone that contained the ATG-start codon were cloned into pDsRED1-N1. Fragments void of a proper translation initiation codon were cloned into pDsRED1-N-(+ATG), since this vector includes an translation initiation start codon. Vector pDsRED1-N1(+ATG) differs from pDsRED1 only with regard to the multiple cloning sites. The sequence that is unique to pDsRED1-N1(+ATG) is shown below; boundaries with pDsRED1-N1 are printed in lower case and correspond to nucleotides N 633 and N 662 in pDsRED1-N1, respectively.

(SEQ ID NO: 839)

5′-attGCCACCATGGGAATTCTGGATCCGGGAgat-3′

C. Deduced Amino Acid Linker Sequences:

Linker sequences between the cloned inserts and RFP vary depending on the vectors and on the restriction endonuclease used for cloning. Deduced linker amino acid sequences (SEQ ID NOS:840 and 841) are listed in the table below; For some constructs, the first N-terminal and/or last C-terminal amino acid corresponds to a linker amino acid introduced by the cloning process but is not represented at that position in the corresponding gene.

TABLE 2

| | |
|---|---|
| pDsRED1-N1, cloning approach: | PDZ domain insert C-term - |
| (fragment) Eco RI or Mfe I/Eco RI | LEU - GLN - SER - THR - |
| (vector) | VAL - PRO - ARG - ALA - |
| | ARG - ASP - PRO - PRO - |
| | VAL - ALA - THR - |
| | red fluorescent protein; |
| pDsRED1-N1(+ATG), cloning approach: | Start codon (MET) - GLY - |
| (fragment) Eco RI/Eco RI (vector) | ILE - PDZ domain gene |
| | insert - LEU - ASP - PRO - |
| | GLY - TYR - PRO - PRO - |
| | VAL - ALA - THR - red |
| | fluorescent protein; |
| pDsRED1-N1(+ATG), cloning approach: | Start codon (MET) - ARG - |
| (fragment) Mfe I/Eco RI (vector) | ILE - PDZ domain gene |
| | insert - LEU - ASP - PRO - |
| | GLY - TYR - PRO - PRO - |
| | VAL - ALA - THR - red |
| | fluorescent protein; |

D. Constructs:

The deduced protein sequence of cloned inserts, primers used to generate DNA fragments by RT-PCR and accession # are given below for each construct. For all constructs, the fusion with RFP was carboxy terminal.

1. Homo sapiens Dishevelled 1 (DVL1)

Acc #: NM_{—}004421
GI: 4758213
Cloning sites for all constructs: Eco RI/Eco RI

Construct (N-P) [Covers the methionine start codon and extends over the C-terminal boundary of the DVL1 PDZ domain];
primers: 308 DVF and 311 DVR;
vector: pDsRED1-N1

aa 1-aa 341

| | |
|---|---|
| MAETKIIYHMDEEETPYLVKLPVAPERVTLADFKNVLSNRPVHAYKFFKSMDQDFG | (SEQ ID NO: 496) |
| VVKEEIFDDNAKLPCFNGRVVSWLVLVEGAHSDAGSQGTDSHTDLPPPLERTGGIG | |
| DSRSPSFQPDVASSRDGMDNETGTESMVSHRRDRARRRNREEAARTNGHPRGDRR | |
| RDVGLPPDSASTALSSELESSSFVDSDEDDSTSRLSSSTEQSTSSRLIRKHKRRRRKQ | |
| RLRQADRASSFSSMTDSTMSLNIITVTLNMERHHFLGICIVGQSNDRGDGGIYIGSIM | |
| KGGAVAADGRIEPGDMLLQVNDVNFENMSNDDAVRVLREIVSQTGPISLTVAKCW | |
| DPT | |

Construct (N) [Covers the methionine start codon and extends to the N-terminal boundary of the DVL1 PDZ domain];
primers: 308 DVF and 345 DVR
vector: pDsRED1-N1

aa 1-aa197

| | |
|---|---|
| MAETKIIYHMDEEETPYLVKLPVAPERVTLADFKNVLSNRPVHAYKFFFKSMDQDF | (SEQ ID NO: 497) |
| GVVKEEIFDDNAKLPCFNGRVVSWLVLVEGAHSDAGSQGTDSHTDLPPPLERTGGI | |
| GDSRSPSFQPDVASSRDGMDNETGTESMVSHRRDRARRRNREEAARTNGHPRGDR | |
| RRDVGLPPDSASTALSSELESSSFVDSDEDG | |

Construct (P) [Consists of the PDZ domain of DVL1];
primers: 344 DLF and 311 DVR;
vector: pDsRED1-N1(+ATG)

aa 246-aa 341

| | |
|---|---|
| SLNIITVTLNMERHHFLGICIVGQSNDRGDGGIYIGSIMKGGAVAADGRIEPGDMLLQ | (SEQ ID NO: 498) |
| VNDVNFENMSNDDAVRVLREIVSQTGPISLTVAKCWDPT | |

Primers:

| 308 DVF (N 128-N 155) | |
|---|---|
| 5′-TCGGAATTCGTCGCGCCATGGCGGAGAC-3′ | (SEQ ID NO: 499) |
| 311 DVR (N 1004-N 1032) | |
| 5′-GGGAATTCGGTCCCAGCACTTGGCCACAG-3′ | (SEQ ID NO: 500) |
| 344 DVF (N 873-N 900) | |
| 5′-CCAGAATTCTCAACATCGTCACTGTCAC-3′ | (SEQ ID NO: 501) |
| 345 DVR (N713-N744) | |
| 5′- | (SEQ ID NO: 502) |
| TCGGAATTCCATCCTCGTCCGAGTCCACAAAG- | |
| 3′ | |

2. KIAA 0751/41.8 KD
Acc #: AB018294
GI: 3882222
Cloning sites for all constructs: (vector) Eco RI/(fragment) Mfe I

Construct (N-J) [includes the third in frame-methionine (putative start) codon in (GI: 3882222) and extends c-terminal of the PDZ domain to the region on sequence divergency between KIAA 0751 (GI: 3882222) and hypothetical 41.8 Kd protein (AF007156/GI: 3882222)];
primers: 318 KIF and 320 KIR;
vector: pDsRED1-N1

aa 389-aa 803

| | |
|---|---|
| MMYFGGHSLEEDLEWSEPQIKDSGVDTCSSTTLNEEHSHSDKHPVTWQPSKDGDR | (SEQ ID NO: 503) |
| LIGRILLNKRLKDGSVPRDSGAMLGLKVVGGKMTESGRLCAFITKVKKGSLADTVG | |
| HLRPGDEVLEWNGRLLQGATFEEVYNIILESKPEPQVELVVSRPIGDIPRIPDSTHAQ | |
| LESSSSSFESQKMDRPSISVTSPMSPGMLRDVPQFLSGQLSIKLWFDKVGHQLIVTIL | |
| GAKDLPSREDGRPRNPYVKIYFLPDRSDKNKRRTKTVKKTLEPKWNQTFIYSPVHR | |
| REFRERMLEITLWDQARVREEESEFLGEILIELETALLDDEPHWYKLQTHDVSSLPLP | |
| HPSPYMPRRQLHGESPTRRLQRSKRISDSEVSDYDCDDGIGVVSDYRHDGRDLQSS | |
| TLSVPEQVMSSNHCSPSGSPHRVDVIGRTT | |

Construct (P) [consists of the PDZ domain of KIAA 0751/41.8 Kd hypothetical protein (GI: 3882222)];
primers: 341 KIF and 319 KIR.
vector pDsRED 1-N1(+ATG)

aa 443-aa 534

| | |
|---|---|
| LKDGSVPRDSGAMLGLKVVGGKMTESGRLCAFITKVKKGSLADTVGHLRPGDEVL | (SEQ ID NO: 504) |
| EWNGRLLQGATFEEVYNIILESKPEPQVELVVSRPIA | |

Primers:

| | |
|---|---|
| 318 KIF (N 1366-N 1393) | |
| 5′-AGACAATTGAGGAAATGATGTACTTTGG-3′ | (SEQ ID NO: 505) |
| 319 KIR (N 1830-N 1857) | |
| 5′-GAACAATTGCAATAGGCCTTGAAACTAC-3′ | (SEQ ID NO: 506) |
| 320 KIR (N 2640-N 2667) | |
| 5′-ACCCAATTGTAGTCCTTCCTATAACATC-3′ | (SEQ ID NO: 507) |
| 341 KIF (N 1567-N 1593) | |
| 5′-ATAGAATTCTAAAAGATGGAAGTGTAC-3′ | (SEQ ID NO: 508) |

3. Homo sapiens PAR6
Acc #: AF265565
GI: 8468608
Cloning sites for all constructs: Eco RI/Eco RI

Construct (N-P) [Covers the methionine start codon and extends over the C-terminal boundary of the PDZ domain];
primers: 322 PAF and 324 PAR;
vector: pDsRED1-N1

aa 1-aa 251

| | |
|---|---|
| MARPQRTPARSPDSIVEVKSKFDAEFRRFALPRASVSGFQEFSRLLRAVHQIPGLDV | (SEQ ID NO: 509) |
| LLGYTDAHGDLLPLTNDDSLHRALASGPPPLRLLVQKREADSSGLAFASNSLQRRK | |
| KGLLLRPVAPLRTRPPLLISLPQDFRQVSSVIDVDLLPETHRRVRLHKHGSDRPLGFY | |
| IRDGMSVRVAPQGLERVPGIFISRLVRGGLAESTGLLAVSDEILEVNGIEVAGKTLDQ | |
| VTDMMVANSHNLIVTVKPANQR | |

Construct (N) [Covers the methionine start codon and extends to the N-terminal boundary of the PDZ domain;
primers: 322 PAF and 343 PAR
vector: pDsRED1-N1

aa 1-aa 147

| | |
|---|---|
| MARPQRTPARSPDSIVEVKSKFDAEFRRFALPRASVSGFQEFSRLLRAVHQIPGLDV | (SEQ ID NO: 510) |
| LLGYTDAHGDLLPLTNDDSLHRALASGPPPLRLLVQKREADSSGLAFASNSLQRRK | |
| KGLLLRPVAPLRTRPPLLISLPQDRQVSSVIDV | |

Construct (P) [Consists of the PDZ domain of PAR6];
primers: 342 PAF and 324 PAR;
vector: pDsRED1-N1(+ATG)

aa 155-aa 251

| | |
|---|---|
| RRVRLHKHGSDRPLGFYIRDGMSVRVAPQGLERVPGIFISRLVRGGLAESTGLLAVS | (SEQ ID NO: 511) |
| DEILEVNGIEVAGKTLDQVTDMMVANSHNLIVTVKPANQR | |

Primers

| | |
|---|---|
| 322 PAF (N 55-N 82) | |
| 5′- | (SEQ ID NO: 512) |
| CCCGAATTCGCCATGGCCCGGCCGCAGAG-3′ | |
| 324 PAR (N 798-N 825) | |
| 5′- | (SEQ ID NO: 513) |
| CGTGAATTCGCTGGTTGGCGGGCTTGAC-3′ | |
| 342 PAF (N 519-N 548) | |
| 5′- | (SEQ ID NO: 514) |
| GAGGAATTCCGACGGGTGCGGCTGCACAAG-3′ | |
| 343 PAR (N 485-N 516) | |
| 5′- | (SEQ ID NO: 515) |
| GCAGAATTCCCACGTCTATGACTGAGGAAAC-3′ | |

4. Homo sapiens Post-Synaptic Density Protein 95 (PSD95)
Acc #: ABU83192
GI: 3318652
Cloning sites for all constructs: Eco RI/Eco RI
Vector: pDsRED1-N1

Construct (N-P3) [Covers the methionine start codon and extends over the C-terminal boundary of PDZ domain 3;
primers: 315 PSF and 304 PSR.

aa 1-aa 442

| | |
|---|---|
| MSQRPRAPRSALWLLAPPLLRWAPPLLTVLHSDLFQALLDILDYYEASLSESQKYRY | (SEQ ID NO: 516) |
| QDEDTPPLEHSPAHLPNQANSPPVIVNTDTLEAPGYELQVNGTEGEMEYEEITLERG | |
| NSGLGFSIAGGTDNPHIGDDPSIFITKIIPGGAAAQDGRLRVNDSILFVNEVDVREVTH | |
| SAAVEALKEAGSIVRLYVMRRKPPAEKVMEIKLIKGPKGLGFSIAGGVGNQHIPGDN | |
| SIYVTKIIEGGAAHKDGRLQIGDKILAVNSVGLEDVMHEDAVAALKNTYDVVYLKV | |
| AKPSNAYLSDSYAPPDITTSYSQHLDNEISHSSYLGTDYPTAMTPTSPRRYSPVAKD | |
| LLGEEDIPREPRRIVIHRGSTGLGFNIVGGEDGEGIFISFILAGGPADLSGELRKGDQIL | |
| SVNGVDLRNASHEQAAIALKNAGQTVTIIAQYKPEEYSR | |

Primers:

| | |
|---|---|
| 315 PSF (N847-N876) | |
| 5′- | (SEQ ID NO: 517) |
| AGAGAATTCAGAGATATGTCCCAGAGACCAAG- | |
| 3′ | |
| 304 PSR (N 2161-N 2189) | |
| 5′- | (SEQ ID NO: 518) |
| CGAGAATTCTGTACTCTTCTGGTTTATAC-3′ | |

5. Homo sapiens hCASK (CASK)
Acc #: AF032119
GI: 2641548
Cloning sites: Eco RI/Eco RI

Construct (P) [Covers the PDZ domain of hCASK];

Note: The amino acid sequence homology between the human hCASK and the mouse mCASK-B is 100% identical.

primers: 336 CAF and 335 CAR;
vector: pDsRED 1-N1(+ATG)

aa 399-aa 572

| | |
|---|---|
| RLVQFQKNTDEPMGITLKMNELNHCIVARIMHGGMIHRQGTLHVGDEIREINGISVA | (SEQ D NO: 519) |
| NQTVEQLQKMLREMRGSITFKIVPSYR | |

Primers

| | |
|---|---|
| 336 CAF (N 1484-N 1512) | |
| 5′- | (SEQ ID NO: 520) |
| CCAGAATTCGGCTGGTACAGTTTCAAAAG-3′ | |
| 325 CAR (N 1722-N 1750) | |
| 5′- | (SEQ ID NO: 521) |
| ACTGAATTCGGTAACTTGGCACAATCTTG-3′ | |

6. Homo sapiens Membrane Protein, Palmitolated 2 (MPP2/DLG2)
Acc #: X82895
GI: 939884
Cloning sites for all constructs: Eco RI/Eco RI

Construct (N-SH3) [Covers the methionine start codon, the PDZ domain and extends to the C-terminal boundary of the MPP2 SH3 domain; the construct is a splice variant of the construct annotated under GI:939884. With respect to GI:939884, the DNA portion N 238 to 309 is missing; this DNA stretch corresponds to AA 51-74. The open reading frame is maintained throughout the deletion].
primers: 305 MF and 306 MR;
vector: pDsRED1-N1

aa 1-aa 317

| | |
|---|---|
| MPVAATNSETAMQQVLDNLGSLPSATGAAELDLIFLRGIMESPIVRSLAKAHERLEE | (SEQ ID NO: 522) |
| TKLEAVRDNNLELVQEILRDLAQLAEQSSTAAELAHILQEPHFQSLLETHDSVASKT | |
| YETPPPSPGLDPTFSNQPVPPDAVRMVGIRKTAGEHLGVTFRVEGGELVIARILHGG | |
| MVAQQGLLHVGDIIKEVNGQPVGSDPRALQELLRNASGSVILKILPSYQEPHLPRQV | |
| FVKCHFDYDPARDSLIPCKEAGLRFNAGDLLQIVNQDDANWWQACHVEGGSAGLI | |
| PSQLLEEKRKG | |

Primers:

| | |
|---|---|
| 305 MF (N 58-N 84) | |
| 5′-AGAGAATTCAGAGCCCTTGCCTCCTTC-3′ | (SEQ ID NO: 523) |
| 306 MR (N 798-N 825) | |
| 5′-TGAGAATTCCTTTCCGCTTCTCCTCCAG-3′ | (SEQ ID NO: 524) |

7. Homo sapiens Tax Interaction Protein 1 (TIP-1)
Acc #: AF028823
GI: 2613001
Cloning sites: Eco RI/Bam H1
(We determined 5′ start site and 5′ full length sequence by 5′ RACE)

Construct (N-C);
vector: pDsRed1-N1

aa 3-aa 125

| | |
|---|---|
| YIPGQPVTAVVQRVEIHKLRQGENLILGFSIGGGIDQDPSQNPFSEDKTDKGIYVTRV | (SEQ ID NO: 525) |
| SEGGPAEIAGLQSGDKIMQVNGWDMTMVTHDQARKRLTKRSEEVVRLLVTRQSL | |
| QKAVQQSML | |

Primer:

| | |
|---|---|
| 1318 TIP R3-1 (N 336-N 356) | |
| 5′-CAGTCCATGCTGTCGGATCCG-3′ | (SEQ ID NO: 526) |
| 1317 TIP R5-1* | |
| 5′-GTCGGAATTCCCTACATCCCG-3′ | (SEQ ID NO: 527) |

### EXAMPLE 3

### Identification of CD95 and TAX Interactions with TIP-1

A. Background

Binding between these molecules was assessed using a modified ELISA. Briefly, a GST-TIP-1 fusion was produced that contained the entire PDZ domain of human TIP-1 (Insert as in EXAMPLE 2). In addition, biotinylated peptides corresponding to the C-terminal 20 amino acids of Tax and CD95 were synthesized and purified by HPLC. Binding between these entities was detected through a colorimetric assay using avidin-HRP to bind the biotin and a peroxidase substrate (G-assay, as described supra). By titrating the amount of peptide and protein added to these reactions, dissociation constants (Kd) were determined as an indication of relative affinity of the peptide and fusion protein association.

B. Peptide Purification

Peptides representing the C-terminal 8 or 20 amino acids of CD95 and Tax were synthesized by standard FMOC chemistry and biotinylated if not used as an unlabeled competitor. Peptides were purified by reverse phase high performance liquid chromatography (HPLC) using a Vydac 218TP C18 Reversed Phase column having the dimensions of 10*25 mm, 5 um. Approximately 40 mg of peptide was dissolved in 2.0 ml of an aqueous solution of 49.9% acetonitrile and 0.1% Tri-Fluoro acetic acid (TFA). This solution was then injected into the HPLC machine through a 25 micron syringe filter (Millipore). Buffers used to get a good separation are (A) distilled water with 0.1% TFA and (B) 0.1% TFA with Acetonitrile. Gradient Segment setup is listed in TABLE 3 below.

TABLE 3

| | | | | Flow rate |
|---|---|---|---|---|
| Time | A | B | C | (ml/min) |
| 0 | 96% | 4% | 0 | 5.00 |
| 30 | 4% | 96% | 0 | 5.00 |

The separation occurs based on the nature of the peptides. A peptide of overall hydrophobic nature will elute off later than a peptide of a hydrophilic nature. Fractions containing the “pure” peptide were collected and checked by Mass Spectrometer (MS). Purified peptides are lyophilized for stability and stored at −80° C. for later use.
C. Construction of GST-TIP-1

DNA representing the putative open reading frame of human TIP-1 was amplified by PCR and cloned into the pGEX-3X vector (Amersham-Pharmacia) to generate a GST-TIP-1 fusion vector. GST-TIP-1 protein was produced by inducing this vector with IPTG in DH5α as recommended by the Pharmacia protocol. Cells were lysed and purified by glutathione-sepharose chromatography according to manufacturer's instructions (Pharmacia). Purified protein was dialyzed against storage buffer (PBS with 25% glycerol) and stored at −20° C. (short term) or −80° C. (long term).

D. “G” Assay for Identification of Interactions Between Peptides and Fusion Proteins

Reagents and Materials
Nunc Polysorp 96 well Immuno-plate (Nunc cat#62409-005)

(Maxisorp plates have been shown to have higher background signal)
PBS pH 7.4 (Gibco BRL cat#16777-148) or

AVC phosphate buffered saline, 8 gm NaCl, 0.29 gm KCl, 1.44 gm Na₂HPO4, 0.24 gm KH₂PO4, add H2O to 1 L and pH 7.4; 0.2 micron filter
2% BSA/PBS (10 g of bovine serum albumin, fraction V (ICN Biomedicals cat#IC15142983) into 500 ml PBS
Goat anti-GST mAb stock @ 5 mg/ml, store at 4° C., (Amersham Pharmacia cat#27-4577-01), dilute 1:1000 in PBS, final concentration 5 ug/ml
HRP-Streptavidin, 2.5 mg/2 ml stock stored at 4° C. (Zymed cat#43-4323), dilute 1:2000 into 2% BSA, final concentration at 0.5 ug/ml
Wash Buffer, 0.2% Tween 20 in 50 mM Tris pH 8.0
TMB ready to use (Dako cat#S1600)
1M H₂SO₄
12w multichannel pipettor,
50 ml reagent reservoirs,
15 ml polypropylene conical tubes
   Protocol
1) Coat plate with 100 ul of 5 ug/ml goat anti GST, O/N @ 4° C.
2) Dump coating antibodies out and tap dry
3) Blocking—Add 200 ul per well 2% BSA, 2 hrs at 4° C.
4) Prepare proteins in 2% BSA at 5 ug/ml

(2 ml per row or per two columns)
5) 3 washes with cold PBS (must be cold through entire experiment)

(at last wash leave PBS in wells until immediately adding next step)
6) Add proteins at 50 ul per well on ice (1 to 2 hrs at 4° C.)
7) Prepare Peptides in 2% BSA (2 ml/row or /columns)
8) 3× wash with cold PBS
9) Add peptides at 50 ul per well on ice (time on/time off)

keep on ice after last peptide has been added for 10 minutes exactly

place at room temp for 20 minutes exactly
10) Prepare 12 ml/plate of HRP-Streptavidin (1:2000 dilution in 2% BSA)
11) 3× wash with cold PBS
12) Add HRP-Streptavidin at 100 ul per well on ice, 20 minutes at 4° C.
13) Turn on plate reader and prepare files
14) 5× wash with Tween wash buffer, avoiding bubbles
15) Using gloves, add TMB substrate at 100 ul per well

incubate in dark at room temp

check plate periodically (5, 10, & 20 minutes)

take early readings, if necessary, at 650 nm (blue)

at 30 minutes, stop reaction with 100 ul of 1M H₂SO₄

take final reading at 450 nm (yellow)

E. Results of Binding Experiments

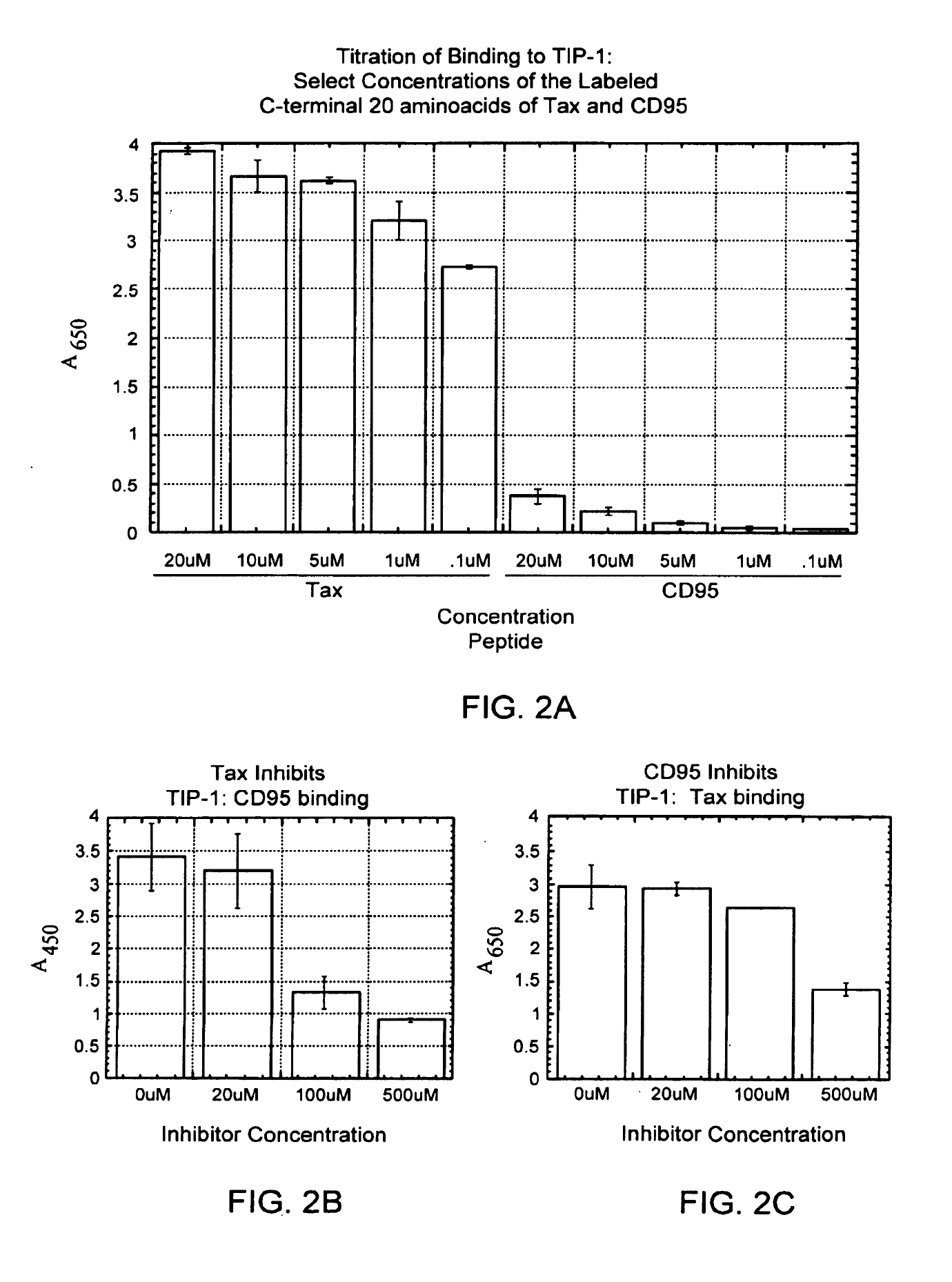

Results of peptides representing the carboxy-terminal 20 amino acids of Tax and CD95 binding to TIP-1 are shown in FIG. 2A. Clearly, Tax binds GST-TIP-1 with much higher affinity than does CD95 at equivalent peptide concentrations and with equivalent amount of GST-TIP-1 fusion protein.

F. Determination of Dissociation Constants for Proteins Interacting with TIP-1

Using the protocol for the ‘G’ assay described above, dissociation constants were determined by titrating the amount of peptide against a set concentration of PDZ-containing protein. Kd values were determined by identifying the peptide concentration that gave half-maximal binding to the PDZ protein. Different concentrations of PDZ-containing protein were plated in order to achieve maximal peptide binding values that were less than the absorbance maximum of the ELISA plate reader. TABLE 4 below shows the Kd values observed for the titrated reactions.

TABLE 4

| | | | | Tax | | CD95 | |
|---|---|---|---|---|---|---|---|
| PDZ | ug/ml | nm | min | OD | Kd | OD | Kd |
| TIP-1 | 0.1 | 450 | 30 | 3.3 | 0.005 | | |
| | 0.3 | 450 | 30 | | | 2.6 | 20.0 |
| | 0.1 | 450 | 30 | 2.1 | 0.006 | | |
| | 0.3 | 450 | 30 | | | 3.5 | 25.0 |
| DLG1(1-2) | 0.1 | 450 | 30 | 3.4 | 0.20 | | |
| | 0.3 | 450 | 30 | | | 2.6 | 15.0 |
| | 0.1 | 450 | 30 | 1.6 | 0.13 | | |
| | 0.3 | 450 | 30 | | | 2.1 | 20.0 |

Table 4 shows the Kd values in uM for the interactions between proteins and peptides in a series of ‘G-Assay’ experiments. Proteins on the left are GST fusions to the PDZ domain(s) of protein indicated. Numbers in parenthesis indicate the number of PDZ domains present in the fusion construct, from the amino-terminus of the first number listed to the carboxyl terminus of the second. PDZ Ligands are listed across the top of the table, representing biotinylated peptides corresponding to the carboxy-terminal 20 amino acids of each protein. The first three columns following the PDZ indicate the concentration of fusion protein plated for the G assay, followed by the wavelength and time of reading from addition of TMB substrate. 450 nm indicates a reaction halted by addition of sulfuric acid and absorbance read at 450 nm. Values beneath each ligand indicate first the maximum absorbance followed by the observed Kd in uM. Numbers in the squares are the average of duplicate or quadruplicate reactions. Blank squares indicate that the Kd for the interaction was not tested under those conditions on the same sample plate. No binding to GST alone is observed.

G. Conclusions and Summary

Peptides corresponding to the PL of Tax bind TIP-1 with much higher affinity than peptides corresponding to the PL of CD95. Comparing dissociation constants (0.006 uM for Tax:TIP-1, 20 uM for CD95:TIP-1), one can see that Tax can bind TIP-1>3000-fold more strongly than CD95. This provides an explanation for potential oncogenicity of Tax. If TIP-1 is a regulator of apoptosis through binding to CD95, then upon HTLV-1 infection of lymphoid cells the Tax oncoprotein should be able to bind TIP-1 and remove it's ability to associate with CD95 at meaningful levels. If CD95 mediated apoptosis requires TIP-1, then the ability of the body to activate apoptotic pathways in HTLV-1 infected cells and hence result in a cancerous condition.

The data presented in TABLE 4 also suggest that affinities between PDZ domains and ligands are not specific to the PDZ domain or the PL individually, but are instead specific for each unique pair. Clearly, both TIP-1 and DLG1 proteins have different dissociation constants for different ligands. Interestingly, we observe that CD95 has similar dissociation constants for both TIP-1 and DLG1. Though CD95 has similar dissociation for both pairs, Tax has different affinities for the same proteins. Hence, if a specific PL bound PDZ ‘A’ with ‘X’ Kd and PDZ ‘B’ with ‘Y’ Kd, one could not assume that another PL that bound PDZ ‘A’ with ‘X’ Kd would bind PDZ ‘B’ with ‘Y’ Kd. This shows the unique and specific nature of PDZ:PL interactions.

### EXAMPLE 4

### TAX and CD95 Competition for TIP-1 Binding In Vitro

The differing affinities of Tax and CD95 peptides for GST-TIP-1 suggest that competition between these two can be a mechanism for the oncogenicity of viral infection. Upon infection, the higher affinity of Tax could preferentially bind TIP-1 protein available in the cell, removing the TIP-1 bound to CD95 (Fas) and thereby rendering the cell less able to undergo apoptosis. In order to test this, competition experiments between Tax and CD95 for TIP-1 binding were performed using the ‘G Assay’, but adding additional unlabeled competitor peptide at step 9 of the ‘G Assay’ presented in EXAMPLE 3 section D.

FIGS. 2B and 2C show the results of these experiments. The graphs show the amount of binding of the biotinylated 20 amino acid peptide in the presence of increasing concentrations of unlabeled 8 amino acid competitor. FIG. 2B shows that 20, 100, and 500 μM Tax is able to compete for binding to TIP-1 with 20 μM labeled CD95. FIG. 2C shows that it takes 100-500 μM unlabeled CD95 peptide to compete for binding of 1 μM Tax to TIP-1. Taken together, a 5-fold excess of Tax is able to compete effectively for TIP-1 binding while it takes nearly a 500-fold excess of CD95 binding to interfere with the binding of Tax to TIP-1. This provides further support for the argument that Tax has a significantly higher affinity for TIP-1 than does CD95.

### EXAMPLE 5

### HPV E6 Oncogene and PDZ Proteins

This example demonstrates the use of PL sequence motifs identified according the to the invention in the prediction of biological function in an oncogenic virus.

Human papilloma virus (HPV) infection plays a role in development of cervical carcinoma. The oncoprotein responsible for this is the early gene E6 from strains 16, 18 and 31. E6 associates with p53 and shunts this tumor suppressor into the ubiquitin proteosomal pathway to affect transformation. Using the PL motifs disclosed herein, we noted that the E6 from oncogenic strains HPV16, 18 and 31 are PDZ ligands (PLs) with the carboxy-terminal sequence of ETQ(V/L). Similarly, the E6 of oncogenic strain HPV66 has the carboxy-terminus ESTV (SEQ ID NO:221), which also matches the consensus PDZ binding motif.

We performed an expanded search of the HPV E6 proteins and discovered HPV70 E6 fits perfectly the described PDZ consensus ETQV (SEQ ID NO:191), identical to HPV18 and 31. We can thus predict that HPV70 is likely oncogenic on the basis that E6 is a PDZ ligand. Other HPV strains with E6 proteins that are potential PLs (based on motifs) include 63 (LYII; SEQ ID NO:528), 66 (ESTV; SEQ ID NO:221), 33 (ETAL; SEQ ID NO:196),52 (VTQV; SEQ ID NO:206),58 (QTQV; SEQ ID NO:216), and 35 (ETEV; SEQ ID NO:201). Strains 77 (QSRQ; SEQ ID NO:226) and 80 (GSIE; SEQ ID NO:529) can also be PLs, although the motif matches less strongly. Others, such as E6 proteins from HPV strain 57 (RTSH; SEQ ID NO:211) and 77 (QSRQ; SEQ ID NO:226) do not appear to be oncogenic and do not match any known consensus for PDZ binding.

To identify PDZ domains that can be bound by oncogenic HPV E6 proteins we synthesized peptides corresponding to the C-termini of several oncogenic and non-oncogenic E6 proteins (TABLE 8). These were run in the ‘G Assay’ (EXAMPLE 3) against a variety of PDZ domains. We found that oncogenic E6 proteins with predicted PLs bound a variety of PDZ domains at varying affinities (TABLE 7 and TABLE 12). In addition, non-oncogenic E6 proteins from strains 57 and 77 did not bind any of the PDZ domains tested (TABLE 7 and TABLE 12 and data not shown).

Inhibitors of the interaction of the PDZ and oncogenic E6 PLs could be identified using the methods of the invention and could be useful for inhibition of E6-mediated transformation. Such inhibitors (e.g., small molecules, peptides or recombinant proteins) could be administered to patients (e.g., by local application to the vaginal vault and the uterine cervix) to treat or prevent cervical carcinoma. Diagnostic assays for oncogenic HPV are carried out using the sequences corresponding to the HPV E6 PL to design polynucleotide (e.g., PCR) or antibody probes that distinguish E6 proteins that are PLs from those that are not PLs.

### EXAMPLE 6

### Ability of Short (>10-Mer) Peptides to Compete with 20-Mers for Binding to PDZs

A. Introduction

The potential for unlabeled 8-mers and 3-mers to compete for binding with biotinylated 20-mers to PDZ domains was examined. Interactions between a PDZ domain and two or more biotinylated peptides mimicking PDZ ligands identified through the ‘G Assay’ were used as model interactions. Short, 3 or 8 amino acid, unlabeled peptides were synthesized by standard techniques and used at variable concentrations with a set concentration of biotinylated 20-mer. Ability of both the 3-mer and 8-mer to inhibit longer peptide binding was observed, making PDZ:PL interactions an attractive target for design of small molecule or peptide therapeutics.

B. Methods

Peptides representing the C-terminal 3, 8 or 20 amino acids of a PDZ ligand were synthesized by standard FMOC chemistry and biotinylated if not used as an unlabeled competitor. Peptides three amino acids in length were acetylated to more properly mimic the peptide bond without introducing an amino-terminal charged group. Peptides were purified by reverse phase high performance liquid chromatography (HPLC) using a Vydac 218TP C18 Reversed Phase column having the dimensions of 10*25 mm, 5 um. Approximately 40 mg of peptide was dissolved in 2.0 ml of and aqueous solution of 49.9% acetonitrile and 0.1% Tri-Fluoro acetic acid (TFA). This solution was then injected into the HPLC machine through a 25 micron syringe filter (Millipore). Buffers used to get a good separation are (A) distilled water with 0.1% TFA and (B) 0.1% TFA with Acetonitrile. Gradient Segment setup is listed in TABLE 5 below.

TABLE 5

| | | | | Flow rate |
|---|---|---|---|---|
| Time | A | B | C | (ml/min) |
| 0 | 95% | 5% | 0 | 5.00 |
| 30 | 5% | 95% | 0 | 5.00 |

“Pure” fractions were collected, checked by mass spectrometry, and lyophilized (for stability). When ready to use, peptides were dissolved to 1 mM concentration in PBS, pH7, or dH2O and further diluted in PBS containing 2% BSA for use in the G Assay.

PDZ domain-containing genes used in these experiments include DLG1 and PSD95:

### Homo sapiens Post-Synaptic Density-95 (PSD-95)

Acc #: U83192
GI#: 3318652
Cloning sites: Bam H1/EcoR1

Construct (N-C);
vector: pGEX-3X
   For sequence, refer to TABLE 9: protein spans from amino terminal end of first PDZ domain to carboxy-terminal end of third PDZ domain in frame with GST in vector.

Primer:

| | |
|---|---|
| 8PSF1 (N1150-N1173) | |
| 5′-TCGGATCCTTGAGGGGGAGATGGA-3′ | (SEQ ID NO: 530) |
| 11PSR2 (N2191-N2168) | |
| 5′-TCGGAATTCGCTATACTCTTCTGG-3′ | (SEQ ID NO: 531) |

### Homo sapiens Discs Large Protein, Isoform 1 (DLG-1)

Acc #: U13897
GI#: 475816
Cloning sites: Bam H1/EcoR1

Construct (N-C);
vector: pGEX-3X
   For sequence, refer to TABLE 9: protein spans from amino terminal end of first PDZ domain to carboxy-terminal end of third PDZ domain in frame with GST in vector.

Primer:

| | |
|---|---|
| 1DF1 (N815-N837) | |
| 5′-TCGGATCCAGGTTAATGGCTCAG-3′ | (SEQ ID NO: 532) |
| 3DR2 (N1850-N1827) | |
| 5′-TCGGAATTCGACGTGACTCTTCGG-3′ | (SEQ ID NO: 533) |

DNA representing the putative open reading frames of human PSD-95 and DLG-1 were amplified by PCR and cloned into the pGEX-3X vector (Amersham-Pharmacia) to generate a GST-fusion vector. GST-fusion proteins were produced as recommended by the Pharmacia protocol by inducing this vector with IPTG in DH5α. Cells were lysed and purified by glutathione-sepharose chromatography according to manufacturer's instructions (Pharmacia). Purified protein was dialyzed against storage buffer (PBS with 25% glycerol) and stored at −20° C. (short term) or −80° C. (long term).

The G Assay was performed as described in EXAMPLE 3 with the exception that when a short competitor was used, 30 ul of competitor peptide (at twice the final concentration) was mixed with 30 ul biotinylated 20-mer (at twice the final concentration) and then added to the well.

PSD-95 and DLG-1 were incubated in the wells at 5 ug/ml as described in the G Assay protocol. Biotinylated 20-mer peptides used were 20 uM CLASP-2, 20 uM CD46, 10 uM CD95, and 10 uM KV1.3 (find sequences of peptides in TABLE 8). Competitors (unlabeled, short peptides) tested against each of the biotinylated peptides were 50 uM 8-mer of CD95, 100 uM 8-mer of CD46, 50 uM 8-mer of CLASP-2, and 1 mM and 500 uM acetylated 3-mer of CLASP-2. To deduce sequences, refer to TABLE 8. All absorbances were read at 450 nm after stopping TMB detection reaction at 30 min. Results were normalized in each group by dividing its A₄₅₀ by the A₄₅₀ of the PDZ/peptide binding in the absence of competitor and converting to percentage by multiplying by 100.

Results

TABLE 6

| PDZ | | Conc | | Conc | % |
|---|---|---|---|---|---|
| protein | Biotinylated 20-mer | uM | Competitor | uM | binding |
| PSD-95 | CLASP-2 | 20 | N/A | N/A | 100 |
| | | | CD95 8-mer | 50 | 92 |
| | | | CD46 8-mer | 100 | 81 |
| | | | CLASP-2 8-mer | 50 | 85 |
| | | | CLASP-2 3-mer | 1000 | 63 |
| | | | CLASP-2 3-mer | 500 | 82 |
| | CD46 | 20 | N/A | N/A | 100 |
| | | | CD95 8-mer | 50 | 100 |
| | | | CD46 8-mer | 100 | 95 |
| | | | CLASP-2 8-mer | 50 | 90 |
| | | | CLASP-2 3-mer | 1000 | 59 |
| | | | CLASP-2 3-mer | 500 | 75 |
| | CD95 | 10 | N/A | N/A | 100 |
| | | | CD95 8-mer | 50 | 75 |
| | | | CD46 8-mer | 100 | 65 |
| | | | CLASP-2 8-mer | 50 | 80 |
| | | | CLASP-2 3-mer | 1000 | 55 |
| | | | CLASP-2 3-mer | 500 | 55 |
| | KV1.3 | 10 | N/A | N/A | 100 |
| | | | CD95 8-mer | 50 | 87 |
| | | | CD46 8-mer | 100 | 71 |
| | | | CLASP-2 8-mer | 50 | 82 |
| | | | CLASP-2 3-mer | 1000 | 50 |
| | | | CLASP-2 3-mer | 500 | 81 |
| DLG-1 | CLASP-2 | 20 | N/A | N/A | 100 |
| | | | CD95 8-mer | 50 | 73 |
| | | | CD46 8-mer | 100 | 90 |
| | | | CLASP-2 8-mer | 50 | 93 |
| | | | CLASP-2 3-mer | 1000 | 59 |
| | | | CLASP-2 3-mer | 500 | 61 |
| | CD46 | 20 | N/A | N/A | 100 |
| | | | CD95 8-mer | 50 | 110 |
| | | | CD46 8-mer | 100 | 90 |
| | | | CLASP-2 8-mer | 50 | 105 |
| | | | CLASP-2 3-mer | 1000 | 45 |
| | | | CLASP-2 3-mer | 500 | 72 |
| | CD95 | 10 | N/A | N/A | 100 |
| | | | CD95 8-mer | 50 | 70 |
| | | | CD46 8-mer | 100 | 68 |
| | | | CLASP-2 8-mer | 50 | 75 |
| | | | CLASP-2 3-mer | 1000 | 46 |
| | | | CLASP-2 3-mer | 500 | 51 |
| | KV1.3 | 10 | N/A | N/A | 100 |
| | | | CD95 8-mer | 50 | 84 |
| | | | CD46 8-mer | 100 | 63 |

All standard errors are within 5% of the value.

TABLE 6 shows that it is possible to have successful competition with 3- and 8-mer unlabeled peptides against 20-mer biotinylated peptides with a 2.5-100 fold excess of unlabeled competitor. Specifically, 1 mM CLASP-2 acetylated 3-mer can successfully reduce labeled ligand binding up to 50% (50-100-fold excess). With DLG-1, the 50 uM CD95 8-mer can successfully reduce binding of CLASP-2 and CD95 labeled ligand approximately 30% at only 2.5 to 5-fold excess.

### EXAMPLE 7

### Antagonists and Agonists of PDZ/PL Interactions

A. Introduction

Many FDA approved drugs have unknown mechanism of function. It is quite possible that some of these drugs function by disrupting or increasing PDZ/PL interactions. This possibility was examined by using the ‘G Assay’ (Example 3 section D). FDA approved drugs were incubated in the presence of the labeled peptide and compared to the same interaction without drug to determine if there was an effect on specific PDZ:PL interactions (drugs added with peptide at step 9 in Example 3D). The primary focus of this experiment was on drugs involved in treatment of depression (amitriptyline, desipramine, trimipramine, benztropine, and nortryptilline) and epilepsy (valproic acid). No modes of action are known for these drugs.

The FDA approved drugs used in this study are listed in TABLE 11. Therapeutic dose was determined by guidelines given in the Physician's Desk Reference and in the assay, 200 times this amount was used. If a dosage range was given, the upper end of the range was used. Each interaction listed in TABLES 10A & B was tested in the ‘G Assay’ (see Example 3) against each of the drugs listed in TABLE 11. The concentration of GST-fusion protein and peptide used in the assay represent the Kd and were determined by titration. These values can be found in TABLE 7. The drugs were added to the peptide before addition to the well containing the PDZ protein. Otherwise, the assay was carried out as described and read at 450 nm after 30 minutes of developing. For the sequences of the PDZs and PLs used in these tests, see TABLES 8 & 9.

B. Results

As can be seen in TABLE 10A, agonist effects can be seen up to 4.3 fold higher than in the absence of drug, as in the case of AF6 and presenilin-1 in the presence of amitriptyline. Antagonistic effects have been demostrated here up to 4.2 fold higher, as in the cases of ZO-2 domain 1 and DNAM-1 in the presence of desipramine or nortryptilline and examples are listed in TABLE 10B.

Many agonist and antagonist effects can be seen when the drugs are incubated with PDZ/PL interactions. These results seem quite reasonable as the antidepressants used are from the tricyclic class and predominantly affect interactions where the peptide is known to function predominantly in the brain, e.g., presenilin 1 & 2 and norepinephrine transporter (NET). These results suggest that small molecules and therapeutic compounds can be used to modulate the binding between PDZ domains and their ligands.

TABLE 10A
Agonists 010726

| PDZ domain | PL | Drug | Change in OD |
|---|---|---|---|
| ZO-3 1/3 | Presenilin (115L) | Amitriptyline | 1.2 to 3.6 |
| ZO-3 1/3 | Presenilin (115L) | Desipramine | 1.2 to 3.3 |
| AF6 | Presenilin (115L) | Amitriptyline | 0.4 to 1.7 |
| DVL2 | Presenilin (115L) | Amitriptyline | 0.3 to 0.9 |
| hSyntenin | Presenilin (115L) | Amitriptyline | 1.1 to 2.7 |
| hSyntenin | Presenilin (115L) | Desipramine | 1.1 to 2.3 |
| hSyntenin | Presenilin (115L) | Trinipramine | 1.1 to 2.2 |
| FLJ10324 | Presenilin 2 (117L) | Desipramine | 0.4 to 0.8 |
| Par 3 3/3 | Presenilin 2 (117L) | Desipramine | 0.6 to 2.1 |
| Mupp-1 7/13 | Presenilin 2 (117L) | Desipramine | 0.5 to 1.0 |
| TIP-1 1/1 | LPAP (30L) | Benztropine | 1.1 to 1.6 |

TABLE 10B
Antagonists 010726

| | | | CHANGE |
|---|---|---|---|
| PDZ (DOMAIN) | PL | DRUG | IN OD |
| ZO-1 2/3 | NET (258L) | Imipramine | 0.8 to 0.4 |
| Atr-P (1/6) | DNAM (22L) | Desiprimine | 4 to 1.5 |
| BAI-1 (2/6) | DNAM (22L) | Desiprimine | 4 to 1.8 |
| ZO-2 (1/3) | DNAM (22L) | Desiprimine | 2.1 to 0.5 |
| ZO-2 (1/3) | DNAM (22L) | Nortryptilline | 2.1 to 0.5 |
| Hemba 1003117 | Presenilin 2 (117L) | Valproic Acid | 1.2 to 0.8 |
| Par 3 (3/3) | Presenilin 2 (117L) | Valproic Acid | 0.6 to 0.2 |
| Mupp-1 (7/13) | Presenilin 2 (117L) | Valproic Acid | 0.5 to 0.2 |
| PTPL-1 (4/5) | Presenilin 2 (117L) | Valproic Acid | 1.4 to 1.1 |

List of interactions and therapeutics for which a modulation of binding was observed. Concentrations at which the GST-PDZ fusion protein and labeled peptide were used can be found in Table 7 or Table 12. Concentration of drug used for each test at can be found in Table 11. ‘Change in OD’ shows the Absorbance of the interaction as measured by the ‘G Assay’ in the absence of drug at the left and the Absorbance of the interaction in the presence of drug at the right.

TABLE 11

| | | Sigma | Mol. | Thera Dose |
|---|---|---|---|---|
| Generic Name | Commercial Name | No. | Weight | 200× mg per mL |
| AMITRIPTYLINE | Elavil tablets and injection | A 8404 | 313.9 | 0.66 |
| HYDROCHLORIDE | | | | |
| ATROPINE SULFATE | Donnatal Elixir/Tablets | A 0257 | 676.8 | 0.0044 |
| BENZTROPINE MESYLATE | Cogetin Injection/Tablet | B 8262 | 403.5 | 0.00428 |
| CROMOLYN SODIUM | Gastrocrom Capsules | C 0399 | 512.3 | 0.88 |
| DESIPRAMINE | Nopramin Tablets | D 3900 | 302.8 | 1.32 |
| HYDROCHLORIDE | | | | |
| Imipramine HCI | | 113-52-0 | 317 | 0.88 |
| NORTRIPTYLINE | PAMELOR CAPSULES | N 7261 | 299.8 | 0.11 |
| HYDROCHLORIDE | | | | |
| TRIMIPRAMINE MALEATE | SURMONTIL CAPSULES | T 3146 | 410.5 | 0.44 |
| VALPROATE SODIUM | DEPACON INJECTION | P 4543 | 166.2 | 3 |
| VALPROIC ACID | DEPAKENE CAPSULES | P 6273 | 144.2 | 2 |

List of drugs used in Example 7. Therapeutic dose was determined by the Physician's Desk Reference. If a range of doses was given, the higher dose was used. In the G Assay, 200 times therapeutic dose was used, as represented in the column.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are incorporated by reference in their entirety for all purposes to the same extent as if each individual publication, patent or patent application were specifically and individually indicated to be so incorporated by reference.

TABLE 7

| | | Peptide | | | Protein | |
|---|---|---|---|---|---|---|
| | | Optimal | | PDZ | Optimal | |
| AVC ID | PL | Conc | PDZ | Domain | Conc | Classification |
| AA01.1 | Clasp-1 | 0 | Mint 1 | 1, 2 | 0 | 1 |
| | Clasp-1 | 0 | KIAA807 | | 0 | 1 |
| | Clasp-1 | 0 | KIAA0807(S) | 1 | 0 | 1 |
| | Clasp-1 | 0 | AIPC | 1 | 0 | 1 |
| AA02.1 | Clasp-2 | 0 | PTPL-1 | 2 | 0 | 1 |
| | Clasp-2 | 0 | PSD95 | 1 | 0 | 1 |
| | Clasp-2 | 0 | Outer Membrane | 1 | 0 | 1 |
| | Clasp-2 | 0 | NeDLG | 2 | 0 | 1 |
| | Clasp-2 | 0 | MUPP-1 | 13 | 0 | 1 |
| | Clasp-2 | 0 | MUPP-1 | 10 | 0 | 1 |
| | Clasp-2 | 0 | Mint 1 | 1, 2 | 0 | 1 |
| | Clasp-2 | 0 | KIAA807 | | 0 | 1 |
| | Clasp-2 | 0 | KIAA1634 | 2 | 0 | 1 |
| | Clasp-2 | 0 | KIAA1634 | 1 | 0 | 1 |
| | Clasp-2 | 0 | INADL | 8 | 0 | 1 |
| | Clasp-2 | 0 | FLJ 10324 | 1 | 0 | 1 |
| | Clasp-2 | 0 | DLG1 | 2 | 0 | 1 |
| | Clasp-2 | 0 | DLG1 | 1 | 0 | 1 |
| | Clasp-2 | 0 | BAI-1 | 5 | 0 | 1 |
| | Clasp-2 | 0 | BAI-1 | 2 | 0 | 1 |
| | Clasp-2 | 0 | AIPC | 1 | 0 | 1 |
| AA06 | CD6 | 0 | KIAA807 | | 0 | 1 |
| | CD6 | 0 | KIAA0807(S) | 1 | 5 | 1 |
| AA07 | CD34 | 0 | KIAA0382 | 1 | 0 | 1 |
| | CD34 | 0 | SHANK | 1 | 0 | 1 |
| | CD34 | 0 | KIAA0147 | 1 | 0 | 1 |
| | CD34 | 0 | PTN-4 | 1 | 0 | 1 |
| | CD34 | 0 | LIM RIL | 1 | 0 | 1 |
| | CD34 | 0 | BAI-1 | 6 | 0 | 1 |
| | CD34 | 0 | KIAA1634 | 5 | 0 | 1 |
| | CD34 | 0 | Atrophin-1 Inter. Prot. | 5 | 0 | 1 |
| AA091 | GAIP (G-alpha interacting | 0 | KIAA1526 | 1 | 0 | 1 |
| | protein) RGS 19 | | | | | |
| AA092 | alpha-1-syntrophin | 0 | KIAA0807(S) | 1 | 0 | 1 |
| AA093 | neurofascin (chicken) | 0 | ZO-2 | 2 | 0 | 1 |
| | neurofascin (chicken) | 0 | ZO-1 | 2 | 0 | 1 |
| | neurofascin (chicken) | 0 | ZO-1 | 1 | 0 | 1 |
| | neurofascin (chicken) | 0 | KIAA1526 | 1 | 0 | 1 |
| AA095 | GluR5-2 (rat) | 0 | KIAA0303 | 1 | 0 | 1 |
| | GluR5-2 (rat) | 0 | KIAA0147 | 1 | 0 | 2 |
| | GluR5-2 (rat) | 0 | PSD95 | 1, 2, 3 | 0 | 5 |
| | GluR5-2 (rat) | 0.1 | PSD95 | 3 | 1 | 5 |
| | GluR5-2 (rat) | 0 | PSD95 | 1 | 0 | 3 |
| | GluR5-2 (rat) | 0 | MUPP-1 | 10 | 0 | 2 |
| | GluR5-2 (rat) | 0 | MUPP-1 | 11 | 0 | 1 |
| | GluR5-2 (rat) | 0.1 | NeDLG | 1, 2 | 1 | 5 |
| | GluR5-2 (rat) | 0 | NeDLG | 3 | 0 | 2 |
| | GluR5-2 (rat) | 0 | NeDLG | 2 | 0 | 1 |
| | GluR5-2 (rat) | 0 | DLG2 | 2 | 0 | 1 |
| | GluR5-2 (rat) | 0 | DLG2 | 1 | 0 | 1 |
| | GluR5-2 (rat) | 0 | KIAA1719 | 3 | 0 | 1 |
| | GluR5-2 (rat) | 0 | DLG1 | 3 | 0 | 1 |
| | GluR5-2 (rat) | 0 | DLG1 | 2 | 0 | 2 |
| | GluR5-2 (rat) | 0 | DLG1 | 1 | 0 | 2 |
| | GluR5-2 (rat) | 0 | DLG1 | 1, 2 | 0 | 5 |
| | GluR5-2 (rat) | 0.15 | KIAA1634 | 1 | 0.1 | 5 |
| | GluR5-2 (rat) | 0.3 | BAI-1 | 2 | 1 | 5 |
| | GluR5-2 (rat) | 0 | atrophin-1 interacting | 1 | 0 | 1 |
| | | | Protein | | | |
| | GluR5-2 (rat) | 0 | KIAA0807(S) | 1 | 0 | 1 |
| AA098L | ropporin | 0 | TIP1 | 1 | 0 | 1 |
| AA10 | CD46 | 0 | KIAA0973 | 1 | 0 | 2 |
| | CD46 | 0 | Mint 1 | 2 | 0 | 1 |
| | CD46 | 0 | BAI-1 | 5 | 0 | 1 |
| AA105 | CX43 (connexin 43) | 0 | ZO-2 | 2 | 5 | 1 |
| | CX43 (connexin 43) | 0 | ZO-1 | 2 | 0 | 2 |
| AA106 | Kir2.1 (inwardly rect. K + channel) | 0 | PSD95 | 1, 2, 3 | 0 | 2 |
| | Kir2.1 (inwardly rect. K + channel) | 0 | NeDLG | 1, 2 | 0 | 1 |
| | Kir2.1 (inwardly rect. K + channel) | 0 | Outer Membrane | 1 | 0 | 4 |
| | Kir2.1 (inwardly rect. K + channel) | 0 | DLG2 | 2 | 0 | 1 |
| | Kir2.1 (inwardly rect. K + channel) | 0 | DLG1 | 2 | 0 | 1 |
| | Kir2.1 (inwardly rect. K + channel) | 5 | DLG1 | 1, 2 | 5 | 2 |
| | Kir2.1 (inwardly rect. K + channel) | 0 | KIAA1634 | 1 | 0 | 1 |
| | Kir2.1 (inwardly rect. K + channel) | 0 | atrophin-1 interacting | 1 | 0 | 1 |
| | | | Protein | | | |
| AA108.1 | GLUR2 (glutamate | 0 | PSD95 | 1, 2, 3 | 0 | 2 |
| | receptor 2 | | | | | |
| | GLUR2 (glutamate | 0 | NeDLG | 1, 2 | 0 | 2 |
| | receptor 2 | | | | | |
| | GLUR2 (glutamate | 0 | KIAA1634 | 1 | 0 | 4 |
| | receptor 2 | | | | | |
| | GLUR2 (glutamate | 0 | KIAA0807(S) | 1 | 0 | 1 |
| | receptor 2 | | | | | |
| | GLUR2 (glutamate | 0 | KIAA0147 | 1 | 0 | 1 |
| | receptor 2 | | | | | |
| | GLUR2 (glutamate | 0 | ENIGMA | 1 | 0 | 1 |
| | receptor 2 | | | | | |
| | GLUR2 (glutamate | 0 | DLG2 | 2 | 0 | 1 |
| | receptor 2 | | | | | |
| | GLUR2 (glutamate | 0 | DLG1 | 2 | 0 | 1 |
| | receptor 2 | | | | | |
| | GLUR2 (glutamate | 0 | DLG1 | 1, 2 | 0 | 2 |
| | receptor 2 | | | | | |
| | GLUR2 (glutamate | 0 | AIPC | 1 | 0 | 2 |
| | receptor 2 | | | | | |
| AA111 | ephrin A2 | 0 | KIAA0382 | 1 | 0 | 1 |
| | ephrin A2 | 0 | MUPP-1 | 11 | 0 | 1 |
| | ephrin A2 | 0 | Mint 1 | 2 | 0 | 1 |
| | ephrin A2 | 0 | KIAA1719 | 6 | 0 | 1 |
| AA112 | GluR delta-2 | 0 | Outer Membrane | 1 | 0 | 2 |
| | GluR delta-2 | 0 | KIAA807 | | 0 | 3 |
| | GluR delta-2 | 0 | KIAA1526 | 1 | 5 | 2 |
| | GluR delta-2 | 4 | KIAA0807(S) | 1 | 0.5 | 4 |
| AA113 | SSTR2 (somatostatin | 0 | GRIP1 | 7 | 0 | 1 |
| | recepor 2) | | | | | |
| | SSTR2 (somatostatin | 0 | KIAA0382 | 1 | 0 | 1 |
| | recepor 2) | | | | | |
| | SSTR2 (somatostatin | 0 | SHANK | 1 | 0 | 1 |
| | recepor 2) | | | | | |
| | SSTR2 (somatostatin | 0 | Mint 1 | 1, 2 | 0 | 1 |
| | recepor 2) | | | | | |
| | SSTR2 (somatostatin | 0 | Mint 1 | 2 | 0 | 1 |
| | recepor 2) | | | | | |
| | SSTR2 (somatostatin | 0 | KIAA807 | | 0 | 2 |
| | recepor 2) | | | | | |
| | SSTR2 (somatostatin | 0 | KIAA1719 | 6 | 0 | 1 |
| | recepor 2) | | | | | |
| | SSTR2 (somatostatin | 0 | KIAA1526 | 1 | 0 | 1 |
| | recepor 2) | | | | | |
| | SSTR2 (somatostatin | 0 | KIAA0807(S) | 1 | 0 | 2 |
| | recepor 2) | | | | | |
| AA114 | GLUR7 (metabotropic | 0 | DLG1 | 2 | 0 | 1 |
| | glutamate receptor) | | | | | |
| | GLUR7 (metabotropic | 0 | KIAA1634 | 1 | 0 | 1 |
| | glutamate receptor) | | | | | |
| | GLUR7 (metabotropic | 0 | PAR3 | 3 | 0 | 2 |
| | glutamate receptor) | | | | | |
| AA115 | presenilin-1 | 0.1 | ZO-3 | 1 | 1 | 5 |
| | presenilin-1 | 0 | ZO-2 | 1 | 0 | 1 |
| | presenilin-1 | 0 | ZO-1 | 1 | 0 | 2 |
| | presenilin-1 | 0 | Unnamed Protein | 2 | 0 | 1 |
| | presenilin-1 | 0 | TIP1 | 1 | 0 | 2 |
| | presenilin-1 | 0 | KIAA0147 | 3 | 0 | 1 |
| | presenilin-1 | 0.2 | INADL | 8 | 5 | 3 |
| | presenilin-1 | 0 | PTPL-1 | 4 | 0 | 4 |
| | presenilin-1 | 0 | INADL | 5 | 0 | 2 |
| | presenilin-1 | 0.2 | INADL | 3 | 0.5 | 5 |
| | presenilin-1 | 0.1 | hSyntenin | 1 | 5 | 3 |
| | presenilin-1 | 0.1 | HEMBA 1003117 | 1 | 0.65 | 5 |
| | presenilin-1 | 0 | MUPP-1 | 10 | 0 | 2 |
| | presenilin-1 | 0 | MUPP-1 | 11 | 0 | 1 |
| | presenilin-1 | 0 | hAPXL | 1 | 0 | 1 |
| | presenilin-1 | 0 | P55T | 1 | 0 | 1 |
| | presenilin-1 | 0 | NOS1 | 1 | 0 | 2 |
| | presenilin-1 | 0.15 | GRIP1 | 6 | 5 | 3 |
| | presenilin-1 | 0.3 | MUPP-1 | 9 | 0.5 | 5 |
| | presenilin-1 | 0 | MUPP-1 | 8 | 0 | 1 |
| | presenilin-1 | 0.03 | MUPP-1 | 7 | 1 | 5 |
| | presenilin-1 | 0 | MUPP-1 | 6 | 0 | 1 |
| | presenilin-1 | 0 | FLJ21687 | 1 | 0 | 1 |
| | presenilin-1 | 0 | FLJ 10324 | 1 | 0 | 5 |
| | presenilin-1 | 0 | MUPP-1 | 2 | 0 | 2 |
| | presenilin-1 | 0 | MPP2 | 1 | 0 | 1 |
| | presenilin-1 | 0.08 | Mint 1 | 1, 2 | 0.5 | 5 |
| | presenilin-1 | 0.1 | Mint 1 | 2 | 1 | 4 |
| | presenilin-1 | 0.1 | Mint 1 | 1 | 5 | 4 |
| | presenilin-1 | 0 | LIM-Mystique | 1 | 0 | 2 |
| | presenilin-1 | 0 | LIM RIL | 1 | 0 | 2 |
| | presenilin-1 | 0.2 | KIAA807 | | 5 | 4 |
| | presenilin-1 | 0.1 | DVL2 | 1 | 0.5 | 4 |
| | presenilin-1 | 0 | KIAA1719 | 6 | 0 | 5 |
| | presenilin-1 | 0 | KIAA1719 | 5 | 0 | 3 |
| | presenilin-1 | 0 | CASK | 1 | 0 | 2 |
| | presenilin-1 | 0 | KIAA1634 | 5 | 0 | 2 |
| | presenilin-1 | 0 | KIAA1634 | 4 | 0 | 2 |
| | presenilin-1 | 0 | BAI-1 | 2 | 0 | 2 |
| | presenilin-1 | 0.2 | Atrophin-1 Inter. Prot. | 5 | 5 | 3 |
| | presenilin-1 | 0 | atrophin-1 interacting | 4 | 0 | 2 |
| | | | Protein | | | |
| | presenilin-1 | 0 | atrophin-1 interacting | 3 | 0 | 1 |
| | | | Protein | | | |
| | presenilin-1 | 0 | KIAA1222 | 1 | 5 | 3 |
| | presenilin-1 | 0 | AIPC | 4 | 0 | 5 |
| | presenilin-1 | 0 | AIPC | 1 | 0 | 5 |
| | presenilin-1 | 0.1 | AF6 | 1 | 0.5 | 3 |
| | presenilin-1 | 0 | PAR3 | 3 | 0 | 5 |
| | presenilin-1 | 0 | KIAA0807(S) | 1 | 0 | 5 |
| | presenilin-1 | 0.3 | ZO-3 | 3 | 5 | 3 |
| AA116 | MINT-2 | 0 | KIAA0382 | 1 | 0 | 1 |
| | MINT-2 | 0 | KIAA0300 | 1 | 0 | 3 |
| | MINT-2 | 0 | PTPL-1 | 4 | 0 | 4 |
| | MINT-2 | 0 | hSyntenin | 1 | 0 | 2 |
| | MINT-2 | 0 | HEMBA 1003117 | 1 | 0 | 3 |
| | MINT-2 | 0 | KIAA1222 | 1 | 0 | 1 |
| | MINT-2 | 0 | MUPP-1 | 11 | 0 | 5 |
| | MINT-2 | 0 | P55T | 1 | 0 | 1 |
| | MINT-2 | 0 | PDZK-1 | 4 | 0 | 1 |
| | MINT-2 | 0 | MUPP-1 | 9 | 0 | 1 |
| | MINT-2 | 0 | MUPP-1 | 7 | 0 | 1 |
| | MINT-2 | 0 | MUPP-1 | 3 | 0 | 1 |
| | MINT-2 | 0 | FLJ 10324 | 1 | 0 | 4 |
| | MINT-2 | 0 | MUPP-1 | 2 | 0 | 1 |
| | MINT-2 | 0 | Mint 1 | 1, 2 | 0 | 5 |
| | MINT-2 | 0 | Mint 1 | 2 | 0 | 1 |
| | MINT-2 | 0 | Mint 1 | 1 | 0 | 2 |
| | MINT-2 | 0 | KIAA807 | | 0 | 3 |
| | MINT-2 | 0 | DVL2 | 1 | 0 | 2 |
| | MINT-2 | 0 | AIPC | 1 | 0 | 1 |
| | MINT-2 | 0 | PAR3 | 3 | 0 | 5 |
| | MINT-2 | 0 | KIAA0807(S) | 1 | 0 | 4 |
| | MINT-2 | 0 | ZO-3 | 3 | 0 | 1 |
| AA117 | presenilin-2 | 0 | ZO-1 | 1 | 0 | 1 |
| | presenilin-2 | 0 | KIAA0751(L) | 1 | 0 | 1 |
| | presenilin-2 | 0 | KIAA0561 | 1 | 0 | 1 |
| | presenilin-2 | 0 | KIAA0300 | 2 | 0 | 1 |
| | presenilin-2 | 0 | KIAA0300 | 1 | 0 | 1 |
| | presenilin-2 | 8 | PTPL-1 | 4 | 3 | 2 |
| | presenilin-2 | 0 | INADL | 3 | 0 | 1 |
| | presenilin-2 | 4 | HEMBA 1003117 | 1 | 0.5 | 4 |
| | presenilin-2 | 0 | NOS1 | 1 | 0 | 1 |
| | presenilin-2 | 0 | MUPP-1 | 9 | 5 | 3 |
| | presenilin-2 | 4 | MUPP-1 | 7 | 5 | 2 |
| | presenilin-2 | 0 | MUPP-1 | 3 | 5 | 3 |
| | presenilin-2 | 1 | FLJ 10324 | 1 | 1 | 5 |
| | presenilin-2 | 6 | Mint 1 | 1, 2 | 2 | 3 |
| | presenilin-2 | 0 | DVL2 | 1 | 0 | 1 |
| | presenilin-2 | 0 | Atrophin-1 Inter. Prot. | 5 | 0 | 1 |
| | presenilin-2 | 0 | AIPC | 4 | 0 | 1 |
| | presenilin-2 | 0 | AIPC | 1 | 0 | 1 |
| | presenilin-2 | 0 | AF6 | 1 | 5 | 3 |
| | presenilin-2 | 2 | PAR3 | 3 | 0.5 | 5 |
| | presenilin-2 | 0 | KIAA0807 (S) | 1 | 0 | 1 |
| AA118 | MINT-1 | 0 | ZO-3 | 1 | 0 | 2 |
| | MINT-1 | 0 | ZO-1 | 1 | 0 | 1 |
| | MINT-1 | 0 | KIAA0382 | 1 | 0 | 1 |
| | MINT-1 | 0 | KIAA0300 | 1 | 0 | 4 |
| | MINT-1 | 0 | INADL | 8 | 0 | 1 |
| | MINT-1 | 0 | PTPL-1 | 4 | 0 | 5 |
| | MINT-1 | 0.8 | hSyntenin | 1 | 5 | 3 |
| | MINT-1 | 0 | HEMBA 1003117 | 1 | 0 | 3 |
| | MINT-1 | 0 | KIAA1222 | 1 | 0 | 1 |
| | MINT-1 | 0 | MUPP-1 | 10 | 0 | 1 |
| | MINT-1 | 0 | MUPP-1 | 11 | 0 | 5 |
| | MINT-1 | 0 | NOS1 | 1 | 0 | 4 |
| | MINT-1 | 0 | PDZK-1 | 4 | 0 | 1 |
| | MINT-1 | 0 | MUPP-1 | 9 | 0 | 3 |
| | MINT-1 | 1.5 | MUPP-1 | 7 | 5 | 3 |
| | MINT-1 | 0 | MUPP-1 | 5 | 0 | 2 |
| | MINT-1 | 0 | MUPP-1 | 3 | 0 | 3 |
| | MINT-1 | 1 | FLJ 10324 | 1 | 1 | 5 |
| | MINT-1 | 0 | MUPP-1 | 2 | 0 | 1 |
| | MINT-1 | 0 | MUPP-1 | 1 | 0 | 1 |
| | MINT-1 | 0 | Mint 1 | 1, 2 | 0 | 5 |
| | MINT-1 | 2 | Mint 1 | 1 | 5 | 3 |
| | MINT-1 | 0 | KIAA807 | | 0 | 4 |
| | MINT-1 | 0 | DVL2 | 1 | 0 | 2 |
| | MINT-1 | 0 | AIPC | 1 | 0 | 1 |
| | MINT-1 | 0 | PAR3 | 3 | 0 | 5 |
| | MINT-1 | 0 | KIAA0807(S) | 1 | 0 | 5 |
| | MINT-1 | 0 | ZO-3 | 3 | 0 | 1 |
| AA121 | CD68 | 0 | X11-beta | 2 | 0 | 1 |
| | CD68 | 0 | SHANK | 1 | 0 | 1 |
| | CD68 | 0 | KIAA0973 | 1 | 0 | 1 |
| | CD68 | 0 | hAPXL | 1 | 0 | 2 |
| | CD68 | 0 | GRIP1 | 6 | 0 | 1 |
| | CD68 | 0 | FLJ 10324 | 1 | 0 | 1 |
| | CD68 | 0 | Mint 1 | 1, 2 | 0 | 1 |
| | CD68 | 0 | Mint 1 | 2 | 0 | 2 |
| | CD68 | 0 | DVL2 | 1 | 0 | 2 |
| | CD68 | 0 | KIAA1719 | 3 | 0 | 1 |
| | CD68 | 0 | KIAA1719 | 6 | 0 | 1 |
| | CD68 | 0 | KIAA1634 | 1 | 0 | 2 |
| | CD68 | 0 | BAI-1 | 2 | 0 | 3 |
| | CD68 | 0 | KIAA0807(S) | 1 | 0 | 5 |
| AA123 | a-actinin 2 | 0 | rat SHANK 3 | 1 | 0 | 1 |
| | a-actinin 2 | 1 | TIP1 | 1 | 0.5 | 5 |
| | a-actinin 2 | 0 | KIAA0380 | 1 | 0 | 1 |
| | a-actinin 2 | 0 | KIAA0316 | 1 | 0 | 1 |
| | a-actinin 2 | 2.5 | TAX IP2 | 1 | 5 | 5 |
| | a-actinin 2 | 0 | Syntrophin gamma-2 | 1 | 0 | 1 |
| | a-actinin 2 | 0 | Syntrophin gamma-1 | 1 | 0 | 1 |
| | a-actinin 2 | 0 | Synt. 1 alpha | 1 | 5 | 3 |
| | a-actinin 2 | 0 | SHANK | 1 | 0 | 1 |
| | a-actinin 2 | 0 | KIAA0147 | 3 | 0 | 3 |
| | a-actinin 2 | 0 | KIAA0147 | 1 | 0 | 2 |
| | a-actinin 2 | 0 | PTPL-1 | 2 | 0 | 1 |
| | a-actinin 2 | 0 | INADL | 3 | 0 | 1 |
| | a-actinin 2 | 0 | KIAA0973 | 1 | 0 | 2 |
| | a-actinin 2 | 0 | hAPXL | 1 | 0 | 5 |
| | a-actinin 2 | 0 | Outer Membrane | 1 | 0 | 1 |
| | a-actinin 2 | 0 | Novel PDZ | 1 | 0 | 2 |
| | a-actinin 2 | 0 | Mint 1 | 1, 2 | 0 | 1 |
| | a-actinin 2 | 0 | Mint 1 | 2 | 0 | 1 |
| | a-actinin 2 | 0 | Erbin | 1 | 0 | 1 |
| | a-actinin 2 | 0 | ENIGMA | 1 | 0 | 5 |
| | a-actinin 2 | 0 | LIM-Mystique | 1 | 0 | 5 |
| | a-actinin 2 | 0 | LIM RIL | 1 | 0 | 5 |
| | a-actinin 2 | 2 | LIM Protein | 1 | 1 | 4 |
| | a-actinin 2 | 0 | KIAA807 | | 0 | 5 |
| | a-actinin 2 | 0 | DLG1 | 2 | 0 | 1 |
| | a-actinin 2 | 0 | DLG1 | 1, 2 | 0 | 1 |
| | a-actinin 2 | 0 | BAI-1 | 6 | 0 | 5 |
| | a-actinin 2 | 2 | KIAA1634 | 5 | 1 | 5 |
| | a-actinin 2 | 0 | BAI-1 | 2 | 0 | 1 |
| | a-actinin 2 | 0 | Atrophin-1 Inter. Prot. | 5 | 0 | 5 |
| | a-actinin 2 | 0 | KIAA1526 | 1 | 0 | 1 |
| | a-actinin 2 | 0 | AIPC | 1 | 0 | 2 |
| | a-actinin 2 | 0 | PAR3 | 3 | 0 | 1 |
| | a-actinin 2 | 0 | KIAA0807(S) | 1 | 0 | 5 |
| AA125 | zona occludens 3 (ZO-3) | 0 | KIAA0382 | 1 | 0 | 2 |
| | zona occludens 3 (ZO-3) | 0 | SHANK | 1 | 0 | 1 |
| | zona occludens 3 (ZO-3) | 0 | PTPL-1 | 2 | 0 | 2 |
| | zona occludens 3 (ZO-3) | 0 | KIAA0973 | 1 | 0 | 2 |
| | zona occludens 3 (ZO-3) | 0 | MUPP-1 | 13 | 0 | 2 |
| | zona occludens 3 (ZO-3) | 0 | hAPXL | 1 | 0 | 2 |
| | zona occludens 3 (ZO-3) | 0 | Novel PDZ | 1 | 0 | 1 |
| | zona occludens 3 (ZO-3) | 0 | MUPP-1 | 9 | 0 | 1 |
| | zona occludens 3 (ZO-3) | 0 | MUPP-1 | 7 | 0 | 1 |
| | zona occludens 3 (ZO-3) | 0 | Mint 1 | 2 | 0 | 2 |
| | zona occludens 3 (ZO-3) | 0 | LIM-Mystique | 1 | 0 | 2 |
| | zona occludens 3 (ZO-3) | 0 | ENIGMA | 1 | 0 | 1 |
| | zona occludens 3 (ZO-3) | 0 | LIM RIL | 1 | 0 | 2 |
| | zona occludens 3 (ZO-3) | 0 | KIAA807 | | 0 | 5 |
| | zona occludens 3 (ZO-3) | 0 | KIAA1634 | 5 | 0 | 1 |
| | zona occludens 3 (ZO-3) | 0 | BAI-1 | 6 | 0 | 2 |
| | zona occludens 3 (ZO-3) | 0 | KIAA1526 | 1 | 0 | 1 |
| | zona occludens 3 (ZO-3) | 0 | AIPC | 1 | 0 | 1 |
| | zona occludens 3 (ZO-3) | 0 | PAR3 | 3 | 0 | 1 |
| | zona occludens 3 (ZO-3) | 0 | KIAA0807(S) | 1 | 0 | 5 |
| AA13 | CD95 (fas) | 0 | PTPL-1 | 4 | 0 | 1 |
| | CD95 (fas) | 0 | PTPL-1 | 2 | 5 | 3 |
| | CD95 (fas) | 0 | Outer Membrane | 1 | 0 | 1 |
| | CD95 (fas) | 0 | FLJ 10324 | 1 | 0 | 1 |
| | CD95 (fas) | 0 | DLG2 | 2 | 0 | 1 |
| | CD95 (fas) | 0 | DLG1 | 2 | 0 | 1 |
| | CD95 (fas) | 0 | BAI-1 | 5 | 5 | 3 |
| | CD95 (fas) | 0 | KIAA1634 | 4 | 0 | 1 |
| | CD95 (fas) | 0 | KIAA1634 | 2 | 0 | 1 |
| | CD95 (fas) | 0 | KIAA1634 | 1 | 0 | 1 |
| | CD95 (fas) | 0 | AIPC | 1 | 0 | 1 |
| AA140 | KIA 1481 | 0 | TIP1 | 1 | 0 | 2 |
| | KIA 1481 | 0 | KIAA0382 | 1 | 0 | 5 |
| | KIA 1481 | 0 | SHANK | 1 | 0 | 5 |
| | KIA 1481 | 0 | SHANK3 | 1 | 0 | 3 |
| | KIA 1481 | 0 | EBP50 | 1 | 0 | 2 |
| | KIA 1481 | 0 | EBP50 | 2 | 0 | 2 |
| | KIA 1481 | 0 | KIAA0147 | 1 | 0 | 2 |
| | KIA 1481 | 0 | INADL | 5 | 0 | 1 |
| | KIA 1481 | 0 | KIAA0973 | 1 | 0 | 2 |
| | KIA 1481 | 0 | KIAA1095 | 1 | 0 | 1 |
| | KIA 1481 | 0.6 | hAPXL | 1 | 0.5 | 5 |
| | KIA 1481 | 0 | Novel PDZ | 2 | 0 | 1 |
| | KIA 1481 | 0 | Novel PDZ | 1 | 0 | 1 |
| | KIA 1481 | 0 | PDZK1 | 2, 3, 4 | 0 | 2 |
| | KIA 1481 | 0 | FLJ00011 | 1 | 0 | 2 |
| | KIA 1481 | 0.8 | Mint 1 | 1, 2 | 5 | 3 |
| | KIA 1481 | 0 | Mint 1 | 2 | 0 | 3 |
| | KIA 1481 | 0 | KIAA807 | | 0 | 5 |
| | KIA 1481 | 0 | KIAA1634 | 5 | 0 | 1 |
| | KIA 1481 | 0 | BAI-1 | 6 | 0 | 2 |
| | KIA 1481 | 0 | BAI-1 | 5 | 5 | 3 |
| | KIA 1481 | 0 | KIAA1634 | 2 | 0 | 1 |
| | KIA 1481 | 0 | KIAA1634 | 1 | 0 | 2 |
| | KIA 1481 | 0 | BAI-1 | 4 | 0 | 1 |
| | KIA 1481 | 0 | BAI-1 | 2 | 0 | 2 |
| | KIA 1481 | 0 | KIAA1526 | 1 | 0 | 2 |
| | KIA 1481 | 0 | PDZ-73 | 2 | 0 | 1 |
| | KIA 1481 | 0 | KIAA0807(S) | 1 | 0 | 5 |
| AA147 | Na+/Pi cotransporter 2 | 0 | rat SHANK 3 | 1 | 0 | 4 |
| | Na+/Pi cotransporter 2 | 0 | ZO-2 | 1 | 0 | 1 |
| | Na+/Pi cotransporter 2 | 0 | Syntrophin gamma-2 | 1 | 0 | 1 |
| | Na+/Pi cotransporter 2 | 0 | SHANK | 1 | 0 | 5 |
| | Na+/Pi cotransporter 2 | 0 | SHANK3 | 1 | 0 | 5 |
| | Na+/Pi cotransporter 2 | 0 | EBP50 | 1 | 0 | 5 |
| | Na+/Pi cotransporter 2 | 0 | EBP50 | 2 | 0 | 2 |
| | Na+/Pi cotransporter 2 | 0 | INADL | 8 | 0 | 1 |
| | Na+/Pi cotransporter 2 | 0 | PIST | 1 | 0 | 1 |
| | Na+/Pi cotransporter 2 | 0 | KIAA0973 | 1 | 0 | 2 |
| | Na+/Pi cotransporter 2 | 0 | MUPP-1 | 10 | 0 | 1 |
| | Na+/Pi cotransporter 2 | 0 | MUPP-1 | 13 | 0 | 1 |
| | Na+/Pi cotransporter 2 | 0 | hAPXL | 1 | 0 | 1 |
| | Na+/Pi cotransporter 2 | 0 | Outer Membrane | 1 | 0 | 1 |
| | Na+/Pi cotransporter 2 | 0 | PDZK1 | 2, 3, 4 | 0 | 1 |
| | Na+/Pi cotransporter 2 | 0 | FLJ 10324 | 1 | 0 | 1 |
| | Na+/Pi cotransporter 2 | 0 | Mint 1 | 2 | 0 | 1 |
| | Na+/Pi cotransporter 2 | 0 | KIAA807 | | 0 | 5 |
| | Na+/Pi cotransporter 2 | 0 | KIAA1526 | 1 | 0 | 1 |
| | Na+/Pi cotransporter 2 | 0 | KIAA0807(S) | 1 | 0 | 5 |
| AA148L | CFTCR (cystic fibrosis | 0 | SHANK | 1 | 0 | 1 |
| | transmembrane | | | | | |
| | conductance regulator) | | | | | |
| | CFTCR (cystic fibrosis | 0 | KIAA807 | | 0 | 1 |
| | transmembrane | | | | | |
| | conductance regulator) | | | | | |
| | CFTCR (cystic fibrosis | 0 | KIAA0807(S) | 1 | 0 | 2 |
| | transmembrane | | | | | |
| | conductance regulator) | | | | | |
| AA152L | ActRIIA | 5 | PTPL-1 | 2 | 5 | 3 |
| | ActRIIA | 5 | KIAA1634 | 2 | 5 | 2 |
| AA161 | MINT-3 | 0 | KIAA0561 | 1 | 0 | 1 |
| | MINT-3 | 0 | KIAA0316 | 1 | 0 | 2 |
| | MINT-3 | 0 | KIAA0973 | 1 | 0 | 2 |
| | MINT-3 | 0 | MUPP-1 | 11 | 0 | 2 |
| | MINT-3 | 0 | MUPP-1 | 3 | 0 | 1 |
| | MINT-3 | 0 | Mint 1 | 1, 2 | 0 | 2 |
| | MINT-3 | 0 | Mint 1 | 2 | 0 | 2 |
| | MINT-3 | 0 | LIM Protein | 1 | 0 | 1 |
| | MINT-3 | 0 | KIAA807 | | 0 | 1 |
| | MINT-3 | 0 | DVL2 | 1 | 0 | 1 |
| | MINT-3 | 0 | AF6 | 1 | 0 | 1 |
| | MINT-3 | 0 | PAR3 | 3 | 0 | 1 |
| | MINT-3 | 0 | KIAA0807(S) | 1 | 0 | 1 |
| AA169L | CAPON (carboxyl-terminal | 0 | PTPL-1 | 4 | 0 | 1 |
| | PDZ ligand of neuronal | | | | | |
| | nitric oxide synthase) | | | | | |
| | mRNa | | | | | |
| | CAPON (carboxyl-terminal | 0 | hAPXL | 1 | 0 | 1 |
| | PDZ ligand of neuronal | | | | | |
| | nitric oxide synthase) | | | | | |
| | mRNA | | | | | |
| | CAPON (carboxyl-terminal | 0 | KIAA807 | | 0 | 1 |
| | PDZ ligand of neuronal | | | | | |
| | nitric oxide synthase) | | | | | |
| | mRNA | | | | | |
| | CAPON (carboxyl-terminal | 0 | AIPC | 1 | 0 | 1 |
| | PDZ ligand of neuronal | | | | | |
| | nitric oxide synthase) | | | | | |
| | mRNA | | | | | |
| | CAPON (carboxyl-terminal | 0 | PAR3 | 3 | 0 | 1 |
| | PDZ ligand of neuronal | | | | | |
| | nitric oxide synthase) | | | | | |
| | mRNA | | | | | |
| | CAPON (carboxyl-terminal | 0 | KIAA0807(S) | 1 | 0 | 1 |
| | PDZ ligand of neuronal | | | | | |
| | nitric oxide synthase) | | | | | |
| | mRNA | | | | | |
| AA172 | RA-GEF (ras/rap1A- | 0 | KIAA0147 | 1 | 0 | 1 |
| | assoc.-GEF) | | | | | |
| | RA-GEF (ras/rap1A- | 0 | PTPL-1 | 2 | 0 | 4 |
| | assoc.-GEF) | | | | | |
| | RA-GEF (ras/rap1A- | 0 | KIAA1634 | 2 | 0 | 2 |
| | assoc.-GEF) | | | | | |
| AA177L | c-kit receptor | 0 | INADL | 8 | 0 | 1 |
| | c-kit receptor | 0 | MUPP-1 | 10 | 0 | 1 |
| | c-kit receptor | 0 | Mint 1 | 2 | 0 | 1 |
| | c-kit receptor | 0 | LIM RIL | 1 | 0 | 1 |
| AA178L | PDZ-binding kinase (PBK) | 0 | TIP1 | 1 | 0 | 1 |
| | PDZ-binding kinase (PBK) | 0 | Syntrophin gamma-1 | 1 | 0 | 1 |
| | PDZ-binding kinase (PBK) | 0 | Synt. 1 alpha | 1 | 0 | 1 |
| | PDZ-binding kinase (PBK) | 6 | PTPL-1 | 2 | 0.5 | 4 |
| | PDZ-binding kinase (PBK) | 0 | PSD95 | 1, 2, 3 | 0 | 1 |
| | PDZ-binding kinase (PBK) | 0 | NeDLG | 1, 2 | 0 | 1 |
| | PDZ-binding kinase (PBK) | 0 | DLG1 | 1, 2 | 0 | 1 |
| | PDZ-binding kinase (PBK) | 7 | KIAA1634 | 2 | 1 | 3 |
| | PDZ-binding kinase (PBK) | 0 | BAI-1 | 3 | 0 | 1 |
| | PDZ-binding kinase (PBK) | 0 | Atrophin-1 Inter. Prot. | 5 | 0 | 1 |
| AA180 | NMDA Glutamate | 0 | TIP1 | 1 | 0 | 5 |
| | Receptor 2C | | | | | |
| | NMDA Glutamate | 0 | KIAA0382 | 1 | 0 | 1 |
| | Receptor 2C | | | | | |
| | NMDA Glutamate | 0 | KIAA0380 | 1 | 0 | 1 |
| | Receptor 2C | | | | | |
| | NMDA Glutamate | 0 | TAX IP2 | 1 | 0 | 4 |
| | Receptor 2C | | | | | |
| | NMDA Glutamate | 0 | Syntrophin gamma-2 | 1 | 0 | 2 |
| | Receptor 2C | | | | | |
| | NMDA Glutamate | 0 | Syntrophin gamma-1 | 1 | 0 | 4 |
| | Receptor 2C | | | | | |
| | NMDA Glutamate | 0 | Synt. 1 alpha | 1 | 0 | 4 |
| | Receptor 2C | | | | | |
| | NMDA Glutamate | 0 | KIAA0147 | 3 | 0 | 1 |
| | Receptor 2C | | | | | |
| | NMDA Glutamate | 0 | KIAA0147 | 2 | 0 | 1 |
| | Receptor 2C | | | | | |
| | NMDA Glutamate | 0 | KIAA0147 | 1 | 0 | 5 |
| | Receptor 2C | | | | | |
| | NMDA Glutamate | 0 | INADL | 8 | 0 | 1 |
| | Receptor 2C | | | | | |
| | NMDA Glutamate | 0 | PTPL-1 | 2 | 0 | 5 |
| | Receptor 2C | | | | | |
| | NMDA Glutamate | 0 | PTN-4 | 1 | 0 | 2 |
| | Receptor 2C | | | | | |
| | NMDA Glutamate | 0 | INADL | 5 | 0 | 1 |
| | Receptor 2C | | | | | |
| | NMDA Glutamate | 0 | INADL | 3 | 0 | 2 |
| | Receptor 2C | | | | | |
| | NMDA Glutamate | 0 | PSD95 | 1, 2, 3 | 0 | 5 |
| | Receptor 2C | | | | | |
| | NMDA Glutamate | 0 | PSD95 | 3 | 0 | 2 |
| | Receptor 2C | | | | | |
| | NMDA Glutamate | 0 | PSD95 | 1 | 0 | 5 |
| | Receptor 2C | | | | | |
| | NMDA Glutamate | 0 | KIAA0973 | 1 | 0 | 1 |
| | Receptor 2C | | | | | |
| | NMDA Glutamate | 0 | KIAA1095 | 1 | 0 | 1 |
| | Receptor 2C | | | | | |
| | NMDA Glutamate | 0 | MUPP-1 | 10 | 0 | 1 |
| | Receptor 2C | | | | | |
| | NMDA Glutamate | 0 | MUPP-1 | 13 | 0 | 5 |
| | Receptor 2C | | | | | |
| | NMDA Glutamate | 0 | NeDLG | 1, 2 | 0 | 5 |
| | Receptor 2C | | | | | |
| | NMDA Glutamate | 0 | hAPXL | 1 | 0 | 1 |
| | Receptor 2C | | | | | |
| | NMDA Glutamate | 0 | Outer Membrane | 1 | 0 | 5 |
| | Receptor 2C | | | | | |
| | NMDA Glutamate | 0 | NOS1 | 1 | 0 | 1 |
| | Receptor 2C | | | | | |
| | NMDA Glutamate | 0 | NeDLG | 3 | 0 | 1 |
| | Receptor 2C | | | | | |
| | NMDA Glutamate | 0 | NeDLG | 2 | 0 | 5 |
| | Receptor 2C | | | | | |
| | NMDA Glutamate | 0 | NeDLG | 1 | 0 | 2 |
| | Receptor 2C | | | | | |
| | NMDA Glutamate | 0 | MUPP-1 | 5 | 0 | 1 |
| | Receptor 2C | | | | | |
| | NMDA Glutamate | 0 | FLJ 11215 | 1 | 0 | 1 |
| | Receptor 2C | | | | | |
| | NMDA Glutamate | 0 | FLJ 00011 | 1 | 0 | 2 |
| | Receptor 2C | | | | | |
| | NMDA Glutamate | 0 | Mint 1 | 1, 2 | 0 | 1 |
| | Receptor 2C | | | | | |
| | NMDA Glutamate | 0 | Mint 1 | 2 | 0 | 1 |
| | Receptor 2C | | | | | |
| | NMDA Glutamate | 0 | LIMK1 | 1 | 0 | 1 |
| | Receptor 2C | | | | | |
| | NMDA Glutamate | 0 | LIM-Mystique | 1 | 0 | 4 |
| | Receptor 2C | | | | | |
| | NMDA Glutamate | 0 | Erbin | 1 | 0 | 4 |
| | Receptor 2C | | | | | |
| | NMDA Glutamate | 0 | LIM RIL | 1 | 0 | 5 |
| | Receptor 2C | | | | | |
| | NMDA Glutamate | 0 | KIAA807 | | 0 | 4 |
| | Receptor 2C | | | | | |
| | NMDA Glutamate | 0 | DLG2 | 2 | 0 | 5 |
| | Receptor 2C | | | | | |
| | NMDA Glutamate | 0 | DLG2 | 1 | 0 | 4 |
| | Receptor 2C | | | | | |
| | NMDA Glutamate | 0 | DLG1 | 2 | 0 | 5 |
| | Receptor 2C | | | | | |
| | NMDA Glutamate | 0 | DLG1 | 1 | 0 | 5 |
| | Receptor 2C | | | | | |
| | NMDA Glutamate | 0 | DLG1 | 1, 2 | 0 | 5 |
| | Receptor 2C | | | | | |
| | NMDA Glutamate | 0 | KIAA1634 | 5 | 0 | 1 |
| | Receptor 2C | | | | | |
| | NMDA Glutamate | 0 | BAI-1 | 6 | 0 | 2 |
| | Receptor 2C | | | | | |
| | NMDA Glutamate | 0 | KIAA1634 | 4 | 0 | 1 |
| | Receptor 2C | | | | | |
| | NMDA Glutamate | 0 | BAI-1 | 5 | 0 | 4 |
| | Receptor 2C | | | | | |
| | NMDA Glutamate | 0 | KIAA1634 | 2 | 0 | 3 |
| | Receptor 2C | | | | | |
| | NMDA Glutamate | 0 | KIAA1634 | 1 | 0 | 5 |
| | Receptor 2C | | | | | |
| | NMDA Glutamate | 0 | BAI-1 | 4 | 0 | 3 |
| | Receptor 2C | | | | | |
| | NMDA Glutamate | 0 | BAI-1 | 3 | 0 | 1 |
| | Receptor 2C | | | | | |
| | NMDA Glutamate | 0 | BAI-1 | 2 | 0 | 4 |
| | Receptor 2C | | | | | |
| | NMDA Glutamate | 0 | Atrophin-1 Inter. Prot. | 5 | 0 | 5 |
| | Receptor 2C | | | | | |
| | NMDA Glutamate | 0 | KIAA1526 | 1 | 0 | 3 |
| | Receptor 2C | | | | | |
| | NMDA Glutamate | 0 | atrophin-1 interacting | 3 | 0 | 2 |
| | Receptor 2C | | Protein | | | |
| | NMDA Glutamate | 0 | atrophin-1 interacting | 1 | 0 | 5 |
| | Receptor 2C | | Protein | | | |
| | NMDA Glutamate | 0 | AIPC | 1 | 0 | 3 |
| | Receptor 2C | | | | | |
| | NMDA Glutamate | 0 | KIAA0807(S) | 1 | 0 | 5 |
| | Receptor 2C | | | | | |
| AA182L | ephrin B2 | 0 | ZO-3 | 1 | 0 | 1 |
| | ephrin B2 | 0 | ZO-2 | 2 | 0 | 1 |
| | ephrin B2 | 0 | ZO-2 | 1 | 0 | 1 |
| | ephrin B2 | 0 | ZO-1 | 2 | 0 | 2 |
| | ephrin B2 | 6 | ZO-1 | 1 | 5 | 3 |
| | ephrin B2 | 0 | X11-beta | 2 | 0 | 1 |
| | ephrin B2 | 0 | X11-beta | 1 | 0 | 2 |
| | ephrin B2 | 0 | TIP1 | 1 | 0 | 2 |
| | ephrin B2 | 0 | KIAA0382 | 1 | 0 | 2 |
| | ephrin B2 | 0 | KIAA0340 | 1 | 0 | 2 |
| | ephrin B2 | 0 | KIAA0300 | 1 | 0 | 2 |
| | ephrin B2 | 0 | Syntrophin gamma-1 | 1 | 0 | 2 |
| | ephrin B2 | 5 | SITAC-18 | 2 | 5 | 3 |
| | ephrin B2 | 4 | SITAC-18 | 1 | 5 | 3 |
| | ephrin B2 | 0 | SIP1 | 2 | 0 | 2 |
| | ephrin B2 | 0 | KIAA0147 | 4 | 0 | 2 |
| | ephrin B2 | 0 | PTPL-1 | 4 | 0 | 2 |
| | ephrin B2 | 0 | PTPL-1 | 2 | 0 | 2 |
| | ephrin B2 | 0 | INADL | 3 | 0 | 2 |
| | ephrin B2 | 0 | PRIL16 | 1, 2 | 0 | 2 |
| | ephrin B2 | 0 | hSyntenin | 2 | 0 | 2 |
| | ephrin B2 | 0 | KIAA0973 | 1 | 0 | 2 |
| | ephrin B2 | 0 | hSyntenin | 1 | 0 | 1 |
| | ephrin B2 | 0 | HEMBA 1003117 | 1 | 0 | 2 |
| | ephrin B2 | 0 | MUPP-1 | 11 | 0 | 2 |
| | ephrin B2 | 0 | hAPXL | 1 | 0 | 1 |
| | ephrin B2 | 0 | Novel PDZ | 1 | 0 | 2 |
| | ephrin B2 | 0 | NeDLG | 3 | 0 | 1 |
| | ephrin B2 | 0 | NeDLG | 2 | 0 | 2 |
| | ephrin B2 | 0 | PDZK-1 | 3 | 0 | 1 |
| | ephrin B2 | 0 | GRIP1 | 6 | 5 | 3 |
| | ephrin B2 | 0 | GRIP1 | 5 | 0 | 1 |
| | ephrin B2 | 0 | GRIP1 | 3 | 0 | 1 |
| | ephrin B2 | 0 | MUPP-1 | 6 | 0 | 2 |
| | ephrin B2 | 0 | MUPP-1 | 4 | 0 | 1 |
| | ephrin B2 | 0 | MUPP-1 | 3 | 0 | 1 |
| | ephrin B2 | 0 | FLJ 10324 | 1 | 0 | 2 |
| | ephrin B2 | 0 | FLJ 00011 | 1 | 0 | 2 |
| | ephrin B2 | 0 | Mint 1 | 1, 2 | 0 | 2 |
| | ephrin B2 | 0 | EZRIN Phos B.P. | 1 | 0 | 1 |
| | ephrin B2 | 3 | Mint 1 | 2 | 5 | 3 |
| | ephrin B2 | 0 | Mint 1 | 1 | 0 | 1 |
| | ephrin B2 | 0 | LIM-Mystique | 1 | 0 | 1 |
| | ephrin B2 | 0 | LIM RIL | 1 | 0 | 2 |
| | ephrin B2 | 0 | KIAA807 | | 0 | 2 |
| | ephrin B2 | 0 | DLG5 | 2 | 0 | 1 |
| | ephrin B2 | 0 | DLG1 | 3 | 0 | 1 |
| | ephrin B2 | 0 | KIAA1719 | 5 | 5 | 4 |
| | ephrin B2 | 0 | CARD14 | 1 | 0 | 1 |
| | ephrin B2 | 0 | KIAA1719 | 1 | 0 | 1 |
| | ephrin B2 | 0 | BAI-1 | 6 | 0 | 2 |
| | ephrin B2 | 0 | KIAA1634 | 2 | 0 | 1 |
| | ephrin B2 | 0 | Atrophin-1 Inter. Prot. | 6 | 0 | 1 |
| | ephrin B2 | 0 | Atrophin-1 Inter. Prot. | 5 | 0 | 2 |
| | ephrin B2 | 5 | KIAA1526 | 1 | 5 | 3 |
| | ephrin B2 | 0 | KIAA1415 | 1 | 0 | 1 |
| | ephrin B2 | 0 | atrophin-1 interacting | 3 | 0 | 1 |
| | | | Protein | | | |
| | ephrin B2 | 0 | KIAA1284 | 1 | 0 | 1 |
| | ephrin B2 | 0 | PDZK-1 | 1 | 0 | 1 |
| | ephrin B2 | 0 | AIPC | 4 | 0 | 1 |
| | ephrin B2 | 0 | AIPC | 3 | 0 | 1 |
| | ephrin B2 | 0 | AIPC | 1 | 0 | 2 |
| | ephrin B2 | 0 | PAR3 | 3 | 0 | 2 |
| | ephrin B2 | 0 | KIAA0807(S) | 1 | 0 | 2 |
| | ephrin B2 | 0 | ZO-3 | 3 | 0 | 1 |
| | ephrin B2 | 0 | ZO-3 | 2 | 0 | 2 |
| AA183L | RhoGAP 1 (PTPL1- | 0 | PTPL-1 | 4 | 0 | 2 |
| | associated) | | | | | |
| AA185L | RGS12 (regulator of G- | 0 | ZO-2 | 1 | 0 | 1 |
| | protein signaling 12 | | | | | |
| | RGS12 (regulator of G- | 0 | ZO-1 | 1 | 0 | 1 |
| | protein signaling 12 | | | | | |
| | RGS12 (regulator of G- | 0 | TIP1 | 1 | 0 | 1 |
| | protein signaling 12 | | | | | |
| | RGS12 (regulator of G- | 0 | PTPL-1 | 4 | 0 | 1 |
| | protein signaling 12 | | | | | |
| | RGS12 (regulator of G- | 0 | PIST | 1 | 0 | 1 |
| | protein signaling 12 | | | | | |
| | RGS12 (regulator of G- | 0 | HEMBA 1003117 | 1 | 0 | 1 |
| | protein signaling 12 | | | | | |
| | RGS12 (regulator of G- | 0 | MUPP-1 | 11 | 0 | 1 |
| | protein signaling 12 | | | | | |
| | RGS12 (regulator of G- | 0 | FLJ 10324 | 1 | 0 | 1 |
| | protein signaling 12 | | | | | |
| | RGS12 (regulator of G- | 0 | DLG1 | 1, 2 | 0 | 1 |
| | protein signaling 12 | | | | | |
| | RGS12 (regulator of G- | 0 | AF6 | 1 | 0 | 1 |
| | protein signaling 12 | | | | | |
| AA190L | ephrin B1 | 0 | PTPL-1 | 4 | 0 | 2 |
| | ephrin B1 | 0 | MUPP-1 | 9 | 0 | 1 |
| | ephrin B1 | 0 | MUPP-1 | 7 | 0 | 1 |
| | ephrin B1 | 0 | MUPP-1 | 3 | 0 | 1 |
| | ephrin B1 | 0 | KIAA807 | | 0 | 1 |
| | ephrin B1 | 0 | KIAA0807(S) | 1 | 0 | 1 |
| AA192L | JAM (junctional adhesion | 0 | PTPL-1 | 4 | 0 | 1 |
| | molecule) | | | | | |
| | JAM (junctional adhesion | 0 | INADL | 3 | 0 | 1 |
| | molecule) | | | | | |
| | JAM (junctional adhesion | 0 | AF6 | 1 | 0 | 1 |
| | molecule) | | | | | |
| AA205L | serotonin receptor 5-HT- | 0 | INADL | 8 | 5 | 1 |
| | 2C | | | | | |
| | serotonin receptor 5-HT- | 0 | MUPP-1 | 10 | 5 | 1 |
| | 2C | | | | | |
| AA206L | CITRON protein | 0 | TIP1 | 1 | 0 | 5 |
| | CITRON protein | 0 | KIAA0380 | 1 | 0 | 1 |
| | CITRON protein | 0 | Synt. 1 alpha | 1 | 0 | 1 |
| | CITRON protein | 0 | INADL | 8 | 0 | 1 |
| | CITRON protein | 0 | KIAA0973 | 1 | 0.5 | 5 |
| | CITRON protein | 0 | MUPP-1 | 10 | 0 | 1 |
| | CITRON protein | 0 | Outer Membrane | 1 | 5 | 4 |
| | CITRON protein | 0 | NeDLG | 3 | 5 | 3 |
| | CITRON protein | 7 | Erbin | 1 | 5 | 4 |
| | CITRON protein | 0 | KIAA807 | | 0 | 4 |
| | CITRON protein | 0 | DLG1 | 2 | 0 | 2 |
| | CITRON protein | 0 | BAI-1 | 5 | 0 | 2 |
| | CITRON protein | 8 | KIAA1634 | 4 | 5 | 3 |
| | CITRON protein | 0 | KIAA1526 | 1 | 0 | 1 |
| | CITRON protein | 1 | KIAA0807(S) | 1 | 0.1 | 4 |
| | CITRON protein | 0 | ZO-3 | 3 | 0 | 1 |
| AA207L | Nedasin (s-form) | 0 | TIP1 | 1 | 0 | 5 |
| | Nedasin (s-form) | 0 | KIAA0380 | 1 | 0 | 1 |
| | Nedasin (s-form) | 0 | INADL | 8 | 0 | 1 |
| | Nedasin (s-form) | 0 | PSD95 | 1, 2, 3 | 0 | 3 |
| | Nedasin (s-form) | 0 | NeDLG | 1, 2 | 0 | 2 |
| | Nedasin (s-form) | 0 | Mint 1 | 1, 2 | 0 | 1 |
| | Nedasin (s-form) | 0 | KIAA807 | | 0 | 2 |
| | Nedasin (s-form) | 0 | DLG1 | 1, 2 | 0 | 3 |
| | Nedasin (s-form) | 0 | BAI-1 | 6 | 0 | 1 |
| | Nedasin (s-form) | 0 | KIAA1634 | 1 | 0 | 1 |
| | Nedasin (s-form) | 0 | BAI-1 | 2 | 0 | 1 |
| AA210L | APC—adenomatous | 0 | TIP1 | 1 | 0 | 3 |
| | polyposis coli protein | | | | | |
| | APC—adenomatous | 0 | KIAA0382 | 1 | 0 | 1 |
| | polyposis coli protein | | | | | |
| | APC—adenomatous | 0 | KIAA0147 | 1 | 0 | 1 |
| | polyposis coli protein | | | | | |
| | APC—adenomatous | 0 | INADL | 8 | 0 | 2 |
| | polyposis coli protein | | | | | |
| | APC—adenomatous | 0 | PSD95 | 1, 2, 3 | 0 | 5 |
| | polyposis coli protein | | | | | |
| | APC—adenomatous | 0 | MUPP-1 | 10 | 0 | 1 |
| | polyposis coli protein | | | | | |
| | APC—adenomatous | 0 | NeDLG | 1, 2 | 0 | 4 |
| | polyposis coli protein | | | | | |
| | APC—adenomatous | 0 | Outer Membrane | 1 | 0 | 2 |
| | polyposis coli protein | | | | | |
| | APC—adenomatous | 0 | FLJ 00011 | 1 | 0 | 1 |
| | polyposis coli protein | | | | | |
| | APC—adenomatous | 0 | KIAA807 | | 0 | 1 |
| | polyposis coli protein | | | | | |
| | APC—adenomatous | 0 | DLG1 | 1, 2 | 0 | 5 |
| | polyposis coli protein | | | | | |
| | APC—adenomatous | 0 | BAI-1 | 5 | 0 | 1 |
| | polyposis coli protein | | | | | |
| | APC—adenomatous | 0 | KIAA1634 | 2 | 0 | 1 |
| | polyposis coli protein | | | | | |
| | APC—adenomatous | 0 | KIAA1634 | 1 | 0 | 1 |
| | polyposis coli protein | | | | | |
| | APC—adenomatous | 0 | BAI-1 | 2 | 0 | 1 |
| | polyposis coli protein | | | | | |
| | APC—adenomatous | 0 | KIAA0807(S) | 1 | 0 | 1 |
| | polyposis coli protein | | | | | |
| AA214L | ErbB-4 receptor | 0 | PTPL-1 | 2 | 0 | 2 |
| | ErbB-4 receptor | 0 | PSD95 | 1, 2, 3 | 0 | 1 |
| | ErbB-4 receptor | 0 | NeDLG | 1, 2 | 0 | 1 |
| | ErbB-4 receptor | 0 | FLJ 10324 | 1 | 0 | 1 |
| | ErbB-4 receptor | 0 | DLG1 | 1, 2 | 0 | 1 |
| | ErbB-4 receptor | 0 | KIAA1634 | 2 | 0 | 1 |
| | ErbB-4 receptor | 0 | BAI-1 | 3 | 0 | 1 |
| AA215 | CKR5_HUMAN | 0 | TIP1 | 1 | 0 | 1 |
| | CKR5_HUMAN | 0 | TAX IP2 | 1 | 0 | 1 |
| | CKR5_HUMAN | 0 | Mint 1 | 1, 2 | 0 | 1 |
| | CKR5_HUMAN | 0 | KIAA1719 | 2 | 0 | 1 |
| | CKR5_HUMAN | 0 | KIAA1719 | 5 | 0 | 1 |
| | CKR5_HUMAN | 0 | KIAA1634 | 1 | 0 | 1 |
| AA216 | NMDA R2C | 0 | PTPL-1 | 2 | 0 | 1 |
| | NMDA R2C | 0 | KIAA1634 | 2 | 0 | 1 |
| AA217 | catenin - delta 2 | 0 | TIP1 | 1 | 0 | 3 |
| | catenin - delta 2 | 0 | Syntrophin gamma-1 | 1 | 0 | 1 |
| | catenin - delta 2 | 0 | KIAA0147 | 4 | 0 | 1 |
| | catenin - delta 2 | 0 | KIAA0147 | 2 | 0 | 3 |
| | catenin - delta 2 | 0 | INADL | 8 | 0 | 2 |
| | catenin - delta 2 | 0 | PTPL-1 | 4 | 0 | 1 |
| | catenin - delta 2 | 0 | PTPL-1 | 2 | 0 | 5 |
| | catenin - delta 2 | 0 | INADL | 5 | 0 | 1 |
| | catenin - delta 2 | 0 | PSD95 | 1, 2, 3 | 0 | 2 |
| | catenin - delta 2 | 0 | PSD95 | 1 | 0 | 1 |
| | catenin - delta 2 | 0 | HEMBA 1003117 | 1 | 0 | 1 |
| | catenin - delta 2 | 0 | Outer Membrane | 1 | 0 | 5 |
| | catenin - delta 2 | 0 | NeDLG | 3 | 0 | 1 |
| | catenin - delta 2 | 0 | FLJ 10324 | 1 | 0 | 3 |
| | catenin - delta 2 | 0 | Mint 1 | 1, 2 | 0 | 5 |
| | catenin - delta 2 | 0 | Mint 1 | 2 | 0 | 3 |
| | catenin - delta 2 | 0 | Erbin | 1 | 0 | 4 |
| | catenin - delta 2 | 0 | LIM-Mystique | 1 | 0 | 5 |
| | catenin - delta 2 | 0 | LIM RIL | 1 | 0 | 2 |
| | catenin - delta 2 | 0 | KIAA807 | | 0 | 4 |
| | catenin - delta 2 | 0 | DLG2 | 2 | 0 | 1 |
| | catenin - delta 2 | 0 | DLG1 | 2 | 0 | 2 |
| | catenin - delta 2 | 0 | DLG1 | 1 | 0 | 1 |
| | catenin - delta 2 | 0 | DLG1 | 1, 2 | 5 | 3 |
| | catenin - delta 2 | 0 | KIAA1634 | 5 | 0 | 3 |
| | catenin - delta 2 | 0 | BAI-1 | 3 | 0 | 1 |
| | catenin - delta 2 | 0 | Atrophin-1 Inter. Prot. | 5 | 0 | 5 |
| | catenin - delta 2 | 0 | KIAA1526 | 1 | 0 | 2 |
| | catenin - delta 2 | 0 | atrophin-1 interacting | 3 | 0 | 1 |
| | | | Protein | | | |
| | catenin - delta 2 | 0 | AIPC | 1 | 0 | 2 |
| | catenin - delta 2 | 0 | PAR3 | 3 | 0 | 1 |
| | catenin - delta 2 | 0 | KIAA0807(S) | 1 | 5 | 3 |
| | catenin - delta 2 | 0 | ZO-3 | 3 | 5 | 3 |
| AA218 | CSPG4 (chondroitin sulfae | 0 | GRIP1 | 7 | 0 | 5 |
| | proteoglycan 4, | | | | | |
| | melanoma-associated) | | | | | |
| | CSPG4 (chondroitin sulfae | 0 | ZO-3 | 1 | 0 | 2 |
| | proteoglycan 4, | | | | | |
| | melanoma-associated) | | | | | |
| | CSPG4 (chondroitin sulfae | 0 | ZO-2 | 2 | 0 | 1 |
| | proteoglycan 4, | | | | | |
| | melanoma-associated) | | | | | |
| | CSPG4 (chondroitin sulfae | 0 | ZO-2 | 1 | 0 | 5 |
| | proteoglycan 4, | | | | | |
| | melanoma-associated) | | | | | |
| | CSPG4 (chondroitin sulfae | 0 | ZO-1 | 2 | 0 | 4 |
| | proteoglycan 4, | | | | | |
| | melanoma-associated) | | | | | |
| | CSPG4 (chondroitin sulfae | 0 | ZO-1 | 1 | 0 | 5 |
| | proteoglycan 4, | | | | | |
| | melanoma-associated) | | | | | |
| | CSPG4 (chondroitin sulfae | 0 | X11-beta | 2 | 0 | 2 |
| | proteoglycan 4, | | | | | |
| | melanoma-associated) | | | | | |
| | CSPG4 (chondroitin sulfae | 0 | TIP1 | 1 | 0 | 1 |
| | proteoglycan 4, | | | | | |
| | melanoma-associated) | | | | | |
| | CSPG4 (chondroitin sulfae | 0 | TIAM-2 | 1 | 0 | 3 |
| | proteoglycan 4, | | | | | |
| | melanoma-associated) | | | | | |
| | CSPG4 (chondroitin sulfae | 0 | KIAA0303 | 1 | 0 | 1 |
| | proteoglycan 4, | | | | | |
| | melanoma-associated) | | | | | |
| | CSPG4 (chondroitin sulfae | 0 | KIAA0300 | 1 | 0 | 2 |
| | proteoglycan 4, | | | | | |
| | melanoma-associated) | | | | | |
| | CSPG4 (chondroitin sulfae | 0 | INADL | 8 | 0 | 3 |
| | proteoglycan 4, | | | | | |
| | melanoma-associated) | | | | | |
| | CSPG4 (chondroitin sulfae | 0 | PTPL-1 | 4 | 0 | 5 |
| | proteoglycan 4, | | | | | |
| | melanoma-associated) | | | | | |
| | CSPG4 (chondroitin sulfae | 0 | INADL | 5 | 0 | 5 |
| | proteoglycan 4, | | | | | |
| | melanoma-associated) | | | | | |
| | CSPG4 (chondroitin sulfae | 0 | INADL | 3 | 0 | 3 |
| | proteoglycan 4, | | | | | |
| | melanoma-associated) | | | | | |
| | CSPG4 (chondroitin sulfae | 0 | hSyntenin | 1 | 0 | 2 |
| | proteoglycan 4, | | | | | |
| | melanoma-associated) | | | | | |
| | CSPG4 (chondroitin sulfae | 0 | HEMBA 1003117 | 1 | 0 | 5 |
| | proteoglycan 4, | | | | | |
| | melanoma-associated) | | | | | |
| | CSPG4 (chondroitin sulfae | 0 | MUPP-1 | 10 | 0 | 4 |
| | proteoglycan 4, | | | | | |
| | melanoma-associated) | | | | | |
| | CSPG4 (chondroitin sulfae | 0 | MUPP-1 | 11 | 0 | 5 |
| | proteoglycan 4, | | | | | |
| | melanoma-associated) | | | | | |
| | CSPG4 (chondroitin sulfae | 0 | hAPXL | 1 | 0 | 3 |
| | proteoglycan 4, | | | | | |
| | melanoma-associated) | | | | | |
| | CSPG4 (chondroitin sulfae | 0 | Outer Membrane | 1 | 0 | 1 |
| | proteoglycan 4, | | | | | |
| | melanoma-associated) | | | | | |
| | CSPG4 (chondroitin sulfae | 0 | NOS1 | 1 | 0 | 2 |
| | proteoglycan 4, | | | | | |
| | melanoma-associated) | | | | | |
| | CSPG4 (chondroitin sulfae | 0 | GRIP1 | 5 | 0 | 1 |
| | proteoglycan 4, | | | | | |
| | melanoma-associated) | | | | | |
| | CSPG4 (chondroitin sulfae | 0 | MUPP-1 | 8 | 0 | 2 |
| | proteoglycan 4, | | | | | |
| | melanoma-associated) | | | | | |
| | CSPG4 (chondroitin sulfae | 0 | MUPP-1 | 5 | 0 | 5 |
| | proteoglycan 4, | | | | | |
| | melanoma-associated) | | | | | |
| | CSPG4 (chondroitin sulfae | 0 | FLJ 10324 | 1 | 0 | 5 |
| | proteoglycan 4, | | | | | |
| | melanoma-associated) | | | | | |
| | CSPG4 (chondroitin sulfae | 0 | MUPP-1 | 2 | 0 | 5 |
| | proteoglycan 4, | | | | | |
| | melanoma-associated) | | | | | |
| | CSPG4 (chondroitin sulfae | 0 | MUPP-1 | 1 | 0 | 2 |
| | proteoglycan 4, | | | | | |
| | melanoma-associated) | | | | | |
| | CSPG4 (chondroitin sulfae | 0 | MUPP-1 | 12 | 0 | 1 |
| | proteoglycan 4, | | | | | |
| | melanoma-associated) | | | | | |
| | CSPG4 (chondroitin sulfae | 0 | Mint 1 | 1, 2 | 0 | 5 |
| | proteoglycan 4, | | | | | |
| | melanoma-associated) | | | | | |
| | CSPG4 (chondroitin sulfae | 0 | Mint 1 | 2 | 0 | 5 |
| | proteoglycan 4, | | | | | |
| | melanoma-associated) | | | | | |
| | CSPG4 (chondroitin sulfae | 0 | Mint 1 | 1 | 0 | 2 |
| | proteoglycan 4, | | | | | |
| | melanoma-associated) | | | | | |
| | CSPG4 (chondroitin sulfae | 0 | LIM-Mystique | 1 | 0 | 2 |
| | proteoglycan 4, | | | | | |
| | melanoma-associated) | | | | | |
| | CSPG4 (chondroitin sulfae | 0 | Erbin | 1 | 0 | 3 |
| | proteoglycan 4, | | | | | |
| | melanoma-associated) | | | | | |
| | CSPG4 (chondroitin sulfae | 0 | LIM RIL | 1 | 0 | 2 |
| | proteoglycan 4, | | | | | |
| | melanoma-associated) | | | | | |
| | CSPG4 (chondroitin sulfae | 0 | KIAA807 | | 0 | 1 |
| | proteoglycan 4, | | | | | |
| | melanoma-associated) | | | | | |
| | CSPG4 (chondroitin sulfae | 0 | DVL2 | 1 | 0 | 5 |
| | proteoglycan 4, | | | | | |
| | melanoma-associated) | | | | | |
| | CSPG4 (chondroitin sulfae | 0 | KIAA1719 | 6 | 0 | 5 |
| | proteoglycan 4, | | | | | |
| | melanoma-associated) | | | | | |
| | CSPG4 (chondroitin sulfae | 0 | KIAA1634 | 5 | 0 | 2 |
| | proteoglycan 4, | | | | | |
| | melanoma-associated) | | | | | |
| | CSPG4 (chondroitin sulfae | 0 | BAI-1 | 6 | 0 | 4 |
| | proteoglycan 4, | | | | | |
| | melanoma-associated) | | | | | |
| | CSPG4 (chondroitin sulfae | 0 | KIAA1634 | 1 | 0 | 5 |
| | proteoglycan 4, | | | | | |
| | melanoma-associated) | | | | | |
| | CSPG4 (chondroitin sulfae | 0 | BAI-1 | 2 | 0 | 2 |
| | proteoglycan 4, | | | | | |
| | melanoma-associated) | | | | | |
| | CSPG4 (chondroitin sulfae | 0 | Atrophin-1 Inter. Prot. | 5 | 0 | 2 |
| | proteoglycan 4, | | | | | |
| | melanoma-associated) | | | | | |
| | CSPG4 (chondroitin sulfae | 0 | atrophin-1 interacting | 3 | 0 | 2 |
| | proteoglycan 4, | | Protein | | | |
| | melanoma-associated) | | | | | |
| | CSPG4 (chondroitin sulfae | 0 | atrophin-1 interacting | 1 | 0 | 1 |
| | proteoglycan 4, | | Protein | | | |
| | melanoma-associated) | | | | | |
| | CSPG4 (chondroitin sulfae | 0 | AIPC | 1 | 0 | 5 |
| | proteoglycan 4, | | | | | |
| | melanoma-associated) | | | | | |
| | CSPG4 (chondroitin sulfae | 0 | AF6 | 1 | 0 | 5 |
| | proteoglycan 4, | | | | | |
| | melanoma-associated) | | | | | |
| | CSPG4 (chondroitin sulfae | 0 | PAR3 | 3 | 0 | 3 |
| | proteoglycan 4, | | | | | |
| | melanoma-associated) | | | | | |
| | CSPG4 (chondroitin sulfae | 0 | KIAA0807(S) | 1 | 0 | 1 |
| | proteoglycan 4, | | | | | |
| | melanoma-associated) | | | | | |
| | CSPG4 (chondroitin sulfae | 0 | ZO-3 | 3 | 0 | 5 |
| | proteoglycan 4, | | | | | |
| | melanoma-associated) | | | | | |
| AA22 | DNAM-1 | 3 | ZO-2 | 1 | 1 | 3 |
| | DNAM-1 | 5 | ZO-1 | 1 | 1 | 2 |
| | DNAM-1 | 0 | TIP1 | 1 | 0 | 1 |
| | DNAM-1 | 5 | SHANK 1 | 1 | 1 | 5 |
| | DNAM-1 | 0 | SHANK 3 | 1 | 0 | 2 |
| | DNAM-1 | 0 | EBP50 | 1 | 0 | 1 |
| | DNAM-1 | 0 | EBP50 | 2 | 0 | 1 |
| | DNAM-1 | 0 | INADL | 8 | 0 | 5 |
| | DNAM-1 | 2.5 | PIST | 1 | 0.5 | 4 |
| | DNAM-1 | 2.5 | MUPP-1 | 10 | 1 | 4 |
| | DNAM-1 | 0 | Outer Membrane | 1 | 0 | 1 |
| | DNAM-1 | 0 | NOS1 | 1 | 0 | 1 |
| | DNAM-1 | 2 | KIAA807 | | 5 | 3 |
| | DNAM-1 | 1 | KIAA1634 | 1 | 0.3 | 5 |
| | DNAM-1 | 4 | BAI-1 | 2 | 0.1 | 5 |
| | DNAM-1 | 3 | atrophin-1 interacting | 1 | 1 | 3 |
| | | | Protein | | | |
| | DNAM-1 | 2 | KIAA0807(S) | 1 | 5 | 3 |
| AA220 | claudin 10 | 0 | DLG1 | 1, 2 | 0 | 1 |
| | claudin 10 | 0 | KIAA1634 | 1 | 0 | 1 |
| AA222 | claudin 18 | 0 | Mint 1 | 1, 2 | 0 | 1 |
| AA223 | claudin 1 | 0 | INADL | 8 | 0 | 1 |
| | claudin 1 | 0 | Mint 1 | 2 | 0 | 1 |
| AA225 | claudin 9 | 0 | Mint 1 | 1, 2 | 0 | 1 |
| AA226 | claudin 7 | 0 | Mint 1 | 1, 2 | 5 | 4 |
| AA227 | claudin 2 | 0 | Mint 1 | 1, 2 | 0 | 2 |
| | claudin 2 | 0 | KIAA807 | | 0 | 1 |
| | claudin 2 | 0 | BAI-1 | 3 | 0 | 1 |
| | claudin 2 | 0 | KIAA1634 | 1 | 0 | 1 |
| AA228 | Nectin 2 | 0 | Mint 1 | 1, 2 | 0 | 2 |
| | Nectin 2 | 0 | KIAA1634 | 1 | 0 | 1 |
| | Nectin 2 | 0 | AF6 | 1 | 0 | 2 |
| AA23.3 | Fas Ligand | 0 | Mint 1 | 1, 2 | 0 | 4 |
| | Fas Ligand | 0 | KIAA807 | | 0 | 5 |
| | Fas Ligand | 0 | KIAA0973 | 1 | 0 | 2 |
| | Fas Ligand | 0 | KIAA0807(S) | 1 | 0 | 5 |
| | Fas Ligand | 0 | KIAA0380 | 1 | 0 | 3 |
| | Fas Ligand | 0 | hAPXL | 1 | 0 | 2 |
| | Fas Ligand | 0 | AIPC | 1 | 0 | 2 |
| AA233L | 5H2B_HUMAN | 0 | KIAA0316 | 1 | 0 | 1 |
| | 5H2B_HUMAN | 0 | PTPL-1 | 4 | 0 | 2 |
| | 5H2B_HUMAN | 0.2 | PTPL-1 | 2 | 0.5 | 5 |
| | 5H2B_HUMAN | 0 | PIST | 1 | 0 | 1 |
| | 5H2B_HUMAN | 0 | HEMBA 1003117 | 1 | 0 | 1 |
| | 5H2B_HUMAN | 0 | FLJ 10324 | 1 | 0 | 2 |
| | 5H2B_HUMAN | 0 | Mint 1 | 1, 2 | 5 | 1 |
| | 5H2B_HUMAN | 0 | Mint 1 | 2 | 5 | 1 |
| | 5H2B_HUMAN | 0 | KIAA807 | | 5 | 1 |
| | 5H2B_HUMAN | 0 | KIAA1634 | 2 | 0 | 5 |
| | 5H2B_HUMAN | 2 | BAI-1 | 3 | 0.5 | 4 |
| | 5H2B_HUMAN | 0 | KIAA0807(S) | 1 | 5 | 1 |
| AA240 | Dopamine transporter | 0 | ZO-1 | 2 | 0 | 1 |
| | (Na+-dependent) | | | | | |
| | Dopamine transporter | 0.4 | PTPL-1 | 4 | 5 | 3 |
| | (Na+-dependent) | | | | | |
| | Dopamine transporter | 0.3 | HEMBA 1003117 | 1 | 5 | 5 |
| | (Na+-dependent) | | | | | |
| | Dopamine transporter | 0.9 | PICK1 | 1 | 5 | 2 |
| | (Na+-dependent) | | | | | |
| | Dopamine transporter | 0.3 | FLJ 10324 | 1 | 1 | 5 |
| | (Na+-dependent) | | | | | |
| | Dopamine transporter | 0.4 | KIAA807 | | 5 | 4 |
| | (Na+-dependent) | | | | | |
| | Dopamine transporter | 0.9 | KIAA1634 | 1 | 5 | 3 |
| | (Na+-dependent) | | | | | |
| | Dopamine transporter | 0.4 | KIAA0807(S) | 1 | 5 | 4 |
| | (Na+-dependent) | | | | | |
| AA243 | A2AA_HUMAN (modified) | 0 | ZO-3 | 2 | 0 | 3 |
| | A2AA_HUMAN (modified) | 0 | ZO-2 | 2 | 0 | 2 |
| | A2AA_HUMAN (modified) | 0 | ZO-1 | 2 | 0 | 4 |
| | A2AA_HUMAN (modified) | 0 | X11-beta | 2 | 0 | 1 |
| | A2AA_HUMAN (modified) | 0 | X11-beta | 1 | 0 | 2 |
| | A2AA_HUMAN (modified) | 0 | Unnamed Protein | 2 | 0 | 1 |
| | A2AA_HUMAN (modified) | 0 | Syntrophin gamma-1 | 1 | 0 | 2 |
| | A2AA_HUMAN (modified) | 0 | SITAC-18 | 2 | 0 | 4 |
| | A2AA_HUMAN (modified) | 0 | SITAC-18 | 1 | 0 | 4 |
| | A2AA_HUMAN (modified) | 0 | PTPL-1 | 2 | 0 | 2 |
| | A2AA_HUMAN (modified) | 0 | PAR3 | 3 | 0 | 2 |
| | A2AA_HUMAN (modified) | 0 | MUPP-1 | 13 | 0 | 1 |
| | A2AA_HUMAN (modified) | 0 | MUPP-1 | 8 | 0 | 1 |
| | A2AA_HUMAN (modified) | 0 | MUPP-1 | 6 | 0 | 2 |
| | A2AA_HUMAN (modified) | 0 | Mint 1 | 1 | 0 | 1 |
| | A2AA_HUMAN (modified) | 0 | LIM-Mystique | 1 | 0 | 1 |
| | A2AA_HUMAN (modified) | 0 | KIAA1719 | 4 | 0 | 3 |
| | A2AA_HUMAN (modified) | 0 | KIAA1526 | 1 | 0 | 4 |
| | A2AA_HUMAN (modified) | 0 | KIAA1284 | 1 | 0 | 1 |
| | A2AA_HUMAN (modified) | 0 | KIAA0807(S) | 1 | 0 | 1 |
| | A2AA_HUMAN (modified) | 0 | KIAA0751(L) | 1 | 0 | 3 |
| | A2AA_HUMAN (modified) | 0 | KIAA0340 | 1 | 0 | 1 |
| | A2AA_HUMAN (modified) | 0 | INADL | 4 | 0 | 1 |
| | A2AA_HUMAN (modified) | 0 | INADL | 3 | 0 | 2 |
| | A2AA_HUMAN (modified) | 0 | HEMBA 1003117 | 1 | 0 | 1 |
| | A2AA_HUMAN (modified) | 0 | hAPXL | 1 | 0 | 1 |
| | A2AA_HUMAN (modified) | 0 | FLJ21687 | 1 | 0 | 1 |
| | A2AA_HUMAN (modified) | 0 | FLJ 10324 | 1 | 0 | 1 |
| | A2AA_HUMAN (modified) | 0 | DLG5 | 2 | 0 | 1 |
| | A2AA_HUMAN (modified) | 0 | CARD14 | 1 | 0 | 1 |
| | A2AA_HUMAN (modified) | 0 | BAI-1 | 6 | 0 | 3 |
| | A2AA_HUMAN (modified) | 0 | Atrophin-1 Inter. Prot. | 6 | 0 | 1 |
| | A2AA_HUMAN (modified) | 0 | Atrophin-1 Inter. Prot. | 5 | 0 | 1 |
| | A2AA_HUMAN (modified) | 0 | AIPC | 1 | 0 | 2 |
| AA244 | A2AB_HUMAN (modified) | 0 | TIP1 | 1 | 0 | 5 |
| | A2AB_HUMAN (modified) | 0 | PSD95 | 1, 2, 3 | 0 | 5 |
| | A2AB_HUMAN (modified) | 0 | KIAA807 | | 0 | 4 |
| | A2AB_HUMAN (modified) | 0 | KIAA0303 | 1 | 0 | 4 |
| | A2AB_HUMAN (modified) | 0 | BAI-1 | 4 | 0 | 5 |
| | A2AB_HUMAN (modified) | 0 | BAI-1 | 2 | 0 | 4 |
| AA245 | A2AC_HUMAN (Modified) | 0 | PTPL-1 | 5 | 0 | 3 |
| | A2AC_HUMAN (Modified) | 0 | MUPP-1 | 4 | 0 | 3 |
| | A2AC_HUMAN (Modified) | 0 | Mint 1 | 2 | 0 | 3 |
| | A2AC_HUMAN (Modified) | 0 | LU1 | 1 | 0 | 4 |
| | A2AC_HUMAN (Modified) | 0 | KIAA1719 | 3 | 0 | 5 |
| | A2AC_HUMAN (Modified) | 0 | KIAA0973 | 1 | 0 | 3 |
| | A2AC_HUMAN (Modified) | 0 | hAPXL | 1 | 0 | 3 |
| | A2AC_HUMAN (Modified) | 0 | DVL2 | 1 | 0 | 3 |
| | A2AC_HUMAN (Modified) | 0 | CARD14 | 1 | 0 | 5 |
| | A2AC_HUMAN (Modified) | 0 | GRIP1 | 5 | 0 | 1 |
| AA248 | SSR4_HUMAN | 0 | PDZK1 | 2, 3, 4 | 0 | 1 |
| | SSR4_HUMAN | 0 | Mint 1 | 1, 2 | 0 | 1 |
| | SSR4_HUMAN | 0 | KIAA807 | | 0 | 1 |
| | SSR4_HUMAN | 0 | DLG1 | 1, 2 | 0 | 1 |
| | SSR4_HUMAN | 0 | BAI-1 | 5 | 0 | 1 |
| | SSR4_HUMAN | 0 | BAI-1 | 4 | 0 | 1 |
| AA25 | FceRlb | 0 | AF6 | 1 | 0 | 2 |
| | FceRlb | 0 | hAPXL | 1 | 0 | 1 |
| | FceRlb | 0 | ENIGMA | 1 | 0 | 2 |
| | FceRlb | 0 | LIM RIL | 1 | 0 | 1 |
| | FceRlb | 0 | LIM Protein | 1 | 0 | 2 |
| AA250 | 5-HT 3A (serotonin | 0 | HEMBA 1003117 | 1 | 0 | 2 |
| | receptor 3A) | | | | | |
| | 5-HT 3A (serotonin | 0 | MPP2 | 1 | 0 | 2 |
| | receptor 3A) | | | | | |
| | 5-HT 3A (serotonin | 0 | CARD14 | 1 | 0 | 2 |
| | receptor 3A) | | | | | |
| AA252 | ACM3_HUMAN | 0 | KIAA807 | | 0 | 1 |
| | ACM3_HUMAN | 0 | KIAA0807(S) | 1 | 0 | 1 |
| | ACM3_HUMAN | 0 | hAPXL | 1 | 0 | 1 |
| | ACM3_HUMAN | 0 | AIPC | 1 | 0 | 1 |
| AA255 | Clasp-5 | 0 | SHANK | 1 | 0 | 1 |
| | Clasp-5 | 0 | KIAA807 | | 0 | 1 |
| | Clasp-5 | 0 | KIAA0807(S) | 1 | 0 | 1 |
| | Clasp-5 | 0 | BAI-1 | 2 | 0 | 1 |
| AA258 | Noradrenaline transporter | 0.4 | ZO-1 | 2 | 5 | 2 |
| | Noradrenaline transporter | 1 | PICK1 | 1 | 5 | 1 |
| | Noradrenaline transporter | 0.6 | PAR3 | 3 | 1 | 4 |
| | Noradrenaline transporter | 0.7 | MUPP-1 | 9 | 5 | 3 |
| | Noradrenaline transporter | 0.8 | MUPP-1 | 7 | 5 | 3 |
| | Noradrenaline transporter | 0.4 | MUPP-1 | 3 | 5 | 4 |
| | Noradrenaline transporter | 0.8 | KIAA1719 | 6 | 5 | 2 |
| | Noradrenaline transporter | 0 | KIAA0380 | 1 | 5 | 1 |
| | Noradrenaline transporter | 0.5 | Mint 1 | 1, 2 | 5 | 3 |
| | Noradrenaline transporter | 1 | KIAA1719 | 5 | 5 | 2 |
| | Noradrenaline transporter | 0.6 | INADL | 3 | 5 | 3 |
| | Noradrenaline transporter | 0.6 | FLJ 10324 | 1 | 5 | 3 |
| | Noradrenaline transporter | 0.6 | AIPC | 1 | 5 | 2 |
| | Noradrenaline transporter | 0.5 | GRIP1 | 6 | 5 | 2 |
| AA261 | GABA transporter 3 | 0 | KIAA0807(S) | 1 | 0 | 1 |
| | GABA transporter 3 | 0 | hAPXL | 1 | 0 | 1 |
| | GABA transporter 3 | 0 | Synt. 1 alpha | 1 | 0 | 1 |
| | GABA transporter 3 | 0 | SHANK | 1 | 5 | 1 |
| | GABA transporter 3 | 0 | PDZK1 | 2, 3, 4 | 0 | 1 |
| | GABA transporter 3 | 0 | KIAA807 | | 0 | 1 |
| AA262 | Glutamate transporter 3 | 0 | X11-beta | 2 | 0 | 1 |
| | Glutamate transporter 3 | 0 | PTPL-1 | 4 | 5 | 1 |
| | Glutamate transporter 3 | 0 | MUPP-1 | 10 | 0 | 1 |
| | Glutamate transporter 3 | 0 | Mint 1 | 1, 2 | 5 | 1 |
| | Glutamate transporter 3 | 0 | Mint 1 | 2 | 0 | 1 |
| | Glutamate transporter 3 | 0 | KIAA807 | | 0 | 1 |
| | Glutamate transporter 3 | 0 | KIAA0807(S) | 1 | 5 | 1 |
| | Glutamate transporter 3 | 0 | hAPXL | 1 | 0 | 1 |
| | Glutamate transporter 3 | 0 | BAI-1 | 4 | 5 | 1 |
| AA264 | Bone Morphogenetic | 0 | MUPP-1 | 9 | 0 | 1 |
| | Protein Receptor | | | | | |
| | Bone Morphogenetic | 0 | MUPP-1 | 7 | 0 | 1 |
| | Protein Receptor | | | | | |
| | Bone Morphogenetic | 0 | MUPP-1 | 3 | 0 | 1 |
| | Protein Receptor | | | | | |
| | Bone Morphogenetic | 0 | KIAA0807(S) | 1 | 0 | 1 |
| | Protein Receptor | | | | | |
| AA268 | PTR2_HUMAN | 0 | PAR3 | 3 | 0 | 1 |
| | PTR2_HUMAN | 0 | hAPXL | 1 | 0 | 1 |
| AA269 | C5AR_HUMAN | 0 | PTPL-1 | 4 | 0 | 1 |
| AA28.1 | CDW125 (modified) | 0 | hAPXL | 1 | 0 | 1 |
| | CDW125 (modified) | 0 | ENIGMA | 1 | 0 | 1 |
| AA29.2 | CDw128B | 0 | KIAA0382 | 1 | 0 | 2 |
| | CDw128B | 0 | SHANK | 1 | 5 | 3 |
| | CDw128B | 0 | KIAA807 | | 5 | 5 |
| | CDw128B | 0 | KIAA0807(S) | 1 | 0 | 5 |
| AA29.3 | IL-8RB | 0 | TIP1 | 1 | 0 | 1 |
| | IL-8RB | 0 | Synt. 1 alpha | 1 | 0 | 1 |
| | IL-8RB | 0 | PDZK1 | 2, 3, 4 | 0 | 1 |
| | IL-8RB | 0 | Novel PDZ | 2 | 0 | 1 |
| | IL-8RB | 0 | MUPP-1 | 13 | 0 | 1 |
| | IL-8RB | 0 | KIAA1634 | 5 | 0 | 1 |
| | IL-8RB | 0 | KIAA1634 | 1 | 0 | 1 |
| | IL-8RB | 0 | KIAA0380 | 1 | 0 | 1 |
| | IL-8RB | 0 | BAI-1 | 6 | 0 | 1 |
| | IL-8RB | 0 | BAI-1 | 2 | 0 | 1 |
| AA30 | LPAP | 0 | Unnamed Protein | 2 | 0 | 3 |
| | LPAP | 0 | KIAA0382 | 1 | 0 | 5 |
| | LPAP | 0 | KIAA0316 | 1 | 0 | 1 |
| | LPAP | 0 | SHANK | 1 | 0 | 3 |
| | LPAP | 0 | SHANK3 | 1 | 0 | 3 |
| | LPAP | 0 | EBP50 | 1 | 0 | 5 |
| | LPAP | 0 | EBP50 | 2 | 0 | 4 |
| | LPAP | 0 | KIAA0147 | 1 | 0 | 3 |
| | LPAP | 0 | PTPL-1 | 2 | 0 | 1 |
| | LPAP | 0 | PIST | 1 | 0 | 1 |
| | LPAP | 0 | HEMBA 1003117 | 1 | 0 | 1 |
| | LPAP | 0 | hAPXL | 1 | 0 | 1 |
| | LPAP | 0 | NOS1 | 1 | 0 | 1 |
| | LPAP | 0 | PDZK1 | 2, 3, 4 | 0 | 3 |
| | LPAP | 0 | GRIP1 | 3 | 0 | 1 |
| | LPAP | 0 | FLJ 10324 | 1 | 0 | 1 |
| | LPAP | 1.5 | FLJ 00011 | 1 | 5 | 4 |
| | LPAP | 0 | Mint 1 | 2 | 0 | 1 |
| | LPAP | 0 | KIAA807 | | 0 | 5 |
| | LPAP | 0 | BAI-1 | 2 | 0 | 2 |
| | LPAP | 0 | Atrophin-1 Inter. Prot. | 5 | 0 | 2 |
| | LPAP | 0 | KIAA1526 | 1 | 0 | 1 |
| AA300 | Traf2 | 0 | KIAA807 | | 0 | 2 |
| | Traf2 | 0 | KIAA0973 | 1 | 0 | 1 |
| | Traf2 | 0 | KIAA0807(S) | 1 | 0 | 4 |
| AA31 | Mannose receptor | 0 | hAPXL | 1 | 0 | 1 |
| | Mannose receptor | 0 | FLJ 00011 | 1 | 0 | 1 |
| | Mannose receptor | 0 | KIAA807 | | 0 | 1 |
| | Mannose receptor | 0 | KIAA0807(S) | 1 | 5 | 1 |
| AA36 | Neuroligin | 0 | ZO-1 | 1 | 0 | 1 |
| | Neuroligin | 0 | TIP1 | 1 | 0 | 1 |
| | Neuroligin | 0.3 | SHANK | 1 | 5 | 2 |
| | Neuroligin | 0 | SHANK3 | 1 | 0 | 3 |
| | Neuroligin | 0 | EBP50 | 1 | 0 | 2 |
| | Neuroligin | 0 | EBP50 | 2 | 0 | 1 |
| | Neuroligin | 0 | INADL | 8 | 0 | 1 |
| | Neuroligin | 0 | PTPL-1 | 4 | 0 | 1 |
| | Neuroligin | 0 | PTPL-1 | 2 | 0 | 1 |
| | Neuroligin | 0 | PSD95 | 1, 2, 3 | 0 | 2 |
| | Neuroligin | 0 | NeDLG | 1, 2 | 0 | 1 |
| | Neuroligin | 0 | NOS1 | 1 | 0 | 1 |
| | Neuroligin | 0 | NeDLG | 3 | 0 | 1 |
| | Neuroligin | 0 | FLJ 10324 | 1 | 0 | 1 |
| | Neuroligin | 0 | Mint 1 | 1, 2 | 0 | 1 |
| | Neuroligin | 0 | KIAA807 | | 0 | 3 |
| | Neuroligin | 0 | DLG1 | 1, 2 | 0 | 2 |
| | Neuroligin | 0 | KIAA1634 | 2 | 0 | 2 |
| | Neuroligin | 0.1 | KIAA1634 | 1 | 1 | 4 |
| | Neuroligin | 0.25 | atrophin-1 interacting | 1 | 5 | 2 |
| | | | Protein | | | |
| AA37 | Glycophorin C | 0 | KIAA1719 | 6 | 5 | 1 |
| | Glycophorin C | 0 | PAR3 | 3 | 0 | 2 |
| AA40 | Dock2 | 0 | KIAA0382 | 1 | 0 | 1 |
| | Dock2 | 0 | SHANK | 1 | 0 | 1 |
| | Dock2 | 0 | SHANK3 | 1 | 0 | 1 |
| | Dock2 | 0 | EBP50 | 1 | 0 | 1 |
| | Dock2 | 0 | EBP50 | 2 | 0 | 2 |
| | Dock2 | 0 | KIAA0147 | 1 | 0 | 1 |
| | Dock2 | 0 | INADL | 3 | 0 | 1 |
| | Dock2 | 0 | HEMBA 1003117 | 1 | 0 | 1 |
| | Dock2 | 0 | hAPXL | 1 | 0 | 2 |
| | Dock2 | 0 | FLJ 10324 | 1 | 0 | 1 |
| | Dock2 | 0 | LIM-Mystique | 1 | 0 | 1 |
| | Dock2 | 0 | LIM RIL | 1 | 0 | 1 |
| | Dock2 | 0 | KIAA1634 | 5 | 0 | 1 |
| | Dock2 | 0 | BAI-1 | 6 | 0 | 1 |
| | Dock2 | 0 | Atrophin-1 Inter. Prot. | 5 | 0 | 1 |
| AA45 | BLR-1 | 0 | SHANK1 | 1 | 0 | 3 |
| | BLR-1 | 0 | SHANK3 | 1 | 0 | 3 |
| | BLR-1 | 0 | EBP50 | 1 | 0 | 3 |
| | BLR-1 | 0 | EBP50 | 2 | 0 | 3 |
| | BLR-1 | 2 | PDZK-1 | 2 | 5 | 1 |
| AA56 | Tax | 0 | TAX IP2 | 1 | 0 | 2 |
| | Tax | 0 | Syntrophin gamma-2 | 1 | 0 | 1 |
| | Tax | 0 | Syntrophin gamma-1 | 1 | 0 | 5 |
| | Tax | 0 | KIAA0147 | 4 | 0 | 1 |
| | Tax | 0 | KIAA0147 | 3 | 0 | 1 |
| | Tax | 0 | KIAA0147 | 2 | 0 | 5 |
| | Tax | 0 | KIAA0147 | 1 | 0.1 | 5 |
| | Tax | 0 | PTPL-1 | 2 | 0 | 2 |
| | Tax | 0 | PTN-4 | 1 | 0 | 2 |
| | Tax | 0 | INADL | 3 | 0 | 1 |
| | Tax | 0 | PSD95 | 3 | 0 | 1 |
| | Tax | 0 | PSD95 | 2 | 0 | 1 |
| | Tax | 0 | PSD95 | 1 | 0 | 5 |
| | Tax | 0 | MUPP-1 | 13 | 0 | 5 |
| | Tax | 0 | Outer Membrane | 1 | 0 | 5 |
| | Tax | 0 | NeDLG | 3 | 1 | 5 |
| | Tax | 0 | NeDLG | 2 | 1 | 5 |
| | Tax | 0 | FLJ 11215 | 1 | 0 | 1 |
| | Tax | 0 | FLJ 10324 | 1 | 0 | 1 |
| | Tax | 0 | FLJ 00011 | 1 | 0 | 1 |
| | Tax | 0 | LIMK1 | 1 | 0 | 1 |
| | Tax | 0 | LIM-Mystique | 1 | 0 | 1 |
| | Tax | 0 | Erbin | 1 | 1 | 5 |
| | Tax | 0 | LIM RIL | 1 | 0 | 1 |
| | Tax | 0 | DLG2 | 2 | 0 | 5 |
| | Tax | 0 | DLG2 | 1 | 0 | 2 |
| | Tax | 0 | DLG1 | 2 | 0 | 5 |
| | Tax | 0 | DLG1 | 1 | 0.5 | 5 |
| | Tax | 0 | Connector Enhancer | 1 | 0 | 1 |
| | Tax | 0 | KIAA1634 | 5 | 0 | 1 |
| | Tax | 0 | BAI-1 | 6 | 0 | 1 |
| | Tax | 0 | KIAA1634 | 4 | 0 | 2 |
| | Tax | 0 | BAI-1 | 5 | 0 | 5 |
| | Tax | 0 | KIAA1634 | 2 | 0 | 2 |
| | Tax | 0 | KIAA1634 | 1 | 0.1 | 5 |
| | Tax | 0 | BAI-1 | 4 | 0 | 2 |
| | Tax | 0 | BAI-1 | 3 | 0 | 1 |
| | Tax | 0 | BAI-1 | 2 | 0.5 | 5 |
| | Tax | 0 | Atrophin-1 Inter. Prot. | 5 | 0 | 3 |
| | Tax | 0 | KIAA1526 | 1 | 0 | 3 |
| | Tax | 0 | atrophin-1 interacting | 3 | 0 | 1 |
| | | | Protein | | | |
| | Tax | 0 | atrophin-1 interacting | 2 | 0 | 1 |
| | | | Protein | | | |
| | Tax | 0 | atrophin-1 interacting | 1 | 0 | 5 |
| | | | Protein | | | |
| | Tax | 0 | AIPC | 1 | 0 | 1 |
| AA58 | PAG | 0 | KIAA0382 | 1 | 0 | 1 |
| | PAG | 0 | KIAA0316 | 1 | 0 | 1 |
| | PAG | 0 | PIST | 1 | 0 | 1 |
| | PAG | 0 | hAPXL | 1 | 0 | 2 |
| | PAG | 0 | Outer Membrane | 1 | 0 | 2 |
| | PAG | 0 | SHANK | 1 | 0 | 4 |
| | PAG | 0 | SHANK3 | 1 | 0 | 2 |
| | PAG | 0 | PDZK1 | 2, 3, 4 | 0 | 1 |
| | PAG | 0 | FLJ 00011 | 1 | 0 | 3 |
| | PAG | 0 | Atrophin-1 Inter. Prot. | 5 | 0 | 1 |
| AA59 | PTEN | 0 | TIP1 | 1 | 0 | 2 |
| | PTEN | 0 | Syntrophin gamma-1 | 1 | 0 | 1 |
| | PTEN | 1.5 | SHANK | 1 | 5 | 3 |
| | PTEN | 0 | INADL | 8 | 0 | 1 |
| | PTEN | 0 | PTPL-1 | 4 | 0 | 1 |
| | PTEN | 0.3 | PTPL-1 | 2 | 1 | 4 |
| | PTEN | 0 | PIST | 1 | 0 | 1 |
| | PTEN | 0 | HEMBA 1003117 | 1 | 0 | 1 |
| | PTEN | 0 | MUPP-1 | 13 | 0 | 5 |
| | PTEN | 0 | GRIP1 | 3 | 0 | 1 |
| | PTEN | 0 | FLJ 10324 | 1 | 0 | 1 |
| | PTEN | 0 | FLJ 00011 | 1 | 0 | 3 |
| | PTEN | 0 | Mint 1 | 1, 2 | 0 | 1 |
| | PTEN | 0 | Mint 1 | 2 | 0 | 1 |
| | PTEN | 0 | KIAA807 | | 0 | 5 |
| | PTEN | 0 | KIAA1634 | 2 | 0 | 5 |
| | PTEN | 0 | BAI-1 | 3 | 0 | 2 |
| | PTEN | 0 | Atrophin-1 Inter. Prot. | 5 | 0 | 2 |
| | PTEN | 0 | AIPC | 1 | 0 | 1 |
| | PTEN | 0.3 | KIAA0807(S) | 1 | 0.5 | 5 |
| AA60 | AKT-1 | 2.5 | TAX IP2 | 1 | 1 | 4 |
| | AKT-1 | 0 | KIAA807 | | 0 | 1 |
| | AKT-1 | 0 | KIAA0807(S) | 1 | 0 | 1 |
| AA66.1 | HPV E6 #66 (modified) | 5 | TIP1 | 1 | 1 | 5 |
| | HPV E6 #66 (modified) | 0 | TAX IP2 | 1 | 0 | 2 |
| | HPV E6 #66 (modified) | 0 | Syntrophin gamma-2 | 1 | 0 | 1 |
| | HPV E6 #66 (modified) | 0 | Syntrophin gamma-1 | 1 | 0 | 1 |
| | HPV E6 #66 (modified) | 0 | Synt. 1 alpha | 1 | 0 | 2 |
| | HPV E6 #66 (modified) | 0 | KIAA0147 | 1 | 0 | 2 |
| | HPV E6 #66 (modified) | 0 | INADL | 8 | 0 | 1 |
| | HPV E6 #66 (modified) | 0 | PTPL-1 | 2 | 0 | 3 |
| | HPV E6 #66 (modified) | 0 | PSD95 | 1, 2, 3 | 0 | 5 |
| | HPV E6 #66 (modified) | 0 | PSD95 | 3 | 0 | 1 |
| | HPV E6 #66 (modified) | 0 | PSD95 | 1 | 0 | 4 |
| | HPV E6 #66 (modified) | 0 | MUPP-1 | 10 | 0 | 1 |
| | HPV E6 #66 (modified) | 0 | MUPP-1 | 13 | 0 | 3 |
| | HPV E6 #66 (modified) | 1 | NeDLG | 1, 2 | 0.5 | 5 |
| | HPV E6 #66 (modified) | 0 | hAPXL | 1 | 0 | 1 |
| | HPV E6 #66 (modified) | 0 | Outer Membrane | 1 | 0 | 5 |
| | HPV E6 #66 (modified) | 3.5 | NeDLG | 2 | 0.5 | 4 |
| | HPV E6 #66 (modified) | 0 | NeDLG | 1 | 0 | 1 |
| | HPV E6 #66 (modified) | 0 | FLJ 10324 | 1 | 0 | 1 |
| | HPV E6 #66 (modified) | 0 | FLJ 00011 | 1 | 0 | 1 |
| | HPV E6 #66 (modified) | 0 | Mint 1 | 1, 2 | 5 | 1 |
| | HPV E6 #66 (modified) | 0 | Mint 1 | 2 | 0 | 1 |
| | HPV E6 #66 (modified) | 0 | Erbin | 1 | 0 | 1 |
| | HPV E6 #66 (modified) | 0 | KIAA807 | | 0 | 2 |
| | HPV E6 #66 (modified) | 0 | DLG2 | 2 | 0 | 5 |
| | HPV E6 #66 (modified) | 0 | DLG2 | 1 | 0 | 1 |
| | HPV E6 #66 (modified) | 0 | DLG1 | 2 | 0 | 5 |
| | HPV E6 #66 (modified) | 0 | DLG1 | 1 | 0 | 4 |
| | HPV E6 #66 (modified) | 5 | DLG1 | 1, 2 | 5 | 5 |
| | HPV E6 #66 (modified) | 0 | BAI-1 | 5 | 5 | 1 |
| | HPV E6 #66 (modified) | 0 | KIAA1634 | 2 | 0 | 1 |
| | HPV E6 #66 (modified) | 0 | KIAA1634 | 1 | 0 | 5 |
| | HPV E6 #66 (modified) | 0 | BAI-1 | 3 | 5 | 1 |
| | HPV E6 #66 (modified) | 3 | BAI-1 | 2 | 0.5 | 5 |
| | HPV E6 #66 (modified) | 0 | Atrophin-1 Inter. Prot. | 5 | 0 | 1 |
| | HPV E6 #66 (modified) | 0 | KIAA1526 | 1 | 0 | 1 |
| | HPV E6 #66 (modified) | 0 | atrophin-1 interacting | 1 | 0 | 5 |
| | | | Protein | | | |
| | HPV E6 #66 (modified) | 0 | AIPC | 1 | 0 | 1 |
| | HPV E6 #66 (modified) | 5 | KIAA0807(S) | 1 | 5 | 4 |
| AA67.1 | HPV E6 #57 (modified) | 0 | TIP1 | 1 | 0 | 0 |
| | HPV E6 #57 (modified) | 0 | KIAA0147 | 1 | 0 | 1 |
| | HPV E6 #57 (modified) | 0 | BAI-1 | 2 | 0 | 0 |
| AA69.1 | HPV E6 E16 (modified) | 0 | TIP1 | 1 | 0 | 3 |
| | HPV E6 E16 (modified) | 0 | BAI-1 | 2 | 0 | 5 |
| AA70.1 | HPV E6 #18 | 0 | TIP1 | 1 | 0 | 4 |
| | HPV E6 #18 | 0 | BAI-1 | 2 | 0 | 5 |
| AA72.1 | HPV E6 33 (modified) | 0 | ZO-2 | 1 | 5 | 1 |
| | HPV E6 33 (modified) | 0 | TIP1 | 1 | 0 | 5 |
| | HPV E6 33 (modified) | 0 | Syntrophin gamma-2 | 1 | 5 | 1 |
| | HPV E6 33 (modified) | 0 | Synt. 1 alpha | 1 | 1 | 3 |
| | HPV E6 33 (modified) | 0 | SHANK | 1 | 5 | 4 |
| | HPV E6 33 (modified) | 0 | SHANK3 | 1 | 0 | 2 |
| | HPV E6 33 (modified) | 0 | EBP50 | 1 | 0 | 2 |
| | HPV E6 33 (modified) | 0 | EBP50 | 2 | 0 | 2 |
| | HPV E6 33 (modified) | 0 | PTN-4 | 1 | 5 | 1 |
| | HPV E6 33 (modified) | 0 | PSD95 | 1, 2, 3 | 0 | 5 |
| | HPV E6 33 (modified) | 5 | PSD95 | 3 | 0.5 | 5 |
| | HPV E6 33 (modified) | 0 | PSD95 | 1 | 5 | 2 |
| | HPV E6 33 (modified) | 0 | PDZK1 | 2, 3, 4 | 5 | 1 |
| | HPV E6 33 (modified) | 0 | Outer Membrane | 1 | 0 | 5 |
| | HPV E6 33 (modified) | 0 | NeDLG | 3 | 5 | 1 |
| | HPV E6 33 (modified) | 0 | NeDLG | 2 | 5 | 2 |
| | HPV E6 33 (modified) | 0 | NeDLG | 1 | 5 | 1 |
| | HPV E6 33 (modified) | 0 | NeDLG | 1, 2 | 0 | 5 |
| | HPV E6 33 (modified) | 0 | MUPP-1 | 13 | 5 | 2 |
| | HPV E6 33 (modified) | 0 | Mint 1 | 2 | 5 | 1 |
| | HPV E6 33 (modified) | 0 | KIAA1634 | 1 | 0 | 5 |
| | HPV E6 33 (modified) | 0 | KIAA1526 | 1 | 5 | 1 |
| | HPV E6 33 (modified) | 5 | KIAA1095 | 1 | 0.5 | 5 |
| | HPV E6 33 (modified) | 0 | KIAA0807(S) | 1 | 0 | 5 |
| | HPV E6 33 (modified) | 0 | KIAA0380 | 1 | 5 | 1 |
| | HPV E6 33 (modified) | 0 | KIAA0316 | 1 | 5 | 2 |
| | HPV E6 33 (modified) | 0 | KIAA0147 | 3 | 5 | 2 |
| | HPV E6 33 (modified) | 0 | KIAA0147 | 1 | 0 | 5 |
| | HPV E6 33 (modified) | 0 | hAPXL | 1 | 1 | 3 |
| | HPV E6 33 (modified) | 0 | FLJ 00011 | 1 | 5 | 1 |
| | HPV E6 33 (modified) | 0 | DLG2 | 2 | 1 | 3 |
| | HPV E6 33 (modified) | 0 | DLG2 | 1 | 5 | 1 |
| | HPV E6 33 (modified) | 5 | DLG1 | 2 | 0.5 | 5 |
| | HPV E6 33 (modified) | 0 | DLG1 | 1 | 1 | 3 |
| | HPV E6 33 (modified) | 0 | BAI-1 | 6 | 5 | 1 |
| | HPV E6 33 (modified) | 0 | BAI-1 | 5 | 5 | 1 |
| | HPV E6 33 (modified) | 0 | BAI-1 | 2 | 0 | 5 |
| | HPV E6 33 (modified) | 0 | Atrophin-1 Inter. Prot. | 5 | 5 | 1 |
| | HPV E6 33 (modified) | 5 | Atrophin-1 Inter. Prot. | 1 | 0.5 | 4 |
| | HPV E6 33 (modified) | 0 | AIPC | 1 | 5 | 1 |
| AA74.1 | HPV E6 52 (modified) | 0 | TIP1 | 1 | 0 | 0 |
| | HPV E6 52 (modified) | 0 | BAI-1 | 2 | 0 | 5 |
| AA75.1 | HPV E6 58 (modified) | 0 | ZO-2 | 1 | 1 | 3 |
| | HPV E6 58 (modified) | 0 | TIP1 | 1 | 0.5 | 4 |
| | HPV E6 58 (modified) | 0 | Synt. 1 alpha | 1 | 5 | 2 |
| | HPV E6 58 (modified) | 0 | PSD95 | 1, 2, 3 | 0 | 5 |
| | HPV E6 58 (modified) | 0 | PSD95 | 3 | 0 | 5 |
| | HPV E6 58 (modified) | 0 | PSD95 | 1 | 0 | 5 |
| | HPV E6 58 (modified) | 0 | PDZK1 | 2, 3, 4 | 5 | 1 |
| | HPV E6 58 (modified) | 5 | Outer Membrane | 1 | 0.5 | 5 |
| | HPV E6 58 (modified) | 5 | NeDLG | 3 | 5 | 2 |
| | HPV E6 58 (modified) | 0 | NeDLG | 2 | 0.5 | 5 |
| | HPV E6 58 (modified) | 0 | NeDLG | 1 | 5 | 1 |
| | HPV E6 58 (modified) | 0 | NeDLG | 1, 2 | 0 | 5 |
| | HPV E6 58 (modified) | 0 | MUPP-1 | 13 | 5 | 1 |
| | HPV E6 58 (modified) | 5 | MUPP-1 | 10 | 3 | 3 |
| | HPV E6 58 (modified) | 0 | Mint 1 | 2 | 5 | 1 |
| | HPV E6 58 (modified) | 0 | KIAA1634 | 5 | 5 | 1 |
| | HPV E6 58 (modified) | 0 | KIAA1634 | 2 | 5 | 1 |
| | HPV E6 58 (modified) | 0 | KIAA1634 | 1 | 0 | 5 |
| | HPV E6 58 (modified) | 0 | KIAA1526 | 1 | 5 | 1 |
| | HPV E6 58 (modified) | 0 | KIAA1095 | 1 | 5 | 1 |
| | HPV E6 58 (modified) | 0 | KIAA0973 | 1 | 5 | 2 |
| | HPV E6 58 (modified) | 0 | KIAA0807(S) | 1 | 0 | 5 |
| | HPV E6 58 (modified) | 0 | KIAA0380 | 1 | 5 | 1 |
| | HPV E6 58 (modified) | 0 | KIAA0147 | 1 | 5 | 2 |
| | HPV E6 58 (modified) | 0 | INADL | 8 | 0.5 | 4 |
| | HPV E6 58 (modified) | 0 | DLG2 | 2 | 0.5 | 5 |
| | HPV E6 58 (modified) | 0 | DLG1 | 2 | 0 | 5 |
| | HPV E6 58 (modified) | 5 | DLG1 | 1 | 0.5 | 5 |
| | HPV E6 58 (modified) | 0 | BAI-1 | 5 | 5 | 2 |
| | HPV E6 58 (modified) | 0 | BAI-1 | 4 | 5 | 2 |
| | HPV E6 58 (modified) | 0 | BAI-1 | 3 | 5 | 2 |
| | HPV E6 58 (modified) | 0 | BAI-1 | 2 | 0 | 5 |
| | HPV E6 58 (modified) | 0 | Atrophin-1 Inter. Prot. | 1 | 0 | 5 |
| AA78.1 | HPV E6 77 (Modified) | 0 | TIP1 | 1 | 0 | 0 |
| | HPV E6 77 (Modified) | 0 | BAI-1 | 2 | 0 | 0 |
| AA80.1 | HPV E6 #35 (modified) | 0 | ZO-2 | 1 | 0 | 2 |
| | HPV E6 #35 (modified) | 0 | ZO-1 | 1 | 0 | 1 |
| | HPV E6 #35 (modified) | 0 | TIP1 | 1 | 0 | 5 |
| | HPV E6 #35 (modified) | 0 | KIAA0382 | 1 | 0 | 2 |
| | HPV E6 #35 (modified) | 0 | KIAA0380 | 1 | 0 | 3 |
| | HPV E6 #35 (modified) | 0 | TAX IP2 | 1 | 0 | 4 |
| | HPV E6 #35 (modified) | 0 | Syntrophin gamma-2 | 1 | 0 | 3 |
| | HPV E6 #35 (modified) | 0 | Syntrophin gamma-1 | 1 | 0 | 4 |
| | HPV E6 #35 (modified) | 0 | Synt. 1 alpha | 1 | 0 | 5 |
| | HPV E6 #35 (modified) | 0 | KIAA0147 | 4 | 0 | 1 |
| | HPV E6 #35 (modified) | 0.35 | KIAA0147 | 3 | 5 | 4 |
| | HPV E6 #35 (modified) | 0 | KIAA0147 | 2 | 0 | 5 |
| | HPV E6 #35 (modified) | 0 | KIAA0147 | 1 | 0 | 5 |
| | HPV E6 #35 (modified) | 0 | INADL | 8 | 0 | 4 |
| | HPV E6 #35 (modified) | 0 | PTPL-1 | 4 | 0 | 1 |
| | HPV E6 #35 (modified) | 0 | PTPL-1 | 2 | 0 | 2 |
| | HPV E6 #35 (modified) | 0 | INADL | 5 | 0 | 1 |
| | HPV E6 #35 (modified) | 0 | PTN-4 | 1 | 0 | 4 |
| | HPV E6 #35 (modified) | 0 | INADL | 3 | 0 | 1 |
| | HPV E6 #35 (modified) | 0 | PSD95 | 1, 2, 3 | 0 | 5 |
| | HPV E6 #35 (modified) | 0 | PSD95 | 3 | 0 | 5 |
| | HPV E6 #35 (modified) | 0 | PSD95 | 1 | 0 | 5 |
| | HPV E6 #35 (modified) | 0 | PIST | 1 | 0 | 1 |
| | HPV E6 #35 (modified) | 0 | KIAA0973 | 1 | 0 | 2 |
| | HPV E6 #35 (modified) | 0 | KIAA1095 | 1 | 0 | 4 |
| | HPV E6 #35 (modified) | 0 | HEMBA 1003117 | 1 | 0 | 1 |
| | HPV E6 #35 (modified) | 0 | MUPP-1 | 10 | 0 | 4 |
| | HPV E6 #35 (modified) | 0 | MUPP-1 | 13 | 0 | 5 |
| | HPV E6 #35 (modified) | 0 | NeDLG | 1, 2 | 0 | 5 |
| | HPV E6 #35 (modified) | 0 | Outer Membrane | 1 | 0 | 5 |
| | HPV E6 #35 (modified) | 0 | NOS1 | 1 | 0 | 1 |
| | HPV E6 #35 (modified) | 0 | NeDLG | 3 | 0 | 5 |
| | HPV E6 #35 (modified) | 0 | NeDLG | 2 | 0 | 5 |
| | HPV E6 #35 (modified) | 0 | NeDLG | 1 | 0 | 5 |
| | HPV E6 #35 (modified) | 0 | GRIP1 | 6 | 0 | 2 |
| | HPV E6 #35 (modified) | 0 | GRIP1 | 3 | 0 | 2 |
| | HPV E6 #35 (modified) | 0 | MUPP-1 | 5 | 0 | 2 |
| | HPV E6 #35 (modified) | 0 | FLJ 12615 (PALS-1) | 1 | 0 | 1 |
| | HPV E6 #35 (modified) | 0 | FLJ 11215 | 1 | 0 | 4 |
| | HPV E6 #35 (modified) | 0 | FLJ 10324 | 1 | 0 | 1 |
| | HPV E6 #35 (modified) | 0.35 | FLJ 00011 | 1 | 5 | 3 |
| | HPV E6 #35 (modified) | 0 | Mint 1 | 1, 2 | 0 | 1 |
| | HPV E6 #35 (modified) | 0 | Mint 1 | 2 | 0 | 2 |
| | HPV E6 #35 (modified) | 0 | LIMK1 | 1 | 0 | 1 |
| | HPV E6 #35 (modified) | 0 | LIM-Mystigue | 1 | 0 | 1 |
| | HPV E6 #35 (modified) | 0.4 | Erbin | 1 | 5 | 2 |
| | HPV E6 #35 (modified) | 0 | LIM RIL | 1 | 0 | 4 |
| | HPV E6 #35 (modified) | 0 | KIAA807 | | 0 | 5 |
| | HPV E6 #35 (modified) | 0.2 | DLG2 | 2 | 0.5 | 5 |
| | HPV E6 #35 (modified) | 0 | DLG2 | 1 | 0 | 5 |
| | HPV E6 #35 (modified) | 0 | DLG1 | 3 | 5 | 3 |
| | HPV E6 #35 (modified) | 0 | DLG1 | 2 | 0 | 5 |
| | HPV E6 #35 (modified) | 0 | DLG1 | 1 | 0 | 5 |
| | HPV E6 #35 (modified) | 0 | KIAA1719 | 5 | 0 | 1 |
| | HPV E6 #35 (modified) | 0 | DLG1 | 1, 2 | 0 | 5 |
| | HPV E6 #35 (modified) | 0 | Connector Enhancer | 1 | 0 | 1 |
| | HPV E6 #35 (modified) | 0 | KIAA1634 | 5 | 0 | 3 |
| | HPV E6 #35 (modified) | 0 | BAI-1 | 6 | 0 | 3 |
| | HPV E6 #35 (modified) | 0 | KIAA1634 | 4 | 0 | 2 |
| | HPV E6 #35 (modified) | 0 | BAI-1 | 5 | 0 | 5 |
| | HPV E6 #35 (modified) | 0 | KIAA1634 | 2 | 0 | 3 |
| | HPV E6 #35 (modified) | 0 | KIAA1634 | 1 | 0 | 5 |
| | HPV E6 #35 (modified) | 0 | BAI-1 | 4 | 0 | 5 |
| | HPV E6 #35 (modified) | 0 | BAI-1 | 3 | 0 | 4 |
| | HPV E6 #35 (modified) | 0 | BAI-1 | 2 | 0 | 5 |
| | HPV E6 #35 (modified) | 0 | Atrophin-1 Inter. Prot. | 5 | 0 | 4 |
| | HPV E6 #35 (modified) | 1 | KIAA1526 | 1 | 5 | 3 |
| | HPV E6 #35 (modified) | 0 | atrophin-1 interacting | 3 | 0 | 4 |
| | | | Protein | | | |
| | HPV E6 #35 (modified) | 0 | KIAA1284 | 1 | 0 | 1 |
| | HPV E6 #35 (modified) | 0.8 | atrophin-1 interacting | 2 | 5 | 1 |
| | | | Protein | | | |
| | HPV E6 #35 (modified) | 0 | atrophin-1 interacting | 1 | 0 | 5 |
| | | | Protein | | | |
| | HPV E6 #35 (modified) | 0 | PDZ-73 | 2 | 0 | 2 |
| | HPV E6 #35 (modified) | 0 | AIPC | 1 | 5 | 1 |
| | HPV E6 #35 (modified) | 0.1 | KIAA0807(S) | 1 | 0.5 | 5 |
| AA82 | Adenovirus E4 Type9 | 0 | ZO-2 | 1 | 0 | 3 |
| | Adenovirus E4 Type9 | 0 | ZO-1 | 1 | 0 | 2 |
| | Adenovirus E4 Type9 | 0 | KIAA0382 | 1 | 0 | 1 |
| | Adenovirus E4 Type9 | 0 | KIAA0300 | 1 | 0 | 1 |
| | Adenovirus E4 Type9 | 0 | INADL | 8 | 0 | 2 |
| | Adenovirus E4 Type9 | 0 | PTPL-1 | 4 | 0 | 4 |
| | Adenovirus E4 Type9 | 0.2 | PTPL-1 | 2 | 5 | 3 |
| | Adenovirus E4 Type9 | 0 | PSD95 | 1, 2, 3 | 0 | 5 |
| | Adenovirus E4 Type9 | 0.1 | PSD95 | 1 | 5 | 4 |
| | Adenovirus E4 Type9 | 0 | PIST | 1 | 0 | 1 |
| | Adenovirus E4 Type9 | 0 | KIAA1222 | 1 | 0 | 1 |
| | Adenovirus E4 Type9 | 0.3 | HEMBA 1003117 | 1 | 5 | 3 |
| | Adenovirus E4 Type9 | 0.1 | MUPP-1 | 11 | 5 | 5 |
| | Adenovirus E4 Type9 | 0 | NeDLG | 1, 2 | 0 | 5 |
| | Adenovirus E4 Type9 | 0.1 | Outer Membrane | 1 | 5 | 5 |
| | Adenovirus E4 Type9 | 0 | NOS1 | 1 | 0 | 5 |
| | Adenovirus E4 Type9 | 0.1 | NeDLG | 2 | 5 | 5 |
| | Adenovirus E4 Type9 | 0 | NeDLG | 1 | 0 | 1 |
| | Adenovirus E4 Type9 | 0 | MUPP-1 | 10 | 0 | 1 |
| | Adenovirus E4 Type9 | 0.1 | FLJ 10324 | 1 | 5 | 3 |
| | Adenovirus E4 Type9 | 0 | FLJ 00011 | 1 | 0 | 1 |
| | Adenovirus E4 Type9 | 0 | Mint 1 | 1, 2 | 0 | 2 |
| | Adenovirus E4 Type9 | 0 | Mint 1 | 2 | 0 | 2 |
| | Adenovirus E4 Type9 | 0 | KIAA807 | | 0 | 4 |
| | Adenovirus E4 Type9 | 0.05 | DLG2 | 2 | 0.5 | 5 |
| | Adenovirus E4 Type9 | 0.03 | DLG1 | 2 | 0.3 | 5 |
| | Adenovirus E4 Type9 | 0.1 | DLG1 | 1 | 0.5 | 4 |
| | Adenovirus E4 Type9 | 0 | DLG1 | 1, 2 | 0 | 5 |
| | Adenovirus E4 Type9 | 0.1 | Connector Enhancer | 1 | 5 | 3 |
| | Adenovirus E4 Type9 | 0 | BAI-1 | 6 | 0 | 1 |
| | Adenovirus E4 Type9 | 0.2 | KIAA1634 | 4 | 5 | 4 |
| | Adenovirus E4 Type9 | 0.15 | KIAA1634 | 2 | 5 | 5 |
| | Adenovirus E4 Type9 | 0.1 | BAI-1 | 4 | 0.3 | 5 |
| | Adenovirus E4 Type9 | 0.075 | BAI-1 | 3 | 0.5 | 5 |
| | Adenovirus E4 Type9 | 0 | KIAA1634 | 1 | 0 | 5 |
| | Adenovirus E4 Type9 | 0.02 | BAI-1 | 2 | 0.3 | 5 |
| | Adenovirus E4 Type9 | 0.1 | atrophin-1 interacting | 3 | 5 | 4 |
| | | | Protein | | | |
| | Adenovirus E4 Type9 | 0.02 | atrophin-1 interacting | 1 | 0.5 | 5 |
| | | | Protein | | | |
| | Adenovirus E4 Type9 | 0.2 | KIAA0807(S) | 1 | 5 | 3 |

TABLE 8

| | | | | | SEQ |
|---|---|---|---|---|---|
| | | | Accession | | ID |
| AVC ID | AVC Name | Sequence | No | GI | NO: |
| AA01.1 | Clasp-1 | VISKATPALPTVSISSSAEV | | | 534 |
| AA02.1 | Clasp-2 | ISGTPTSTMVHGMTSSSSVV | | | 535 |
| AA06 | CD6 | SPQPDSTDNDDYDDISAA | x60992 | | 536 |
| AA07 | CD34 | QATSRNGHSARQHVVADTEL | m81104 | | 537 |
| AA091 | GAIP (G-alpha interacting protein) RGS 19 | SSPTYRALLLQGPSQSSSEA | p49795 and | 1730186 | 538 |
| | | | X91809 | and | |
| | | | | 1107697 | |
| AA092 | alpha-1-syntrophin | IVFIHSFLSAKVTRLGLLA | 2209282A | 1588680 | 539 |
| AA093 | neurofascin (chicken) | TEGNESSEATSPVNAIYSLA | CAA46330 | 63660 | 540 |
| AA095 | GluR5-2 (rat) | SFTSILTCHQRRTQRKETVA | M83561 | 204389 | 541 |
| AA098L | ropporin | GPDGIITVNDFTQNPRVQLE | AAG27712 | 11037716 | 542 |
| AA10 | CD46 | KKGTYLTDETHREVKFTSL | M58050 | | 543 |
| AA105 | CX43 (connexin 43) | PSSRASSRASSRPRPDDLEI | P17302 | | 544 |
| AA106 | Kir2.1 (inwardly rect. K+ channel) | LHNQASVPLEPRPLRRESEI | af153818S1 | 8132299 | 545 |
| | | | and | | |
| | | | AH009400 | | |
| AA108.1 | GLUR2 (glutamate receptor 2-modified) | GGGGGSGGGGGSGIESVKI | | | 546 |
| AA111 | ephrin A2 | RIAYSLLGLKDQVNTVGIPI | P29317 and | 125333 | 547 |
| | | | XP_002088 | and | |
| | | | | 11427699 | |
| AA112 | GluR delta-2 | QPTPTLGLNLGNDPDRGTSI | AAC39579 | 2853315 | 548 |
| AA113 | SSTR2 (somatostatin recepor 2) | LNETTETQRTLLNGDLQTSI | XM_012697 | 12740762 | 549 |
| AA114 | GLUR7 (metabotropic glutamate receptor) | VDPNSPAAKKKYVSYNNLVI | XP_010942 | 12729188 | 550 |
| AA115 | presenilin-1 | ATDYLVQPFMDQLAFHQFYI | XP_007441 | 11435042 | 551 |
| AA116 | MINT-2 | KTMPAAMFRLLTGQETPLYI | AAC05306 | 2625029 | 552 |
| AA117 | presenilin-2 | STDNLVRPFMDTLASHQLYI | NP_036618 | 7108360 | 553 |
| AA118 | MINT-1 | KTMPAAMYRLLTAQEQPVYI | 35430 | 6225060 | 554 |
| AA121 | CD68 | ALVLIAFCIIRRRPSAYQAL | s57235 | | 555 |
| AA123 | a-actinin 2 | VPGALDYAAFSSALYGESDL | p35609 | 543742 | 556 |
| AA125 | zona occludens 3 (ZO-3) | VHDAESSDEDGYDWGPATDL | NP_055243 | 10092691 | 557 |
| AA13 | CD95 | KDITSDSENSNFRNEIQSLV | | | 558 |
| AA140 | KIA 1481 | PIPAGGCTFSGIFPTLTSPL | AB040914 | 7959222 | 559 |
| AA147 | Na+/Pi cotransporter 2 | PPATPSPRLALPAHHNATRL | Q06495 | 730113 | 560 |
| AA148L | CFTCR (cystic fibrosis transmembrane | KPQIAALKEETEEEVQDTRL | AAC13657 | 306538 | 561 |
| | conductance regulator) | | | | |
| AA152L | ActRIIA | IVTVVTMVTNVDFPPKESSL | BAA06548 | 1321632 | 562 |
| AA161 | MINT-3 | KTMPAATYRLLTGQEQPVYL | 96018 | 6226953 | 563 |
| AA169L | CAPON (carboxyl-terminal PDZ ligand of | LLNVLQRQELGDGLDDEIAV | AF037070 | 2895554 | 564 |
| | neuronal nitric oxide synthase) mRNA | | | | |
| AA172 | RA-GEF (ras/rap1A-assoc-GEF) | PYQSQGFSTEEDEDEQVSAV | NP_055062 | 7657261 | 565 |
| AA177L | c-kit receptor | INSVGSTASSSQPLLVHDDV | TVHUKT | 66811 | 566 |
| AA178L | PDZ-binding kinase (PBK) | EDPKDRPSAAHIVEALETDV | XP_005110 | 11424184 | 567 |
| AA180 | NMDA Glutamate Receptor 2C (cysteine-free) | TQGFPGPATWRRISSLESEV | | | 568 |
| AA182L | ephrin B2 | ILNSIQVMRAQMNQIQSVEV | 1F0MA | 9256876 | 569 |
| AA183L | RhoGAP 1 (PTPL1-associated) | PRLKRMQQFEDLEDEIPQFV | NP_004806 | 4758882 | 570 |
| | | | and | and | |
| | | | NM_004815 | 4758881 | |
| AA185L | RGS12 (regulator of G-protein signaling 12 | GPVPGEPAKPKTSAHHATFV | 14924 | 3914623 | 571 |
| AA190L | ephrin B1 | PVYIVQEMPPQSPANIYYKV | XP_010388 | 11421689 | 572 |
| AA192L | JAM (junctional adhesion molecule) | YSQPSARSEGEFKQTSSFLV | Q9Y624 | 10720061 | 573 |
| AA205L | serotonin receptor 5-HT-2C | ENLELPVNPSSVVSERISSV | XP_013121 | 12743533 | 574 |
| AA206L | CITRON protein | AGAVRTPLSQVNKVWDQSSV | O14578 | 6225217 | 575 |
| AA207L | Nedasin (s-form) | RNIEEVYVGGKQVVPFSSSV | AAAF13301 | 6469320 | 576 |
| AA210L | APC-adenomatous polyposis coli protein | ESSGTQSPKRHSGSYLVTSV | P25054 | 114033 | 577 |
| AA214L | ErbB-4 receptor | SLKPGTVLPPPPYRHRNTVV | g15303 | 3913590 | 578 |
| AA215 | CKR5 (HIV Co-receptor) | ERASSVYTRSTGEQEISVGL | P51681 | | 579 |
| AA216 | NMDA R2C | HPTDITGLPNLSDPSVSTVV | AAB59360 | 292283 | 580 |
| AA217 | catenin - delta 2 | PYSELNYETSHYPASPDSWV | NP_001323 | 11034811 | 581 |
| AA218 | CSPG4 (chondroitin sulfae proteoglycan 4, | ELLQFCRTPNPALKNGQYWV | NM_001897 | 4503098 | 582 |
| | melanoma-associated) | | and X96753 | and | |
| | | | | 1617313 | |
| AA22 | DNAM-1 | TREDIYVNYPTFSRRPKTRV | | | 583 |
| AA220 | claudin 10 | GGEDFKTTNPSKQFDKNAYV | XP_007076 | | 584 |
| AA222 | claudin 18 | DGGARTEDEVQSYPSKHDYV | XP_003116 | | 585 |
| AA223 | claudin 1 | SYPTPRPYPKPAPSSGKDYV | XP_003151 | | 586 |
| AA225 | claudin 9 | LGYSIPSRSGASGLDKRDYV | XP_012519 | | 587 |
| AA226 | claudin 7 | KAGYRAPRSYPKSNSSKEYV | AAH01055 | | 588 |
| AA227 | claudin 2 | PGQPPKVKSEFNSYSLTGYV | XP_010309 | 11420901 | 589 |
| AA228 | Nectin 2 | SSPDSSYQGKGFVMSRAMYV | g92692 | 12643789 | 590 |
| AA23.3 | Fas Ligand | SSKSKSSEESQTFFGLYKL | | | 591 |
| AA233L | serotonin receptor 5HT-2B | DTLLLTENEGDKTEEQVSYV | P41595 | | 592 |
| AA240 | Dopamine transporter | RELVDRGEVRQFTLRHWLKV | Q01959 | 266667 | 593 |
| AA243 | alpha-2A Adrenergic receptor | HDFRRAFKKILARGDRKRIV | P08913 | | 594 |
| AA244 | alpha-2B Adrenergic receptor | QDFRRAFRRILARPWTQTAW | P18089 | | 595 |
| AA245 | alpha-2C Adrenergic receptor | DFRPSFKHILFRRARRGFRQ | P18825 | | 596 |
| AA248 | somatostatin receptor 4 | EALQPEPGRKRIPLTRTTTF | P31391 | | 597 |
| AA25 | FceRlb | YSATYSELEDPGEMSPPIDL | | | 598 |
| AA250 | Serotonin receptor 3a | LAVLAYSITLVMLWSIWQYA | NP_000860 | 4504543 | 599 |
| AA252 | muscarinic Ach receptor M4 | QQYQQRQSVIFHKRAPEQAL | P20309 | | 600 |
| AA255 | Clasp-5 | RDSFHRSSFRKAETQLSQGS | | | 601 |
| AA258 | noradrenaline transporter | HHLVAQRDIRQFQLQHWLAI | M65015 | 189257 | 602 |
| AA261 | GABA transporter 3 | DAKLKSDGTIAAITEKETHF | XM_003161 | 12729857 | 603 |
| AA262 | glutamate transporter 3 | NGGFAVDKSDTISFTQTSQF | | 11352332 | 604 |
| AA264 | bone morphogenetic protein receptor | TALRIKKTLAKMVESQDVKI | XM_015818 | 13646025 | 605 |
| AA268 | parathyroid hormone receptor 2 | RPMESNPDTEGAQGETEDVL | P49190 | | 606 |
| AA269 | C5 Anaphylatoxin receptor | ESKSFTRSTVDTMAQKTQAV | P21730 | | 607 |
| AA28.1 | CDW125 (modified) | EVIGYIEKPGVETLEDSVF | | | 608 |
| AA29.2 | CDw128B | KDSRPSFVGSSSGHTSTTL | | | 609 |
| AA29.3 | IL-8RA | ARHRVTSYTSSSVNVSSNL | | | 610 |
| AA30 | LPAP | AWDDSARAAGGQGLHVTAL | | | 611 |
| | LPAP | AAWDDSARAAGGQGLHVTAL | | | 612 |
| AA300 | TRAF2 | NSYVRDDAIFIKAIVDLTGL | XM_011774 | 14737659 | 613 |
| AA31 | Mannose Receptor | GTSDMKDLVGNIEQNEHSVI | | | 614 |
| AA36 | Neuroligin | TFAAGFNSTGLPHSTTRV | | | 615 |
| AA37 | Glycophorin C | QGDPALQDAGDSSRKEYFI | | | 616 |
| AA40 | DOCK2 | LASKSAEEGKQIPDSLSTDL | | | 617 |
| AA45 | BLR-1 | PSWRRSSLSESENATSLTTF | | | 618 |
| AA56 | TAX | QISPGGLEPPSEKHFRETEV | | | 619 |
| AA58 | PAG | KENDYESISDLQQGRDITRL | | | 620 |
| AA59 | PTEN | DSDPENEPFDEDQHTQITKV | | | 621 |
| AA60 | AKT1 | VDSERRPHFPQFSYSASSTA | | | 622 |
| AA66.1 | HPV E6 #66 (cysteine-free) | TGSALQAWRHTSRQATESTV | | | 623 |
| AA67.1 | HPV E6 #57 (cysteine-free) | HAMNAAPRAMENAPALRTSH | | | 624 |
| AA69.1 | HPV E6 #16 (Modified) | TGRGMSGGRSSRTRRETQL | | | 625 |
| AA70.1 | HPV E6 #18 | SGGNRARQERLQRRRETQV | | | 626 |
| AA72.1 | HPV E6 33 (modified) | AAGGRSARGGRLQGRRETAL | | | 627 |
| AA74.1 | HPV E6 52 (modified) | SEGGRPTRGPRLQGRRVTQV | | | 628 |
| AA75.1 | HPV E6 58 (modified) | AVGGRPARGGRLQGRRQTQV | | | 629 |
| AA78.1 | HPV E6 77 (modified) | GGGRGSGLAGGSRGGGQSRQ | | | 630 |
| AA80.1 | HPV E6 #35 (cysteine-free) | GRWTGRAMSAWKPTRRETEV | | | 631 |
| AA82 | AdenoE4 typ9 | VGTLLLERVIFPSVKIATLV | | | 632 |

TABLE 9

| | | Domain | | SEQ ID |
|---|---|---|---|---|
| Gene Name | GI | Number | Sequence | NO: |
| 26s subunit | 9184389 | 1 | RDMAEAHKEAMSRKLGQSESQGPPRAFAKVNSISPG | 633 |
| p27 | | | SPSIAGLQVDDEIVEFGSVNTQNFQSLHNIGSVVQHS | |
| | | | EGALAPTILLSVSM | |
| AF6 | 430993 | 1 | LRKEPEIITVTLKKQNGMGLSIVAAKGAGQDKLGIYVK | 634 |
| | | | SVVKGGAADVDGRLAAGDQLLSVDGRSLVGLSQER | |
| | | | AAELMTRTSSVVTLEVAKQG | |
| AIPC | 12751451 | 1 | LIRPSVISIIGLYKEKGKGLGFSIAGGRDCIRGQMGIFV | 635 |
| | | | KTIFPNGSAAEDGRLKEGDEILDVNGIPIKGLTFQEAIH | |
| | | | TFKQIRSGLFVLTVRTKLVSPSLTNSS | |
| AIPC | 12751451 | 2 | GISSLGRKTPGPKDRIVMEVTLNKEPRVGLGIGACCL | 636 |
| | | | ALENSPPGIYIHSLAPGSVAKMESNLSRGDQILEVNSV | |
| | | | NVRHAALSKVHAILSKCPPGPVRLVIGRHPNPKVSEQ | |
| | | | EMDEVIARSTYQESKEANSS | |
| AIPC | 12751451 | 3 | QSENEEDVCFIVLNRKEGSGLGFSVAGGTDVEPKSIT | 637 |
| | | | VHRVFSQGAASQEGTMNRGDFLLSVNGASLAGLAH | |
| | | | GNVLKVLHQAQLHKDALVVIKKGMDQPRPSNSS | |
| AIPC | 12751451 | 4 | LGRSVAVHDALCVEVLKTSAGLGLSLDGGKSSVTGD | 638 |
| | | | GPLVIKRVYKGGAAEQAGIIEAGDEILAINGKPLVGLM | |
| | | | HFDAWNIMKSVPEGPVQLLIRKHRNSS | |
| alpha | 2773059 | 1 | QTVILPGPAAWGFRLSGGIDFNQPLVITRITPGSKAAA | 639 |
| actinin-2 | | | ANLCPGDVILAIDGFGTESMTHADGQDRIKAAEFIV | |
| associated | | | | |
| LIM protein | | | | |
| APXL-1 | 13651263 | 1 | ILVEVQLSGGAPWGFTLKGGREHGEPLVITKIEEGSK | 640 |
| | | | AAAVDKLLAGDEIVGINDIGLSGFRQEAICLVKGSHKT | |
| | | | LKLVVKRNSS | |
| Atrophin-1 | 2947231 | 1 | REKPLFTRDASQLKGTFLSTTLKKSNMGFGFTIIGGDE | 641 |
| Interacting | | | PDEFLQVKSVIPDGPAAQDGKMETGDVIVYINEVCVL | |
| Protein | | | GHTHADVVKLFQSVPIGQSVNLVLCRGYP | |
| Atrophin-1 | 2947231 | 2 | LSGATQAELMTLTIVKGAQGFGFTIADSPTGQRVKQIL | 642 |
| Interacting | | | DIQGCPGLCEGDLIVEINQQNVQNLSHTEVVDILKDCP | |
| Protein | | | IGSETSLIIHRGGFF | |
| Atrophin-1 | 2947231 | 3 | HYKELDVHLRRMESGFGFRILGGDEPGQPILIGAVIAM | 643 |
| Interacting | | | GSADRDGRLHPGDELVYVDGIPVAGKTHRYVIDLMH | |
| Protein | | | HAARNGQVNLTVRRKVLCG | |
| Atrophin-1 | 2947231 | 4 | EGRGISSHSLQTSDAVIHRKENEGFGFVIISSLNRPES | 644 |
| Interacting | | | GSTITVPHKIGRIIDGSPADRCAKLKVGDRILAVNGQSI | |
| Protein | | | INMPHADIVKLIKDAGLSVTLRIIPQEEL | |
| Atrophin-1 | 2947231 | 5 | LSDYRQPQDFDYFTVDMEKGAKGFGFSIRGGREYKM | 645 |
| Interacting | | | DLYVLRLAEDGPAIRNGRMRVGDQIIEINGESTRDMT | |
| Protein | | | HARAIELIKSGGRRVRLLLKRGTGQ | |
| Atrophin-1 | 2947231 | 6 | HESVIGRNPEGQLGFELKGGAENGQFPYLGEVKPGK | 646 |
| Interacting | | | VAYESGSKLVSEELLLEVNETPVAGLTIRDVLAVIKHC | |
| Protein | | | KDPLRLKCVKQGGIHR | |
| BAI-1 | 3370997 | 1 | IQKKNHWTSRVHECTVKRGPQGELGVTVLGGAEHG | 647 |
| Associated | | | EFPYVGAVAAVEAAGLPGGGEGPRLGEGELLLEVQG | |
| Protein | | | VRVSGLPRYDVLGVIDSCKEAVTFKAVRQGGR | |
| BAI-1 | 3370997 | 2 | PSELKGKFIHTKLRKSSRGFGFTVVGGDEPDEFLQIK | 648 |
| Associated | | | SLVLDGPAALDGKMETGDVIVSVNDTCVLGHTHAQV | |
| Protein | | | VKIFQSIPIGASVDLELCRGYPLPFDPDDPN | |
| BAI-1 | 3370997 | 3 | PATQPELITVHIVKGPMGFGFTIADSPGGGGQRVKQI | 649 |
| Associated | | | VDSPRCRGLKEGDLIVEVNKKNVQALTHNQVVDMLV | |
| Protein | | | ECPKGSEVTLLVQRGGNLS | |
| BAI-1 | 3370997 | 4 | PDYQEQDIFLWRKETGFGFRILGGNEPGEPIYIGHIVP | 650 |
| Associated | | | LGAADTDGRLRSGDELICVDGTPVIGKSHQLVVQLMQ | |
| Protein | | | QAAKQGHVNLTVRRKVVFAVPKTENSS | |
| BAI-1 | 3370997 | 5 | GVVSTVVQPYDVEIRRGENEGFGFVIVSSVSRPEAGT | 651 |
| Associated | | | TFAGNACVAMPHKIGRIIEGSPADRCGKLKVGDRILAV | |
| Protein | | | NGCSITNKSHSDIVNLIKEAGNTVTLRIIPGDESSNA | |
| BAI-1 | 3370997 | 6 | QATQEQDFYTVELERGAKGFGFSLRGGREYNMDLYV | 652 |
| Associated | | | LRLAEDGPAERCGKMRIGDEILEINGETTKNMKHSRAI | |
| Protein | | | ELIKNGGRRVRLFLKRG | |
| CARD11 | 12382772 | 1 | NLMFRKFSLERPFRPSVTSVGHVRGPGPSVQHTTLN | 653 |
| | | | GDSLTSQLTLLGGNARGSFVHSVKPGSLAEKAGLRE | |
| | | | GHQLLLLEGCIRGERQSVPLDTCTKEEAHWTIQRCS | |
| | | | GPVTLHYKVNHEGYRKLV | |
| CARD14 | 13129123 | 1 | ILSQVTMLAFQGDALLEQISVIGGNLTGIFIHRVTPGSA | 654 |
| | | | ADQMALRPGTQIVMVDYEASEPLFKAVLEDTTLEEAV | |
| | | | GLLRRVDGFCCLSVKVNTDGYKRL | |
| CASK | 3087815 | 1 | TRVRLVQFQKNTDEPMGITLKMNELNHCIVARIMHGG | 655 |
| | | | MIHRQGTLHVGDEIREINGISVANQTVEQLQKMLREM | |
| | | | RGSITFKIVPSYRTQS | |
| Connector | 3930780 | 1 | LEQKAVLEQVQLDSPLGLEIHTTSNCQHFVSQVDTQV | 656 |
| Enhancer | | | PTDSRLQIQPGDEVVQINEQVVVGWPRKNMVRELLR | |
| | | | EPAGLSLVLKKIPIP | |
| Cytohesin | 3192908 | 1 | QRKLVTVEKQDNETFGFEIQSYRPQNQNACSSEMFT | 657 |
| Binding | | | LICKIQEDSPAHCAGLQAGDVLANINGVSTEGFTYKQ | |
| Protein | | | VVDLIRSSGNLLTIETLNG | |
| DLG1 | 475816 | 1 | IQVNGTDADYEYEEITLERGNSGLGFSIAGGTDNPHIG | 658 |
| | | | DDSSIFITKIITGGAAAQDGRLRVNDCILQVNEVDVRD | |
| | | | VTHSKAVEALKEAGSIVRLYVKRRN | |
| DLG1 | 475816 | 2 | IQLIKGPKGLGFSIAGGVGNQHIPGDNSIYVTKIIEGGA | 659 |
| | | | AHKDGKLQIGDKLLAVNNVCLEEVTHEEAVTALKNTS | |
| | | | DFVYLKVAKPTSMYMNDGN | |
| DLG1 | 475816 | 3 | ILHRGSTGLGFNIVGGEDGEGIFISFILAGGPADLSGEL | 660 |
| | | | RKGDRIISVNSVDLRAASHEQAAAALKNAGQAVTIVA | |
| | | | QYRPEEYSR | |
| DLG2 | 12736552 | 1 | ISYVNGTEIEYEFEEITLERGNSGLGFSIAGGTDNPHIG | 661 |
| | | | DDPGIFITKIIPGGAAAEDGRLRVNDCILRVNEVDVSE | |
| | | | VSHSKAVEALKEAGSIVRLYVRRR | |
| DLG2 | 12736552 | 2 | ISVVEIKLFKGPKGLGFSIAGGVGNQHIPGDNSIYVTKII | 662 |
| | | | DGGAAQKDGRLQVGDRLLMVNNYSLEEVTHEEAVAI | |
| | | | LKNTSEVVYLKVGNPTTI | |
| DLG2 | 12736552 | 3 | IWAVSLEGEPRKVVLHKGSTGLGFNIVGGEDGEGIFV | 663 |
| | | | SFILAGGPADLSGELQRGDQILSVNGIDLRGASHEQA | |
| | | | AAALKGAGQTVTIIAQYQPED | |
| DLG5 | 3650451 | 1 | GIPYVEEPRHVKVQKGSEPLGISIVSGEKGGIYVSKVT | 664 |
| | | | VGSIAHQAGLEYGDQLLEFNGINLRSATEQQARLIIGQ | |
| | | | QCDTITILAQYNPHVHQLRNSSZLTD | |
| DLG5 | 3650451 | 2 | GILAGDANKKTLEPRVVFIKKSQLELGVHLCGGNLHG | 665 |
| | | | VFVAEVEDDSPAKGPDGLVPGDLILEYGSLDVRNKTV | |
| | | | EEVYVEMLKPRDGVRLKVQYRPEEFIVTD | |
| DVL1 | 2291005 | 1 | LNIVTVTLNMERHHFLGISIVGQSNDRGDGGIYIGSIM | 666 |
| | | | KGGAVAADGRIEPGDMLLQVNDVNFENMSNDDAVR | |
| | | | VLREIVSQTGPISLTVAKCW | |
| DVL2 | 2291007 | 1 | LNIITVTLNMEKYNFLGISIVGQSNERGDGGIYIGSIMK | 667 |
| | | | GGAVAADGRIEPGDMLLQVNDMNFENMSNDDAVRV | |
| | | | LRDIVHKPGPIVLTVAKCWDPSPQNS | |
| DVL3 | 6806886 | 1 | IITVTLNMEKYNFLGISIVGQSNERGDGGIYIGSIMKGG | 668 |
| | | | AVAADGRIEPGDMLLQVNEINFENMSNDDAVRVLREI | |
| | | | VHKPGPITLTVAKCWDPSP | |
| ELFIN 1 | 2957144 | 1 | TTQQIDLQGPGPWGFRLVGRKDFEQPLAISRVTPGS | 669 |
| | | | KAALANLCIGDVITAIDGENTSNMTHLEAQNRIKGCTD | |
| | | | NLTLTVARSEHKVWSPLV | |
| ENIGMA | 561636 | 1 | IFMDSFKVVLEGPAPWGFRLQGGKDFNVPLSISRLTP | 670 |
| | | | GGKAAQAGVAVGDWVLSIDGENAGSLTHIEAQNKIR | |
| | | | ACGERLSLGLSRAQPV | |
| ERBIN | 8923908 | 1 | QGHELAKQEIRVRVEKDPELGFSISGGVGGRGNPFR | 671 |
| | | | PDDDGIFVTRVQPEGPASKLLQPGDKIIQANGYSFINI | |
| | | | EHGQAVSLLKTFQNTVELIIVREVSS | |
| EZRIN | 3220018 | 1 | ILCCLEKGPNGYGFHLHGEKGKLGQYIRLVEPGSPAE | 672 |
| Binding | | | KAGLLAGDRLVEVNGENVEKETHQQVVSRIRAALNA | |
| Protein 50 | | | VRLLVVDPEFIVTD | |
| EZRIN | 3220018 | 2 | IRLCTMKKGPSGYGFNLHSDKSKPGQFIRSVDPDSPA | 673 |
| Binding | | | EASGLRAQDRIVEVNGVCMEGKQHGDVVSAIRAGGD | |
| Protein 50 | | | ETKLLVVDRETDEFFMNSS | |
| FLJ00011 | 10440352 | 1 | KNPSGELKTVTLSKMKQSLGISISGGIESKVQPMVKIE | 674 |
| | | | KIFPGGAAFLSGALQAGFELVAVDGENLEQVTHQRA | |
| | | | VDTIRRAYRNKAREPMELVVRVPGPSPRPSPSD | |
| FLJ11215 | 11436365 | 1 | EGHSHPRVVELPKTEEGLGFNIMGGKEQNSPIYISRII | 675 |
| | | | PGGIADRHGGLKRGDQLLSVNGVSVEGEHHEKAVEL | |
| | | | LKAAQGKVKLVVRYTPKVLEEME | |
| FLJ12615 | 10434209 | 1 | GQYGGETVKIVRIEKARDIPLGATVRNEMDSVIISRIVK | 676 |
| | | | GGAAEKSGLLHEGDEVLEINGIEIRGKDVNEVFDLLSD | |
| | | | MHGTLTFVLIPSQQIKPPPA | |
| FLJ20075 | 7019938 | 1 | ILAHVKGIEKEVNVYKSEDSLGLTITDNGVGYAFIKRIK | 677 |
| | | | DGGVIDSVKTICVGDHIESINGENIVGWRHYDVAKKLK | |
| | | | ELKKEELFTMKLIEPKKAFEI | |
| FLJ21687 | 10437836 | 1 | KPSQASGHFSVELVRGYAGFGLTLGGGRDVAGDTPL | 678 |
| | | | AVRGLLKDGPAQRCGRLEVGDLVLHINGESTQGLTH | |
| | | | AQAVERIRAGGPQLHLVIRRPLETHPGKPRGV | |
| GRIP 1 | 4539083 | 1 | VVELMKKEGTTLGLTVSGGIDKDGKPRVSNLRQGGIA | 679 |
| | | | ARSDQLDVGDYIKAVNGINLAKFRHDEIISLLKNVGER | |
| | | | VVLEVEYE | |
| GRIP 1 | 4539083 | 2 | RSSVIFRTVEVTLHKEGNTFGFVIRGGAHDDRNKSRP | 680 |
| | | | VVITCVRPGGPADREGTIKPGDRLLSVDGIRLLGTTHA | |
| | | | EAMSILKQCGQEAALLIEYDVSVMDSVATASGNSS | |
| GRIP 1 | 4539083 | 3 | HVATASGPLLVEVAKTPGASLGVALTTSMCCNKQVIVI | 681 |
| | | | DKIKSASIADRCGALHVGDHILSIDGTSMEYCTLAEAT | |
| | | | QFLANTTDQVKLEILPHHQTRLALKGPNSS | |
| GRIP 1 | 4539083 | 4 | TETTEVVLTADPVTGFGIQLOGSVFATETLSSPPLISYI | 682 |
| | | | EADSPAERCGVLQIGDRVMAINGIPTEDSTFEEASQL | |
| | | | LRDSSITSKVTLEIEFDVAES | |
| GRIP 1 | 4539083 | 5 | AESVIPSSGTFHVKLPKKHNVELGITISSPSSRKPGDP | 683 |
| | | | LVISDIKKGSVAHRTGTLELGDKLLAIDNIRLDNCSME | |
| | | | DAVQILQQCEDLVKLKIRKDEDNSD | |
| GRIP 1 | 4539083 | 6 | IYTVELKRYGGPLGITISGTEEPFDPIIISSLTKGGLAER | 684 |
| | | | TGAIHIGDRILAINSSSLKGKPLSEAIHLLQMAGETVTL | |
| | | | KIKKQTDAQSA | |
| GRIP 1 | 4539083 | 7 | IMSPTPVELHKVTLYKDSDMEDFGFSVADGLLEKGVY | 685 |
| | | | VKNIRPAGPGDLGGLKPYDRLLQVNHVRTRDFDCCL | |
| | | | VVPLIAESGNKLDLVISRNPLA | |
| GTPase | 2389008 | 1 | LALPRDGQGRLGFEVDAEGFVTHVERFTFAETAGLR | 686 |
| Activating | | | PGARLLRVCGQTLPSLRPEAAAQLLRSAPKVCVTV | |
| Enzyme | | | | |
| Guanine | 6650765 | 1 | AKAKWRQVVLQKASRESPLQFSLNGGSEKGFGIFVE | 687 |
| Exchange | | | GVEPGSKAADSGLKRGDQIMEVNGQNFENITFMKAV | |
| Factor | | | EILRNNTHLALTVKTNIFVFKEL | |
| HEMBA | 10436367 | 1 | LENVIAKSLLIKSNEGSYGFGLEDKNKVPIIKLVEKGSN | 688 |
| 1000505 | | | AEMAGMEVGKKIFAINGDLVFMRPFNEVDCFLKSCLN | |
| | | | SRKPLRVLVSTKP | |
| HEMBA | 10436367 | 2 | PRETVKIPDSADGLGFQIRGFGPSVVHAVGRGTVAAA | 689 |
| 1000505 | | | AGLHPGQCIIKVNGINVSKETHASVIAHVTACRKYRRP | |
| | | | TKQDSIQ | |
| HEMBA | 7022001 | 1 | EDFCYVFTVELERGPSGLGMGLIDGMHTHLGAPGLYI | 690 |
| 1003117 | | | QTLLPGSPAAADGRLSLGDRILEVNGSSLLGLGYLRA | |
| | | | VDLIRHGGKKMRFLVAKSDVETAKKI | |
| INADL | 2370148 | 1 | IWQIEYIDIERPSTGGLGFSVVALRSQNLGKVDIFVKD | 691 |
| | | | VQPGSVADRDQRLKENDQILAINHTPLDQNISHQQAI | |
| | | | ALLQQTTGSLRLIVAREPVHTKSSTSSSE | |
| INADL | 2370148 | 2 | PGHVEEVELINDGSGLGFGIVGGKTSGVVVRTIVPGG | 692 |
| | | | LADRDGRLQTGDHILKIGGTNVQGMTSEQVAQVLRN | |
| | | | CGNSS | |
| INADL | 2370148 | 3 | PGSDSSLFETYNVELVRKDGQSLGIRIVGYVGTSHTG | 693 |
| | | | EASGIYVKSIIPGSAAYHNGHIQVNDKIVAVDGVNIQG | |
| | | | FANHDVVEVLRNAGQVVHLTLVRRKTSSSTSRIHRD | |
| INADL | 2370148 | 4 | NSDDAELQKYSKLLPIHTLRLGVEVDSFDGHHYISSIV | 694 |
| | | | SGGPVDTLGLLQPEDELLEVNGMQLYGKSRREAVSF | |
| | | | LKEVPPPFTLVCCRRLFDDEAS | |
| INADL | 2370148 | 5 | LSSPEVKIVELVKDCKGLGFSILDYQDPLDPTRSVIVIR | 695 |
| | | | SLVADGVAERSGGLLPGDRLVSVNEYCLDNTSLAEA | |
| | | | VEILKAVPPGLVHLGICKPLVEFIVTD | |
| INADL | 2370148 | 6 | PNFSHWGPPRIVEIFREPNVSLGISIVVGQTVIKRLKN | 696 |
| | | | GEELKGIFIKQVLEDSPAGKTNALKTGDKILEVSGVDL | |
| | | | QNASHSEAVEAIKNAGNPVVFIVQSLSSTPRVIPNVH | |
| | | | NKANSS | |
| INADL | 2370148 | 7 | PGELHIIELEKDKNGLGLSLAGNKDRSRMSIFVVGINP | 697 |
| | | | EGPAAADGRMRIGDELLEINNQILYGRSHQNASAIIKT | |
| | | | APSKVKLVFIRNEDAVNQMANSS | |
| INADL | 2370148 | 8 | PATCPIVPGQEMIIEISKGRSGLGLSIVGGKDTPLNAIVI | 698 |
| | | | HEVYEEGAAARDGRLWAGDQILEVNGVDLRNSSHE | |
| | | | EAITALRQTPQKVRLVVY | |
| KIAA0147 | 1469875 | 1 | ILTLTILRQTGGLGISIAGGKGSTPYKGDDEGIFISRVS | 699 |
| | | | EEGPAARAGVRVGDKLLEVNGVALQGAEHHEAVEAL | |
| | | | RGAGTAVQMRVWRERMVEPENAEFIVTD | |
| KIAA0147 | 1469875 | 2 | PLRQRHVACLARSERGLGFSIAGGKGSTPYRAGDAG | 700 |
| | | | IFVSRIAEGGAAHRAGTLQVGDRVLSINGVDVTEARH | |
| | | | DHAVSLLTAASPTIALLLEREAGG | |
| KIAA0147 | 1469875 | 3 | ILEGPYPVEEIRLPRAGGPLGLSIVGGSDHSSHPFGV | 701 |
| | | | QEPGVFISKVLPRGLAARSGLRVGDRILAVNGQDVRD | |
| | | | ATHQEAVSALLRPCLELSLLVRRDPAEFIVTD | |
| KIAA0147 | 1469875 | 4 | RELCIQKAPGERLGISIRGGARGHAGNPRDPTDEGIFI | 702 |
| | | | SKVSPTGAAGRDGRLRVGLRLLEVNQQSLLGLTHGE | |
| | | | AVQLLRSVGDTLTVLVCDGFEASTDAALEVS | |
| KIAA0303 | 2224546 | 1 | PHQPIVIHSSGKNYGFTIRAIRVYVGDSDIYTVHHIVW | 703 |
| | | | NVEEGSPACQAGLKAGDLITHINGEPVHGLVHTEVIEL | |
| | | | LLKSGNKVSITTTPF | |
| KIAA0313 | 7657260 | 1 | ILACAAKAKRRLMTLTKPSREAPLPFILLGGSEKGFGI | 704 |
| | | | FVDSVDSGSKATEAGLKRGDQILEVNGQNFENIQLSK | |
| | | | AMEILRNNTHLSITVKTNLFVFKELLTNSS | |
| KIAA0316 | 6683123 | 1 | IPPAPRKVEMRRDPVLGFGFVAGSEKPVVVRSVTPG | 705 |
| | | | GPSEGKLIPGDQIVMINDEPVSAAPRERVIDLVRSCKE | |
| | | | SILLTVIQPYPSPK | |
| KIAA0340 | 2224620 | 1 | LNKRTTMPKDSGALLGLKVVGGKMTDLGRLGAFITKV | 706 |
| | | | KKGSLADVVGHLRAGDEVLEWNGKPLPGATNEEVY | |
| | | | NIILESKSEPQVEIIVSRPIGDIPRIHRD | |
| KIAA0380 | 2224700 | 1 | QRCVIIQKDQHGFGFTVSGDRIVLVQSVRPGGAAMK | 707 |
| | | | AGVKEGDRIIKVNGTMVTNSSHLEVVKLIKSGAYVALT | |
| | | | LLGSS | |
| KIAA0382 | 7662087 | 1 | ILVQRCVIIQKDDNGFGLTVSGDNPVFVQSVKEDGAA | 708 |
| | | | MRAGVQTGDRIIKVNGTLVTHSNHLEVVKLIKSGSYV | |
| | | | ALTVQGRPPGNSS | |
| KIAA0440 | 2662160 | 1 | SVEMTLRRNGLGQLGFHVNYEGIVADVEPYGYAWQ | 709 |
| | | | AGLRQGSRLVEICKVAVATLSHEQMIDLLRTSVTVKV | |
| | | | VIIPPHD | |
| KIAA0545 | 14762850 | 1 | LKVMTSGWETVDMTLRRNGLGQLGFHVKYDGTVAE | 710 |
| | | | VEDYGFAWQAGLRQGSRLVEICKVAVVTLTHDQMID | |
| | | | LLRTSVTVKVVIIPPFEDGTPRRGW | |
| KIAA0559 | 3043641 | 1 | HYIFPHARIKITRDSKDHTVSGNGLGIRIVGGKEIPGHS | 711 |
| | | | GEIGAYIAKILPGGSAEQTGKLMEGMQVLEWNGIPLT | |
| | | | SKTYEEVQSIISQQSGEAEICVRLDLNML | |
| KIAA0561 | 3043645 | 1 | LCGSLRPPIVIHSSGKKYGFSLRAIRVYMGDSDVYTV | 712 |
| | | | HHVVWSVEDGSPAQEAGLRAGDLITHINGESVLGLV | |
| | | | HMDVVELLLKSGNKISLRTTALENTSIKVG | |
| KIAA0613 | 3327039 | 1 | SYSVTLTGPGPWGFRLQGGKDFNMPLTISRITPGSKA | 713 |
| | | | AQSQLSQGDLVVAIDGVNTDTMTHLEAQNKIKSASYN | |
| | | | LSLTLQKSKNSS | |
| KIAA0751 | 12734165 | 1 | ISRDSGAMLGLKVVGGKMTESGRLCAFITKVKKGSLA | 714 |
| | | | DTVGHLRPGDEVLEWNGRLLQGATFEEVYNIILESKP | |
| | | | EPQVELVVSRPIAIHRD | |
| KIAA0807 | 3882334 | 1 | ISALGSMRPPIIIHRAGKKYGFTLRAIRVYMGDSDVYT | 715 |
| | | | VHHMVWHVEDGGPASEAGLROGDLITHVNGEPVHG | |
| | | | LVHTEVVELILKSGNKVAISTTPLENSS | |
| KIAA0858 | 4240204 | 1 | FSDMRISINQTPGKSLDFGFTIKWDIPGIFVASVEAGS | 716 |
| | | | PAEFSQLQVDDEIIAINNTKFSYNDSKEWEEAMAKAQ | |
| | | | ETGHLVMDVRRYGKAGSPE | |
| KIAA0902 | 4240292 | 1 | QSAHLEVIQLANIKPSEGLGMYIKSTYDGLHVITGTTE | 717 |
| | | | NSPADRCKKIHAGDEVIQVNHQTVVGWQLKNLVNAL | |
| | | | REDPSGVILTLKKRPQSMLTSAPA | |
| KIAA0967 | 4589577 | 1 | ILTQTLIPVRHTVKIDKDTLLQDYGFHISESLPLTVVAVT | 718 |
| | | | AGGSAHGKLFPGDQILQMNNEPAEDLSWERAVDILR | |
| | | | EAEDSLSITVVRCTSGVPKSSNSS | |
| KIAA0973 | 4589589 | 1 | GLRSPITIQRSGKKYGFTLRAIRVYMGDTDVYSVHHIV | 719 |
| | | | WHVEEGGPAQEAGLCAGDLITHVNGEPVHGMVHPE | |
| | | | VVELILKSGNKVAVTTTPFE | |
| KIAA1095 | 5889526 | 1 | QGEETKSLTLVLHRDSGSLGFNIIGGRPSVDNHDGSS | 720 |
| | | | SEGIFVSKIVDSGPAAKEGGLQIHDRIIEVNGRDLSRA | |
| | | | THDQAVEAFKTAKEPIVVQVLRRTPRTKMFTP | |
| KIAA1095 | 5889526 | 2 | QEMDREELELEEVDLYRMNSQDKLGLTVCYRTDDED | 721 |
| | | | DIGIYISEIDPNSIAAKDGRIREGDRIIQINGIEVQNREE | |
| | | | AVALLTSEENKNFSLLIARPELQLD | |
| KIAA1202 | 6330421 | 1 | RSFQYVPVQLQGGAPWGFTLKGGLEHCEPLTVSKIE | 722 |
| | | | DGGKAALSQKMRTGDELVNINGTPLYGSRQEALILIK | |
| | | | GSFRILKLIVRRRNAPVS | |
| KIAA1222 | 6330610 | 1 | ILEKLELFPVELEKDEDGLGISIIGMGVGADAGLEKLGI | 723 |
| | | | FVKTVTEGGAAQRDGRIQVNDQIVEVDGISLVGVTQN | |
| | | | FAATVLRNTKGNVRFVIGREKPGQVS | |
| KIAA1284 | 6331369 | 1 | KDVNVYVNPKKLTVIKAKEQLKLLEVLVGIIHQTKWS | 724 |
| | | | WRRTGKQGDGERLVVHGLLPGGSAMKSGQVLIGDV | |
| | | | LVAVNDVDVTTENIERVLSCIPGPMQVKLTFENAYDV | |
| | | | KRET | |
| KIAA1389 | 7243158 | 1 | TRGCETVEMTLRRNGLGQLGFHVNFEGIVADVEPFG | 725 |
| | | | FAWKAGLRQGSRLVEICKVAVATLTHEQMIDLLRTSV | |
| | | | TVKVVIIQPHDDGSPRR | |
| KIAA1415 | 7243210 | 1 | VENILAKRLLILPQEEDYGFDIEEKNKAVVVKSVQRGS | 726 |
| | | | LAEVAGLQVGRKIYSINEDLVFLRPFSEVESILNQSFC | |
| | | | SRRPLRLLVATKAKEIIKIP | |
| KIAA1526 | 5817166 | 1 | PDSAGPGEVRLVSLRRAKAHEGLGFSIRGGSEHGVG | 727 |
| | | | IYVSLVEPGSLAEKEGLRVGDQILRVNDKSLARVTHA | |
| | | | EAVKALKGSKKLVLSVYSAGRIPGGYVTNH | |
| KIAA1526 | 5817166 | 2 | LQGGDEKKVNLVLGDGRSLGLTIRGGAEYGLGIYITG | 728 |
| | | | VDPGSEAEGSGLKVGDQILEVNWRSFLNILHDEAVRL | |
| | | | LKSSRHLILTVKDVGRLPHARTTVDE | |
| KIAA1526 | 5817166 | 3 | WTSGAHVHSGPCEEKCGHPGHRQPLPRIVTIQRGGS | 729 |
| | | | AHNCGQLKVGHVILEVNGLTLRGKEHREAARIIAEAFK | |
| | | | TKDRDYIDFLDSL | |
| KIAA1620 | 10047316 | 1 | ELRRAELVEIIVETEAQTGVSGINVAGGGKEGIFVREL | 730 |
| | | | REDSPAARSLSLQEGDQLLSARVFFENFKYEDALRLL | |
| | | | QCAEPYKVSFCLKRTVPTGDLALRP | |
| KIAA1634 | 10047344 | 1 | PSQLKGVLVRASLKKSTMGFGFTIIGGDRPDEFLQVK | 731 |
| | | | NVLKDGPAAQDGKIAPGDVIVDINGNCVLGHTHADVV | |
| | | | QMFQLVPVNQYVNLTLCRGYPLPDDSED | |
| KIAA1634 | 10047344 | 2 | ASSGSSQPELVTIPLIKGPKGFGFAIADSPTGQKVKMI | 732 |
| | | | LDSQWCQGLQKGDIIKEIYHQNVQNLTHLQVVEVLKQ | |
| | | | FPVGADVPLLILRGGPPSPTKTAKM | |
| KIAA1634 | 10047344 | 3 | LYEDKPPLTNTFLISNPRTTADPRILYEDKPPNTKDLD | 733 |
| | | | VFLRKQESGFGFRVLGGDGPDQSIYIGAIIPLGAAEKD | |
| | | | GRLRAADELMCIDGIPVKGKSHKQVLDLMTTAARNG | |
| | | | HVLLTVRRKIFYGEKQPEDDSGSPGIHRELT | |
| KIAA1634 | 10047344 | 4 | PAPQEPYDVVLQRKENEGFGFVILTSKNKPPPGVIPH | 734 |
| | | | KIGRVIEGSPADRCGKLKVGDHISAVNGQSIVELSHD | |
| | | | NIVQLIKDAGVTVTLTVIAEEEHHGPPS | |
| KIAA1634 | 10047344 | 5 | QNLGCYPVELERGPRGFGFSLRGGKEYNMGLFILRL | 735 |
| | | | AEDGPAIKDGRIHVGDQIVEINGEPTQGITHTRAIELIQ | |
| | | | AGGNKVLLLLRPGTGLIPDHGLA | |
| KIAA1719 | 1267982 | 0 | ITVVELIKKEGSTLGLTISGGTDKDGKPRVSNLRPGGL | 736 |
| | | | AARSDLLNIGDYIRSVNGIHLTRLRHDEIITLLKNVGER | |
| | | | VVLEVEY | |
| KIAA1719 | 1267982 | 1 | ILDVSLYKEGNSFGFVLRGGAHEDGHKSRPLVLTYVR | 737 |
| | | | PGGPADREGSLKVGDRLLSVDGIPLHGASHATALATL | |
| | | | RQCSHEALFQVEYDVATP | |
| KIAA1719 | 1267982 | 2 | IHTVANASGPLMVEIVKTPGSALGISLTTTSLRNKSVITI | 738 |
| | | | DRIKPASVVDRSGALHPGDHILSIDGTSMEHCSLLEAT | |
| | | | KLLASISEKVRLEILPVPQSQRPL | |
| KIAA1719 | 1267982 | 3 | IQIVHTETTEVVLCGDPLSGFGLQLQGGIFATETLSSP | 739 |
| | | | PLVCFIEPDSPAERCGLLQVGDRVLSINGIATEDGTME | |
| | | | EANQLLRDAALAHKVVLEVEFDVAESV | |
| KIAA1719 | 1267982 | 4 | IQFDVAESVIPSSGTFHVKLPKKRSVELGITISSASRKR | 740 |
| | | | GEPLIISDIKKGSVAHRTGTLEPGDKLLAIDNIRLDNCP | |
| | | | MEDAVQILRQCEDLVKLKIRKDEDN | |
| KIAA1719 | 1267982 | 5 | IQTTGAVSYTVELKRYGGPLGITISGTEEPFDPIVISGL | 741 |
| | | | TKRGLAERTGAIHVGDRILAINNVSLKGRPLSEAIHLL | |
| | | | QVAGETVTLKIKKQLDR | |
| KIAA1719 | 1267982 | 6 | ILEMEELLLPTPLEMHKVTLHKDPMRHDFGFSVSDGL | 742 |
| | | | LEKGVYVHTVRPDGPAHRGGLQPFDRVLQVNHVRT | |
| | | | RDFDCCLAVPLLAEAGDVLELIISRKPHTAHSS | |
| LIM | 12734250 | 1 | MALTVDVAGPAPWGFRITGGRDFHTPIMVTKVAERG | 743 |
| Mystique | | | KAKDADLRPGDIIVAINGESAEGMLHAEAQSKIRQSPS | |
| | | | PLRLQLDRSQATSPGQT | |
| LIM Protein | 3108092 | 1 | SNYSVSLVGPAPWGFRLQGGKDFNMPLTISSLKDGG | 744 |
| | | | KAAQANVRIGDVVLSIDGINAQGMTHLEAQNKIKGCT | |
| | | | GSLNMTLQRAS | |
| LIM-RIL | 1085021 | 1 | IHSVTLRGPSPWGFRLVGRDFSAPLTISRVHAGSKAS | 745 |
| | | | LAALCPGDLIQAINGESTELMTHLEAQNRIKGCHDHLT | |
| | | | LSVSRPE | |
| LIMK1 | 4587498 | 1 | TLVEHSKLYCGHCYYQTVVTPVIEQILPDSPGSHLPHT | 746 |
| | | | VTLVSIPASSHGKRGLSVSIDPPHGPPGCGTEHSHTV | |
| | | | RVQGVDPGCMSPDVKNSIHVGDRILEINGTPIRNVPL | |
| | | | DEIDLLIQETSRLLQLTLEHD | |
| LIMK2 | 1805593 | 1 | PYSVTLISMPATTEGRRGFSVSVESACSNYATTVQVK | 747 |
| | | | EVNRMHISPNNRNAIHPGDRILEINGTPVRTLRVEEVE | |
| | | | DAISQTSQTLQLLIEHD | |
| LU-1 | U52111 | 1 | VCYRTDDEEDLGIYVGEVNPNSIAAKDGRIREGDRIIQI | 748 |
| | (acc. #) | | NGVDVQNREEAVAILSQEENTNISLLVARPESQLA | |
| MINT1 | 2625024 | 1 | SENCKdVFIEKQKGEILGVVIVESGWGSILPTVIIANMM | 749 |
| | | | HGGPAEKSGKLNIGDQIMSINGTSLVGLPLSTCQSIIK | |
| | | | GLKNQSRVKLNIVRCPPVNSS | |
| MINT1 | 2625024 | 2 | LRCPPVTTVLIRRPDLRYQLGFSVQNGIICSLMRGGIA | 750 |
| | | | ERGGVRVGHRIIEINGQSVVATPHEKIVHILSNAVGEIH | |
| | | | MKTMPAAMYRLLNSS | |
| MINT3 | 3169808 | 1 | LSNSDNCREVHLEKRRGEGLGVALVESGWGSLLPTA | 751 |
| | | | VIANLLHGGPAERSGALSIGDRLTAINGTSLVGLPLAA | |
| | | | CQAAVRETKSQTSVTLSIVHCPPVTTAIM | |
| MINT3 | 3169808 | 2 | LVHCPPVTTAIIHRPHAREQLGFCVEDGIICSLLRGGIA | 752 |
| | | | ERGGIRVGHRIIEINGQSVVATPHARIIELLTEAYGEVHI | |
| | | | KTMPAATYRLLTG | |
| MPP1 | 189785 | 1 | RKVRLIQFEKVTEEPMGITLKLNEKQSCTVARILHGGM | 753 |
| | | | IHRQGSLHVGDEILEINGTNVTNHSVDQLQKAMKETK | |
| | | | GMISLKVIPNQ | |
| MPP2 | 939884 | 1 | PVPPDAVRMVGIRKTAGEHLGVTFRVEGGELVIARIL | 754 |
| | | | HGGMVAQQGLLHVGDIIKEVNGQPVGSDPRALQELL | |
| | | | RNASGSVILKILPNYQ | |
| MUPP1 | 2104784 | 1 | QGRHVEVFELLKPPSGGLGFSVVGLRSENRGELGIF | 755 |
| | | | VQEIQEGSVAHRDGRLKETDQILAINGQALDQTITHQ | |
| | | | QAISILQKAKDTVQLVIARGSLPQLV | |
| MUPP1 | 2104784 | 2 | PVHWQHMETIELVNDGSGLGFGIIGGKATGVIVKTILP | 756 |
| | | | GGVADQHGRLCSGDHILKIGDTDLAGMSSEQVAQVL | |
| | | | RQCGNRVKLMIARGAIEERTAPT | |
| MUPP1 | 2104784 | 3 | QESETFDVELTKNVQGLGITIAGYIGDKKLEPSGIFVK | 757 |
| | | | SITKSSAVEHDGRIQIGDQIIAVDGTNLQGFTNQQAVE | |
| | | | VLRHTGQTVLLTLMRRGMKQEA | |
| MUPP1 | 2104784 | 4 | LNYEIVVAHVSKFSENSGLGISLEATVGHHFIRSVLPE | 758 |
| | | | GPVGHSGKLFSGDELLEVNGITLLGENHQDVVNILKE | |
| | | | LPIEVTMVCCRRTVPPT | |
| MUPP1 | 2104784 | 5 | WEAGIQHIELEKGSKGLGFSILDYQDPIDPASTVIIIRSL | 759 |
| | | | VPGGIAEKDGRLLPGDRLMFVNDVNLENSSLEEAVE | |
| | | | ALKGAPSGTVRIGVAKPLPLSPEE | |
| MUPP1 | 2104784 | 6 | RNVSKESFERTINIAKGNSSLGMTVSANKDGLGMIVR | 760 |
| | | | SIIHGGAISRDGRIAIGDCILSINEESTISVTNAQARAML | |
| | | | RRHSLIGPDIKITYVPAEHLEE | |
| MUPP1 | 2104784 | 7 | LNWNQPRRVELWREPSKSLGISIVGGRGMGSRLSN | 761 |
| | | | GEVMRGIFIKHVLEDSPAGKNGTLKPGDRIVEVDGMD | |
| | | | LRDASHEQAVEAIRKAGNPVVFMVQSIINRPRKSPLP | |
| | | | SLL | |
| MUPP1 | 2104784 | 8 | LTGELHMIELEKGHSGLGLSLAGNKDRSRMSVFIVGI | 762 |
| | | | DPNGAAGKDGRLQIADELLEINGQILYGRSHQNASSII | |
| | | | KCAPSKVKIIFIRNKDAVNQ | |
| MUPP1 | 2104784 | 9 | LSSFKNVQHLELPKDQGGLGIAISEEDTLSGVIIKSLTE | 763 |
| | | | HGVAATDGRLKVGDQILAVDDEIVVGYPIEKFISLLKT | |
| | | | AKMTVKLTIHAENPDSQ | |
| MUPP1 | 2104784 | 10 | LPGCETTIEISKGRTGLGLSIVGGSDTLLGAIIIHEVYEE | 764 |
| | | | GAACKDGRLWAGDQILEVNGIDLRKATHDEAINVLRQ | |
| | | | TPQRVRLTLYRDEAPYKE | |
| MUPP1 | 2104784 | 11 | KEEEVCDTLTIELQKKPGKGLGLSIVGKRNDTGVFVS | 765 |
| | | | DIVKGGIADADGRLMQGDQILMVNGEDVRNATQEAV | |
| | | | AALLKCSLGTVTLEVGRIKAGPFHS | |
| MUPP1 | 2104784 | 12 | LQGLRTVEMKKGPTDSLGISIAGGVGSPLGDVPIFIAM | 766 |
| | | | MHPTGVAAQTQKLRVGDRIVTICGTSTEGMTHTQAV | |
| | | | NLLKNASGSIEMQVVAGGDVSV | |
| MUPP1 | 2104784 | 13 | LGPPQCKSITLERGPDGLGFSIVGGYGSPHGDLPIYV | 767 |
| | | | KTVFAKGAASEDGRLKRGDQIIAVNGQSLEGVTHEEA | |
| | | | VAILKRTKGTVTLMVLS | |
| NeDLG | 10863920 | 1 | IQYEEIVLERGNSGLGFSIAGGIDNPHVPDDPGIFITKII | 768 |
| | | | PGGAAAMDGRLGVNDCVLRVNEVEVSEVVHSRAVE | |
| | | | ALKEAGPVVRLVVRRRQN | |
| NeDLG | 10863920 | 2 | ITLLKGPKGLGFSIAGGIGNQHIPGDNSIYITKIIEGGAA | 769 |
| | | | QKDGRLQIGDRLLAVNNTNLQDVRHEEAVASLKNTS | |
| | | | DMVYLKVAKPGSLE | |
| NeDLG | 10863920 | 3 | ILLHKGSTGLGFNIVGGEDGEGIFVSFILAGGPADLSG | 770 |
| | | | ELRRGDRILSVNGVNLRNATHEQAAAALKRAGQSVTI | |
| | | | VAQYRPEEYSRFESKIHDLREQMMNSSMSSGSGSLR | |
| | | | TSEKRSLE | |
| NOS1 | 642525 | 1 | IQPNVISVRLFKRKVGGLGFLVKERVSKPPVIISDLIRG | 771 |
| | | | GAAEQSGLIQAGDIILAVNGRPLVDLSYDSALEVLRGI | |
| | | | ASETHVVLILRGP | |
| novel PDZ | 7228177 | 1 | QANSDESDIIHSVRVEKSPAGRLGFSVRGGSEHGLGI | 772 |
| gene | | | FVSKVEEGSSAERAGLCVGDKITEVNGLSLESTTMGS | |
| | | | AVKVLTSSSRLHMMVRRMGRVPGIKFSKEKNSS | |
| novel PDZ | 7228177 | 2 | PSDTSSEDGVRRIVHLYTTSDDFCLGFNIRGGKEFGL | 773 |
| gene | | | GIYVSKVDHGGLAEENGIKVGDQVLAANGVRFDDISH | |
| | | | SQAVEVLKGQTHIMLTIKETGRYPAYKEMNSS | |
| Novel Serine | 1621243 | 1 | KIKKFLTESHDRQAKGKAITKKKYIGIRMMSLTSSKAK | 774 |
| Protease | | | ELKDRHRDFPDVISGAYIIEVIPDTPAEAGGLKENDVII | |
| | | | SINGQSVVSANDVSDVIKRESTLNMVVRRGNEDIMIT | |
| | | | V | |
| Outer | 7023825 | 1 | LLTEEEINLTRGPSGLGFNIVGGTDQQYVSNDSGIYVS | 775 |
| Membrane | | | RIKENGAAALDGRLQEGDKILSVNGQDLKNLLHQDAV | |
| | | | DLFRNAGYAVSLRVQHRLQVQNGIHS | |
| p55T | 12733367 | 1 | PVDAIRILGIHKRAGEPLGVTFRVENNDLVIARILHGG | 776 |
| | | | MIDRQGLLHVGDIIKEVNGHEVGNNPKELQELLKNISG | |
| | | | SVTLKILPSYRDTITPQQ | |
| PAR3 | 8037914 | 1 | DDMVKLVEVPNDGGPLGIHVVPFSARGGRTLGLLVK | 777 |
| | | | RLEKGGKAEHENLFRENDCIVRINDGDLRNRRFEQA | |
| | | | QHMFRQAMRTPIIWFHVVPAA | |
| PAR3 | 8037914 | 2 | GKRLNIQLKKGTEGLGFSITSRDVTIGGSAPIYVKNILP | 778 |
| | | | RGAAIQDGRLKAGDRLIEVNGVDLVGKSQEEVVSLLR | |
| | | | STKMEGTVSLLVFRQEDA | |
| PAR3 | 8037914 | 3 | TPDGTREFLTFEVPLNDSGSAGLGVSVKGNRSKENH | 779 |
| | | | ADLGIFVKSIINGGAASKDGRLRVNDQLIAVNGESLLG | |
| | | | KTNQDAMETLRRSMSTEGNKRGMIQLIVA | |
| PAR6 | 2613011 | 1 | LPETHRRVRLHKHGSDRPLGFYIRDGMSVRVAPQGL | 780 |
| | | | ERVPGIFISRLVRGGLAESTGLLAVSDEILEVNGIEVA | |
| | | | GKTLDQVTDMMVANSHNLIVTVKPANQR | |
| PAR6 | 13537118 | 1 | IDVDLVPETHRRVRLHRHGCEKPLGFYIRDGASVRVT | 781 |
| GAMMA | | | PHGLEKVPGIFISRMVPGGLAESTGLLAVNDEVLEVN | |
| | | | GIEVAGKTLDQVTDMMIANSHNLIVTVKPANQRNNVV | |
| PDZ-73 | 5031978 | 1 | RSRKLKEVRLDRLHPEGLGLSVRGGLEFGCGLFISHL | 782 |
| | | | IKGGQADSVGLQVGDEIVRINGYSISSCTHEEVINLIRT | |
| | | | KKTVSIKVRHIGLIPVKSSPDEFH | |
| PDZ-73 | 5031978 | 2 | IPGNRENKEKKVFISLVGSRGLGCSISSGPIQKPGIFIS | 783 |
| | | | HVKPGSLSAEVGLEIGDQIVEVNGVDFSNLDHKEAVN | |
| | | | VLKSSRSLTISIVAAAGRELFMTDEF | |
| PDZ-73 | 5031978 | 3 | PEQIMGKDVRLLRIKKEGSLDLALEGGVDSPIGKVVV | 784 |
| | | | SAVYERGAAERHGGIVKGDEIMAINGKIVTDYTLAEAD | |
| | | | AALQKAWNQGGDWIDLVVAVCPPKEYDD | |
| PDZK1 | 2944188 | 1 | LTSTFNPRECKLSKQEGQNYGFFLRIEKDTEGHLVRV | 785 |
| | | | VEKCSPAEKAGLQDGDRVLRINGVFVDKEEHMQVVD | |
| | | | LVRKSGNSVTLLVLDGDSYEKAGSPGIHRD | |
| PDZK1 | 2944188 | 2 | RLCYLVKEGGSYGFSLKTVQGKKGVYMTDITPQGVA | 786 |
| | | | MRAGVLADDHLIEVNGENVEDASHEEVVEKVKKSGS | |
| | | | RVMFLLVDKETDKREFIVTD | |
| PDZK1 | 2944188 | 3 | QFKRETASLKLLPHQPRIVEMKKGSNGYGFYLRAGS | 787 |
| | | | EQKGQIIKDIDSGSPAEEAGLKNNDLVVAVNGESVET | |
| | | | LDHDSWEMIRKGGDQTSLLVVDKETDNMYRLAEFIV | |
| | | | TD | |
| PDZK1 | 2944188 | 4 | PDTTEEVDHKPKLCRLAKGENGYGFHLNAIRGLPGSF | 788 |
| | | | IKEVQKGGPADLAGLEDEDVIIEVNGVNVLDEPYEKV | |
| | | | VDRIQSSGKNVTLLVZGKNSS | |
| PICK1 | 4678411 | 1 | PTVPGKVTLQKDAQNLIGISIGGGAQYCPCLYIVQVFD | 789 |
| | | | NTPAALDGTVAAGDEITGVNGRSIKGKTKVEVAKMIQ | |
| | | | EVKGEVTIHYNKLQ | |
| PIST | 98374330 | 1 | SQGVGPIRKVLLLKEDHEGLGISITGGKEHGVPILISEI | 790 |
| | | | HPGQPADRCGGLHVGDAILAVNGVNLRDTKHKEAVTI | |
| | | | LSQQRGEIEFEVVYVAPEVDSD | |
| prlL16 | 1478492 | 1 | IHVTILHKEEGAGLGFSLAGGADLENKVITVHRVFPNG | 791 |
| | | | LASQEGTIQKGNEVLSINGKSLKGTTHHDALAILRQAR | |
| | | | EPRQAVIVTRKLTPEEFIVTD | |
| prlL16 | 1478492 | 2 | TAEATVCTVTLEKMSAGLGFSLEGGKGSLHGDKPLTI | 792 |
| | | | NRIFKGAASEQSETVQPGDEILQLGGTAMQGLTRFEA | |
| | | | WNIIKALPDGPVTIVIRRKSLQSK | |
| PSD95 | 3318652 | 1 | LEYEeITLERGNSGLGFSIAGGTDNPHIGDDPSIFITKII | 793 |
| | | | PGGAAAQDGRLRVNDSILFVNEVDVREVTHSAAVEA | |
| | | | LKEAGSIVRLYVMRRKPPAENSS | |
| PSD95 | 3318652 | 2 | HVMRRKPPAEKVMEIKLIKGPKGLGFSIAGGVGNQHI | 794 |
| | | | PGDNSIYVTKIIEGGAAHKDGRLQIGDKILAVNSVGLE | |
| | | | DVMHEDAVAALKNTYDVVYLKVAKPSNAYL | |
| PSD95 | 3318652 | 3 | REDIPREPRRIVIHRGSTGLGFNIVGGEDGEGIFISFIL | 795 |
| | | | AGGPADLSGELRKGDQILSVNGVDLRNASHEQAAIAL | |
| | | | KNAGQTVTIIAQYKPEFIVTD | |
| PTN-3 | 179912 | 1 | LIRITPDEDGKFGFNLKGGVDQKMPLVVSRINPESPA | 796 |
| | | | DTCIPKLNEGDQIVLINGRDISEHTHDQVVMFIKASRE | |
| | | | SHSRELALVIRRR | |
| PTN-4 | 190747 | 1 | IRMKPDENGRFGFNVKGGYDQKMPVIVSRVAPGTPA | 797 |
| | | | DLCVPRLNEGDQVVLINGRDIAEHTHDQVVLFIKASC | |
| | | | ERHSGELMLLVRPNA | |
| PTPL1 | 515030 | 1 | PEREITLVNLKKDAKYGLGFQIIGGEKMGRLDLGIFISS | 798 |
| | | | VAPGGPADFHGCLKPGDRLISVNSVSLEGVSHHAAIE | |
| | | | ILQNAPEDVTLVISQPKEKISKVPSTPVHL | |
| PTPL1 | 515030 | 2 | GDIFEVELAKNDNSLGISVTGGVNTSVRHGGIYVKAVI | 799 |
| | | | PQGAAESDGRIHKGDRVLAVNGVSLEGATHKQAVET | |
| | | | LRNTGQVVHLLLEKGQSPTSK | |
| PTPL1 | 515030 | 3 | TEENTFEVKLFKNSSGLGFSFSREDNLIPEQINASIVR | 800 |
| | | | VKKLFAGQPAAESGKIDVGDVILKVNGASLKGLSQQE | |
| | | | VISALRGTAPEVFLLLCRPPPGVLPEIDT | |
| PTPL1 | 515030 | 4 | ELEVELLITLIKSEKASLGFTVTKGNQRIGCYVHDVIQD | 801 |
| | | | PAKSDGRLKPGDRLIKVNDTDVTNMTHTDAVNLLRAA | |
| | | | SKTVRLVIGRVLELPRIPMLPH | |
| PTPL1 | 515030 | 5 | MLPHLLPDITLTCNKEELGFSLCGGHDSLYQVVYISDI | 802 |
| | | | NPRSVAAIEGNLQLLDVIHYVNGVSTQGMTLEEVNRA | |
| | | | LDMSLPSLVLKATRNDLPV | |
| RGS12 | 3290015 | 1 | RPSPPRVRSVEVARGRAGYGFTLSGQAPCVLSCVM | 803 |
| | | | RGSPADFVGLRAGDQILAVNEINVKKASHEDVVKLIG | |
| | | | KCSGVLHMVIAEGVGRFESCS | |
| Rhophilin- | 14279408 | 1 | ISFSANKRWTPPRSIRFTAEEGDLGFTLRGNAPVQVH | 804 |
| like | | | FLDPYCSASVAGAREGDYIVSIQLVDCKWLTLSEVMK | |
| | | | LLKSFGEDEIEMKVVSLLDSTSSMHNKSAT | |
| Serine | 2738914 | 1 | RGEKKNSSSGISGSQRRYIGVMMLTLSPSILAELQLR | 805 |
| Protease | | | EPSFPDVQHGVLIHKVILGSPAHRAGLRPGDVILAIGE | |
| | | | QMVQNAEDVYEAVRTQSQLAVQIRRGRETLTLYV | |
| Shank 1 | 6049185 | 1 | EEKTVVLQKKDNEGFGFVLRGAKADTPIEEFTPTPAF | 806 |
| | | | PALQYLESVDEGGVAWQAGLRTGDFLIEVNNENVVK | |
| | | | VGHRQVVNMIRQGGNHLVLKVVTVTRNLDPDDTARK | |
| | | | KA | |
| Shank 3 | * | 1 | SDYVIDDKVAVLQKRDHEGFGFVLRGAKAETPIEEFT | 807 |
| | | | PTPAFPALQYLESVDVEGVAWRAGLRTGDFLIEVNG | |
| | | | VNVVKVGHKQVVALIRQGGNRLVMKVVSVTRKPEED | |
| | | | G | |
| SIP1 | 2047327 | 1 | IRLCRLVRGEQGYGFHLHGEKGRRGQFIRRVEPGSP | 808 |
| | | | AEAAALRAGDRLVEVNGVNVEGETHHQVVQRIKAVE | |
| | | | GQTRLLVVDQN | |
| SIP1 | 2047327 | 2 | IRHLRKGPQGYGFNLHSDKSRPGQYIRSVDPGSPAA | 809 |
| | | | RSGLRAQDRLIEVNGQNVEGLRHAEVVASIKAREDEA | |
| | | | RLLVVDPETDE | |
| SITAC-18 | 8886071 | 1 | PGVREIHLCKDERGKTGLRLRKVDQGLFVQLVQANT | 810 |
| | | | PASLVGLRFGDQLLQIDGRDCAGWSSHKAHQVVKKA | |
| | | | SGDKIVVVVRDRPFQRTVTM | |
| SITAC-18 | 8886071 | 2 | PFQRTVTMHKDSMGHVGFVIKKGKIVSLVKGSSAAR | 811 |
| | | | NGLLTNHYVCEVDGQNVIGLKDKKIMEILATAGNVVTL | |
| | | | TIIPSVIYEHIVEFIV | |
| SYNTENIN | 2795862 | 1 | LEIKQGIREVILCKDQDGKIGLRLKSIDNGIFVQLVQAN | 812 |
| | | | SPASLVGLRFGDQVLQINGENCAGWSSDKAHKVLKQ | |
| | | | AFGEKITMRIHRD | |
| SYNTENIN | 2795862 | 2 | RDRPFERTITMHKDSTGHVGFIFKNGKITSIVKDSSAA | 813 |
| | | | RNGLLTEHNICEINGQNVIGLKDSQIADILSTSGNSS | |
| Syntrophin 1 | 1145727 | 1 | QRRRVTVRKADAGGLGISIKGGRENKMPILISKIFKGL | 814 |
| alpha | | | AADQTEALFVGDAILSVNGEDLSSATHDEAVQVLKKT | |
| | | | GKEVVLEVKYMKDVSPYFK | |
| Syntrophin | 476700 | 1 | IRVVKQEAGGLGISIKGGRENRMPILISKIFPGLAADQS | 815 |
| beta 2 | | | RALRLGDAILSVNGTDLRQATHDQAVQALKRAGKEVL | |
| | | | LEVKFIREFIVTD | |
| Syntrophin | 9507162 | 1 | EPFYSGERTVTIRRQTVGGFGLSIKGGAEHNIPVVVS | 816 |
| gamma 1 | | | KISKEQRAELSGLLFIGDAILQINGINVRKCRHEEVVQV | |
| | | | LRNAGEEVTLTVSFLKRAPAFLKLP | |
| Syntrophin | 9507164 | 1 | SHQGRNRRTVTLRRQPVGGLGLSIKGGSEHNVPVVI | 817 |
| gamma 2 | | | SKIFEDQAADQTGMLFVGDAVLQVNGIHVENATHEEV | |
| | | | VHLLRNAGDEVTITVEYLREAPAFLK | |
| TAX2-like | 3253116 | 1 | RGETKEVEVTKTEDALGLTITDNGAGYAFIKRIKEGSII | 818 |
| protein | | | NRIEAVCVGDSIEAINDHSIVGCRHYEVAKMLRELPKS | |
| | | | QPFTLRLVQPKRAF | |
| TIAM 1 | 4507500 | 1 | HSIHIEKSDTAADTYGFSLSSVEEDGIRRLYVNSVKET | 819 |
| | | | GLASKKGLKAGDEILEINNRAADALNSSMLKDFLSQP | |
| | | | SLGLLVRTYPELE | |
| TIAM 2 | 6912703 | 1 | PLNVYDVQLTKTGSVCDFGFAVTAQVDERQHLSRIFI | 820 |
| | | | SDVLPDGLAYGEGLRKGNEIMTLNGEAVSDLDLKQM | |
| | | | EALFSEKSVGLTLIARPPDTKATL | |
| TIP1 | 2613001 | 1 | QRVEIHKLRQGENLILGFSIGGGIDQDPSQNPFSEDK | 821 |
| | | | TDKGIYVTRVSEGGPAEIAGLQIGDKIMQVNGWDMT | |
| | | | MVTHDQARKRLTKRSEEVVRLLVTRQSLQK | |
| TIP2 | 2613003 | 1 | RKEVEVFKSEDALGLTITDNGAGYAFIKRIKEGSVIDHI | 822 |
| | | | HLISVGDMIEAINGQSLLGCRHYEVARLLKELPRGRTF | |
| | | | TLKLTEPRK | |
| TIP33 | 2613007 | 1 | HSHPRVVELPKTDEGLGFNVMGGKEQNSPIYISRIIPG | 823 |
| | | | GVAERHGGLKRGDQLLSVNGVSVEGEHHEKAVELLK | |
| | | | AAKDSVKLVVRYTPKVL | |
| TIP43 | 2613011 | 1 | ISNQKRGVKVLKQELGGLGISIKGGKENKMPILISKIFK | 824 |
| | | | GLAADQTQALYVGDAILSVNGADLRDATHDEAVQALK | |
| | | | RAGKEVLLEVKYMREATPYV | |
| X-11 beta | 3005559 | 1 | IHFSNSENCKELQLEKHKGEILGVVVVESGWGSILPT | 825 |
| | | | VILANMMNGGPAARSGKLSIGDQIMSINGTSLVGLPLA | |
| | | | TCQGIIKGLKNQTQVKLNIVSCPPVTTVLIKRNSS | |
| X-11 beta | 3005559 | 2 | IPPVTTVLIKRPDLKYQLGFSVQNGIICSLMRGGIAERG | 826 |
| | | | GVRVGHRIIEINGQSVVATAHEKIVQALSNSVGEIHMK | |
| | | | TMPPAMFRLLTGQENSS | |
| ZO-1 | 292937 | 1 | IWEQHTVTLHRAPGFGFGIAISGGRDNPHFQSGETSI | 827 |
| | | | VISDVLKGGPAEGQLQENDRVAMVNGVSMDNVEHA | |
| | | | FAVQQLRKSGKNAKITIRRKKKVQIPNSS | |
| ZO-1 | 292937 | 2 | ISSQPAKPTKVTLVKSRKNEEYGLRLASHIFVKEISQD | 828 |
| | | | SLAARDGNIQEGDVVLKINGTVTENMSLTDAKTLIERS | |
| | | | KGKLKMVVQRDRATLLNSS | |
| ZO-1 | 292937 | 3 | IRMKLVKFRKGDSVGLRLAGGNDVGIFVAGVLEDSPA | 829 |
| | | | AKEGLEEGDQILRVNNVDFTNIIREEAVLFLLDLPKGE | |
| | | | EVTILAQKKKDVFSN | |
| ZO-2 | 12734763 | 1 | LIWEQYTVTLQKDSKRGFGIAVSGGRDNPHFENGET | 830 |
| | | | SIVISDVLPGGPADGLLQENDRVVMVNGTPMEDVLHS | |
| | | | FAVQQLRKSGKVAAIVVKRPRKV | |
| ZO-2 | 12734763 | 2 | RVLLMKSRANEEYGLRLGSQIFVKEMTRTGLATKDG | 831 |
| | | | NLHEGDIILKINGTVTENMSLTDARKLIEKSRGKLQLVV | |
| | | | LRDS | |
| ZO-2 | 12734763 | 3 | HAPNTKMVRFKKGDSVGLRLAGGNDVGIFVAGIQEG | 832 |
| | | | TSAEQEGLQEGDQILKVNTQDFRGLVREDAVLYLLEI | |
| | | | PKGEMVTILAQSRADVY | |
| ZO-3 | 10092690 | 1 | IPGNSTIWEQHTATLSKDPRRGFGIAISGGRDRPGGS | 833 |
| | | | MVVSDVVPGGPAEGRLQTGDHIVMVNGVSMENATS | |
| | | | AFAIQILKTCTKMANITVKRPRRIHLPAEFIVTD | |
| ZO-3 | 10092690 | 2 | QDVQMKPVKSVLVKRRDSEEFGVKLGSQIFIKHITDS | 834 |
| | | | GLAARHRGLQEGDLILQINGVSSQNLSLNDTRRLIEKS | |
| | | | EGKLSLLVLRDRGQFLVNIPNSS | |
| ZO-3 | 10092690 | 3 | RGYSPDTRVVRFLKGKSIGLRLAGGNDVGIFVSGVQA | 835 |
| | | | GSPADGQGIQEGDQILQVNDVPFQNLTREEAVQFLL | |
| | | | GLPPGEEMELVTQRKQDIFWKMVQSEFIVTD | |

*: No GI number for this PDZ domain containing protein - it was computer cloned by J. S. using rat Shank3 seq against human genomic clone AC000036.
“In silico spliced together nt6400-6496, 6985-7109, 7211-7400 to create hypothetical human Shank3.”

TABLE 12

| | | Peptide | | | Protein | |
|---|---|---|---|---|---|---|
| | | Optimal | | PDZ | Optimal | |
| AVC ID | PL | Conc | PDZ | Domain | Conc | Classification |
| AA02.1 | Clasp-2 | 0 | PSD95 | 1, 2, 3 | 0 | 2 |
| | Clasp-2 | 0 | NeDLG | 1, 2 | 0 | 2 |
| AA10 | CD46 | 0 | Mint 1 | 1, 2 | 0 | 1 |
| | CD46 | 0 | KIAA807 | | 0 | 4 |
| | CD46 | 0 | KIAA0807(S) | 1 | 0 | 5 |
| AA13 | CD95 (fas) | 0 | PSD95 | 1, 2, 3 | 0 | 1 |
| | CD95 (fas) | 0 | NeDLG | 1, 2 | 0 | 1 |
| | CD95 (fas) | 0 | DLG1 | 1, 2 | 0 | 2 |
| AA22 | DNAM-1 | 0 | PSD95 | 1, 2, 3 | 0 | 2 |
| | DNAM-1 | 0 | NeDLG | 1, 2 | 0 | 2 |
| | DNAM-1 | 0 | DLG1 | 1, 2 | 0 | 1 |
| AA29.3 | IL-8RB | 0 | PSD95 | 1, 2, 3 | 0 | 1 |
| | IL-8RB | 0 | KIAA0807(S) | 1 | 0 | 1 |
| AA216 | NMDA R2C | 0 | PSD95 | 1, 2, 3 | 0 | 1 |
| | NMDA R2C | 0 | NeDLG | 1, 2 | 0 | 2 |
| | NMDA R2C | 0 | DLG1 | 1, 2 | 0 | 1 |
| AA07 | CD34 | 0 | KIAA807 | | 0 | 5 |
| | CD34 | 0 | KIAA0807(S) | 1 | 0 | 3 |
| AA30 | LPAP | 0 | KIAA0807(S) | 1 | 0 | 5 |
| | LPAP | 0 | Mint 1 | 1, 2 | 0 | 1 |
| | LPAP | 5 | TIP1 | 1 | 5 | 5 |
| AA36 | Neuroligin | 0 | KIAA0807(S) | 1 | 0 | 3 |
| AA40 | Dock2 | 0 | KIAA0807(S) | 1 | 0 | 4 |
| | Dock2 | 0 | KIAA807 | | 0 | 5 |
| AA45 | BLR-1 | 0 | KIAA807 | | 0 | 2 |
| | BLR-1 | 1 | KIAA0807(S) | 1 | 0.3 | 2 |
| | BLR-1 | 0 | PDZK1 | 2, 3, 4 | 0 | 1 |
| | BLR-1 | 0 | KIAA0561 | 1 | 0 | 1 |
| AA56 | Tax | 0 | TIP1 | 1 | 0 | 5 |
| | Tax | 0 | KIAA0807(S) | 1 | 0 | 5 |
| | Tax | 0 | KIAA807 | | 0 | 5 |
| | Tax | 0 | DLG1 | 1, 2 | 0 | 5 |
| | Tax | 0 | PSD95 | 1, 2, 3 | 0 | 5 |
| | Tax | 0 | NeDLG | 1, 2 | 0 | 5 |
| AA58 | PAG | 0 | KIAA807 | | 0 | 5 |
| | PAG | 0.35 | KIAA0807(S) | 1 | 0.5 | 5 |

1. A method of modulating a biological function of a cell, comprising introducing into the cell an agent that alters binding between a PDZ protein and a PL protein in the cell, whereby the biological function is modulated in the cell, and wherein the PDZ protein and PL protein are a binding pair as specified in Table 12.

2. The method of claim 1, wherein the PDZ protein is a protein kinase, a guanalyte kinase, a tyrosine phosphatase or a serine phosphatase.

3. The method of claim 1, wherein the PDZ protein is a LIM protein or a guanine exchange factor.

4. The method of claim 1, wherein the PDZ protein is viral oncogene interacting protein.

5. The method of claim 1, wherein the PL protein is a T-cell surface receptor or a B-cell surface receptor.

6. The method of claim 1, wherein the PL protein is a natural killer cell surface receptor, a monocyte cell surface receptor, or a granulocyte cell surface receptor.

7. The method of claim 1, wherein the PL protein is an endothelial cell surface receptor.

8. The method of claim 1, wherein the PL protein is a G-protein linked receptor or a regulator of G-protein signaling.

9. The method of claim 1, wherein the PL protein is an adhesion protein or a tight junction integral membrane protein.

10. The method of claim 1, wherein the PL protein is a viral oncogene.

11. The method of claim 1, wherein the PL protein is neuron membrane transport protein.

12. The method of claim 1, wherein the PL protein is a receptor kinase.

13. The method of claim 1, wherein the PDZ protein is an ion channel or transporter protein.

14. The method of claim 1, wherein the PL protein is a tumor suppressor protein.

15. The method of claim 1, wherein the agent is a polypeptide comprising at least the two carboxy-terminal residues of the PL protein.

16. The method of claim 15, wherein the agent comprises at least the three carboxy-terminal residues of the PL protein.

17. The method of claim 1, wherein the agent is a small molecule or peptide mimetic of at least the two carboxy terminal residues of the PL protein.

18. The method of claim 1, wherein the agent is an antagonist that inhibits binding between the PDZ protein and PL protein binding pair.

19. The method of claim 1, wherein the agent is an agonist that promotes binding between the PDZ protein and the PL protein binding pair.

20. The method of claim 1, wherein the method is conducted in vitro.

21. A method of determining whether a test compound is a modulator of binding between a PDZ protein and a PL protein, comprising:
(a) contacting under suitable binding conditions (i) a PDZ-domain polypeptide having a sequence from the PDZ protein, and (ii) a PL peptide, wherein
the PL peptide comprises a C-terminal sequence of the PL protein,
the PDZ-domain polypeptide and the PL peptide are a binding pair as specified in Table 12; and
contacting is performed in the presence of the test compound; and
(b) detecting formation of a complex between the PDZ-domain polypeptide and the PL peptide, wherein
(i) presence of the complex at a level that is statistically significantly higher in the presence of the test compound than in the absence of test compound is an indication that the test compound is an agonist, and
(ii) presence of the complex at a level that is statistically significantly lower in the presence of the test compound than in the absence of test compound is an indication that the test compound is an antagonist.

22. The method of claim 21, wherein complex is detected in both the absence and presence of test compound.

23. A modulator of binding between a PDZ protein and a PL protein, wherein the modulator is
(a) a peptide comprising at least 3 residues of a C-terminal sequence of a PL protein, and wherein the PDZ protein and the PL protein are a binding pair as specified in Table 12; or
(b) a peptide mimetic of the peptide of section (a); or
(c) a small molecule having similar functional activity as the peptide of section (a) with respect to the PDZ and PL protein binding pair.

24. The modulator of claim 23 that is an agonist.

25. The modulator of claim 23 that is an antagonist.

26. A pharmaceutical composition comprising a modulator of claim 23.

27. A method of treating a disease correlated with binding between a PDZ protein and a PL protein, the method comprising administering a therapeutically effective amount of a modulator of claim 23.

28. The method of claim 27, wherein the disease is selected from the group consisting of a neurological disease, an immune response disease, a muscular disease, and a cancer.

29. The method of claim 27, wherein the modulator is administered to a non-human animal.
